(19) United States
(12) Patent Application Publication
(10) Pub. No.: US 2022/0133822 A1
(43) Pub. Date: May 5, 2022

(54) NOVEL BACTERIOPHAGE THAT LYSES ACINETOBACTER GENUS BACTERIA HAVING RESISTANCE TO ANTIBIOTICS

(71) Applicant: Microbiotix Co., Ltd, Seoul (KR)

(72) Inventors: Dong Eun YONG, Seoul (KR); Jong Soo JEON, Seoul (KR)

(21) Appl. No.: 17/085,350

(22) Filed: Oct. 30, 2020

(51) Int. Cl. A61K 35/76; C12N 7/00; A23L 29/00

(52) U.S. Cl. CPC A61K 35/76; C12N 2795/10132; A23L 29/065; C12N 7/00

(57) ABSTRACT

The present invention relates to a novel bacteriophage that lyses Acinetobacter genus bacteria, in particular, Acinetobacter genus bacteria having resistance to antibiotics. The bacteriophage of the present invention can be used in various fields, such as antibiotic composition, feed additive composition, feed, disinfectant, cleaning agent, and a composition for prevention or treatment of an infectious disease caused by Acinetobacter genus bacteria.

### REFERENCE TO ELECTRONIC SEQUENCE LISTING

A computer readable form of the Sequence Listing is filed with this application by electronic submission and is incorporated into this application by reference in its entirety. The Sequence Listing is contained in the file created on Oct. 30, 2020, having the file name “20-1735-US_Sequence-Listing_ST25.txt” and is 208 kilobytes in size.

### BACKGROUND OF THE INVENTION

### 1. Field of the Invention

The present invention relates to a novel bacteriophage that lyses Acinetobacter genus bacteria, in particular, Acinetobacter genus bacteria having resistance to antibiotics.

### 2. Description of the Related Art

Bacterial infection is one of the most common and fatal causes of human disease. Since penicillin, numerous types of antibiotics have been developed and used to combat bacteria that have invaded a living body from the outside. However, in recent years, strains having tolerance to these antibiotics have emerged, which is considered a big problem. Bacterial species, such as Enterococcus faecalis, Mycobacterium tuberculosis, and Pseudomonas aeruginosa, which may pose a threat to life, have developed resistance to all antibiotics known to date (Stuart B. Levy, Scientific American (1998): 46-53).

Tolerance to antibiotics is a phenomenon distinguished from resistance to antibiotics. This phenomenon was first discovered in Pneumococcus sp. in the 1970s and provided an important clue for the mechanism of action of penicillin (Tomasz et al., Nature, 227, (1970): 138-140). Conventional chemical antibiotics, such as penicillin and cephalosporin, exhibit an antibiotic action by inhibiting microbial cell wall or protein synthesis. However, the species showing tolerance stop growing in the presence of antibiotics at typical concentrations, and do not end up in death. Tolerance develops due to the fact that when antibiotics inhibit a bacterial cell wall synthetase, bacterial autolytic enzymes such as autolysin are not activated. This fact explains that penicillin kills bacteria by activating their endogenous hydrolytic enzymes, whereas bacteria survive treatment with antibiotics through inhibition of activity of such bacterial autolytic enzymes. Accordingly, there is an urgent need for development of antibiotics having a new mechanism of action capable of combating these resistant strains, and antibiotic peptides showing different antibiotic mechanisms from conventional chemical antibiotics have attracted attention as new concept-based next-generation antibiotics (Zasloff, M. Curr Opin Immunol 4 (1992): 3-7; Boman, H. G., Cell, 65.205 (1991); Boman, H. G. J Intern Med. 254.3 (2003): 197-215; Hancock, R. E., & Scott, M. G., Proc. Natl. Acad. Sci. U.S.A. 97 (2000): 8856-8861, Zasloff, M., Nature 415 (2002): 389-395). In the present specification, the term “tolerance” is interchangeably used with “resistance”.

On the other hand, Acinetobacter baumannii is a gram-negative aerobic coccobacillus and has been an important cause of hospital infections in many hospitals. In particular, recently, infection with multi-drug-resistant Acinetobacter baumannii (MRAB) showing resistance to aminoglycoside, cephalosporin, fluoroquinolone, beta-lactamase inhibitors, and carbapenem has been increasing.

In 2010, at the University of Tokyo Hospital, 46 people were infected with Acinetobacter bacteria and 10 of them died. This incident aroused awareness about MRAB, which is highly antibiotic-resistant and of which the number has been rapidly increasing worldwide in the last decade, and spurred development of antibiotics. Acinetobacter bacteria themselves are commonly present in water or soil, or even in human skin. In healthy people, infection with Acinetobacter bacteria does not cause illness. However, in a case where people with decreased immunity are infected with Acinetobacter bacteria, they may die of pneumonia or sepsis. Starting from the 1990s, the number of Acinetobacter bacteria began to increase in the United States, Europe, and the like; and starting from 2000, even types thereof which there are almost no antibiotics available to combat have emerged.

Typically, multi-drug-resistant Acinetobacter baumannii (MRAB) refers to a strain that is resistant to all three types of drugs such as aminoglycoside, fluoroquinolone, and carbapenem. For Acinetobacter bacteria which are major causative bacteria of medical-related infections, due to multi-drug resistance thereof, carbapenem has been almost the only effective antibacterial agent. However, as the number of strains that are resistant even to carbapenem has increased over the past 10 years, great limitations are imposed on treatment of infections with Acinetobacter bacteria.

Recently, Pseudomonas aeruginosa has a tolerance of about 20%, whereas Acinetobacter bacteria has a tolerance that has rapidly increased and surpassed 50% in most large hospitals. An increase in tolerance to carbapenem has led to an increase in number of Acinetobacter bacteria. As a result, according to a 2010 Korean nationwide survey of medical-related infection rates in intensive care units, Acinetobacter bacteria beat Pseudomonas aeruginosa, and thus took the third place, in terms of frequency of causative bacteria, following methicillin-resistant Staphylococcus aureus (MRSA) and Enterococcus sp. Accordingly, there is an urgent need for development of a therapeutic agent for Acinetobacter bacteria from the viewpoint that such bacteria have high frequency and high mortality rate among causative agents of critically ill infections in Korea.

### SUMMARY OF THE INVENTION

An object of the present invention is to provide a novel bacteriophage that has specific infectivity on and killing ability against Acinetobacter genus bacteria, in particular, Acinetobacter genus bacteria having resistance to antibiotics.

Another object of the present invention is to provide a composition for preventing or treating an infectious disease caused by Acinetobacter genus bacteria, in particular, Acinetobacter genus bacteria having resistance to antibiotics, or a food composition for ameliorating the same disease, the composition comprising a novel bacteriophage that has specific infectivity on and killing ability against the Acinetobacter genus bacteria.

However, the technical problem to be achieved by the present invention is not limited to the above-mentioned problems, and other problems that are not mentioned will be clearly understood by those skilled in the art from the following description.

According to an embodiment of the present invention, there is provided a bacteriophage that has a specific killing ability against Acinetobacter genus bacteria.

As used herein, the term “bacteriophage” refers to a bacteria-specific virus which infects a specific bacterium so that growth of the bacterium is prevented or inhibited, the virus containing single- or double-stranded DNA or RNA as a genetic material.

In the present invention, the Acinetobacter genus bacteria may be at least any one selected from, but is not limited to, the group consisting of Acinetobacter baumannii, Acinetobacter calcoaceticus, Acinetobacter haemolyticus, Acinetobacter junii, Acinetobacter johnsonii, Acinetobacter lwoffii, Acinetobacter radioresistens, Acinetobacter ursingii, Acinetobacter schindleri, Acinetobacter parvus, Acinetobacter baylyi, Acinetobacter bouvetii, Acinetobacter towneri, Acinetobacter tandoii, Acinetobacter grimontii, Acinetobacter tjernbergiae, and Acinetobacter gerneri.

In the present invention, the bacteriophage has a specific killing ability against Acinetobacter genus bacteria; and among these Acinetobacter genus bacteria, the bacteriophage has a specific killing ability, against Acinetobacter genus bacteria having resistance to antibiotics.

As used herein, the “resistance to antibiotics” means that resistance develops against specific antibiotics so that the antibiotics do not exert pharmacological efficacy thereof. For the purpose of the present invention, the antibiotics may be antibiotics having a structure of carbapenem. Specifically, the antibiotics may be at least one selected from, but are not limited to, the group consisting of amikacin, ampicillin, ampicillin-sulbactam, aztreonam, ciprofloxacin, ceftazidime, cefazolin, ertapenem, cefepime, cefoxitin, cefotaxime, gentamicin, levofloxacin, minocycline, imipenem, meropenem, piperacillin, piperacillin-tazobactam, cortrimoxa, and tigecycline. For the purpose of the present invention, the Acinetobacter genus bacteria, preferably Acinetobacter baumannii, may have resistance to antibiotics, and the resistance to antibiotics may develop by production of carbapenemase that decomposes carbapenem and thus prevents an effect thereof from exerting.

In an embodiment of the present invention, the bacteriophage may be a bacteriophage obtained by collecting a sample from a hospital sewage treatment plant and performing isolation from the sample, which is designated bacteriophage YMC14/01/P117_ABA_BP and has been deposited at the Korean Culture Center of Microorganisms under the accession number KFCC11800P on Nov. 15, 2018.

It was identified that the bacteriophage YMC14/01/P117_ABA_BP of the present invention belongs to the family Myoviridae which has a long tail with a hexagonal head, and whole-genome sequencing thereof showed that it has a size of 44,653 bp and has a total of 78 ORFs.

In addition, in the present invention, the bacteriophage YMC14/01/P117_ABA_BP may include, as all or part of the entire gene, a nucleotide sequence represented by SEQ ID NO: 1.

In addition, the bacteriophage YMC14/01/P117_ABA_BP of the present invention may consist of a nucleotide sequence represented by SEQ ID NO: 1, and a functional equivalent of the nucleotide sequence. The functional equivalent refers to a sequence obtained by modification or substitution of the nucleotide sequence represented by SEQ ID NO: 1, which has a sequence homology of 70% or higher, preferably 80% or higher, more preferably 90% or higher, and even more preferably 95% or higher to the nucleotide sequence represented by SEQ ID NO: 1, and exhibits substantially the same physiological activity as the nucleotide sequence represented by SEQ ID NO: 1.

In addition, the bacteriophage YMC14/01/P117_ABA_BP provided by the present invention may include any one protein of SEQ ID NOs: 2 to 4. In the present invention, each of SEQ ID NOs: 2 to 4 is an open reading frame (ORF) of the bacteriophage. A protein represented by SEQ ID NO: 2 may be an amino acid sequence of a lysozyme-like domain; a protein represented by SEQ ID NO: 3 may be an amino acid sequence of a putative tail-fiber/lysozyme protein; and a protein represented by SEQ ID NO: 4 may be an amino acid sequence of a putative endolysin protein. More specifically, SEQ ID NO: 2 may be an amino acid sequence of ORF7; SEQ ID NO: 3 may be an amino acid sequence of ORF8; and SEQ ID NO: 4 may be an amino acid sequence of ORF74.

In addition, the bacteriophage YMC14/01/P117_ABA_BP provided by the present invention may include a genome represented by any one of SEQ ID NOs: 5 to 7. Here, SEQ ID NO: 5 may be a nucleotide sequence of a genome coding for ORF7; SEQ ID NO: 6 may be a nucleotide sequence of a genome coding for ORF8; and SEQ ID NO: 7 may be a nucleotide sequence of a genome coding for ORF74.

In another embodiment of the present invention, the bacteriophage may be a bacteriophage obtained by collecting a sample from a hospital sewage treatment plant and performing isolation from the sample, which is designated bacteriophage YMC16/12/R4637_ABA_BP and has been deposited at the Korean Culture Center of Microorganisms under the accession number KFCC11801P on Nov. 15, 2018.

It was identified that the bacteriophage YMC16/12/R4637_ABA_BP of the present invention belongs to the family Myoviridae which has a long tail with a hexagonal head, and whole-genome sequencing thereof showed that it has a size of 42,555 bp and has a total of 78 ORFs.

In addition, in the present invention, the bacteriophage YMC16/12/R4637_ABA_BP may include, as all or part of the entire gene, a nucleotide sequence represented by SEQ ID NO: 8.

In addition, the bacteriophage YMC16/12/R4637_ABA_BP of the present invention may consist of a nucleotide sequence represented by SEQ ID NO: 8, and a functional equivalent of the nucleotide sequence. The functional equivalent refers to a sequence obtained by modification or substitution of the nucleotide sequence represented by SEQ ID NO: 8, which has a sequence homology of 70% or higher, preferably 80% or higher, more preferably 90% or higher, and even more preferably 95% or higher to the nucleotide sequence represented by SEQ ID NO: 8, and exhibits substantially the same physiological activity as the nucleotide sequence represented by SEQ ID NO: 8.

In addition, the bacteriophage YMC16/12/R4637_ABA_BP provided by the present invention may include a protein of SEQ ID NO: 9 or 10. In the present invention, SEQ ID NO: 9 or 10 may be an open reading frame (ORF) of the bacteriophage. A protein represented by SEQ ID NO: 9 may be an amino acid sequence of a putative lysozyme family protein, and a protein represented by SEQ ID NO: 10 may be an amino acid sequence of a lysozyme-like domain. More specifically, SEQ ID NO: 9 may be an amino acid sequence of ORF37, and SEQ ID NO: 10 may be an amino acid sequence of ORF49.

In addition, the bacteriophage YMC16/12/R4637_ABA_BP provided by the present invention may include a genome of SEQ ID NO: 11 or 12. Here, SEQ ID NO: 11 may be a nucleotide sequence of a genome coding for ORF37, and SEQ ID NO: 12 may be a nucleotide sequence of a genome coding for ORF49.

In yet another embodiment of the present invention, the bacteriophage may be a bacteriophage obtained by collecting a sample from a hospital sewage treatment plant and performing isolation from the sample, which is designated bacteriophage YMC16/01/R2016_ABA_BP and has been deposited at the Korean Culture Center of Microorganisms under the accession number KFCC11803P on Nov. 15, 2018.

It was identified that the bacteriophage YMC16/01/R2016_ABA_BP of the present invention belongs to the family Myoviridae which has a long tail with a hexagonal head, and whole-genome sequencing thereof showed that it has a size of 44,576 bp and has a total of 76 ORFs.

In addition, in the present invention, the bacteriophage YMC16/01/R2016_ABA_BP may include, as all or part of the entire gene, a nucleotide sequence represented by SEQ ID NO: 13.

In addition, the bacteriophage YMC16/01/R2016_ABA_BP of the present invention may consist of a nucleotide sequence represented by SEQ ID NO: 13, and a functional equivalent of the nucleotide sequence. The functional equivalent refers to a sequence obtained by modification or substitution of the nucleotide sequence represented by SEQ ID NO: 13, which has a sequence homology of 70% or higher, preferably 80% or higher, more preferably 90% or higher, and even more preferably 95% or higher to the nucleotide sequence represented by SEQ ID NO: 13, and exhibits substantially the same physiological activity as the nucleotide sequence represented by SEQ ID NO: 13.

In addition, the bacteriophage YMC16/01/R2016_ABA_BP provided by the present invention may include any one protein of SEQ ID NOs: 14 to 16. In the present invention, each of SEQ ID NOs: 14 to 16 is an open reading frame (ORF) of the bacteriophage. SEQ ID NO: 14 may be an amino acid sequence of a putative tail-fiber/lysozyme protein; SEQ ID NO: 15 may be an amino acid sequence of a lysozyme-like domain; and SEQ ID NO: 16 may be an amino acid sequence of a putative endolysin protein. More specifically, SEQ ID NO: 14 may be an amino acid sequence of ORF8; SEQ ID NO: 15 may be an amino acid sequence of ORF9; and SEQ ID NO: 16 may be an amino acid sequence of ORF21.

In addition, the bacteriophage YMC16/01/R2016_ABA_BP provided by the present invention may include a genome represented by any one of SEQ ID NOs: 17 to 19. Here, SEQ ID NO: 17 may be a nucleotide sequence of a genome coding for ORF8; SEQ ID NO: 18 may be a nucleotide sequence of a genome coding for ORF9; and SEQ ID NO: 19 may be a nucleotide sequence of a genome coding for ORF21.

In the present invention, the bacteriophage YMC14/01/P117_ABA_BP; the bacteriophage YMC16/12/R4637_ABA_BP; and the bacteriophage YMC16/01/R2016_ABA_BP have excellent stability against heat and pH.

The bacteriophage YMC14/01/P117_ABA_BP; the bacteriophage YMC16/12/R4637_ABA_BP; and the bacteriophage YMC16/01/R2016_ABA_BP, of the present invention, maintains their lytic activity in a range of 4° C. to 60° C.; however, the temperature range is not limited thereto.

In addition, the bacteriophage YMC14/01/P117_ABA_BP; the bacteriophage YMC16/12/R4637_ABA_BP; and the bacteriophage YMC16/01/R2016_ABA_BP, of the present invention, maintains their lytic activity in a range of pH 3.0 to pH 11.0 and preferably in a range of pH 5.0 to pH 10.0; however, the pH range is not limited thereto.

In the bacteriophage YMC14/01/P117_ABA_BP; the bacteriophage YMC16/12/R4637_ABA_BP; and the bacteriophage YMC16/01/R2016_ABA_BP, of the present invention, their Acinetobacter genus bacteria-specific lytic activity, acid resistance, and base resistance as described above allow these bacteriophages to be applied, at various pH ranges, to a composition for preventing or treating an infectious disease caused by Acinetobacter genus bacteria, and to a variety of products, each of which comprises such a bacteriophage as an active ingredient.

According to yet another embodiment of the present invention, there is provided a composition for preventing, ameliorating, or treating a disease caused by Acinetobacter genus bacteria, the composition comprising, as an active ingredient, the bacteriophage YMC14/01/P117_ABA_BP; the bacteriophage YMC16/12/R4637_ABA_BP; or the bacteriophage YMC16/01/R2016_ABA_BP.

Details of the bacteriophage and the Acinetobacter genus bacteria in the composition of the present invention overlap with those as described above for the bacteriophage; and thus, detailed descriptions thereof will be omitted.

In the present invention, the bacteriophage YMC14/01/P117_ABA_BP; the bacteriophage YMC16/12/R4637_ABA_BP; and the bacteriophage YMC16/01/R2016_ABA_BP specifically kill Acinetobacter genus bacteria, in particular, Acinetobacter genus bacteria having resistance to antibiotics, and thus are effective in treatment of various diseases caused by Acinetobacter genus bacteria.

In the present invention, the infectious disease caused by Acinetobacter genus bacteria may be, but is not limited to, a disease selected from the group consisting of hepatitis C, hand-foot-and-mouth disease, gonorrhea, chlamydia, chancroid, genital herpes, condylomata acuminata, vancomycin-resistant Staphylococcus aureus infection, vancomycin-resistant Enterococci infection, methicillin-resistant Staphylococcus aureus infection, multi-drug-resistant Pseudomonas aeruginosa infection, multi-drug-resistant Acinetobacter baumannii infection, carbapenem-resistant Enterobacteriaceae infection, intestinal infection, acute respiratory infection, and Enterovirus infection.

The composition of the present invention may contain the bacteriophage in an amount of 1×10³ to 1×10¹⁰ PFU/mL and preferably 1×10⁶ to 1×10⁹ PFU/mL. The term “plaque forming unit (PFU)”, as used herein, refers to a unit used to quantify plaque formation by bacteriophage.

In the present invention, the term “prevention” refers to any act of suppressing or delaying onset of a disease by administration of a composition.

In the present invention, the term “treatment” refers to any act of ameliorating symptoms of the disease, or suppressing or alleviating and beneficially altering the disease, by the administration of the composition.

The composition of the present invention can be used as a pharmaceutical composition, a food composition, or a cosmetic composition.

According to still yet another embodiment of the present invention, there is provided an antibiotic composition, comprising, as an active ingredient, the bacteriophage YMC14/01/P117_ABA_BP; the bacteriophage YMC16/12/R4637_ABA_BP; or the bacteriophage YMC16/01/R2016_ABA_BP.

In the present invention, the term “antibiotic composition” refers to a preparation that is applied to an animal in the form of a medicament to kill bacteria, and is a general term for antiseptics, bacteriocidal agents, antibiotics, and antibacterial agents.

The bacteriophage YMC14/01/P117_ABA_BP; the bacteriophage YMC16/12/R4637_ABA_BP; and the bacteriophage YMC16/01/R2016_ABA_BP, of the present invention, have very high specificity for Acinetobacter genus bacteria as compared with conventional antibiotics, and at the same time, also act on antibiotic-resistant bacteria, which allows these bacteriophages to kill only particular pathogenic bacteria without killing beneficial bacteria. In addition, these bacteriophages do not induce drug tolerance or resistance, which allows such bacteriophages to be advantageously used as novel antibiotics having a long life cycle as compared with conventional antibiotics.

According to still yet another embodiment of the present invention, there is provided a feed additive composition, comprising, as an active ingredient, the bacteriophage YMC14/01/P117_ABA_BP; the bacteriophage YMC16/12/R4637_ABA_BP; or the bacteriophage YMC16/01/R2016_ABA_BP.

In general, feed additive antibiotics used in livestock and fishery industries are used for the purpose of preventing diseases, and administration of antibiotics for preventive purposes is problematic in that likelihood of developing resistant bacteria increases and the antibiotics remaining in livestock may be delivered to humans. In a case where the antibiotics are absorbed, through meat, into a human body, resistance to antibiotics may be caused, which leads to spread of disease. In addition, there are many types of antibiotics to be mixed with feed and fed, which may cause a problem that probability of developing multi-drug-resistant bacteria increases. Thus, as new feed additive antibiotics which are more ecologically-friendly and can solve the problems arising from use of conventional antibiotics, the bacteriophage YMC14/01/P117_ABA_BP; the bacteriophage YMC16/12/R4637_ABA_BP; or the bacteriophage YMC16/01/R2016_ABA_BP, of the present invention, can be used.

In addition, the present invention may provide a feed containing the feed additive composition, and the feed of the present invention may be prepared by separately preparing the bacteriophage in the form of a feed additive and mixing it with the feed, or by directly adding the bacteriophage at the time of preparing the feed. The bacteriophage in the feed of the present invention may be in a liquid or dried form, preferably in a dried powder form. Examples of a drying method may include, but are not limited to, air drying, natural drying, spray drying, and freeze drying. The bacteriophage of the present invention may be added in a powder form and mixed at a component ratio of 0.05% to 10% by weight and preferably 0.10% to 2% by weight with respect to a total weight of the feed. In addition, the feed may further contain, in addition to the bacteriophage of the present invention, conventional additives that can increase preservability of the feed.

To the feed additive composition of the present invention may be further added other non-pathogenic microorganisms. The microorganism that may be added may be selected from the group consisting of Bacillus subtilis that can produce proteases, lipolytic enzymes, and sugar-converting enzymes, Lactobacillus sp. having physiological activity and ability to decompose organic matters under anaerobic conditions such as in the stomach of cattle, filamentous fungi such as Aspergillus oryzae having effects of increasing weight of livestock, increasing milk production, and increasing digestive and absorption rate of feed, and yeast such as Saccharomyces cerevisiae.

Examples of the feed containing the bacteriophage YMC14/01/P117_ABA_BP; the bacteriophage YMC16/12/R4637_ABA_BP; or the bacteriophage YMC16/01/R2016_ABA_BP, of the present invention, may include, but are not limited to, plant-based feeds, such as grains, nuts, food processing by-products, algae, fibers, pharmaceutical by-products, oils and fats, starches, meals, and grain by-products, and animal-based feeds such as proteins, minerals, oils and fats, minerals, single-cell proteins, zooplanktons, and food wastes.

The feed additive composition of the present invention may further contain binders, emulsifiers, preservatives, and the like which are added to prevent quality deterioration; and amino acids, vitamins, enzymes, probiotics, flavoring agents, non-protein nitrogen compounds, silicate agents, buffers, coloring agents, extractants, oligosaccharides, and the like which are added to the feed to increase utility thereof. In addition to these ingredients, the feed additive composition of the present invention may further contain feed mixtures and the like.

According to still yet another embodiment of the present invention, there is provided a drinking water additive, comprising the bacteriophage YMC14/01/P117_ABA_BP; the bacteriophage YMC16/12/R4637_ABA_BP; or the bacteriophage YMC16/01/R2016_ABA_BP.

The drinking water additive of the present invention may be used in such a manner that the bacteriophage YMC14/01/P117_ABA_BP; the bacteriophage YMC16/12/R4637_ABA_BP; or the bacteriophage YMC16/01/R2016_ABA_BP, or a composition containing the same is separately prepared in the form of a drinking water additive and mixed with a feed or drinking water, or may be used in such a manner that it is directly added at the time of preparing drinking water. In a case where the drinking water additive is supplied by being mixed with drinking water, an effect of continuously decreasing the number of Acinetobacter genus bacteria is exhibited.

In the present invention, for the drinking water, there is no particular limitation and drinking water commonly used in the art may be used.

According to still yet another embodiment of the present invention, there is provided a disinfectant, comprising the bacteriophage YMC14/01/P117_ABA_BP; the bacteriophage YMC16/12/R4637_ABA_BP; or the bacteriophage YMC16/01/R2016_ABA_BP.

The bacteriophage YMC14/01/P117_ABA_BP; the bacteriophage YMC16/12/R4637_ABA_BP; or the bacteriophage YMC16/01/R2016_ABA_BP, of the present invention, has a specific killing ability against Acinetobacter genus bacteria. Thus, in the present invention, the disinfectant that comprises the bacteriophage YMC14/01/P117_ABA_BP; the bacteriophage YMC16/12/R4637_ABA_BP; or the bacteriophage YMC16/01/R2016_ABA_BP can be effectively used as a disinfectant for hospitals and health care to prevent hospital infections, and can also be used as a general household disinfectant, a disinfectant for foods, cooking places, and facilities, a disinfectant for buildings such as poultry farms and livestock houses, animal body, various products for animal growth and development such as drinking water, straw litter, eggbox panels, transport vehicle, and tableware, or the like.

According to still yet another embodiment of the present invention, there is provided a cleaning agent, comprising the bacteriophage YMC14/01/P117_ABA_BP; the bacteriophage YMC16/12/R4637_ABA_BP; or the bacteriophage YMC16/01/R2016_ABA_BP.

The bacteriophage YMC14/01/P117_ABA_BP; the bacteriophage YMC16/12/R4637_ABA_BP; or the bacteriophage YMC16/01/R2016_ABA_BP, of the present invention, has a specific killing ability against Acinetobacter genus bacteria, and thus can also be used to clean an individual's skin surface or every body part, or the like which has been exposed or likely to be exposed to Acinetobacter genus bacteria.

In the present invention, the pharmaceutical composition may be characterized by being in the form of capsules, tablets, granules, injections, ointments, powders, or beverages, and the pharmaceutical composition may be characterized by being targeted to humans.

The pharmaceutical composition of the present invention may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, excipients, or diluents suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition according to the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

In the present invention, the “parenteral” includes subcutaneous, intradermal, intravenous, intramuscular, intraarticular, intrabursal, intrasternal, intradural, intralesional, and intracranial injection or infusion techniques. The pharmaceutical composition of the present invention may also be administered in the form of suppositories for rectal administration.

The pharmaceutical composition of the present invention may vary widely depending on a variety of factors, including activity of a certain compound used, the patient's age, body weight, general health status, sex, diet, time of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the present invention in any way. The pharmaceutical composition according to the present invention may be formulated in the form of pills, sugar-coated tablets, capsules, liquids, gels, syrups, slurries, or suspensions.

In the present invention, the cosmetic composition may be prepared in the form of skin softeners, nourishing lotions, nourishing essences, massage creams, cosmetic bath water additives, body lotions, body milks, bath oil, baby oil, baby powders, shower gels, shower creams, sun screen lotions, sun screen creams, suntan creams, skin lotions, skin creams, UV blocking cosmetics, cleansing milks, hair removing agents (for cosmetic purposes), face and body lotions, face and body creams, skin whitening creams, hand lotions, hair lotions, cosmetic creams, Jasmine oil, bath soaps, liquid soaps, cosmetic soaps, shampoos, hand cleaners, medicinal soaps (for non-medical purposes), cream soaps, facial washes, body cleansers, scalp cleansers, hair rinses, toilet soaps, tooth whitening gels, toothpastes, and the like. To this end, the composition of the present invention may further contain either a solvent which is conventionally used for the preparation of cosmetic compositions, or a suitable carrier, excipient, or diluent.

The type of solvent that may further be added to the cosmetic composition of the present invention is not particularly limited, and examples of the solvent may include water, saline, DMSO, or a combination thereof. In addition, examples of the carrier, excipient, or diluent include, but are not limited to, purified water, oil, wax, fatty acids, fatty acid alcohol, fatty acid esters, surfactants, humectants, thickeners, antioxidants, viscosity stabilizers, chelating agents, buffers, lower alcohol, and the like. In addition, the cosmetic composition of the present invention may, if necessary, contain whitening agents, moisturizing agents, vitamins, UV blocking agents, fragrances, dyes, antibiotics, antibacterial agents, and antifungal agents.

Examples of the oil may include hydrogenated vegetable oil, castor oil, cottonseed oil, olive oil, palm kernel oil, jojoba oil, and avocado oil, and examples of the wax may include beeswax, spermaceti, carnauba wax, candelilla wax, montan wax, ceresin wax, liquid paraffin, and lanolin.

Examples of the fatty acids may include stearic acid, linoleic acid, linolenic acid, and oleic acid; examples of the fatty acid alcohol may include cetyl alcohol, octyl dodecanol, oleyl alcohol, panthenol, lanolin alcohol, stearyl alcohol, and hexadecanol; and examples of the fatty acid esters may include isopropyl myristate, isopropyl palmitate, and butyl stearate. Examples of the surfactants may include cationic surfactants, anionic surfactants, and nonionic surfactants, which are known in the art. Among these, if possible, surfactants derived from natural products are preferred.

In addition, the cosmetic composition of the present invention may contain humectants, thickeners, antioxidants, and the like, which are widely known in the cosmetic field, and the types and amounts thereof are as known in the art.

The food composition of the present invention may be prepared in the form of various foods, for example, beverages, gums, tea, vitamin complexes, powders, granules, tablets, capsules, confections, rice cakes, bread, and the like. The food composition of the present invention is composed of a plant extract having little toxicity and side effects, and thus can be used without worries in a case of being ingested for a long time for preventive purposes. In a case where the bacteriophage of the present invention is contained in the food composition, the amount thereof to be added may be 0.10% to 50% of a total weight of the food composition.

Here, in a case where the food composition is prepared in the form of a beverage, there is no particular limitation except that the beverage contains the food composition at an indicated proportion, and the beverage may contain various flavoring agents or natural carbohydrates, or the like as additional ingredients similarly to conventional beverages. That is, examples of the natural carbohydrates may include monosaccharides such as glucose, disaccharides such as fructose, polysaccharides such as sucrose, conventional sugars such as dextrin and cyclodextrin, and sugar alcohol such as xylitol, sorbitol, and erythritol. Examples of the flavoring agents may include natural flavoring agents (thaumatin, stevia extracts (such as rebaudioside A), glycyrrhizin, and the like) and synthetic flavoring agents (saccharin, aspartame, and the like).

In addition, the food composition of the present invention may contain various nutrients, vitamins, minerals (electrolytes), flavorings such as synthetic flavorings and natural flavorings, colorants, pectic acid and salts thereof, alginic acid and salts thereof, organic acids, protective colloidal thickeners, pH adjusting agents, stabilizers, preservatives, glycerin, alcohol, carbonizing agents used in carbonated beverages, and the like.

These ingredients may be used individually or in combination. The proportion of such additives is not so important, and is generally selected from the range of 0.1 to about 50 parts by weight per 100 parts by weight of the food composition of the present invention.

According to still yet another embodiment of the present invention, there is provided a method for preventing, ameliorating, or treating a disease caused by Acinetobacter genus bacteria, comprising a step of administering, to an individual, the bacteriophage YMC14/01/P117_ABA_BP; the bacteriophage YMC16/12/R4637_ABA_BP; or the bacteriophage YMC16/01/R2016_ABA_BP.

As used herein, the “individual” refers to a patient who is infected or suspected of being infected with Acinetobacter genus bacteria, in which the patient needs appropriate treatment of a disease caused by Acinetobacter genus bacteria or is expected to need such treatment. The type of the individual is not particularly limited and may be selected, for example, from the group consisting of human, rat, mouse, guinea pig, hamster, rabbit, monkey, dog, cat, cow, horse, pig, sheep, and goat, with the human being preferred. However, the type of individual is not limited thereto.

Details of the bacteriophage and the Acinetobacter genus bacteria in the prevention, amelioration, or treatment method of the present invention overlap with those as described above for the bacteriophage; and thus, detailed descriptions thereof will be omitted.

In the present invention, the bacteriophage YMC14/01/P117_ABA_BP; the bacteriophage YMC16/12/R4637_ABA_BP; and the bacteriophage YMC16/01/R2016_ABA_BP specifically kill Acinetobacter genus bacteria, in particular, Acinetobacter genus bacteria having resistance to antibiotics, and thus are effective in treatment of various diseases caused by the Acinetobacter genus bacteria.

In the present invention, the infectious disease caused by Acinetobacter genus bacteria may be, but is not limited to, a disease selected from the group consisting of hepatitis C, hand-foot-and-mouth disease, gonorrhea, chlamydia, chancroid, genital herpes, condylomata acuminata, vancomycin-resistant Staphylococcus aureus infection, vancomycin-resistant Enterococci infection, methicillin-resistant Staphylococcus aureus infection, multi-drug-resistant Pseudomonas aeruginosa infection, multi-drug-resistant Acinetobacter baumannii infection, carbapenem-resistant Enterobacteriaceae infection, intestinal infection, acute respiratory infection, and Enterovirus infection.

Dosages, schedules, and routes of administration of the bacteriophage provided by the present invention may be determined depending on size and condition of an individual, and according to standard pharmaceutical practice. Exemplary routes of administration include intravenous, intraarterial, intraperitoneal, intrapulmonary, intravesicular, intramuscular, intratracheal, subcutaneous, intraocular, intrathecal, or transdermal administration.

In the present invention, a dose of bacteriophage administered to an individual may vary depending on, for example, specific type of bacteriophage administered, route of administration, and specific type and stage of a disease to be treated. The dose should be sufficient to bring about desired responses such as therapeutic responses to a disease, without severe toxicity or adverse events. In some embodiments, an amount of bacteriophage to be administered is a therapeutically effective amount. In some embodiments, the amount of bacteriophage is an amount sufficient to decrease disease symptoms by any one of at least about 10%, 20%, 30%, 40%, 50%, 60%, 70%, 80%, 90%, 95%, or 100%, as compared with disease symptom levels in the same individual before treatment, or as compared with corresponding activity in another individual having not received treatment. Standard methods such as in vitro assays using purified enzymes, cell-based assays, and experiments with animal models or humans may be used to measure a magnitude of effects.

The novel bacteriophage provided by the present invention has a specific killing ability against Acinetobacter genus bacteria, in particular, Acinetobacter genus bacteria having resistance to antibiotics, as compared with chemical substances such as conventional antibiotics.

In addition, from the viewpoint that the bacteriophage of the present invention does not infect other hosts such as humans, animals, and plants, other than bacteria, the following advantages are obtained: it is possible to solve problems of antibiotic-resistant bacteria due to overuse and misuse of antibiotics, problems of residual antibiotics in food, and problems of a wide host range.

Accordingly, the bacteriophage of the present invention can be used in various fields, such as antibiotic composition, feed additive composition, feed, disinfectant, cleaning agent, and a composition of prevention or treatment of an infectious disease caused by Acinetobacter genus bacteria.

### BRIEF DESCRIPTION OF THE DRAWINGS

FIG. 1 illustrates a photograph, taken with an electron microscope, of the bacteriophage YMC14/01/P117_ABA_BP in Example 1 of the present invention.

FIG. 2 graphically illustrates results obtained by evaluating adsorption capacity of the bacteriophage YMC14/01/P117_ABA_BP against Acinetobacter genus bacteria having resistance to antibiotics in Example 1 of the present invention.

FIG. 3 illustrates a one-step growth curve of the lytic bacteriophage YMC14/01/P117_ABA_BP against Acinetobacter genus bacteria having resistance to antibiotics in Example 1 of the present invention.

FIG. 4 graphically illustrates ex vivo lytic ability of the bacteriophage YMC14/01/P117_ABA_BP against Acinetobacter genus bacteria having resistance to antibiotics in Example 1 of the present invention.

FIG. 5 graphically illustrates results obtained by subjecting Galleria mellonella larvae, which has been infected with Acinetobacter genus bacteria having resistance to antibiotics, to treatment with the bacteriophage YMC14/01/P117_ABA_BP, and then observing changes in survival of the Galleria mellonella larvae, in Example 1 of the present invention.

FIG. 6 graphically illustrates pH stability of the lytic bacteriophage YMC14/01/P117_ABA_BP against Acinetobacter genus bacteria having resistance to antibiotics in Example 1 of the present invention.

FIG. 7 graphically illustrates temperature stability of the lytic bacteriophage YMC14/01/P117_ABA_BP against Acinetobacter genus bacteria having resistance to antibiotics in Example 1 of the present invention.

FIG. 8 illustrates results obtained by whole-genome sequencing of the bacteriophage YMC14/01/P117_ABA_BP in Example 1 of the present invention.

FIG. 9 illustrates a photograph, taken with an electron microscope, of the bacteriophage YMC16/12/R4637_ABA_BP in Example 2 of the present invention.

FIG. 10 graphically illustrates results obtained by evaluating adsorption capacity of the bacteriophage YMC16/12/R4637_ABA_BP against Acinetobacter genus bacteria having resistance to antibiotics in Example 2 of the present invention.

FIG. 11 illustrates a one-step growth curve of the lytic bacteriophage YMC16/12/R4637_ABA_BP against Acinetobacter genus bacteria having resistance to antibiotics in Example 2 of the present invention.

FIG. 12 graphically illustrates results obtained by subjecting Galleria mellonella larvae, which has been infected with Acinetobacter genus bacteria having resistance to antibiotics, to treatment with the bacteriophage YMC16/12/R4637_ABA_BP, and then observing changes in survival of the Galleria mellonella larvae, in Example 2 of the present invention.

FIG. 13 graphically illustrates pH stability of the lytic bacteriophage YMC16/12/R4637_ABA_BP against Acinetobacter genus bacteria having resistance to antibiotics in Example 2 of the present invention.

FIG. 14 graphically illustrates temperature stability of the lytic bacteriophage YMC16/12/R4637_ABA_BP against Acinetobacter genus bacteria having resistance to antibiotics in Example 2 of the present invention.

FIG. 15 illustrates results obtained by whole-genome sequencing of the bacteriophage YMC16/12/R4637_ABA_BP in Example 2 of the present invention.

FIG. 16 illustrates a photograph, taken with an electron microscope, of the bacteriophage YMC16/01/R2016_ABA_BP in Example 3 of the present invention.

FIG. 17 graphically illustrates results obtained by evaluating adsorption capacity of the bacteriophage YMC16/01/R2016_ABA_BP against Acinetobacter genus bacteria having resistance to antibiotics in Example 3 of the present invention.

FIG. 18 illustrates a one-step growth curve of the lytic bacteriophage YMC16/01/R2016_ABA_BP against Acinetobacter genus bacteria having resistance to antibiotics in Example 3 of the present invention.

FIG. 19 graphically illustrates results obtained by subjecting Galleria mellonella larvae, which has been infected with Acinetobacter genus bacteria having resistance to antibiotics, to treatment with the bacteriophage YMC16/01/R2016_ABA_BP, and then observing changes in survival of the Galleria mellonella larvae, in Example 3 of the present invention.

FIG. 20 graphically illustrates pH stability of the lytic bacteriophage YMC16/01/R2016_ABA_BP against Acinetobacter genus bacteria having resistance to antibiotics in Example 3 of the present invention.

FIG. 21 graphically illustrates temperature stability of the lytic bacteriophage YMC16/01/R2016_ABA_BP against Acinetobacter genus bacteria having resistance to antibiotics in Example 3 of the present invention.

FIG. 22 illustrates results obtained by whole-genome sequencing of the bacteriophage YMC16/01/R2016_ABA_BP in Example 3 of the present invention.

### DESCRIPTION OF THE PREFERRED EMBODIMENTS

Hereinafter, the present invention will be described in detail by way of the following examples. However, the following examples are only illustrative of the present invention, and the scope of the present invention is not limited by the following examples.

### EXAMPLES

### [Example 1] Bacteriophage YMC14/01/P117 ABA_BP

1. Isolation of Clinical Specimens and Selection of Antibiotic-Resistant Strains

As shown in Table 1 below, Acinetobacter baumannii strains were isolated from blood, clinical specimens, and the like obtained from the intensive care unit (ICU) of a university hospital, and cultured. Strain identification was performed using a kit such as ATB 32 GN system (bioMérieux, Marcy l'Etoile, France). Subsequently, for antibiotic susceptibility test, a CLSI disk diffusion test method, in which culture is performed overnight at 37° C. in outside air using Mueller-Hinton agar, was used; and for test antibiotics, amikacin, ampicillin-sulbactam, ceftazidime, ciprofloxacin, colistin, cefepime, cefotaxime, gentamicin, imipenem, levofloxacin, meropenem, minocycline, piperacillin, piperacillin-tazobactam, cortrimoxa, and tigecycline were used. The susceptibility results were read based on the Clinical and Laboratory Standards Institute (CLSI, 2016). Antibiotic resistance profiles of the collected Acinetobacter baumannii strains are shown in Table 2 below. In Table 2 below, S, I, and R are the results obtained by evaluating susceptibility to the antibacterial agents, in which ‘S’ means susceptible, ‘I’ means intermediate, and ‘R’ means resistant.

TABLE 1

| Host strain | Origin of sample | Host strain | Origin of sample |
|---|---|---|---|
| YMC14/01/R130 | Sputum (pneumonia) | YMC14/01/R2429 | Tracheal aspirate |
| | | | (pneumonia) |
| YMC14/01/R160 | Sputum (pneumonia) | YMC14/01/P728 | Decubitus ulcer |
| YMC14/01/C29 | Ascites (drainage) | YMC14/01/R2572 | Tracheal aspirate |
| | | | (pneumonia) |
| YMC14/01/P31 | Swab or drainage tube, | YMC14/01/R2855 | Sputum (pneumonia) |
| | abdomen | | |
| YMC14/01/U313 | Random urine | YMC14/01/R2945 | Sputum (pneumonia) |
| YMC14/01/R198 | Tracheal aspirate | YMC14/01/P727 | Swab or drainage tube, |
| | (pneumonia) | | abdomen |
| YMC14/01/R324 | Sputum (pneumonia) | YMC14/01/R3129 | Sputum (pneumonia) |
| YMC14/01/R257 | Sputum (pneumonia) | YMC14/01/R3007 | Sputum (pneumonia) |
| YMC14/01/R270 | Sputum (pneumonia) | YMC14/01/R3317 | Sputum (pneumonia) |
| YMC14/01/P122 | Swab or drainage tube, | YMC14/01/R3474 | Sputum (pneumonia) |
| | pelvis | | |
| YMC14/01/P117 | Decubitus ulcer | YMC14/01/R3574 | Tracheal tube tip |
| YMC14/01/U318 | Random urine | YMC14/02/P47 | Bile, PTBD |
| YMC14/01/P212 | | YMC14/02/R542 | Sputum (pneumonia) |
| YMC14/01/R443 | Sputum (pneumonia) | YMC14/02/U1607 | Random urine |
| YMC14/01/R451 | Tracheal aspirate | YMC14/02/R1860 | Sputum (pneumonia) |
| | (pneumonia) | | |
| YMC14/01/R560 | Sputum (pneumonia) | YMC14/02/L18 | Bronchoalveolar lavage |
| YMC14/01/R617 | Sputum (pneumonia) | YMC14/02/R2417 | Sputum (pneumonia) |
| YMC14/01/R671 | Tracheal aspirate | YMC14/02/R2668 | Sputum (pneumonia) |
| | (pneumonia) | | |
| YMC14/01/R732 | Sputum (pneumonia) | YMC14/02/R2599 | Tracheal aspirate |
| | | | (pneumonia) |
| YMC14/01/R767 | Sputum (pneumonia) | YMC14/02/R2781 | Tracheal aspirate |
| | | | (pneumonia) |
| YMC14/01/L8 | Bronchoalveolar lavage | YMC14/02/R2758 | Mouth |
| YMC14/01/R905 | Sputum (pneumonia) | YMC14/02/R3106 | Sputum (pneumonia) |
| YMC14/01/R904 | Sputum (pneumonia) | YMC14/02/R3419 | Sputum (pneumonia) |
| YMC14/01/R941 | Tracheal aspirate | YMC14/03/R217 | Sputum (pneumonia) |
| | (pneumonia) | | |
| YMC14/01/R958 | Sputum (pneumonia) | YMC14/03/R122 | Sputum (pneumonia) |
| YMC14/01/P224 | Swab or drainage tube, hip | YMC14/03/R380 | Tracheal aspirate |
| | | | (pneumonia) |
| YMC14/01/R1006 | Sputum (pneumonia) | YMC14/03/R618 | Sputum (pneumonia) |
| YMC14/01/R921 | Sputum (pneumonia) | YMC14/03/L9 | Bronchoalveolar lavage |
| YMC14/01/R1659 | Tracheal aspirate | YMC14/03/P471 | Swab or drainage tube, |
| | (pneumonia) | | hand |
| YMC14/01/R1722 | Tracheal aspirate | YMC14/03/R2144 | Sputum (pneumonia) |
| | (pneumonia) | | |
| YMC14/01/R1752 | Sputum (surveillance) | YMC14/03/U4616 | Random urine |
| YMC14/01/R1199 | Tracheal tube tip | YMC14/04/R1080 | Sputum (pneumonia) |
| YMC14/01/R2036 | | YMC14/04/R1078 | Sputum (pneumonia) |

TABLE 2

| Host | | Ampicillin- | | | | | | | |
|---|---|---|---|---|---|---|---|---|---|
| strain | Amikacin | sulbactam | Ceftazidime | Ciprofloxacin | Colistin | Cefepime | Cefotaxime | Gentamicin | Imipenem |
| YMC14/01/ | 6 R | 16 I | =64 R | =4 R | =0.5 S | =64 R | =64 R | =16 R | =16 R |
| R130 | | | | | | | | | |
| YMC14/01/ | 6 R | =32 R | =64 R | =4 R | =0.5 S | =64 R | =64 R | =16 R | =16 R |
| R160 | | | | | | | | | |
| YMC14/01/ | 6 R | =32 R | =64 R | =4 R | =0.5 S | =64 R | =64 R | =16 R | =16 R |
| C29 | | | | | | | | | |
| YMC14/01/ | 6 R | =32 R | =64 R | =4 R | =0.5 S | =64 R | =64 R | =16 R | =16 R |
| P31 | | | | | | | | | |
| YMC14/01/ | 6 R | =2 S | =64 R | =4 R | =0.5 S | =64 R | =64 R | =16 R | =16 R |
| U313 | | | | | | | | | |
| YMC14/01/ | 6 R | =32 R | =64 R | =4 R | =0.5 S | =64 R | =64 R | =16 R | =16 R |
| R198 | | | | | | | | | |
| YMC14/01/ | 6 R | 8 S | =64 R | =4 R | =0.5 S | =64 R | =64 R | =16 R | =16 R |
| R324 | | | | | | | | | |
| YMC14/01/ | 23 S | =32 R | =64 R | =4 R | =0.5 S | =64 R | =64 R | =1 S | =16 R |
| R257 | | | | | | | | | |
| YMC14/01/ | 6 R | =32 R | =64 R | =4 R | =0.5 S | =64 R | =64 R | =16 R | =16 R |
| R270 | | | | | | | | | |
| YMC14/01/ | 6 R | =32 R | =64 R | =4 R | =0.5 S | 32 R | =64 R | =16 R | =16 R |
| P122 | | | | | | | | | |
| YMC14/01/ | 6 R | =32 R | =64 R | =4 R | =0.5 S | =64 R | =64 R | =16 R | =16 R |
| P117 | | | | | | | | | |
| YMC14/01/ | 6 R | =32 R | =64 R | =4 R | =0.5 S | =64 R | =64 R | =16 R | =16 R |
| U318 | | | | | | | | | |
| YMC14/01/ | | | | | | | | | |
| P212 | | | | | | | | | |
| YMC14/01/ | 6 R | =32 R | =64 R | =4 R | =0.5 S | =64 R | =64 R | =16 R | =16 R |
| R443 | | | | | | | | | |
| YMC14/01/ | 6 R | =32 R | =64 R | =4 R | =0.5 S | =64 R | =64 R | =16 R | =16 R |
| R451 | | | | | | | | | |
| YMC14/01/ | 8 R | =32 R | =64 R | =4 R | =0.5 S | =64 R | =64 R | 2 S | =16 R |
| R560 | | | | | | | | | |
| YMC14/01/ | 6 R | =32 R | 16 I | =4 R | =0.5 S | =64 R | =64 R | =16 R | =16 R |
| R617 | | | | | | | | | |
| YMC14/01/ | 6 R | =32 R | =64 R | =4 R | =0.5 S | =64 R | =64 R | =16 R | =16 R |
| R671 | | | | | | | | | |
| YMC14/01/ | 6 R | =32 R | =64 R | =4 R | =0.5 S | =64 R | =64 R | =16 R | =16 R |
| R732 | | | | | | | | | |
| YMC14/01/ | 6 R | 16 I | =64 R | =4 R | =0.5 S | =64 R | =64 R | =16 R | =16 R |
| R767 | | | | | | | | | |
| YMC14/01/ | 6 R | =32 R | =64 R | =4 R | =0.5 S | =64 R | =64 R | =16 R | =16 R |
| L8 | | | | | | | | | |
| YMC14/01/ | 6 R | =32 R | =64 R | =4 R | =0.5 S | =64 R | =64 R | =16 R | =16 R |
| R905 | | | | | | | | | |
| YMC14/01/ | 6 R | =32 R | =64 R | =4 R | =0.5 S | =64 R | =64 R | =16 R | =16 R |
| R904 | | | | | | | | | |
| YMC14/01/ | 6 R | =32 R | =64 R | =4 R | =0.5 S | =64 R | =64 R | =16 R | =16 R |
| R941 | | | | | | | | | |
| YMC14/01/ | 6 R | 16 I | =64 R | =4 R | =0.5 S | =64 R | =64 R | =16 R | =16 R |
| R958 | | | | | | | | | |
| YMC14/01/ | 6 R | 16 I | =64 R | =4 R | =0.5 S | =64 R | =64 R | =16 R | =16 R |
| P224 | | | | | | | | | |
| YMC14/01/ | 6 R | 16 I | =64 R | =4 R | =0.5 S | =64 R | =64 R | =16 R | =16 R |
| R1006 | | | | | | | | | |
| YMC14/01/ | 23 S | 16 I | =64 R | =4 R | =0.5 S | 16 I | =64 R | 8 I | =16 R |
| R921 | | | | | | | | | |
| YMC14/01/ | 6 R | 16 I | =64 R | =4 R | =0.5 S | =64 R | =64 R | =16 R | =16 R |
| R1659 | | | | | | | | | |
| YMC14/01/ | 6 R | 16 I | =64 R | =4 R | =0.5 S | =64 R | =64 R | =16 R | =16 R |
| R1722 | | | | | | | | | |
| YMC14/01/ | 6 R | 16 I | =64 R | =4 R | =0.5 S | =64 R | =64 R | =16 R | =16 R |
| R1752 | | | | | | | | | |
| YMC14/01/ | 6 R | 16 I | =64 R | =4 R | =0.5 S | =64 R | =64 R | =16 R | =16 R |
| R1199 | | | | | | | | | |
| YMC14/01/ | | | | | | | | | |
| R2036 | | | | | | | | | |
| YMC14/01/ | 6 R | 16 I | =64 R | =4 R | =0.5 S | =64 R | =64 R | =16 R | =16 R |
| R2429 | | | | | | | | | |
| YMC14/01/ | 6 R | 16 I | =64 R | =4 R | =0.5 S | =64 R | =64 R | =16 R | =16 R |
| P728 | | | | | | | | | |
| YMC14/01/ | 6 R | 16 I | =64 R | =4 R | =0.5 S | =64 R | =64 R | =16 R | =16 R |
| R2572 | | | | | | | | | |
| YMC14/01/ | 6 R | 16 I | =64 R | =4 R | =0.5 S | 32 R | =64 R | =16 R | =16 R |
| R2855 | | | | | | | | | |
| YMC14/01/ | 6 R | 4 S | =64 R | =4 R | =0.5 S | =64 R | =64 R | =16 R | =16 R |
| R2945 | | | | | | | | | |
| YMC14/01/ | 6 R | =32 R | =64 R | =4 R | =0.5 S | =64 R | =64 R | =16 R | =16 R |
| P727 | | | | | | | | | |
| YMC14/01/ | 6 R | =32 R | =64 R | =4 R | =0.5 S | =64 R | =64 R | =16 R | =16 R |
| R3129 | | | | | | | | | |
| YMC14/01/ | 6 R | 16 I | =64 R | =4 R | =0.5 S | =64 R | =64 R | =16 R | =16 R |
| R3007 | | | | | | | | | |
| YMC14/01/ | 6 R | 16 I | =64 R | =4 R | =0.5 S | =64 R | =64 R | =16 R | =16 R |
| R3317 | | | | | | | | | |
| YMC14/01/ | 6 R | =32 R | =64 R | =4 R | =0.5 S | =64 R | =64 R | =16 R | =16 R |
| R3474 | | | | | | | | | |
| YMC14/01/ | 6 R | 16 I | =64 R | =4 R | =0.5 S | =64 R | =64 R | =16 R | =16 R |
| R3574 | | | | | | | | | |
| YMC14/02/ | 6 R | =32 R | =64 R | =4 R | =0.5 S | =64 R | =64 R | =16 R | =16 R |
| P47 | | | | | | | | | |
| YMC14/02/ | | | | | | | | | |
| R542 | | | | | | | | | |
| YMC14/02/ | 20 S | 16 I | =64 R | =4 R | =0.5 S | =64 R | =64 R | =16 R | =16 R |
| U1607 | | | | | | | | | |
| YMC14/02/ | 27 S | 16 I | =64 R | =4 R | =0.5 S | 8 S | =64 R | 8 I | =16 R |
| R1860 | | | | | | | | | |
| YMC14/02/ | 6 R | 16 I | =64 R | =4 R | =0.5 S | =64 R | =64 R | =16 R | =16 R |
| L18 | | | | | | | | | |
| YMC14/02/ | 6 R | 16 I | =64 R | =4 R | =0.5 S | =64 R | =64 R | =16 R | =16 R |
| R2417 | | | | | | | | | |
| YMC14/02/ | 6 R | 8 S | =64 R | =4 R | =0.5 S | =64 R | =64 R | =16 R | =16 R |
| R2668 | | | | | | | | | |
| YMC14/02/ | 20 S | =32 R | =64 R | =4 R | =0.5 S | =64 R | =64 R | 2 S | =16 R |
| R2599 | | | | | | | | | |
| YMC14/02/ | 6 R | =32 R | =64 R | =4 R | =0.5 S | =64 R | =64 R | =16 R | =16 R |
| R2781 | | | | | | | | | |
| YMC14/02/ | 6 R | 8 S | =64 R | =4 R | =0.5 S | =64 R | =64 R | =16 R | =16 R |
| R2758 | | | | | | | | | |
| YMC14/02/ | 6 R | 8 S | =64 R | =4 R | =0.5 S | =64 R | =64 R | =16 R | =16 R |
| R3106 | | | | | | | | | |
| YMC14/02/ | 6 R | 16 I | =64 R | =4 R | =0.5 S | =64 R | =64 R | =16 R | =16 R |
| R3419 | | | | | | | | | |
| YMC14/03/ | 6 R | 16 I | =64 R | =4 R | =0.5 S | =64 R | =64 R | =16 R | =16 R |
| R217 | | | | | | | | | |
| YMC14/03/ | 17 S | =32 R | =64 R | =4 R | =0.5 S | =64 R | =64 R | =16 R | =16 R |
| R122 | | | | | | | | | |
| YMC14/03/ | 6 R | 8 S | =64 R | =4 R | =0.5 S | =64 R | =64 R | =16 R | =16 R |
| R380 | | | | | | | | | |
| YMC14/03/ | 21 S | 8 S | =64 R | =4 R | =0.5 S | =64 R | =64 R | 2 S | =16 R |
| R618 | | | | | | | | | |
| YMC14/03/ | 20 S | 16 I | =64 R | =4 R | =0.5 S | =64 R | =64 R | =16 R | =16 R |
| L9 | | | | | | | | | |
| YMC14/03/ | 22 S | 8 S | =64 R | =4 R | =0.5 S | =64 R | =64 R | 2 S | =16 R |
| P471 | | | | | | | | | |
| YMC14/03/ | 6 R | 16 I | =64 R | =4 R | =0.5 S | =64 R | =64 R | =16 R | =16 R |
| R2144 | | | | | | | | | |
| YMC14/03/ | 6 R | 16 I | =64 R | =4 R | =0.5 S | =64 R | =64 R | =16 R | =16 R |
| U4616 | | | | | | | | | |
| YMC14/04/ | 20 S | 4 S | =64 R | =4 R | =0.5 S | 32 R | =64 R | 2 S | =16 R |
| R1080 | | | | | | | | | |
| YMC14/04/ | 20 S | =32 R | =64 R | =4 R | =0.5 S | =64 R | =64 R | 2 S | =16 R |
| R1078 | | | | | | | | | |

| Host | | | | | Piperacillin- | | |
|---|---|---|---|---|---|---|---|
| strain | Levofloxacin | Meropenem | Minocycline | Piperacillin | tazobactam | Cortrimoxa | Tigecycline |
| YMC14/01/ | =8 R | =16 R | =1 S | =128 R | =128 R | =320 R | 2 S |
| R130 | | | | | | | |
| YMC14/01/ | =8 R | =16 R | 8 I | =128 R | =128 R | =320 R | 1 S |
| R160 | | | | | | | |
| YMC14/01/ | =8 R | =16 R | 4 S | =128 R | =128 R | =320 R | 1 S |
| C29 | | | | | | | |
| YMC14/01/ | =8 R | =16 R | =1 S | =128 R | =128 R | 160 R | 1 S |
| P31 | | | | | | | |
| YMC14/01/ | =8 R | =16 R | 2 S | =128 R | =128 R | =320 R | =8 R |
| U313 | | | | | | | |
| YMC14/01/ | =8 R | =16 R | 8 I | =128 R | =128 R | =320 R | 2 S |
| R198 | | | | | | | |
| YMC14/01/ | =8 R | =16 R | =1 S | =128 R | =128 R | 160 R | 2 S |
| R324 | | | | | | | |
| YMC14/01/ | =8 R | =16 R | 8 I | =128 R | =128 R | =320 R | 2 S |
| R257 | | | | | | | |
| YMC14/01/ | =8 R | =16 R | =1 S | =128 R | =128 R | =320 R | 2 S |
| R270 | | | | | | | |
| YMC14/01/ | 4 I | =16 R | 2 S | =128 R | =128 R | =20 S | 1 S |
| P122 | | | | | | | |
| YMC14/01/ | 4 I | =16 R | =1 S | =128 R | =128 R | 160 R | 2 S |
| P117 | | | | | | | |
| YMC14/01/ | =8 R | =16 R | 8 I | =128 R | =128 R | =320 R | 2 S |
| U318 | | | | | | | |
| YMC14/01/ | | | | | | | |
| P212 | | | | | | | |
| YMC14/01/ | =8 R | =16 R | 8 I | =128 R | =128 R | =320 R | 1 S |
| R443 | | | | | | | |
| YMC14/01/ | =8 R | =16 R | =16 R | =128 R | =128 R | =320 R | 2 S |
| R451 | | | | | | | |
| YMC14/01/ | =8 R | =16 R | =16 R | =128 R | =128 R | 160 R | =8 R |
| R560 | | | | | | | |
| YMC14/01/ | =8 R | =16 R | =16 R | =128 R | =128 R | =320 R | 2 S |
| R617 | | | | | | | |
| YMC14/01/ | =8 R | =16 R | 8 I | =128 R | =128 R | =320 R | 2 S |
| R671 | | | | | | | |
| YMC14/01/ | =8 R | =16 R | =1 S | =128 R | =128 R | =320 R | =8 R |
| R732 | | | | | | | |
| YMC14/01/ | =8 R | =16 R | =1 S | =128 R | =128 R | 160 R | 1 S |
| R767 | | | | | | | |
| YMC14/01/ | =8 R | =16 R | =1 S | =128 R | =128 R | =320 R | =0.5 S |
| L8 | | | | | | | |
| YMC14/01/ | =8 R | =16 R | 8 I | =128 R | =128 R | =320 R | 2 S |
| R905 | | | | | | | |
| YMC14/01/ | =8 R | =16 R | 8 I | =128 R | =128 R | =20 S | 2 S |
| R904 | | | | | | | |
| YMC14/01/ | =8 R | =16 R | =1 S | =128 R | =128 R | =320 R | =8 R |
| R941 | | | | | | | |
| YMC14/01/ | =8 R | =16 R | 4 S | =128 R | =128 R | =320 R | =8 R |
| R958 | | | | | | | |
| YMC14/01/ | =8 R | =16 R | 2 S | =128 R | =128 R | =320 R | 4 I |
| P224 | | | | | | | |
| YMC14/01/ | =8 R | =16 R | 2 S | =128 R | =128 R | =320 R | 2 S |
| R1006 | | | | | | | |
| YMC14/01/ | =8 R | =16 R | 2 S | =128 R | =128 R | 40 S | 2 S |
| R921 | | | | | | | |
| YMC14/01/ | =8 R | =16 R | =1 S | =128 R | =128 R | =320 R | 2 S |
| R1659 | | | | | | | |
| YMC14/01/ | =8 R | =16 R | =1 S | =128 R | =128 R | =320 R | 2 S |
| R1722 | | | | | | | |
| YMC14/01/ | =8 R | =16 R | =1 S | =128 R | =128 R | =20 S | 2 S |
| R1752 | | | | | | | |
| YMC14/01/ | =8 R | =16 R | =1 S | =128 R | =128 R | 160 R | 2 S |
| R1199 | | | | | | | |
| YMC14/01/ | | | | | | | |
| R2036 | | | | | | | |
| YMC14/01/ | =8 R | =16 R | =1 S | =128 R | =128 R | 160 R | 2 S |
| R2429 | | | | | | | |
| YMC14/01/ | =8 R | =16 R | =1 S | =128 R | =128 R | 160 R | 2 S |
| P728 | | | | | | | |
| YMC14/01/ | =8 R | =16 R | 2 S | =128 R | =128 R | =320 R | 4 I |
| R2572 | | | | | | | |
| YMC14/01/ | =8 R | =16 R | =1 S | =128 R | =128 R | =320 R | 1 S |
| R2855 | | | | | | | |
| YMC14/01/ | =8 R | =16 R | =1 S | =128 R | =128 R | 160 R | 2 S |
| R2945 | | | | | | | |
| YMC14/01/ | =8 R | =16 R | =1 S | =128 R | =128 R | =320 R | =8 R |
| P727 | | | | | | | |
| YMC14/01/ | =8 R | =16 R | =1 S | =128 R | =128 R | =20 S | 4 I |
| R3129 | | | | | | | |
| YMC14/01/ | =8 R | =16 R | =1 S | =128 R | =128 R | =20 S | 2 S |
| R3007 | | | | | | | |
| YMC14/01/ | =8 R | =16 R | =1 S | =128 R | =128 R | 160 R | 2 S |
| R3317 | | | | | | | |
| YMC14/01/ | 4 I | =16 R | =1 S | =128 R | =128 R | 160 R | 1 S |
| R3474 | | | | | | | |
| YMC14/01/ | =8 R | =16 R | =1 S | =128 R | =128 R | 160 R | 2 S |
| R3574 | | | | | | | |
| YMC14/02/ | =8 R | =16 R | =1 S | =128 R | =128 R | =320 R | 4 I |
| P47 | | | | | | | |
| YMC14/02/ | | | | | | | |
| R542 | | | | | | | |
| YMC14/02/ | =8 R | =16 R | =1 S | =128 R | =128 R | =20 S | 2 S |
| U1607 | | | | | | | |
| YMC14/02/ | =8 R | =16 R | =1 S | =128 R | =128 R | =320 R | 4 I |
| R1860 | | | | | | | |
| YMC14/02/ | =8 R | =16 R | =1 S | =128 R | =128 R | 160 R | 2 S |
| L18 | | | | | | | |
| YMC14/02/ | =8 R | =16 R | =1 S | =128 R | =128 R | =320 R | 2 S |
| R2417 | | | | | | | |
| YMC14/02/ | =8 R | =16 R | =1 S | =128 R | =128 R | =320 R | =8 R |
| R2668 | | | | | | | |
| YMC14/02/ | =8 R | =16 R | =1 S | =128 R | =128 R | =20 S | 2 S |
| R2599 | | | | | | | |
| YMC14/02/ | =8 R | =16 R | =1 S | =128 R | =128 R | 160 R | 2 S |
| R2781 | | | | | | | |
| YMC14/02/ | =8 R | =16 R | =1 S | =128 R | =128 R | =320 R | =8 R |
| R2758 | | | | | | | |
| YMC14/02/ | =8 R | =16 R | =1 S | =128 R | =128 R | =320 R | 2 S |
| R3106 | | | | | | | |
| YMC14/02/ | =8 R | =16 R | =1 S | =128 R | =128 R | =320 R | =8 R |
| R3419 | | | | | | | |
| YMC14/03/ | =8 R | =16 R | =1 S | =128 R | =128 R | 160 R | 2 S |
| R217 | | | | | | | |
| YMC14/03/ | =8 R | =16 R | =1 S | =128 R | =128 R | =20 S | =8 R |
| R122 | | | | | | | |
| YMC14/03/ | =8 R | =16 R | =1 S | =128 R | =128 R | =320 R | =8 R |
| R380 | | | | | | | |
| YMC14/03/ | =8 R | =16 R | =1 S | =128 R | =128 R | =20 S | 2 S |
| R618 | | | | | | | |
| YMC14/03/ | =8 R | =16 R | =1 S | =128 R | =128 R | 160 R | 2 S |
| L9 | | | | | | | |
| YMC14/03/ | =8 R | =16 R | =1 S | =128 R | =128 R | =20 S | 2 S |
| P471 | | | | | | | |
| YMC14/03/ | =8 R | =16 R | =1 S | =128 R | =128 R | 160 R | 2 S |
| R2144 | | | | | | | |
| YMC14/03/ | =8 R | =16 R | =1 S | =128 R | =128 R | 160 R | 2 S |
| U4616 | | | | | | | |
| YMC14/04/ | =8 R | =16 R | =1 S | =128 R | =128 R | =20 S | 2 S |
| R1080 | | | | | | | |
| YMC14/04/ | =8 R | =16 R | =1 S | =128 R | =128 R | =20 S | 1 S |
| R1078 | | | | | | | |

As shown in Table 2, the collected 66 Acinetobacter baumannii strains were found to be multi-drug-resistant strains having resistance to various antibiotics.

2. Collection of Bacteriophage Specimens

2-1. Collection of Specimens to Construct Phage Bank

Raw water was obtained by causing sewage to pass through a first sedimentation tank at the sewage treatment facility of the Severance Hospital (Korea), and then removing suspended substances and sediments therefrom. The sewage was limited to sewage that was present at a preliminary stage of a chemical treatment facility. To the collected sample was added 58 g of sodium chloride per L. Then, centrifugation was performed at 10,000 g for 10 minutes and filtration was performed through a 220 nm Millipore filter. To the obtained filtrate was added polyethylene glycol (PEG, molecular weight of 8000) at 10% w/v, and the resultant was stored refrigerated at 4° C. for 12 hours. The filtrate stored refrigerated for 12 hours was centrifuged at 12,000 g for 20 minutes, and the precipitate was resuspended in phage dilution buffer (SM buffer). To the resuspension was then added the same amount of chloroform, and the resultant was stored frozen. This was repeated three times to collect 300 mL of bacteriophage suspension.

2-2. Selection of Lytic Phage and Measurement of Lysis Titer

Separation and purification of lytic phage were performed by a spot test method (Mazzocco A et al. In Bacteriophages, Clokie and Kropinski AM, eds. Humana Press. 2009). The obtained strains were inoculated on MacConkey Agar medium and then cultured overnight at 35° C. in outside air. After the culture, strains susceptible to phage were selected by observing formation of clear plaque. The susceptible strains were inoculated on MacConkey Agar medium and cultured at 35° C. for 12 hours. A suspension of each strain was prepared in a 1 ml saline tube with a turbidity of 0.5 McFarland, and mixed with H top agar (3 ml), 100 μl of sensitive bacteria, and a phage solution (each of 1 μl, 10 μl, and 50 μl). The mixture was applied to LB agar, and then cultured at 35° C. for 12 hours. Plaque was observed, and then the plaque was collected with a Pasteur pipette. The collected plaque was diluted in SM buffer solution, and repeatedly purified three times using the susceptible strain suspension again. The thus obtained pure bacteriophage, YMC14/01/P117_ABA_BP, was diluted in SM buffer solution, and repeatedly purified three times using the susceptible strain suspension again. The thus obtained pure bacteriophage, YMC14/01/P117_ABA_BP, was diluted in SM buffer solution and stored.

Each of the 32 antibiotic-resistant Acinetobacter baumannii strains identified in item no. 1. above was inoculated on MacConkey Agar medium and cultured. Then, the bacteriophage YMC14/01/P117_ABA_BP, which had been purified by the above process, was inoculated in an amount of 5 μl into each smeared resistant strain. Then, plaque formation was checked and a titer range thereof was checked. The lysis of each strain is shown in Table 3 below. In Table 3 below, an evaluation result of plaque activity against the collected strains is indicated by + and −, in which ‘+’ means clear plaque and ‘-’ means that lysis has not occurred.

TABLE 3

| Host strain | Lysis | Host strain | Lysis |
|---|---|---|---|
| YMC14/01/R130 | + | YMC14/01/R3317 | + |
| YMC14/01/P31 | + | YMC14/01/R3474 | + |
| YMC14/01/U313 | ++ | YMC14/01/R3574 | + |
| YMC14/01/R324 | + | YMC14/02/P47 | + |
| YMC14/01/R270 | + | YMC14/02/R542 | + |
| YMC14/01/P117 | + | YMC14/02/U1607 | + |
| YMC14/01/R732 | + | YMC14/02/R1860 | + |
| YMC14/01/R767 | + | YMC14/02/L18 | + |
| YMC14/01/R904 | + | YMC14/02/R2417 | + |
| YMC14/01/R941 | + | YMC14/02/R2668 | + |
| YMC14/01/P224 | + | YMC14/02/R2599 | + |
| YMC14/01/R1006 | + | YMC14/02/R2781 | + |
| YMC14/01/R921 | + | YMC14/02/R2758 | + |
| YMC14/01/R1659 | + | YMC14/02/R3106 | + |
| YMC14/01/R1722 | + | YMC14/02/R3419 | + |
| YMC14/01/R1752 | + | YMC14/03/R217 | + |
| YMC14/01/R1199 | + | YMC14/03/R122 | + |
| YMC14/01/R2036 | ++ | YMC14/03/R380 | + |
| YMC14/01/R2429 | + | YMC14/03/R618 | + |
| YMC14/01/P728 | + | YMC14/03/L9 | + |
| YMC14/01/R2572 | + | YMC14/03/P471 | + |
| YMC14/01/R2855 | + | YMC14/03/R2144 | + |
| YMC14/01/R2945 | + | YMC14/03/U4616 | + |
| YMC14/01/P727 | + | YMC14/04/R1080 | + |
| YMC14/01/R3129 | + | YMC14/04/R1078 | + |
| YMC14/01/R3007 | + | | |

As shown in Table 3, it was found that the bacteriophage YMC14/01/P117_ABA_BP according to the present invention lyses antibiotic-resistant Acinetobacter baumannii strains.

3. Electron Microscopic Analysis of Lytic Bacteriophage Against Antibiotic-Resistant Acinetobacter baumannii Strains

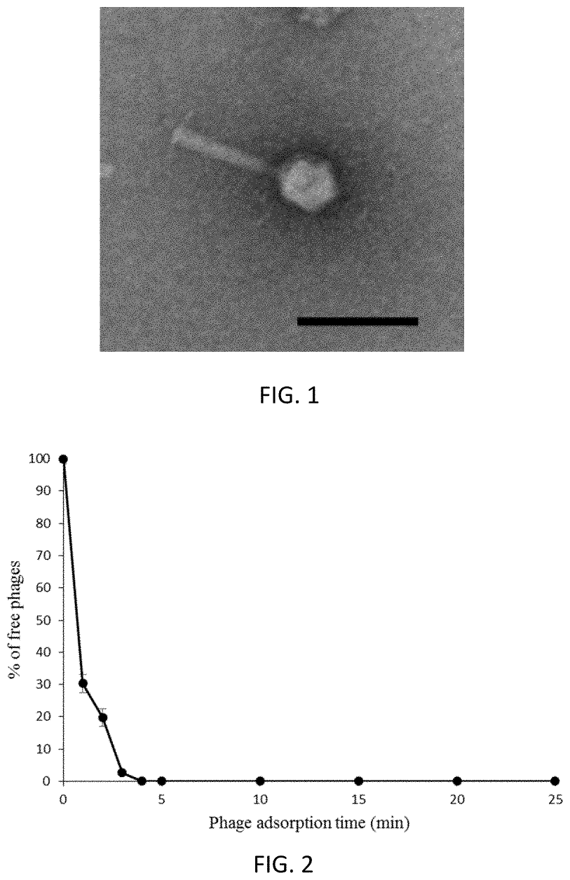

The bacteriophage YMC14/01/P117_ABA_BP purified by the method of item no. 2. above was inoculated and cultured in culture medium (20 ml of LB medium) for susceptible strains, and then filtered through a 220 nm Millipore filter. To the supernatant was added polyethylene glycol (MW 8,000) in an amount of 10% (w/v), and then the resultant was stored refrigerated overnight. Subsequently, centrifugation was performed for 20 minutes at 12,000 g, and then a shape of the bacteriophage YMC14/01/P117_ABA_BP was analyzed using an energy-filtering transmission electron microscope. The result is illustrated in FIG. 1.

As illustrated in FIG. 1, in a case where classification is made on a shape basis, the bacteriophage YMC14/01/P117_ABA_BP according to the present invention was classified as belonging to the family Myoviridae that has a long tail with a hexagonal head.

4. Analysis of Adsorption Capacity and One-Step Growth Curve of Bacteriophage

The antibiotic-resistant Acinetobacter baumannii strain was cultured to an OD value of 0.5. To the Acinetobacter baumannii strain was then added the bacteriophage YMC14/01/P117_ABA_BP purified in item no. 2. above at an MOI of 0.001 and culture was performed at room temperature. Then, sample was collected 1 ml each at 1, 2, 3, 4, and 5 minutes, diluted in LB medium, and then adsorption capacity of the bacteriophage was evaluated through plaque analysis. The results are illustrated in FIG. 2.

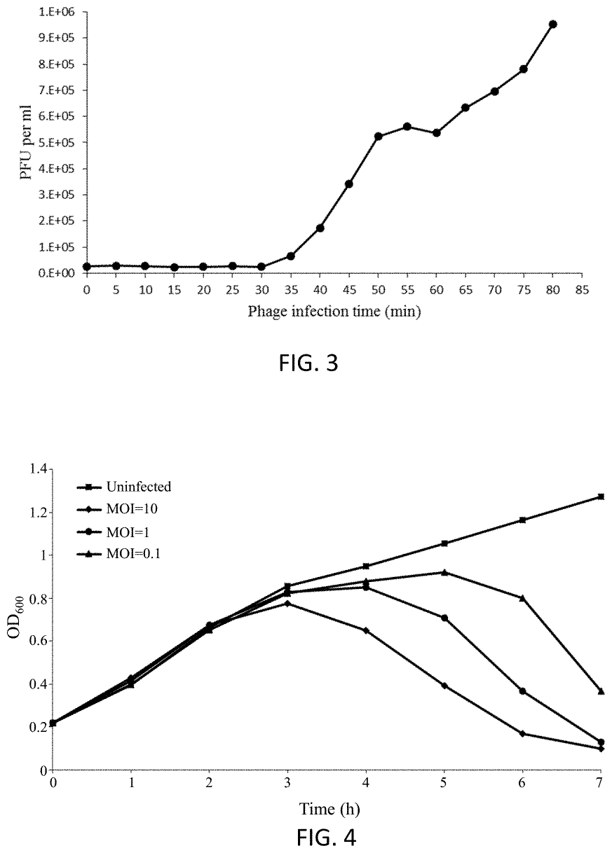

In addition, the antibiotic-resistant Acinetobacter baumannii strain was cultured to an OD value of 0.3, and then centrifuged at 7,000 g for 5 minutes at 4° C., to precipitate the cells. Then, the cells were diluted in 0.5 ml of LB medium. To the dilute was added the bacteriophage YMC14/01/P117_ABA_BP purified in item no. 2. above at an MOI of 0.001 (titer of 108 pfu/cell), and culture was performed at 37° C. for 5 minutes. The cultured mixed sample was centrifuged at 13,000 g for 1 minute to obtain a pellet. The obtained pellet was diluted in 10 ml of LB medium and cultured at 37° C. Samples were collected every 10 minutes during the culture, and a one-step growth curve of the bacteriophage was evaluated through plaque analysis. The results are illustrated in FIG. 3.

As illustrated in FIG. 2, about 99% of the bacteriophage YMC14/01/P117_ABA_BP was adsorbed to the Acinetobacter baumannii strain within 4 minutes after inoculation of the bacteriophage.

In addition, as illustrated in FIG. 3, the one-step growth curve showed a high burst size of approximately 38.08 PFU/infected cells.

From the above results, it can be seen that the bacteriophage YMC14/01/P117_ABA_BP according to the present invention can be adsorbed in a relatively short time to an antibiotic-resistant Acinetobacter baumannii strain and can show a high burst size of 38.08 PFU/infected cells, indicating that this bacteriophage exerts a lytic effect on an antibiotic-resistant strain.

5. Verification of Ex Vivo Lysis Ability of Bacteriophage Against Antibiotic-Resistant Acinetobacter Genus Bacteria

The antibiotic-resistant Acinetobacter baumannii strain at 1×10⁹ CFU/ml was treated with the prepared bacteriophage YMC14/01/P117_ABA_BP in an amount of 1×10⁸ CFU/ml (MOT: 0.1), 1×10⁹ PFU/ml (MOT: 1), or 1×10¹⁰ PFU/ml (MOT: 10), respectively, and OD values (wavelength of 600 nm) were measured over time. Here, as a negative control, treatment with PBS+SM buffer was performed. The values are illustrated in FIG. 4.

As illustrated in FIG. 4, in a case where the Acinetobacter baumannii strain is treated with the bacteriophage YMC14/01/P117_ABA_BP, an OD value decreased, in which the OD value further decreased as an MOI value increased, and in particular, the highest lysis ability was observed when the MOI was 10.

From the above results, it can be seen that the bacteriophage YMC14/01/P117_ABA_BP according to the present invention has lytic properties against an antibiotic-resistant Acinetobacter baumannii strain.

6. Verification of In Vivo Lysis Ability of Bacteriophage Against Antibiotic-Resistant Acinetobacter Genus Bacteria

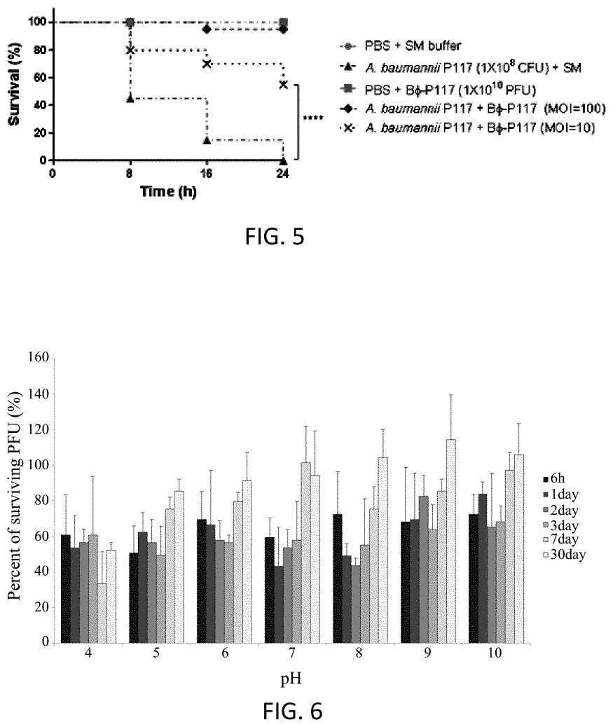

200 third- to fourth-instar Galleria mellonella larvae were prepared, and then divided into groups, each containing 10 larvae. Each larva was injected through its proleg with a carbapenem-resistant Acinetobacter baumannii strain at a minimum lethal dose (MLD), and then subjected to mixed inoculation with the bacteriophage YMC14/01/P117_ABA_BP purified in item no. 2. above at an MOI of 10 or an MOI of 100. Then, survival of the larvae was checked every 12 or 24 hours until 72 hours, and the results are illustrated in FIG. 5.

As illustrated in FIG. 5, it was found that in a case where the larvae injected with the carbapenem-resistant Acinetobacter baumannii strain are treated with the bacteriophage YMC14/01/P117_ABA_BP according to the present invention, survival of the larvae increases, in which the survival of the larvae further increases as the MOI value increases. In addition, it was found that even in a case where the larvae are injected with only the bacteriophage YMC14/01/P117_ABA_BP without injection of the carbapenem-resistant Acinetobacter baumannii strain, no toxicity is seen when survival thereof is compared with that of a healthy control group.

From the above results, it can be seen that the bacteriophage YMC14/01/P117_ABA_BP according to the present invention also has lytic properties in vivo against an antibiotic-resistant Acinetobacter baumannii strain, and thus can effectively prevent, ameliorate, or treat an infectious disease caused by the Acinetobacter baumannii strain.

7. Evaluation of Stability of Bacteriophage Against Antibiotic-Resistant Acinetobacter baumannii Strain

It was identified whether the bacteriophage YMC14/01/P117_ABA_BP according to the present invention maintains stability without being destroyed under alkaline and temperature conditions.

1 μl of the bacteriophage YMC14/01/P117_ABA_BP purified by the method of item no. 2 above was added to 40 μl of SM buffer, which had been adjusted to a pH of 4, 5, 6, 7, 8, 9, or 10, and then incubated at 37° C. for 1 hour. Then, plaque analysis was performed with the antibiotic-resistant Acinetobacter baumannii bacteria using the method of item no. 4 above. The results are illustrated in FIG. 6.

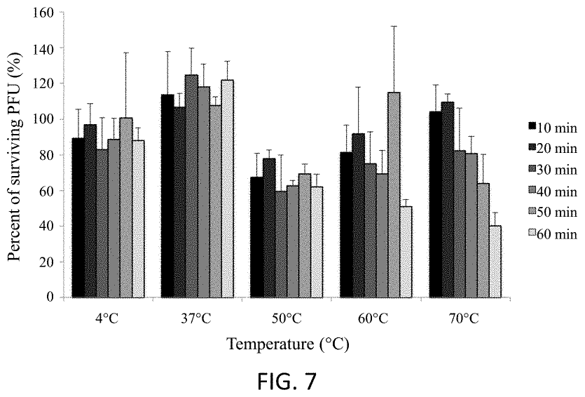

In addition, during 1-hour incubation of the bacteriophage YMC14/01/P117_ABA_BP solution at 4° C., 37° C., 50° C., 60° C., and 70° C., respectively, each sample was collected every 10 minutes and plaque analysis was performed with the Acinetobacter baumannii strain using the method of item no. 4 above. The results are illustrated in FIG. 7.

As illustrated in FIG. 6, the bacteriophage YMC14/01/P117_ABA_BP according to the present invention exhibited high stability in all conditions which are acidic, neutral, and alkaline.

In addition, as illustrated in FIG. 7, the bacteriophage YMC14/01/P117_ABA_BP exhibited very high stability up to a temperature as high as 70° C.

8. Whole-Genome Sequencing of Bacteriophage Against Antibiotic-Resistant Acinetobacter Genus Bacteria

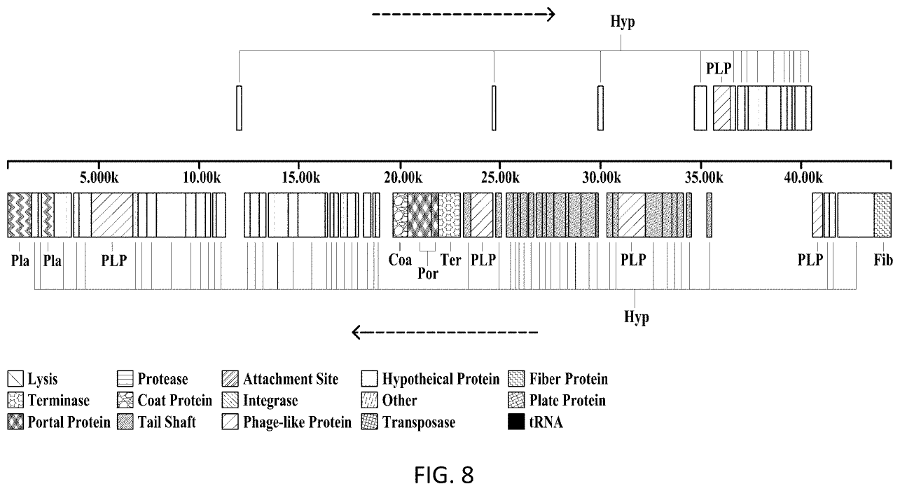

To characterize the bacteriophage YMC14/01/P117_ABA_BP according to the present invention, whole-genome sequencing thereof was performed through the Illumina sequencer (Roche) based on a whole-genome sequencing method which is obvious to those skilled in the art. The results are shown in FIG. 8 and Table 4.

TABLE 4

| | | | | | | | | | NCBI | |
|---|---|---|---|---|---|---|---|---|---|---|
| | | | Initi- | | | | | | blastP | NCBI-Bank |
| Genome | Range | | ation | | Length | Putative | Annotation | E- | identity | accession |
| no. | Start | End | codon | Strand | (bp) | function | source | value | (%) | number |
| ORF1 | 445 | 1629 | ATG | − | 1185 | Putative baseplate | Acinetobacter phage | 0 | 99 | AFV51558.1 |
| | | | | | | J-like protein | IME-AB2 | | | |
| ORF2 | 1626 | 1979 | ATG | − | 354 | Hypothetical protein | Acinetobacter phage | 3E−81 | 99 | ADO14451.1 |
| | | | | | | | AB1 | | | |
| ORF3 | 2125 | 2796 | ATG | − | 672 | Putative baseplate | Acinetobacter phage | 2E−157 | 100 | YP_009055472.1 |
| | | | | | | assembly protein | YMC-13-01-C62 | | | |
| ORF4 | 2753 | 3643 | GTG | − | 891 | Hypothetical protein | Acinetobacter phage | 0 | 94 | ADO14453.1 |
| | | | | | | | AB1 | | | |
| ORF5 | 3752 | 4093 | ATG | − | 342 | Hypothetical protein | Acinetobacter phage | 5E−60 | 99 | ARB06749.1 |
| | | | | | | | WCHABP12 | | | |
| ORF6 | 4032 | 4628 | ATG | − | 597 | Hypothetical protein | Acinetobacter phage | 2E−139 | 98 | ADO14454.1 |
| | | | | | | | AB1 | | | |
| ORF7 | 4636 | 6684 | ATG | − | 2049 | Lysozyme like | Acinetobacter phage | 0 | 100 | YP_009055475.1 |
| | | | | | | domain protein | YMC-13-01-C62 | | | |
| ORF8 | 6687 | 6929 | GTG | − | 243 | Putative tail- | Acinetobacter phage | 3E−52 | 99 | YP_009055476.1 |
| | | | | | | fiber/lysozyme | YMC-13-01-C62 | | | |
| | | | | | | protein | | | | |
| ORF9 | 6929 | 7354 | ATG | − | 426 | Hypothetical protein | Acinetobacter phage | 1E−37 | 46 | ADO14372.1 |
| | | | | | | | AB1 | | | |
| ORF10 | 7400 | 7949 | ATG | − | 550 | Hypothetical protein | Acinetobacter phage | 2E−59 | 58 | ADO14373.1 |
| | | | | | | | AB1 | | | |
| ORF11 | 7862 | 9325 | ATG | − | 1464 | Hypothetical protein | Acinetobacter phage | 0 | 98 | ADO14374.1 |
| | | | | | | | AB1 | | | |
| ORF12 | 9315 | 9809 | ATG | − | 495 | Hypothetical protein | Acinetobacter phage | 3E−110 | 93 | ADO14375.1 |
| | | | | | | | AB1 | | | |
| ORF13 | 9806 | 10276 | ATG | − | 471 | Hypothetical protein | Acinetobacter phage | 3E−108 | 96 | ADO14377.1 |
| | | | | | | | AB1 | | | |
| ORF14 | 10354 | 10635 | ATG | − | 282 | Putative capsid | Acinetobacter phage | 3E−55 | 100 | YP_009055482.1 |
| | | | | | | protein | YMC-13-01-C62 | | | |
| ORF15 | 10683 | 10868 | ATG | − | 186 | Hypothetical protein | Acinetobacter phage | 8E−31 | 90 | ADO14379.1 |
| | | | | | | | AB1 | | | |
| ORF16 | 10865 | 11371 | ATG | − | 507 | Putative RNA | Acinetobacter phage | 5E−98 | 94 | ARB06827.1 |
| | | | | | | polymerase | IME-AB2 | | | |
| ORF17 | 11878 | 12102 | ATG | + | 225 | Hypothetical protein | Acinetobacter phage | 4E−47 | 100 | AFV51493.1 |
| | | | | | | | IME-AB2 | | | |
| ORF18 | 12270 | 12545 | ATG | − | 276 | Hypothetical protein | Acinetobacter phage | 2E−58 | 98 | YP_006383783.1 |
| | | | | | | | AP22 | | | |
| ORF19 | 12561 | 13010 | ATG | − | 450 | Hypothetical protein | Acinetobacter phage | 1E−84 | 80 | ADO14383.1 |
| | | | | | | | AB1 | | | |
| ORF20 | 13010 | 13348 | ATG | − | 339 | Hypothetical protein | Acinetobacter phage | 3E−21 | 43 | ADO14384.1 |
| | | | | | | | AB1 | | | |
| ORF21 | 13428 | 14447 | ATG | − | 1020 | Hypothetical protein | Acinetobacter phage | 0 | 100 | YP_009055489.1 |
| | | | | | | | YMC-13-01-C62 | | | |
| ORF22 | 14457 | 14936 | ATG | − | 480 | Hypothetical protein | Acinetobacter phage | 2E−31 | 44 | ADO14387.1 |
| | | | | | | | AB1 | | | |
| ORF23 | 14944 | 16278 | ATG | − | 1335 | Hypothetical protein | Acinetobacter phage | 0 | 81 | ADO14388.1 |
| | | | | | | | AB1 | | | |
| ORF24 | 16492 | 16698 | ATG | − | 207 | Hypothetical protein | Acinetobacter phage | 1E−43 | 100 | YP_009055493.1 |
| | | | | | | | YMC-13-01-C62 | | | |
| ORF25 | 16688 | 16963 | ATG | − | 276 | Hypothetical protein | Acinetobacter phage | 6E−61 | 100 | YP_009055494.1 |
| | | | | | | | YMC-13-01-C62 | | | |
| ORF26 | 17062 | 17424 | ATG | − | 363 | Hypothetical protein | Acinetobacter phage | 2E−84 | 100 | YP_009055495.1 |
| | | | | | | | YMC-13-01-C62 | | | |
| ORF27 | 17421 | 17813 | ATG | − | 393 | Hypothetical protein | Acinetobacter phage | 1E−89 | 100 | AJT61472.1 |
| | | | | | | | YMC11/12/R1215 | | | |
| ORF28 | 17806 | 18228 | ATG | − | 423 | | | | | |
| ORF29 | 18218 | 18571 | ATG | − | 354 | Hypothetical protein | Acinetobacter phage | 4E−83 | 100 | YP_009055498.1 |
| | | | | | | | YMC-13-01-C62 | | | |
| ORF30 | 18653 | 18763 | ATG | − | 111 | Hypothetical protein | Acinetobacter phage | 3E−27 | 100 | YP_009055499.1 |
| | | | | | | | YMC-13-01-C62 | | | |
| 0RF31 | 18800 | 18964 | ATG | − | 165 | Hypothetical protein | Acinetobacter phage | 7E−32 | 100 | YP_009055500.1 |
| | | | | | | | YMC-13-01-C62 | | | |
| ORF32 | 19654 | 20424 | ATG | − | 771 | Putative head | Acinetobacter phage | 0 | 99 | ASJ78923.1 |
| | | | | | | protein | AbP2 | | | |
| ORF33 | 20427 | 21557 | ATG | − | 1131 | Putative portal | Acinetobacter phage | 0 | 95 | ARB06806.1 |
| | | | | | | protein | WCHABP12 | | | |
| ORF34 | 21574 | 21930 | ATG | − | 357 | Putative portal | Acinetobacter phage | 9E−80 | 99 | ARB06806.1 |
| | | | | | | protein | WCHABP12 | | | |
| ORF35 | 21934 | 23235 | ATG | − | 1302 | Putative phage | Acinetobacter phage | 0 | 94 | YP_006383766.1 |
| | | | | | | terminase | AP22 | | | |
| | | | | | | large subunit | | | | |
| ORF36 | 23204 | 23569 | ATG | − | 366 | DNA binding domain | uncultured | 2E−12 | 41 | BAQ88996.1 |
| | | | | | | | Mediterranean | | | |
| | | | | | | | phage uvMED | | | |
| ORF37 | 23562 | 24755 | GTG | − | 1194 | ParB/sulfiredoxin | Vibrio phage | 4E−138 | 58 | AUR95847.1 |
| | | | | | | | 1.213.O._10N.222.54.F10 | | | |
| ORF38 | 24807 | 25070 | ATG | − | 264 | Hypothetical protein | Acinetobacter phage | 2E−32 | 96 | ASJ78929.1 |
| | | | | | | | AbP2 | | | |
| ORF39 | 25175 | 25354 | ATG | − | 180 | Hypothetical protein | Acinetobacter phage | 7E−09 | 49 | YP_009055426.1 |
| | | | | | | | YMC-13-01-C62 | | | |
| ORF40 | 25357 | 25683 | ATG | − | 327 | Hypothetical protein | Acinetobacter phage | 4E−74 | 100 | AJT61457.1 |
| | | | | | | | YMC11/12/R1215 | | | |
| ORF41 | 26010 | 26348 | ATG | − | 339 | Hypothetical protein | Acinetobacter phage | 2E−79 | 100 | YP_009055430.1 |
| | | | | | | | YMC-13-01-C62 | | | |
| ORF42 | 26421 | 26660 | ATG | − | 240 | Hypothetical protein | Acinetobacter phage | 5E−44 | 96 | ADO14411.1 |
| | | | | | | | AB1 | | | |
| ORF43 | 26801 | 27088 | ATG | − | 288 | Hypothetical protein | Acinetobacter phage | 2E−59 | 96 | ADO14413.1 |
| | | | | | | | AB1 | | | |
| ORF44 | 27069 | 27329 | ATG | − | 261 | Hypothetical protein | Acinetobacter phage | 3E−56 | 100 | YP_009055433.1 |
| | | | | | | | YMC-13-01-C62 | | | |
| ORF45 | 27326 | 27712 | ATG | − | 387 | Hypothetical protein | Acinetobacter phage | 1E−21 | 42 | ADO14414.1 |
| | | | | | | | AB1 | | | |
| ORF46 | 27699 | 28280 | ATG | − | 582 | Hypothetical protein | Acinetobacter phage | 5E−141 | 100 | YP_009055435.1 |
| | | | | | | | YMC-13-01-C62 | | | |
| ORF47 | 28277 | 28441 | ATG | − | 165 | Hypothetical protein | Acinetobacter phage | 2E−24 | 89 | ADO14416.1 |
| | | | | | | | AB1 | | | |
| ORF48 | 28438 | 29013 | ATG | − | 576 | Hypothetical protein | Acinetobacter phage | 1E−137 | 98 | ADO14417.1 |
| | | | | | | | AB1 | | | |
| ORF49 | 29010 | 29777 | ATG | − | 768 | Hypothetical protein | Acinetobacter phage | 2E−134 | 79 | ADO14418.1 |
| | | | | | | | AB1 | | | |
| ORF50 | 29765 | 29878 | ATG | − | 114 | Hypothetical protein | Acinetobacter phage | 2E−16 | 92 | ADO14419.1 |
| | | | | | | | AB1 | | | |
| ORF51 | 29875 | 30087 | ATG | − | 213 | Putative | Acinetobacter phage | 2E−41 | 99 | AFV51531.1 |
| | | | | | | bacteriophage- | IME-AB2 | | | |
| | | | | | | associated | | | | |
| | | | | | | immunity protein | | | | |
| ORF52 | 30159 | 30308 | ATG | − | 150 | Hypothetical protein | Acinetobacter phage | 0.32 | 41 | YP_009055440.1 |
| | | | | | | | YMC-13-01-C62 | | | |
| ORF53 | 30305 | 30598 | ATG | − | 294 | Hypothetical protein | Acinetobacter phage | 3E−58 | 91 | ADO14421.1 |
| | | | | | | | AB1 | | | |
| ORF54 | 30595 | 30864 | ATG | − | 270 | Hypothetical protein | Acinetobacter phage | 4E−35 | 63 | ADO14422.1 |
| | | | | | | | AB1 | | | |
| ORF55 | 30875 | 32218 | ATG | − | 1344 | Putative replicative | Acinetobacter phage | 0 | 99 | YP_009055443.1 |
| | | | | | | DNA helicase | YMC-13-01-C62 | | | |
| ORF56 | 32224 | 33090 | ATG | − | 867 | Putative primosomal | Acinetobacter phage | 0 | 99 | AFV51535.1 |
| | | | | | | protein | IME-AB2 | | | |
| ORF57 | 33083 | 33562 | ATG | − | 480 | Hypothetical protein | Acinetobacter phage | 2E−113 | 100 | YP_009055445.1 |
| | | | | | | | YMC-13-01-C62 | | | |
| ORF58 | 33575 | 33787 | ATG | − | 213 | Hypothetical protein | Acinetobacter phage | 2E−38 | 87 | ADO14425.1 |
| | | | | | | | AB1 | | | |
| ORF59 | 33802 | 34137 | ATG | − | 336 | Hypothetical protein | Acinetobacter phage | 8E−76 | 100 | YP_009055447.1 |
| | | | | | | | YMC-13-01-C62 | | | |
| ORF60 | 34321 | 34509 | ATG | − | 189 | Hypothetical protein | Acinetobacter phage | 1E−21 | 86 | ADO14428.1 |
| | | | | | | | AB1 | | | |
| ORF61 | 34703 | 35290 | ATG | + | 588 | Putative HNH homing | Acinetobacter phage | 3E−61 | 50 | ASJ78942.1 |
| | | | | | | endonuclease | AbP2 | | | |
| ORF62 | 35343 | 35537 | ATG | − | 195 | Hypothetical protein | Acinetobacter phage | 3E−14 | 52 | ADO14431.1 |
| | | | | | | | AB1 | | | |
| ORF63 | 35637 | 36449 | ATG | + | 813 | Putative | Acinetobacter phage | 0 | 100 | YP_009055451.1 |
| | | | | | | transcriptional | YMC-13-01-C62 | | | |
| | | | | | | regulator | | | | |
| ORF64 | 36504 | 36773 | ATG | + | 270 | Hypothetical protein | Acinetobacter phage | 6E−47 | 88 | ADO14434.1 |
| | | | | | | | AB1 | | | |
| ORF65 | 36866 | 37198 | ATG | + | 333 | Hypothetical protein | Acinetobacter phage | 1E−68 | 94 | ADO14435.1 |
| | | | | | | | AB1 | | | |
| ORF66 | 37198 | 37380 | ATG | + | 183 | Hypothetical protein | Acinetobacter phage | 2E−36 | 100 | YP_009055454.1 |
| | | | | | | | YMC-13-01-C62 | | | |
| ORF67 | 37377 | 38276 | ATG | + | 900 | Hypothetical protein | Psychrobacter phage | 1E−70 | 43 | YP_007673324.1 |
| | | | | | | | pOW20-A | | | |
| ORF68 | 38273 | 39028 | ATG | + | 756 | Hypothetical protein | Acinetobacter phage | 2E−169 | 97 | ADO14438.1 |
| | | | | | | | AB1 | | | |
| ORF69 | 39029 | 39322 | ATG | + | 294 | Hypothetical protein | Acinetobacter phage | 7E−61 | 96 | ADO14439.1 |
| | | | | | | | AB1 | | | |
| ORF70 | 39319 | 39540 | ATG | + | 222 | Hypothetical protein | Acinetobacter phage | 2E−07 | 43 | YP_009055458.1 |
| | | | | | | | YMC-13-01-C62 | | | |
| ORF71 | 39537 | 39698 | ATG | + | 162 | Hypothetical protein | Acinetobacter phage | 3E−29 | 96 | ADO14441.1 |
| | | | | | | | AB1 | | | |
| ORF72 | 39686 | 40258 | ATG | + | 573 | Putative nucleoside | Acinetobacter phage | 5E−71 | 64 | AFV51550.1 |
| | | | | | | triphosphate | IME-AB2 | | | |
| | | | | | | pyrophosphohydrolase | | | | |
| ORF73 | 40251 | 40481 | ATG | + | 231 | rIIB lysis inhibitor | Caulobacter phage | 1.6 | 33 | AXQ68725.1 |
| | | | | | | | CcrPW | | | |
| ORF74 | 40574 | 41182 | ATG | − | 609 | Putative endolysin | Acinetobacter phage | 5E−143 | 98 | ARB06760.1 |
| | | | | | | | WCHABP12 | | | |
| ORF75 | 41169 | 41444 | ATG | − | 276 | Hypothetical protein | Acinetobacter phage | 1E−56 | 95 | ADO14445.1 |
| | | | | | | | AB1 | | | |
| ORF76 | 41428 | 41748 | ATG | − | 321 | Hypothetical protein | Acinetobacter phage | 1E−53 | 95 | ADO14446.1 |
| | | | | | | | AB1 | | | |
| ORF77 | 41824 | 43647 | ATG | − | 1824 | Putative tail fiber | Acinetobacter phage | 2E−77 | 88 | ARQ94726.1 |
| | | | | | | protein | WCHABP1 | | | |
| ORF78 | 43649 | 44494 | GTG | − | 846 | Putative tail fiber | Acinetobacter phage | 0 | 99 | YP_009203603.1 |
| | | | | | | protein | YMC11/12/R2315 | | | |

As shown in FIG. 8 and Table 4, the bacteriophage YMC14/01/P117_ABA_BP contained linear dsDNA and was composed of 78 ORFs.

As a result of comparing the sequence of the bacteriophage YMCT4/01/P117_ABA_BP according to the present invention with sequences of the existing bacteriophages, no bacteriophage having similarity to the bacteriophage according to the present invention was detected. From the above results, it can be seen that the bacteriophage YMCT4/01/P117_ABA_BP according to the present invention corresponds to a novel bacteriophage that has not been previously discovered.

### [Example 2] Bacteriophage YMC16/12/R4637 ABA_BP

1. Isolation of Clinical Specimens and Selection of Antibiotic-Resistant Strains

As shown in Table 5 below, Acinetobacter baumannii strains were isolated from blood, clinical specimens, and the like obtained from the intensive care unit (ICU) of a university hospital, and cultured. Strain identification was performed using a kit such as ATB 32 GN system (bioMérieux, Marcy l'Etoile, France). Subsequently, for antibiotic susceptibility test, a CLSI disk diffusion test method, in which culture is performed overnight at 37° C. in outside air using Mueller-Hinton agar, was used; and for test antibiotics, amikacin, ampicillin-sulbactam, ceftazidime, ciprofloxacin, colistin, cefepime, cefotaxime, gentamicin, imipenem, levofloxacin, meropenem, minocycline, piperacillin, piperacillin-tazobactam, cortrimoxa, and tigecycline were used. The susceptibility results were read based on the Clinical and Laboratory Standards Institute (CLSI, 2016). Antibiotic resistance profiles of the collected Acinetobacter baumannii strains are shown in Table 6 below. In Table 6 below, S, I, and R are the results obtained by evaluating susceptibility to the antibacterial agents, in which ‘S’ means susceptible, ‘I’ means intermediate, and ‘R’ means resistant.

TABLE 5

| Host strain | Origin of sample | Host strain | Origin of sample |
|---|---|---|---|
| YMC16/12/R12914 | Sputum (pneumonia) | YMC16/01/R198 | Sputum (pneumonia) |
| YMC16/12/B11422 | Catheter blood | YMC16/01/R353 | Tracheal aspirate |
| | | | (pneumonia) |
| YMC16/12/B11449 | Blood | YMC16/01/R405 | Sputum (pneumonia) |
| YMC16/12/B10832 | Blood | YMC16/01/R397 | Sputum (pneumonia) |
| YMC16/12/B13325 | Catheter blood | YMC16/01/R380 | Tracheal aspirate |
| | | | (pneumonia) |
| YMC17/01/P518 | Swab or drainage tube, hip | YMC16/12/R4637 | Swab or drainage tube, |
| | | | abdomen |
| YMC17/01/B8053 | Catheter blood | YMC17/01/R2812 | Tracheal tube tip |
| YMC17/01/B10087 | Catheter blood | YMC17/02/R541 | Tracheal aspirate |
| | | | (pneumonia) |
| YMC17/01/B12075 | Catheter blood | YMC17/02/R2392 | Sputum (pneumonia) |
| YMC17/02/B14 | Blood | YMC17/03/R348 | Sputum (pneumonia) |
| YMC17/01/B13454 | Blood | YMC17/03/R5305 | |
| YMC17/02/B87 | Blood | YMC17/03/R3095 | |
| YMC17/02/B721 | Blood | YMC17/03/R3428 | |
| YMC17/02/B4520 | Catheter blood | YMC17/03/R4607 | Sputum (pneumonia) |
| YMC17/02/B4039 | Blood | YMC17/03/P971 | Swab or drainage tube, hip |
| YMC17/02/B4864 | Blood | YMC16/03/R4461 | Tracheal aspirate |
| | | | (pneumonia) |
| YMC17/02/P523 | Decubitus ulcer | YMC16/05/R2210 | Sputum (pneumonia) |
| YMC17/02/B8414 | Peritoneal-blood bottle | YMC16/07/R2512 | Bronchoalveolar lavage |
| YMC17/03/R585 | Sputum (pneumonia) | YMC16/09/R2471 | Tracheal aspirate |
| | | | (pneumonia) |
| YMC17/03/B4730 | Catheter blood | YMC16/10/R2537 | Sputum (pneumonia) |
| YMC17/03/B5000 | Catheter blood | YMC16/12/P503 | Swab or drainage tube, |
| | | | chest |
| YMC17/03/R1888 | Sputum (pneumonia) | YMC15/02/T28 | Another catheter tip |
| YMC17/03/R3279 | Sputum (pneumonia) | YMC15/02/R436 | Tracheal aspirate |
| | | | (pneumonia) |
| YMC17/03/R4077 | Tracheal aspirate | YMC15/03/R1604 | Tracheal aspirate |
| | (pneumonia) | | (pneumonia) |
| YMC17/04/R488 | Sputum (pneumonia) | YMC15/09/R1869 | Sputum (pneumonia) |
| YMC17/04/R640 | Sputum (pneumonia) | YMC14/06/R2359 | Sputum (pneumonia) |
| YMC/17/05/R1095 | Tracheal aspirate | YMC14/08/T90 | Another catheter tip |
| | (pneumonia) | | |
| YMC16/01/P11 | Swab or drainage tube, hip | YMC14/08/R1169 | Sputum (pneumonia) |
| YMC16/01/R123 | Tracheal tube tip | | |

TABLE 6

| Host | | Ampicillin- | | | | | | | |
|---|---|---|---|---|---|---|---|---|---|
| strain | Amikacin | sulbactam | Ceftazidime | Ciprofloxacin | Colistin | Cefepime | Cefotaxime | Gentamicin | Imipenem |
| YMC16/12/ | | | | | | | | | |
| R12914 | | | | | | | | | |
| YMC16/12/ | 6 R | =32 R | =64 R | =4 R | =0.5 S | =64 R | =64 R | =16 R | =16 R |
| B11422 | | | | | | | | | |
| YMC16/12/ | 6 R | =32 R | =64 R | =4 R | =0.5 S | =64 R | =64 R | =16 R | =16 R |
| B11449 | | | | | | | | | |
| YMC16/12/ | | | | | | | | | |
| B10832 | | | | | | | | | |
| YMC16/12/ | 6 R | 16 I | =64 R | =4 R | =0.5 S | =64 R | =64 R | =16 R | =16 R |
| B13325 | | | | | | | | | |
| YMC17/01/ | 6 R | 16 I | =64 R | =4 R | =0.5 S | =64 R | =64 R | =16 R | =16 R |
| P518 | | | | | | | | | |
| YMC17/01/ | 6 R | 8 S | =64 R | =4 R | =0.5 S | =64 R | =64 R | =16 R | =16 R |
| B8053 | | | | | | | | | |
| YMC17/01/ | 6 R | 16 I | =64 R | =4 R | =0.5 S | =64 R | =64 R | =16 R | =16 R |
| B10087 | | | | | | | | | |
| YMC17/01/ | 22 S | 16 I | =64 R | =4 R | 22 S | =64 R | =64 R | =1 S | =16 R |
| B12075 | | | | | | | | | |
| YMC17/02/ | 6 R | =32 R | =64 R | =4 R | =0.5 S | =64 R | =64 R | =16 R | =16 R |
| B14 | | | | | | | | | |
| YMC17/01/ | 6 R | =32 R | =64 R | =4 R | =0.5 S | =64 R | =64 R | =16 R | =16 R |
| B13454 | | | | | | | | | |
| YMC17/02/ | 20 S | 16 I | =64 R | =4 R | =0.5 S | =64 R | =64 R | 2 S | =16 R |
| B87 | | | | | | | | | |
| YMC17/02/ | 6 R | =32 R | =64 R | =4 R | =0.5 S | =64 R | =64 R | =16 R | =16 R |
| B721 | | | | | | | | | |
| YMC17/02/ | 6 R | =32 R | =64 R | =4 R | =0.5 S | =64 R | =64 R | =16 R | =16 R |
| B4520 | | | | | | | | | |
| YMC17/02/ | 6 R | =32 R | =64 R | =4 R | =0.5 S | =64 R | =64 R | =16 R | =16 R |
| B4039 | | | | | | | | | |
| YMC17/02/ | 25 S | =2 S | 4 S | =0.25 S | =0.5 S | 2 S | 8 S | =1 S | =0.25 S |
| B4864 | | | | | | | | | |
| YMC17/02/ | 21 S | 16 I | =64 R | =4 R | =0.5 S | =64 R | =64 R | 4 S | =16 R |
| P523 | | | | | | | | | |
| YMC17/02/ | 6 R | 16 I | =64 R | =4 R | =0.5 S | =64 R | =64 R | =16 R | =16 R |
| B8414 | | | | | | | | | |
| YMC17/03/ | 6 R | =32 R | =64 R | =4 R | =16 R | =64 R | =64 R | =16 R | =16 R |
| R585 | | | | | | | | | |
| YMC17/03/ | 6 R | 16 I | =64 R | =4 R | =0.5 S | =64 R | =64 R | =16 R | =16 R |
| B4730 | | | | | | | | | |
| YMC17/03/ | 6 R | =32 R | =64 R | =4 R | =0.5 S | =64 R | =64 R | =16 R | =16 R |
| B5000 | | | | | | | | | |
| YMC17/03/ | 6 R | 8 S | =64 R | =4 R | =16 R | =64 R | =64 R | =16 R | =16 R |
| R1888 | | | | | | | | | |
| YMC17/03/ | 6 R | 16 I | =64 R | =4 R | =16 R | =64 R | =64 R | =16 R | =16 R |
| R3279 | | | | | | | | | |
| YMC17/03/ | 6 R | 16 I | =64 R | =4 R | =0.5 S | =64 R | =64 R | =16 R | =16 R |
| R4077 | | | | | | | | | |
| YMC17/04/ | 6 R | 16 I | =64 R | =4 R | 8 R | =64 R | =64 R | =16 R | =16 R |
| R488 | | | | | | | | | |
| YMC17/04/ | 6 R | 16 I | =64 R | =4 R | 8 R | =64 R | =64 R | =16 R | =16 R |
| R640 | | | | | | | | | |
| YMC/17/05/ | 6 R | 16 I | =64 R | =4 R | =16 R | =64 R | =64 R | =16 R | =16 R |
| R1095 | | | | | | | | | |
| YMC16/01/ | 6 R | =32 R | =64 R | =4 R | =0.5 S | =64 R | =64 R | =16 R | =16 R |
| P11 | | | | | | | | | |
| YMC16/01/ | 6 R | =32 R | =64 R | =4 R | =0.5 S | =64 R | =64 R | =16 R | =16 R |
| R123 | | | | | | | | | |
| YMC16/01/ | 6 R | 8 S | =64 R | =4 R | =0.5 S | =64 R | =64 R | =16 R | =16 R |
| R198 | | | | | | | | | |
| YMC16/01/ | 6 R | 16 I | =64 R | =4 R | =0.5 S | =64 R | =64 R | =16 R | =16 R |
| R353 | | | | | | | | | |
| YMC16/01/ | 6 R | 16 I | =64 R | =4 R | =0.5 S | =64 R | =64 R | =16 R | =16 R |
| R405 | | | | | | | | | |
| YMC16/01/ | 6 R | =32 R | =64 R | =4 R | =0.5 S | =64 R | =64 R | =16 R | =16 R |
| R397 | | | | | | | | | |
| YMC16/01/ | | =32 R | =64 R | =4 R | =0.5 S | =64 R | =64 R | =16 R | =16 R |
| R380 | | | | | | | | | |
| YMC16/12/ | 6 R | =32 R | =64 R | =4 R | =16 R | =64 R | =64 R | =16 R | =16 R |
| R4637 | | | | | | | | | |
| YMC17/01/ | 6 R | =32 R | =64 R | =4 R | =16 R | =64 R | =64 R | =16 R | =16 R |
| R2812 | | | | | | | | | |
| YMC17/02/ | 6 R | =32 R | =64 R | =4 R | =16 R | =64 R | =64 R | =16 R | =16 R |
| R541 | | | | | | | | | |
| YMC17/02/ | 6 R | =32 R | =64 R | =4 R | 4 R | =64 R | =64 R | =16 R | =16 R |
| R2392 | | | | | | | | | |
| YMC17/03/ | 6 R | 16 I | 32 R | =4 R | 4 R | =64 R | =64 R | =16 R | =16 R |
| R348 | | | | | | | | | |
| YMC17/03/ | | | | | | | | | |
| R5305 | | | | | | | | | |
| YMC17/03/ | | | | | | | | | |
| R3095 | | | | | | | | | |
| YMC17/03/ | | | | | | | | | |
| R3428 | | | | | | | | | |
| YMC17/03/ | 6 R | 16 I | =64 R | =4 R | =0.5 S | =64 R | =64 R | =16 R | =16 R |
| R4607 | | | | | | | | | |
| YMC17/03/ | 6 R | 8 S | =64 R | =4 R | =16 R | =64 R | =64 R | =16 R | =16 R |
| P971 | | | | | | | | | |
| YMC16/03/ | 6 R | 4 S | =64 R | =4 R | =16 R | =64 R | 32 I | =16 R | =16 R |
| R4461 | | | | | | | | | |
| YMC16/05/ | | 16 I | =64 R | =4 R | =16 R | =64 R | =64 R | =16 R | =16 R |
| R2210 | | | | | | | | | |
| YMC16/07/ | 6 R | 16 I | =64 R | =4 R | =16 R | =64 R | =64 R | =16 R | =16 R |
| R2512 | | | | | | | | | |
| YMC16/09/ | 6 R | =32 R | =64 R | =4 R | =16 R | =64 R | =64 R | =16 R | =16 R |
| R2471 | | | | | | | | | |
| YMC16/10/ | 6 R | 16 I | =64 R | =4 R | =16 R | =64 R | =64 R | =16 R | =16 R |
| R2537 | | | | | | | | | |
| YMC16/12/ | 6 R | =32 R | =64 R | =4 R | =16 R | =64 R | =64 R | =16 R | =16 R |
| P503 | | | | | | | | | |
| YMC15/02/ | 6 R | 16 I | =64 R | =4 R | =16 R | 32 R | =64 R | =16 R | =16 R |
| T28 | | | | | | | | | |
| YMC15/02/ | 6 R | =32 R | =64 R | =4 R | 8 R | =64 R | =64 R | =16 R | =16 R |
| R436 | | | | | | | | | |
| YMC15/03/ | 6 R | 16 I | =64 R | =4 R | =16 R | =64 R | =64 R | =16 R | =16 R |
| R1604 | | | | | | | | | |
| YMC15/09/ | 6 R | 16 I | =64 R | =4 R | =16 R | 32 R | =64 R | =16 R | =16 R |
| R1869 | | | | | | | | | |
| YMC14/06/ | 6 R | 8 S | =64 R | =4 R | =16 R | =64 R | =64 R | =16 R | =16 R |
| R2359 | | | | | | | | | |
| YMC14/08/ | 6 R | 8 S | =64 R | =4 R | =16 R | =64 R | =64 R | =16 R | =16 R |
| T90 | | | | | | | | | |
| YMC14/08/ | 6 R | 16 I | =64 R | =4 R | =16 R | =64 R | =64 R | =16 R | =16 R |
| R1169 | | | | | | | | | |

| Host | | | | | Piperacillin- | | |
|---|---|---|---|---|---|---|---|
| strain | Levofloxacin | Meropenem | Minocycline | Piperacillin | tazobactam | Cortrimoxa | Tigecycline |
| YMC16/12/ | | | | | | | |
| R12914 | | | | | | | |
| YMC16/12/ | =8 R | =16 R | =1 S | =128 R | =128 R | 160 R | 1 S |
| B11422 | | | | | | | |
| YMC16/12/ | =8 R | =16 R | =1 S | =128 R | =128 R | =320 R | 2 S |
| B11449 | | | | | | | |
| YMC16/12/ | | | | | | | |
| B10832 | | | | | | | |
| YMC16/12/ | =8 R | =16 R | =1 S | =128 R | =128 R | =320 R | 1 S |
| B13325 | | | | | | | |
| YMC17/01/ | =8 R | =16 R | =1 S | =128 R | =128 R | 160 R | 2 S |
| P518 | | | | | | | |
| YMC17/01/ | =8 R | =16 R | =1 S | =128 R | =128 R | =320 R | =8 R |
| B8053 | | | | | | | |
| YMC17/01/ | =8 R | =16 R | =1 S | =128 R | =128 R | 160 R | 2 S |
| B10087 | | | | | | | |
| YMC17/01/ | =8 R | =16 R | =1 S | =128 R | =128 R | =20 S | 2 S |
| B12075 | | | | | | | |
| YMC17/02/ | =8 R | =16 R | 8 I | =128 R | =128 R | =320 R | 2 S |
| B14 | | | | | | | |
| YMC17/01/ | =8 R | =16 R | =1 S | =128 R | =128 R | 160 R | 2 S |
| B13454 | | | | | | | |
| YMC17/02/ | =8 R | =16 R | 8 I | =128 R | =128 R | =320 R | 2 S |
| B87 | | | | | | | |
| YMC17/02/ | =8 R | =16 R | =1 S | =128 R | =128 R | =320 R | 2 S |
| B721 | | | | | | | |
| YMC17/02/ | =8 R | =16 R | =1 S | =128 R | =128 R | 160 R | 1 S |
| B4520 | | | | | | | |
| YMC17/02/ | =8 R | =16 R | =1 S | =128 R | =128 R | 80 R | =0.5 S |
| B4039 | | | | | | | |
| YMC17/02/ | =0.12 S | =0.25 S | =1 S | 8 S | =4 S | =20 S | =0.5 S |
| B4864 | | | | | | | |
| YMC17/02/ | =8 R | =16 R | =1 S | =128 R | =128 R | =20 S | 1 S |
| P523 | | | | | | | |
| YMC17/02/ | =8 R | =16 R | 2 S | =128 R | =128 R | =320 R | =8 R |
| B8414 | | | | | | | |
| YMC17/03/ | =8 R | =16 R | =16 R | =128 R | =128 R | =320 R | 4 I |
| R585 | | | | | | | |
| YMC17/03/ | =8 R | =16 R | =1 S | =128 R | =128 R | =320 R | =8 R |
| B4730 | | | | | | | |
| YMC17/03/ | =8 R | =16 R | 8 I | =128 R | =128 R | =320 R | 1 S |
| B5000 | | | | | | | |
| YMC17/03/ | =8 R | =16 R | =1 S | =128 R | =128 R | =320 R | =8 R |
| R1888 | | | | | | | |
| YMC17/03/ | =8 R | =16 R | =1 S | =128 R | =128 R | =320 R | 1 S |
| R3279 | | | | | | | |
| YMC17/03/ | =8 R | =16 R | 2 S | =128 R | =128 R | =320 R | =8 R |
| R4077 | | | | | | | |
| YMC17/04/ | =8 R | =16 R | =1 S | =128 R | =128 R | 160 R | 2 S |
| R488 | | | | | | | |
| YMC17/04/ | =8 R | =16 R | =1 S | =128 R | =128 R | =320 R | =8 R |
| R640 | | | | | | | |
| YMC/17/05/ | =8 R | =16 R | 2 S | =128 R | =128 R | =320 R | 4 I |
| R1095 | | | | | | | |
| YMC16/01/ | =8 R | =16 R | =1 S | =128 R | =128 R | =320 R | 2 S |
| P11 | | | | | | | |
| YMC16/01/ | =8 R | =16 R | =1 S | =128 R | =128 R | 160 R | 2 S |
| R123 | | | | | | | |
| YMC16/01/ | =8 R | =16 R | =1 S | =128 R | =128 R | 160 R | 2 S |
| R198 | | | | | | | |
| YMC16/01/ | =8 R | =16 R | =1 S | =128 R | =128 R | 160 R | 2 S |
| R353 | | | | | | | |
| YMC16/01/ | =8 R | =16 R | =1 S | =128 R | =128 R | =320 R | =8 R |
| R405 | | | | | | | |
| YMC16/01/ | =8 R | =16 R | =1 S | =128 R | =128 R | 160 R | 1 S |
| R397 | | | | | | | |
| YMC16/01/ | =8 R | =16 R | 4 S | =128 R | =128 R | =20 S | 2 S |
| R380 | | | | | | | |
| YMC16/12/ | =8 R | =16 R | =16 R | =128 R | =128 R | =320 R | 4 I |
| R4637 | | | | | | | |
| YMC17/01/ | =8 R | =16 R | =16 R | =128 R | =128 R | =320 R | 4 I |
| R2812 | | | | | | | |
| YMC17/02/ | =8 R | =16 R | =16 R | =128 R | =128 R | =320 R | 4 I |
| R541 | | | | | | | |
| YMC17/02/ | =8 R | =16 R | =1 S | =128 R | =128 R | =320 R | 2 S |
| R2392 | | | | | | | |
| YMC17/03/ | =8 R | =16 R | =1 S | =128 R | =128 R | =320 R | =0.5 S |
| R348 | | | | | | | |
| YMC17/03/ | | | | | | | |
| R5305 | | | | | | | |
| YMC17/03/ | | | | | | | |
| R3095 | | | | | | | |
| YMC17/03/ | | | | | | | |
| R3428 | | | | | | | |
| YMC17/03/ | =8 R | =16 R | =16 R | =128 R | =128 R | =320 R | 2 S |
| R4607 | | | | | | | |
| YMC17/03/ | =8 R | =16 R | =1 S | =128 R | =128 R | =320 R | =8 R |
| P971 | | | | | | | |
| YMC16/03/ | =8 R | =16 R | =1 S | =128 R | =128 R | =320 R | 4 I |
| R4461 | | | | | | | |
| YMC16/05/ | =8 R | =16 R | =1 S | =128 R | =128 R | =320 R | 2 S |
| R2210 | | | | | | | |
| YMC16/07/ | =8 R | =16 R | =1 S | =128 R | =128 R | =320 R | 2 S |
| R2512 | | | | | | | |
| YMC16/09/ | =8 R | =16 R | =1 S | =128 R | =128 R | 160 R | 1 S |
| R2471 | | | | | | | |
| YMC16/10/ | =8 R | =16 R | =1 S | =128 R | =128 R | 160 R | 1 S |
| R2537 | | | | | | | |
| YMC16/12/ | =8 R | =16 R | =16 R | =128 R | =128 R | =320 R | 2 S |
| P503 | | | | | | | |
| YMC15/02/ | =8 R | =16 R | =1 S | =128 R | =128 R | =320 R | 2 S |
| T28 | | | | | | | |
| YMC15/02/ | =8 R | =16 R | =16 R | =128 R | =128 R | =320 R | 4 I |
| R436 | | | | | | | |
| YMC15/03/ | =8 R | =16 R | =1 S | =128 R | =128 R | =320 R | 2 S |
| R1604 | | | | | | | |
| YMC15/09/ | =8 R | =16 R | =1 S | =128 R | =128 R | =320 R | 4 I |
| R1869 | | | | | | | |
| YMC14/06/ | =8 R | =16 R | =1 S | =128 R | =128 R | =320 R | 2 S |
| R2359 | | | | | | | |
| YMC14/08/ | =8 R | =16 R | 2 S | =128 R | =128 R | =320 R | 2 S |
| T90 | | | | | | | |
| YMC14/08/ | =8 R | =16 R | =1 S | =128 R | =128 R | =320 R | =8 R |
| R1169 | | | | | | | |

As shown in Table 6, the collected 57 Acinetobacter baumannii strains were found to be multi-drug-resistant strains having resistance to various antibiotics.

2. Collection of Bacteriophage Specimens

2-1. Collection of Specimens to Construct Phage Bank

Raw water was obtained by causing sewage to pass through a first sedimentation tank at the sewage treatment facility of the Severance Hospital (Korea), and then removing suspended substances and sediments therefrom. The sewage was limited to sewage that was present at a preliminary stage of a chemical treatment facility. To the collected sample was added 58 g of sodium chloride per L. Then, centrifugation was performed at 10,000 g for 10 minutes and filtration was performed through a 220 nm Millipore filter. To the obtained filtrate was added polyethylene glycol (PEG, molecular weight of 8000) at 10% w/v, and the resultant was stored refrigerated at 4° C. for 12 hours. The filtrate stored refrigerated for 12 hours was centrifuged at 12,000 g for 20 minutes, and the precipitate was resuspended in phage dilution buffer (SM buffer). To the resuspension was then added the same amount of chloroform, and the resultant was stored frozen. This was repeated three times to collect 300 mL of bacteriophage suspension.

2-2. Selection of Lytic Phage and Measurement of Lysis Titer

Separation and purification of lytic phage were performed by a spot test method (Mazzocco A et al. In Bacteriophages, Clokie and Kropinski AM, eds. Humana Press. 2009). The obtained strains were inoculated on MacConkey Agar medium and then cultured overnight at 35° C. in outside air. After the culture, strains susceptible to phage were selected by observing formation of clear plaque. The susceptible strains were inoculated on MacConkey Agar medium and cultured at 35° C. for 12 hours. A suspension of each strain was prepared in a 1 ml saline tube with a turbidity of 0.5 McFarland, and mixed with H top agar (3 ml), 100 μl of sensitive bacteria, and a phage solution (each of 1 μl, 10 μl, and 50 μl). The mixture was applied to LB agar, and then cultured at 35° C. for 12 hours. Plaque was observed, and then the plaque was collected with a Pasteur pipette. The collected plaque was diluted in SM buffer solution, and repeatedly purified three times using the susceptible strain suspension again. The thus obtained pure bacteriophage, YMC16/12/R4637_ABA_BP, was diluted in SM buffer solution, and repeatedly purified three times using the susceptible strain suspension again. The thus obtained pure bacteriophage, YMC16/12/R4637_ABA_BP, was diluted in SM buffer solution and stored.

Each of the antibiotic-resistant Acinetobacter baumannii strains identified in item no. 1. above was inoculated on MacConkey Agar medium and cultured. Then, the bacteriophage YMC16/12/R4637_ABA_BP, which had been purified by the above process, was inoculated in an amount of 5 μl into each smeared resistant strain. Then, plaque formation was checked and a titer range thereof was checked. The lysis of each strain is shown in Table 7 below.

In Table 7 below, an evaluation result of plaque activity against the collected strains is indicated by + and −, in which ‘+’ means clear plaque and ‘-’ means that lysis has not occurred.

TABLE 7

| Host strain | Lysis | Host strain | Lysis |
|---|---|---|---|
| YMC16/12/B13325 | + | YMC16/01/R397 | + |
| YMC17/01/P518 | + | YMC17/03/R348 | + |
| YMC17/01/B8053 | + | YMC17/03/R3095 | + |
| YMC17/01/B10087 | + | YMC17/03/R3428 | ++ |
| YMC17/01/B12075 | + | YMC17/03/P971 | + |
| YMC17/02/B4520 | + | YMC16/03/R4461 | ++ |
| YMC17/02/P523 | + | YMC16/05/R2210 | ++ |
| YMC17/02/B8414 | + | YMC16/07/R2512 | ++ |
| YMC17/03/R1888 | + | YMC16/09/R2471 | ++ |
| YMC17/03/R3279 | + | YMC16/10/R2537 | + |
| YMC17/03/R4077 | + | YMC15/02/T28 | + |
| YMC17/04/R640 | + | YMC15/03/R1604 | ++ |
| YMC/17/05/R1095 | + | YMC15/09/R1869 | + |
| YMC16/01/P11 | + | YMC14/06/R2359 | + |
| YMC16/01/R123 | + | YMC14/08/T90 | ++ |
| YMC16/01/R198 | + | YMC14/08/R1169 | + |
| YMC16/01/R353 | + | | |

As shown in Table 7, it was found that the bacteriophage YMC16/12/R4637_ABA_BP according to the present invention lyses antibiotic-resistant Acinetobacter baumannii strains.

3. Electron Microscopic Analysis of Lytic Bacteriophage Against Antibiotic-Resistant Acinetobacter baumannii Strains

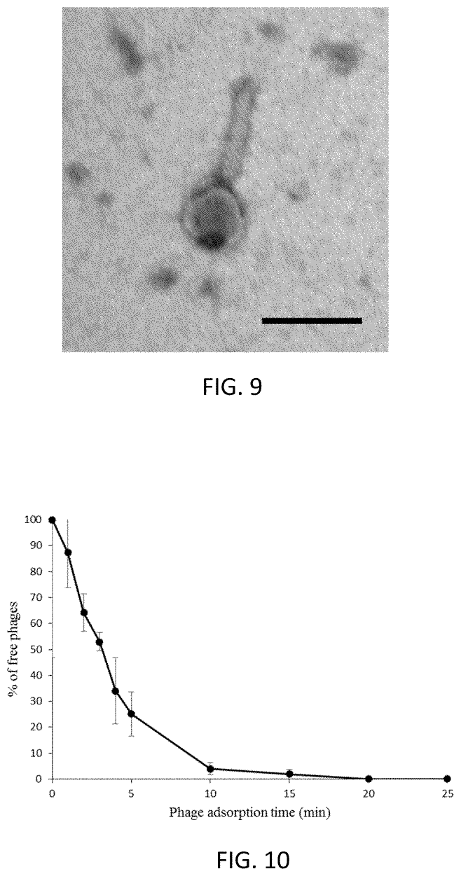

The bacteriophage YMC16/12/R4637_ABA_BP purified by the method of item no. 2. above was inoculated and cultured in culture medium (20 ml of LB medium) for susceptible strains, and then filtered through a 220 nm Millipore filter. To the supernatant was added polyethylene glycol (MW 8,000) in an amount of 10% (w/v), and then the resultant was stored refrigerated overnight. Subsequently, centrifugation was performed for 20 minutes at 12,000 g, and then a shape of the bacteriophage YMC16/12/R4637_ABA_BP was analyzed using an energy-filtering transmission electron microscope. The result is illustrated in FIG. 9.

As illustrated in FIG. 9, in a case where classification is made on a shape basis, the bacteriophage YMC16/12/R4637_ABA_BP according to the present invention was classified as belonging to the family Myoviridae that has a long tail with a hexagonal head.

4. Analysis of Adsorption Capacity and One-Step Growth Curve of Bacteriophage

The antibiotic-resistant Acinetobacter baumannii strain was cultured to an OD value of 0.5. To the Acinetobacter baumannii strain was then added the bacteriophage YMC16/12/R4637_ABA_BP purified in item no. 2. above at an MOI of 0.001 and culture was performed at room temperature. Then, sample was collected 1 ml each at 1, 2, 3, 4, and 5 minutes, diluted in LB medium, and then adsorption capacity of the bacteriophage was evaluated through plaque analysis. The results are illustrated in FIG. 10.

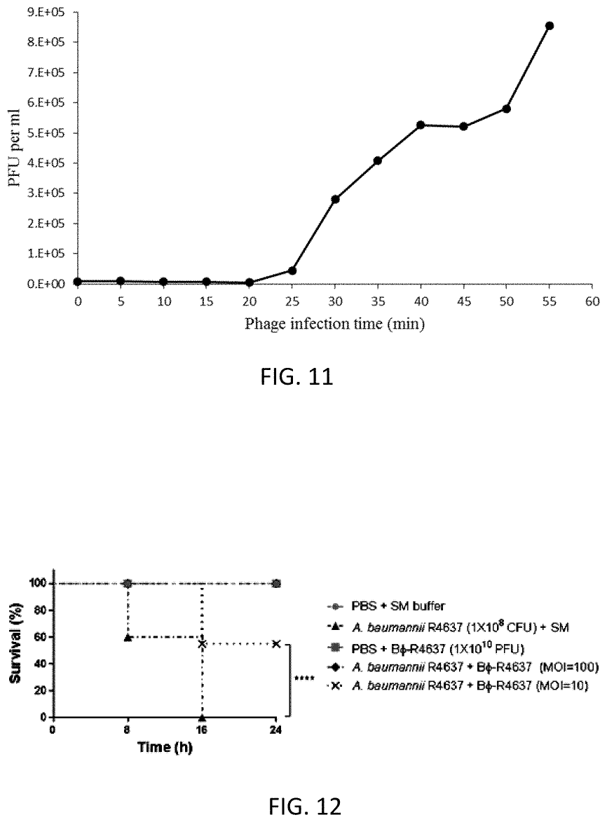

In addition, the antibiotic-resistant Acinetobacter baumannii strain was cultured to an OD value of 0.3, and then centrifuged at 7,000 g for 5 minutes at 4° C., to precipitate the cells. Then, the cells were diluted in 0.5 ml of LB medium. To the dilute was added the bacteriophage YMC16/12/R4637_ABA_BP purified in item no. 2. above at an MOI of 0.001 (titer of 10′ pfu/cell), and culture was performed at 37° C. for 5 minutes. The cultured mixed sample was centrifuged at 13,000 g for 1 minute to obtain a pellet. The obtained pellet was diluted in 10 ml of LB medium and cultured at 37° C. Samples were collected every 10 minutes during the culture, and a one-step growth curve of the bacteriophage was evaluated through plaque analysis. The results are illustrated in FIG. 11.

As illustrated in FIG. 10, about 95% of the bacteriophage YMC16/12/R4637_ABA_BP was adsorbed to the Acinetobacter baumannii strain within 10 minutes after inoculation of the bacteriophage.

In addition, as illustrated in FIG. 11, the one-step growth curve showed a high burst size of approximately 106 PFU/infected cells.

From the above results, it can be seen that the bacteriophage YMC16/12/R4637_ABA_BP according to the present invention can be adsorbed in a relatively short time to an antibiotic-resistant Acinetobacter baumannii strain and can show a high burst size of 106 PFU/infected cells, indicating that this bacteriophage exerts a lytic effect on an antibiotic-resistant strain.

5. Verification of In Vivo Lysis Ability of Bacteriophage Against Antibiotic-Resistant Acinetobacter Genus Bacteria

200 third- to fourth-instar Galleria mellonella larvae were prepared, and then divided into groups, each containing 10 larvae. Each larva was injected through its proleg with a carbapenem-resistant Acinetobacter baumannii strain at a minimum lethal dose (MLD), and then subjected to mixed inoculation with the bacteriophage YMC16/12/R4637_ABA_BP purified in item no. 2. above at an MOI of 10 or an MOI of 100. Then, survival of the larvae was checked every 12 or 24 hours until 72 hours, and the results are illustrated in FIG. 12.

As illustrated in FIG. 12, it was found that in a case where the larvae injected with the carbapenem-resistant Acinetobacter baumannii strain are treated with the bacteriophage YMC16/12/R4637_ABA_BP according to the present invention, survival of the larvae increases, in which the survival of the larvae further increases as the MOI value increases. In addition, it was found that even in a case where the larvae are injected with only the bacteriophage YMC16/12/R4637_ABA_BP without injection of the carbapenem-resistant Acinetobacter baumannii strain, no toxicity is seen when survival thereof is compared with that of a healthy control group.

From the above results, it can be seen that the bacteriophage YMC16/12/R4637_ABA_BP according to the present invention also has lytic properties in vivo against an antibiotic-resistant Acinetobacter baumannii strain, and thus can effectively prevent, ameliorate, or treat an infectious disease caused by the Acinetobacter baumannii strain.

6. Evaluation of Stability of Bacteriophage Against Antibiotic-Resistant Acinetobacter Baumannii Strain

It was identified whether the bacteriophage YMC16/12/R4637_ABA_BP according to the present invention maintains stability without being destroyed under alkaline and temperature conditions.

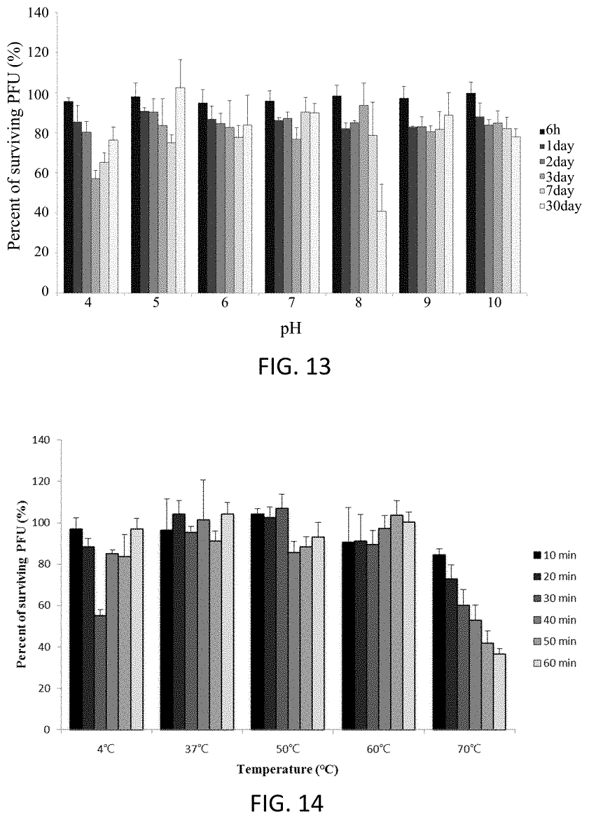

1 μl of the bacteriophage YMC16/12/R4637_ABA_BP purified by the method of item no. 2 above was added to 40 μl of SM buffer, which had been adjusted to a pH of 4, 5, 6, 7, 8, 9, or 10, and then incubated at 37° C. for 1 hour. Then, plaque analysis was performed with the antibiotic-resistant Acinetobacter baumannii bacteria using the method of item no. 4 above. The results are illustrated in FIG. 13.

In addition, during 1-hour incubation of the bacteriophage YMC16/12/R4637_ABA_BP solution at 4° C., 37° C., 50° C., 60° C., and 70° C., respectively, each sample was collected every 10 minutes and plaque analysis was performed with the Acinetobacter baumannii strain using the method of item no. 4 above. The results are illustrated in FIG. 14.

As illustrated in FIG. 13, the bacteriophage YMC16/12/R4637_ABA_BP according to the present invention exhibited high stability in all conditions which are acidic, neutral, and alkaline.

In addition, as illustrated in FIG. 14, the bacteriophage YMC16/12/R4637_ABA_BP exhibited very high stability up to a temperature as high as 60° C.

7. Whole-Genome Sequencing of Bacteriophage Against Antibiotic-Resistant Acinetobacter Genus Bacteria

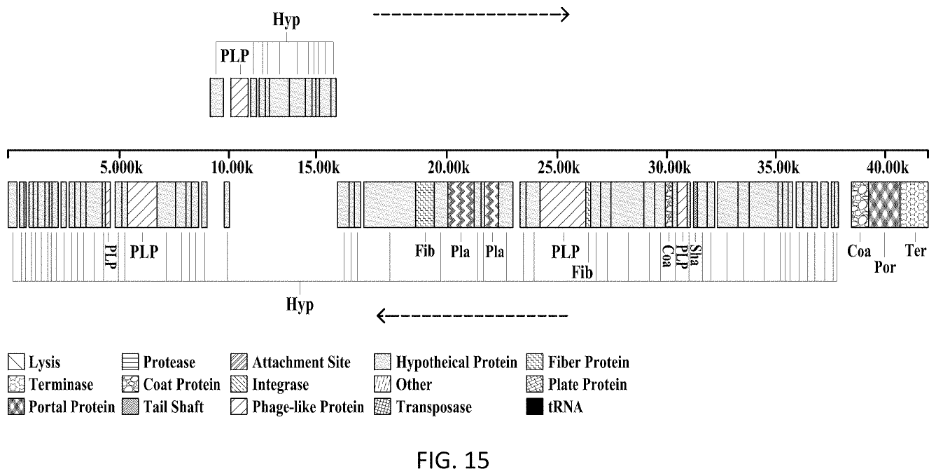

To characterize the bacteriophage YMC16/12/R4637_ABA_BP according to the present invention, whole-genome sequencing thereof was performed through the Illumina sequencer (Roche) based on a whole-genome sequencing method which is obvious to those skilled in the art. The results are shown in FIG. 15 and Table 8.

TABLE 8

| | | | | | | | | | NCBI | |
|---|---|---|---|---|---|---|---|---|---|---|
| | | | Initi- | | | | | | blastP | NCBI-Bank |
| Genome | Range | | ation | | Length | Putative | Annotation | E- | identity | accession |
| no. | Start | End | codon | Strand | (bp) | function | source | value | (%) | number |
| ORF1 | 5 | 394 | ATG | − | 390 | Hypothetical protein | Acinetobacter phage | 3E−88 | 100 | YP_009055422.1 |
| | | | | | | | YMC-13-01-C62 | | | |
| ORF2 | 448 | 630 | ATG | − | 183 | Hypothetical protein | Acinetobacter phage | 4 | 88 | ADO14405.1 |
| | | | | | | | AB1 | | | |
| ORF3 | 627 | 800 | ATG | − | 174 | | | | | |
| ORF4 | 815 | 1057 | ATG | − | 243 | Hypothetical protein | Acinetobacter phage | 1E−47 | 93 | ADO14406.1 |
| | | | | | | | AB1 | | | |
| ORF5 | 1054 | 1251 | ATG | − | 198 | Fis family | Acinetobacter phage | 3E−33 | 91 | ARB06798.1 |
| | | | | | | transcriptional | WCHABP12 | | | |
| | | | | | | regulator | | | | |
| ORF6 | 1254 | 1580 | ATG | − | 327 | Hypothetical protein | Acinetobacter phage | 4E−74 | 100 | AJT61457.1 |
| | | | | | | | YMC11/12/R1215 | | | |
| ORF7 | 1580 | 1795 | GTG | − | 216 | Hypothetical protein | Acinetobacter phage | 2E−44 | 99 | AFV51519.1 |
| | | | | | | | IME-AB2 | | | |
| ORF8 | 1907 | 2254 | ATG | − | 348 | Hypothetical protein | Acinetobacter phage | 2E−78 | 99 | YP_009055430.1 |
| | | | | | | | YMC-13-01-C62 | | | |
| ORF9 | 2318 | 2557 | ATG | − | 240 | Hypothetical protein | Acinetobacter phage | 2E−45 | 100 | YP_009055431.1 |
| | | | | | | | YMC-13-01-C62 | | | |
| ORF10 | 2698 | 2985 | GTG | − | 288 | tRNA endonuclease- | Vibrio phage | 2E−17 | 48 | AUR89331.1 |
| | | | | | | like domain protein | 1.122.A._10N.286.46.F8 | | | |
| ORF11 | 2966 | 3226 | ATG | − | 261 | Hypothetical protein | Acinetobacter phage | 3E−56 | 100 | YP_009055433.1 |
| | | | | | | | YMC-13-01-C62 | | | |
| ORF12 | 3223 | 3483 | ATG | − | 261 | Hypothetical protein | Acinetobacter phage | 6E−08 | 40 | ADO14414.1 |
| | | | | | | | AB1 | | | |
| ORF13 | 3480 | 4235 | ATG | − | 756 | Hypothetical protein | Acinetobacter phage | 2E−134 | 79 | ADO14418.1 |
| | | | | | | | AB1 | | | |
| ORF14 | 4345 | 4557 | ATG | − | 213 | Putative | Acinetobacter phage | 2E−41 | 99 | AFV51531.1 |
| | | | | | | bacteriophage- | IME-AB2 | | | |
| | | | | | | associated | | | | |
| | | | | | | immunity protein | | | | |
| ORF15 | 4629 | 4778 | ATG | − | 150 | Hypothetical protein | Acinetobacter phage | 0.32 | 41 | YP_009055440.1 |
| | | | | | | | YMC-13-01-C62 | | | |
| ORF16 | 4775 | 5068 | ATG | − | 294 | Hypothetical protein | Acinetobacter phage | 3E−58 | 91 | ADO14421.1 |
| | | | | | | | AB1 | | | |
| ORF17 | 5068 | 5334 | ATG | − | 267 | Hypothetical protein | Acinetobacter phage | 4E−35 | 63 | ADO14422.1 |
| | | | | | | | AB1 | | | |
| ORF18 | 5345 | 6688 | ATG | − | 1344 | Putative replicative | Acinetobacter phage | 0 | 99 | YP_ 009055443.1 |
| | | | | | | DNA helicase | YMC-13-01-C62 | | | |
| ORF19 | 6694 | 7560 | ATG | − | 867 | Putative primosomal | Acinetobacter phage | 0 | 98 | AFV51535.1 |
| | | | | | | protein | IME-AB2 | | | |
| ORF20 | 7553 | 8032 | ATG | − | 480 | Putative HNH | Pseudomonas phage | 9E−07 | 35 | YP_007237225.1 |
| | | | | | | endonuclease | AF | | | |
| ORF21 | 8045 | 8257 | ATG | − | 213 | Hypothetical protein | Acinetobacter phage | 2E−38 | 87 | ADO14425.1 |
| | | | | | | | AB1 | | | |
| ORF22 | 8272 | 8607 | ATG | − | 336 | Hypothetical protein | Acinetobacter phage | 8E−76 | 100 | YP_009055447.1 |
| | | | | | | | YMC-13-01-C62 | | | |
| ORF23 | 8791 | 8979 | ATG | − | 189 | Hypothetical protein | Acinetobacter phage | 1E−21 | 86 | ADO14428.1 |
| | | | | | | | AB1 | | | |
| ORF24 | 9173 | 9760 | ATG | + | 588 | Putative HNH | Acinetobacter phage | 3E−61 | 50 | ASJ78942.1 |
| | | | | | | homing | AbP2 | | | |
| | | | | | | endonuclease | | | | |
| ORF25 | 9813 | 10007 | ATG | − | 195 | Hypothetical protein | Acinetobacter phage | 3E−14 | 52 | ADO14431.1 |
| | | | | | | | AB1 | | | |
| ORF26 | 10107 | 10919 | ATG | + | 813 | Putative | Acinetobacter phage | 0 | 100 | YP_009055451.1 |
| | | | | | | transcriptional | YMC-13-01-C62 | | | |
| | | | | | | regulator | | | | |
| ORF27 | 10986 | 11243 | ATG | + | 258 | Hypothetical protein | Acinetobacter phage | 6E−47 | 88 | ADO14434.1 |
| | | | | | | | AB1 | | | |
| ORF28 | 11336 | 11668 | ATG | + | 333 | Hypothetical protein | Acinetobacter phage | 1E−68 | 94 | ADO14435.1 |
| | | | | | | | AB1 | | | |
| ORF29 | 11668 | 11850 | ATG | + | 183 | Hypothetical protein | Acinetobacter phage | 2E−36 | 100 | YP_009055454.1 |
| | | | | | | | YMC-13-01-C62 | | | |
| ORF30 | 11847 | 12746 | ATG | + | 900 | Hypothetical protein | Psychrobacter phage | 8E−71 | 43 | YP_007673324.1 |
| | | | | | | | pOW20-A | | | |
| ORF31 | 12743 | 13498 | ATG | + | 756 | Hypothetical protein | Acinetobacter phage | 5E−166 | 95 | ADO14438.1 |
| | | | | | | | AB1 | | | |
| ORF32 | 13499 | 13792 | ATG | + | 294 | Hypothetical protein | Acinetobacter phage | 2E−61 | 96 | ADO14439.1 |
| | | | | | | | AB1 | | | |
| ORF33 | 13789 | 13971 | ATG | + | 183 | | | | | |
| ORF34 | 13968 | 14132 | ATG | + | 165 | Hypothetical protein | Acinetobacter phage | 1E−27 | 92 | ADO14441.1 |
| | | | | | | | AB1 | | | |
| ORF35 | 14132 | 14653 | ATG | + | 522 | Putative nucleoside | Acinetobacter phage | 1E−68 | 64 | AFV51550.1 |
| | | | | | | triphosphate | IME-AB2 | | | |
| | | | | | | pyrophospho- | | | | |
| | | | | | | hydrolase | | | | |
| ORF36 | 14646 | 14876 | ATG | + | 231 | Hypothetical protein | Acinetobacter phage | 3E−39 | 82 | ADO14443.1 |
| | | | | | | | AB1 | | | |
| ORF37 | 14975 | 15487 | ATG | − | 513 | Putative lysozyme | Acinetobacter phage | 3E−119 | 99 | AFV51552.1 |
| | | | | | | family protein | IME-AB2 | | | |
| ORF38 | 15477 | 15749 | ATG | − | 273 | Hypothetical protein | Acinetobacter phage | 1E−54 | 94 | ADO14445.1 |
| | | | | | | | AB1 | | | |
| ORF39 | 15733 | 16053 | GTG | − | 321 | Hypothetical protein | Acinetobacter phage | 1E−68 | 95 | ADO14446.1 |
| | | | | | | | AB1 | | | |
| ORF40 | 16127 | 18541 | ATG | − | 2415 | Putative tail fiber | Acinetobacter phage | 0 | 90 | YP_009146765.1 |
| | | | | | | | YMC13/03/R2096 | | | |
| ORF41 | 18543 | 19412 | GTG | − | 870 | Putative tail fiber | Acinetobacter phage | 3E−155 | 79 | ASJ78889.1 |
| | | | | | | protein | AbP2 | | | |
| ORF42 | 19390 | 20016 | ATG | − | 627 | Hypothetical protein | Acinetobacter phage | 1E−146 | 97 | ADO14449.1 |
| | | | | | | | AB1 | | | |
| ORF43 | 20016 | 21200 | ATG | − | 1185 | Putative baseplate | Acinetobacter phage | 0 | 99 | ARQ94729.1 |
| | | | | | | J-like protein | WCHABP1 | | | |
| ORF44 | 21197 | 21550 | ATG | − | 354 | Hypothetical protein | Acinetobacter phage | 1E−80 | 99 | YP_009055470.1 |
| | | | | | | | YMC-13-01-C62 | | | |
| ORF45 | 21696 | 22340 | ATG | − | 645 | Putative baseplate | Acinetobacter phage | 1E−152 | 97 | ARQ94731.1 |
| | | | | | | assembly protein | WCHABP1 | | | |
| ORF46 | 22321 | 23211 | ATG | − | 891 | Hypothetical protein | Acinetobacter phage | 0 | 94 | ADO14453.1 |
| | | | | | | | AB1 | | | |
| ORF47 | 23321 | 23596 | GTG | − | 276 | Hypothetical protein | Acinetobacter phage | 2E−59 | 100 | ASJ78898.1 |
| | | | | | | | AbP2 | | | |
| ORF48 | 23593 | 24210 | ATG | − | 618 | Hypothetical protein | Acinetobacter phage | 7E−131 | 92 | ADO14454.1 |
| | | | | | | | AB1 | | | |
| ORF49 | 24218 | 26308 | ATG | − | 2091 | Lysozyme like | Acinetobacter phage | 0 | 74 | YP 006383794.1 |
| | | | | | | domain | AP22 | | | |
| ORF50 | 26311 | 26523 | ATG | − | 213 | Putative tail-fiber | Acinetobacter phage | 6E−44 | 99 | YP_009291892.1 |
| | | | | | | protein | LZ35 | | | |
| ORF51 | 26553 | 26978 | ATG | − | 426 | Hypothetical protein | Acinetobacter phage | 1E−37 | 46 | ADO14372.1 |
| | | | | | | | AB1 | | | |
| ORF52 | 27024 | 27473 | ATG | − | 450 | Hypothetical protein | Acinetobacter phage | 3E−105 | 97 | ADO14373.1 |
| | | | | | | | AB1 | | | |
| ORF53 | 27486 | 28949 | ATG | − | 1464 | Hypothetical protein | Acinetobacter phage | 0 | 95 | ADO14374.1 |
| | | | | | | | AB1 | | | |
| ORF54 | 28939 | 29433 | ATG | − | 495 | Hypothetical protein | Acinetobacter phage | 3E−110 | 93 | ADO14375.1 |
| | | | | | | | AB1 | | | |
| ORF55 | 29430 | 29948 | ATG | − | 519 | Hypothetical protein | Acinetobacter phage | 3E−108 | 96 | ADO14377.1 |
| | | | | | | | AB1 | | | |
| ORF56 | 29978 | 30259 | ATG | − | 282 | Putative capsid | Acinetobacter phage | 3E−55 | 100 | YP_009055482.1 |
| | | | | | | protein | YMC-13-01-C62 | | | |
| ORF57 | 30307 | 30492 | ATG | − | 186 | Hypothetical protein | Acinetobacter phage | 8E−31 | 90 | ADO14379.1 |
| | | | | | | | AB1 | | | |
| ORF58 | 30489 | 30941 | ATG | − | 453 | Putative RNA | Acinetobacter phage | 5E−104 | 100 | YP_009055484.1 |
| | | | | | | polymerase | YMC-13-01-C62 | | | |
| ORF59 | 30970 | 31155 | ATG | − | 186 | Hypothetical protein | Acinetobacter phage | 2E−35 | 100 | YP_009055485.1 |
| | | | | | | | YMC-13-01-C62 | | | |
| ORF60 | 31229 | 31375 | ATG | − | 147 | Lambda family tail | Acinetobacter phage | 1E−24 | 100 | YP_009055486.1 |
| | | | | | | tape measure | YMC-13-01-C62 | | | |
| | | | | | | protein | | | | |
| ORF61 | 31421 | 31843 | ATG | − | 423 | Hypothetical protein | Acinetobacter phage | 1E−95 | 97 | ADO14383.1 |
| | | | | | | | AB1 | | | |
| ORF62 | 31843 | 32181 | ATG | − | 339 | Hypothetical protein | Acinetobacter phage | 3E−21 | 43 | ADO14384.1 |
| | | | | | | | AB1 | | | |
| ORF63 | 32261 | 33280 | ATG | − | 1020 | Hypothetical protein | Acinetobacter phage | 0 | 100 | YP_009055489.1 |
| | | | | | | | YMC-13-01-C62 | | | |
| ORF64 | 33290 | 33769 | ATG | − | 480 | Hypothetical protein | Acinetobacter phage | 1E−30 | 43 | ADO14387.1 |
| | | | | | | | AB1 | | | |
| ORF65 | 33777 | 35111 | ATG | − | 1335 | Hypothetical protein | Acinetobacter phage | 0 | 81 | ADO14388.1 |
| | | | | | | | AP22 | | | |
| ORF66 | 35111 | 35275 | ATG | − | 165 | Hypothetical protein | Acinetobacter phage | 1E−30 | 100 | YP_009055492.1 |
| | | | | | | | YMC-13-01-C62 | | | |
| ORF67 | 35325 | 35531 | ATG | − | 207 | Hypothetical protein | Acinetobacter phage | 1E−43 | 100 | YP_009055493.1 |
| | | | | | | | YMC-13-01-C62 | | | |
| ORF68 | 35521 | 35796 | ATG | − | 276 | Hypothetical protein | Acinetobacter phage | 6E−61 | 100 | YP_009055494.1 |
| | | | | | | | YMC-13-01-C62 | | | |
| ORF69 | 35895 | 36257 | ATG | − | 363 | Hypothetical protein | Acinetobacter phage | 2E−84 | 100 | YP_009055495.1 |
| | | | | | | | YMC-13-01-C62 | | | |
| ORF70 | 36254 | 36646 | ATG | − | 393 | Hypothetical protein | Acinetobacter phage | 1E−89 | 100 | AJT61472.1 |
| | | | | | | | YMC11/12/R1215 | | | |
| ORF71 | 36639 | 37061 | ATG | − | 423 | | | | | |
| ORF72 | 37051 | 37404 | ATG | − | 354 | Hypothetical protein | Acinetobacter phage | 4E−83 | 100 | YP_009055498.1 |
| | | | | | | | YMC-13-01-C62 | | | |
| ORF73 | 37486 | 37632 | ATG | − | 147 | Hypothetical protein | Acinetobacter phage | 3E−27 | 100 | YP_009055499.1 |
| | | | | | | | YMC-13-01-C62 | | | |
| ORF74 | 37633 | 37797 | ATG | − | 165 | Hypothetical protein | Acinetobacter phage | 7E−32 | 100 | YP_009055500.1 |
| | | | | | | | YMC-13-01-C62 | | | |
| ORF75 | 38487 | 39257 | ATG | − | 771 | Putative head | Acinetobacter phage | 0 | 99 | YP_009203553.1 |
| | | | | | | protein | AbP2 | | | |
| ORF76 | 39260 | 40690 | ATG | − | 1431 | Putative portal | Acinetobacter phage | 0 | 96 | ARB06806.1 |
| | | | | | | protein | WCHABP12 | | | |
| ORF77 | 40694 | 41971 | ATG | − | 1278 | Putative terminase | Acinetobacter phage | 0 | 99 | YP_009055504.1 |
| | | | | | | large subunit | YMC-13-01-C62 | | | |
| ORF78 | 41968 | 42522 | TGT | − | 555 | Coil containing | Vibrio phage | 2E−32 | 35 | AUR98010.1 |
| | | | | | | protein | 1.246.O._10N.261.54.E10 | | | |

As shown in FIG. 15 and Table 8, the bacteriophage YMC16/12/R4637_ABA_BP contained linear dsDNA and was composed of 78 ORFs.

As a result of comparing the sequence of the bacteriophage YMCT16/12/R4637_ABA_BP according to the present invention with sequences of the existing bacteriophages, no bacteriophage having similarity to the bacteriophage according to the present invention was detected. From the above results, it can be seen that the bacteriophage YMCT6/12/R4637_ABA_BP according to the present invention corresponds to a novel bacteriophage that has not been previously discovered.

### [Example 3] Bacteriophage YMC16/01/R2016 ABA_BP

1. Isolation of Clinical Specimens and Selection of Antibiotic-Resistant Strains

As shown in Table 9 below, Acinetobacter baumannii strains were isolated from blood, clinical specimens, and the like obtained from the intensive care unit (ICU) of a university hospital, and cultured. Strain identification was performed using a kit such as ATB 32 GN system (bioMérieux, Marcy l'Etoile, France). Subsequently, for antibiotic susceptibility test, a CLSI disk diffusion test method, in which culture is performed overnight at 37° C. in outside air using Mueller-Hinton agar, was used; and for test antibiotics, amikacin, ampicillin-sulbactam, ceftazidime, ciprofloxacin, colistin, cefepime, cefotaxime, gentamicin, imipenem, levofloxacin, meropenem, minocycline, piperacillin, piperacillin-tazobactam, cortrimoxa, and tigecycline were used. The susceptibility results were read based on the Clinical and Laboratory Standards Institute (CLSI, 2016). Antibiotic resistance profiles of the collected Acinetobacter baumannii strains are shown in Table 10 below. In Table 10 below, S, 1, and R are the results obtained by evaluating susceptibility to the antibacterial agents, in which ‘S’ means susceptible, TI means intermediate, and ‘R’ means resistant.

TABLE 9

| Host strain | Origin of sample | Host strain | Origin of sample |
|---|---|---|---|
| YMC16/12/R12914 | Sputum (pneumonia) | YMC16/01/R198 | Tracheal aspirate |
| | | | (pneumonia) |
| YMC16/12/B11422 | Catheter blood | YMC16/01/R353 | Sputum (pneumonia) |
| YMC16/12/B11449 | Blood | YMC16/01/R405 | Sputum (pneumonia) |
| YMC16/12/B10832 | Blood | YMC16/01/R397 | Sputum (pneumonia) |
| YMC16/12/B13325 | Catheter blood | YMC16/01/R380 | Sputum (pneumonia) |
| YMC17/01/P518 | Swab or drainage tube, hip | YMC16/12/R4637 | Sputum (pneumonia) |
| YMC17/01/B8053 | Catheter blood | YMC17/01/R2812 | Sputum (pneumonia) |
| YMC17/01/B10087 | Catheter blood | YMC17/02/R541 | Sputum (pneumonia) |
| YMC17/01/B12075 | Catheter blood | YMC17/02/R2392 | Sputum (pneumonia) |
| YMC17/02/B14 | Blood | YMC17/03/R348 | Sputum (pneumonia) |
| YMC17/01/B13454 | Blood | YMC17/03/R5305 | |
| YMC17/02/B87 | Blood | YMC17/03/R3095 | |
| YMC17/02/B721 | Blood | YMC17/03/R3428 | |
| YMC17/02/B4520 | Catheter blood | YMC17/03/R4607 | Sputum (pneumonia) |
| YMC17/02/B4039 | Blood | YMC17/03/P971 | Swab or drainage tube, hip |
| YMC17/02/B4864 | Blood | YMC16/03/R4461 | Tracheal aspirate |
| | | | (pneumonia) |
| YMC17/02/P523 | Decubitus ulcer | YMC16/05/R2210 | Sputum (pneumonia) |
| YMC17/02/B8414 | Peritoneal-blood bottle | YMC16/07/R2512 | Bronchoalveolar lavage |
| YMC17/03/R585 | Sputum (pneumonia) | YMC16/09/R2471 | Tracheal aspirate |
| | | | (pneumonia) |
| YMC17/03/B4730 | Catheter blood | YMC16/10/R2537 | Sputum (pneumonia) |
| YMC17/03/B5000 | Catheter blood | YMC16/12/P503 | Swab or drainage tube, |
| | | | chest |
| YMC17/03/R1888 | Sputum (pneumonia) | YMC15/02/T28 | Another catheter tip |
| YMC17/03/R3279 | Sputum (pneumonia) | YMC15/02/R436 | Tracheal aspirate |
| | | | (pneumonia) |
| YMC17/03/R4077 | Tracheal aspirate | YMC15/03/R1604 | Tracheal aspirate |
| | (pneumonia) | | (pneumonia) |
| YMC17/04/R488 | Sputum (pneumonia) | YMC15/09/R1869 | Sputum (pneumonia) |
| YMC17/04/R640 | Sputum (pneumonia) | YMC14/06/R2359 | Sputum (pneumonia) |
| YMC/17/05/R1095 | Tracheal aspirate | YMC14/08/T90 | Another catheter tip |
| | (pneumonia) | | |
| YMC16/01/P11 | Swap or drainage tube, | YMC14/08/R1169 | Sputum (pneumonia) |
| | abdomen | | |
| YMC16/01/R123 | Tracheal tube tip | | |

TABLE 10

| Host | | Ampicillin- | | | | | | | |
|---|---|---|---|---|---|---|---|---|---|
| strain | Amikacin | sulbactam | Ceftazidime | Ciprofloxacin | Colistin | Cefepime | Cefotaxime | Gentamicin | Imipenem |
| YMC16/12/ | | | | | | | | | |
| R12914 | | | | | | | | | |
| YMC16/12/ | 6 R | =32 R | =64 R | =4 R | =0.5 S | =64 R | =64 R | =16 R | =16 R |
| B11422 | | | | | | | | | |
| YMC16/12/ | 6 R | =32 R | =64 R | =4 R | =0.5 S | =64 R | =64 R | =16 R | =16 R |
| B11449 | | | | | | | | | |
| YMC16/12/ | | | | | | | | | |
| B10832 | | | | | | | | | |
| YMC16/12/ | 6 R | 16 I | =64 R | =4 R | =0.5 S | =64 R | =64 R | =16 R | =16 R |
| B13325 | | | | | | | | | |
| YMC17/01/ | 6 R | 16 I | =64 R | =4 R | =0.5 S | =64 R | =64 R | =16 R | =16 R |
| P518 | | | | | | | | | |
| YMC17/01/ | 6 R | 8 S | =64 R | =4 R | =0.5 S | =64 R | =64 R | =16 R | =16 R |
| B8053 | | | | | | | | | |
| YMC17/01/ | 6 R | 16 I | =64 R | =4 R | =0.5 S | =64 R | =64 R | =16 R | =16 R |
| B10087 | | | | | | | | | |
| YMC17/01/ | 22 S | 16 I | =64 R | =4 R | 22 S | =64 R | =64 R | =1 S | =16 R |
| B12075 | | | | | | | | | |
| YMC17/02/ | 6 R | =32 R | =64 R | =4 R | =0.5 S | =64 R | =64 R | =16 R | =16 R |
| B14 | | | | | | | | | |
| YMC17/01/ | 6 R | =32 R | =64 R | =4 R | =0.5 S | =64 R | =64 R | =16 R | =16 R |
| B13454 | | | | | | | | | |
| YMC17/02/ | 20 S | 16 I | =64 R | =4 R | =0.5 S | =64 R | =64 R | 2 S | =16 R |
| B87 | | | | | | | | | |
| YMC17/02/ | 6 R | =32 R | =64 R | =4 R | =0.5 S | =64 R | =64 R | =16 R | =16 R |
| B721 | | | | | | | | | |
| YMC17/02/ | 6 R | =32 R | =64 R | =4 R | =0.5 S | =64 R | =64 R | =16 R | =16 R |
| B4520 | | | | | | | | | |
| YMC17/02/ | 6 R | =32 R | =64 R | =4 R | =0.5 S | =64 R | =64 R | =16 R | =16 R |
| B4039 | | | | | | | | | |
| YMC17/02/ | 25 S | =2 S | 4 S | =0.25 S | =0.5 S | 2 S | 8 S | =1 S | =0.25 S |
| B4864 | | | | | | | | | |
| YMC17/02/ | 21 S | 16 I | =64 R | =4 R | =0.5 S | =64 R | =64 R | 4 S | =16 R |
| P523 | | | | | | | | | |
| YMC17/02/ | 6 R | 16 I | =64 R | =4 R | =0.5 S | =64 R | =64 R | =16 R | =16 R |
| B8414 | | | | | | | | | |
| YMC17/03/ | 6 R | =32 R | =64 R | =4 R | =16 R | =64 R | =64 R | =16 R | =16 R |
| R585 | | | | | | | | | |
| YMC17/03/ | 6 R | 16 I | =64 R | =4 R | =0.5 S | =64 R | =64 R | =16 R | =16 R |
| B4730 | | | | | | | | | |
| YMC17/03/ | 6 R | =32 R | =64 R | =4 R | =0.5 S | =64 R | =64 R | =16 R | =16 R |
| B5000 | | | | | | | | | |
| YMC17/03/ | 6 R | 8 S | =64 R | =4 R | =16 R | =64 R | =64 R | =16 R | =16 R |
| R1888 | | | | | | | | | |
| YMC17/03/ | 6 R | 16 I | =64 R | =4 R | =16 R | =64 R | =64 R | =16 R | =16 R |
| R3279 | | | | | | | | | |
| YMC17/03/ | 6 R | 16 I | =64 R | =4 R | =0.5 S | =64 R | =64 R | =16 R | =16 R |
| R4077 | | | | | | | | | |
| YMC17/04/ | 6 R | 16 I | =64 R | =4 R | 8 R | =64 R | =64 R | =16 R | =16 R |
| R488 | | | | | | | | | |
| YMC17/04/ | 6 R | 16 I | =64 R | =4 R | 8 R | =64 R | =64 R | =16 R | =16 R |
| R640 | | | | | | | | | |
| YMC/17/05/ | 6 R | 16 I | =64 R | =4 R | =16 R | =64 R | =64 R | =16 R | =16 R |
| R1095 | | | | | | | | | |
| YMC16/01/ | 6 R | =32 R | =64 R | =4 R | =0.5 S | =64 R | =64 R | =16 R | =16 R |
| P11 | | | | | | | | | |
| YMC16/01/ | 6 R | =32 R | =64 R | =4 R | =0.5 S | =64 R | =64 R | =16 R | =16 R |
| R123 | | | | | | | | | |
| YMC16/01/ | 6 R | 8 S | =64 R | =4 R | =0.5 S | =64 R | =64 R | =16 R | =16 R |
| R198 | | | | | | | | | |
| YMC16/01/ | 6 R | 16 I | =64 R | =4 R | =0.5 S | =64 R | =64 R | =16 R | =16 R |
| R353 | | | | | | | | | |
| YMC16/01/ | 6 R | 16 I | =64 R | =4 R | =0.5 S | =64 R | =64 R | =16 R | =16 R |
| R405 | | | | | | | | | |
| YMC16/01/ | 6 R | =32 R | =64 R | =4 R | =0.5 S | =64 R | =64 R | =16 R | =16 R |
| R397 | | | | | | | | | |
| YMC16/01/ | | =32 R | =64 R | =4 R | =0.5 S | =64 R | =64 R | =16 R | =16 R |
| R380 | | | | | | | | | |
| YMC16/12/ | 6 R | =32 R | =64 R | =4 R | =16 R | =64 R | =64 R | =16 R | =16 R |
| R4637 | | | | | | | | | |
| YMC17/01/ | 6 R | =32 R | =64 R | =4 R | =16 R | =64 R | =64 R | =16 R | =16 R |
| R2812 | | | | | | | | | |
| YMC17/02/ | 6 R | =32 R | =64 R | =4 R | =16 R | =64 R | =64 R | =16 R | =16 R |
| R541 | | | | | | | | | |
| YMC17/02/ | 6 R | =32 R | =64 R | =4 R | 4 R | =64 R | =64 R | =16 R | =16 R |
| R2392 | | | | | | | | | |
| YMC17/03/ | 6 R | 16 I | 32 R | =4 R | 4 R | =64 R | =64 R | =16 R | =16 R |
| R348 | | | | | | | | | |
| YMC17/03/ | | | | | | | | | |
| R5305 | | | | | | | | | |
| YMC17/03/ | | | | | | | | | |
| R3095 | | | | | | | | | |
| YMC17/03/ | | | | | | | | | |
| R3428 | | | | | | | | | |
| YMC17/03/ | 6 R | 16 I | =64 R | =4 R | =0.5 S | =64 R | =64 R | =16 R | =16 R |
| R4607 | | | | | | | | | |
| YMC17/03/ | 6 R | 8 S | =64 R | =4 R | =16 R | =64 R | =64 R | =16 R | =16 R |
| P971 | | | | | | | | | |
| YMC16/03/ | 6 R | 4 S | =64 R | =4 R | =16 R | =64 R | 32 I | =16 R | =16 R |
| R4461 | | | | | | | | | |
| YMC16/05/ | | 16 I | =64 R | =4 R | =16 R | =64 R | =64 R | =16 R | =16 R |
| R2210 | | | | | | | | | |
| YMC16/07/ | 6 R | 16 I | =64 R | =4 R | =16 R | =64 R | =64 R | =16 R | =16 R |
| R2512 | | | | | | | | | |
| YMC16/09/ | 6 R | =32 R | =64 R | =4 R | =16 R | =64 R | =64 R | =16 R | =16 R |
| R2471 | | | | | | | | | |
| YMC16/10/ | 6 R | 16 I | =64 R | =4 R | =16 R | =64 R | =64 R | =16 R | =16 R |
| R2537 | | | | | | | | | |
| YMC16/12/ | 6 R | =32 R | =64 R | =4 R | =16 R | =64 R | =64 R | =16 R | =16 R |
| P503 | | | | | | | | | |
| YMC15/02/ | 6 R | 16 I | =64 R | =4 R | =16 R | 32 R | =64 R | =16 R | =16 R |
| T28 | | | | | | | | | |
| YMC15/02/ | 6 R | =32 R | =64 R | =4 R | 8 R | =64 R | =64 R | =16 R | =16 R |
| R436 | | | | | | | | | |
| YMC15/03/ | 6 R | 16 I | =64 R | =4 R | =16 R | =64 R | =64 R | =16 R | =16 R |
| R1604 | | | | | | | | | |
| YMC15/09/ | 6 R | 16 I | =64 R | =4 R | =16 R | 32 R | =64 R | =16 R | =16 R |
| R1869 | | | | | | | | | |
| YMC14/06/ | 6 R | 8 S | =64 R | =4 R | =16 R | =64 R | =64 R | =16 R | =16 R |
| R2359 | | | | | | | | | |
| YMC14/08/ | 6 R | 8 S | =64 R | =4 R | =16 R | =64 R | =64 R | =16 R | =16 R |
| T90 | | | | | | | | | |
| YMC14/08/ | 6 R | 16 I | =64 R | =4 R | =16 R | =64 R | =64 R | =16 R | =16 R |
| R1169 | | | | | | | | | |

| Host | | | | | Piperacillin- | | |
|---|---|---|---|---|---|---|---|
| strain | Levofloxacin | Meropenem | Minocycline | Piperacillin | tazobactam | Cortrimoxa | Tigecycline |
| YMC16/12/ | | | | | | | |
| R12914 | | | | | | | |
| YMC16/12/ | =8 R | =16 R | =1 S | =128 R | =128 R | 160 R | 1 S |
| B11422 | | | | | | | |
| YMC16/12/ | =8 R | =16 R | =1 S | =128 R | =128 R | =320 R | 2 S |
| B11449 | | | | | | | |
| YMC16/12/ | | | | | | | |
| B10832 | | | | | | | |
| YMC16/12/ | =8 R | =16 R | =1 S | =128 R | =128 R | =320 R | 1 S |
| B13325 | | | | | | | |
| YMC17/01/ | =8 R | =16 R | =1 S | =128 R | =128 R | 160 R | 2 S |
| P518 | | | | | | | |
| YMC17/01/ | =8 R | =16 R | =1 S | =128 R | =128 R | =320 R | =8 R |
| B8053 | | | | | | | |
| YMC17/01/ | =8 R | =16 R | =1 S | =128 R | =128 R | 160 R | 2 S |
| B10087 | | | | | | | |
| YMC17/01/ | =8 R | =16 R | =1 S | =128 R | =128 R | =20 S | 2 S |
| B12075 | | | | | | | |
| YMC17/02/ | =8 R | =16 R | 8 I | =128 R | =128 R | =320 R | 2 S |
| B14 | | | | | | | |
| YMC17/01/ | =8 R | =16 R | =1 S | =128 R | =128 R | 160 R | 2 S |
| B13454 | | | | | | | |
| YMC17/02/ | =8 R | =16 R | 8 I | =128 R | =128 R | =320 R | 2 S |
| B87 | | | | | | | |
| YMC17/02/ | =8 R | =16 R | =1 S | =128 R | =128 R | =320 R | 2 S |
| B721 | | | | | | | |
| YMC17/02/ | =8 R | =16 R | =1 S | =128 R | =128 R | 160 R | 1 S |
| B4520 | | | | | | | |
| YMC17/02/ | =8 R | =16 R | =1 S | =128 R | =128 R | 80 R | =0.5 S |
| B4039 | | | | | | | |
| YMC17/02/ | =0.12 S | =0.25 S | =1 S | 8 S | =4 S | =20 S | =0.5 S |
| B4864 | | | | | | | |
| YMC17/02/ | =8 R | =16 R | =1 S | =128 R | =128 R | =20 S | 1 S |
| P523 | | | | | | | |
| YMC17/02/ | =8 R | =16 R | 2 S | =128 R | =128 R | =320 R | =8 R |
| B8414 | | | | | | | |
| YMC17/03/ | =8 R | =16 R | =16 R | =128 R | =128 R | =320 R | 4 I |
| R585 | | | | | | | |
| YMC17/03/ | =8 R | =16 R | =1 S | =128 R | =128 R | =320 R | =8 R |
| B4730 | | | | | | | |
| YMC17/03/ | =8 R | =16 R | 8 I | =128 R | =128 R | =320 R | 1 S |
| B5000 | | | | | | | |
| YMC17/03/ | =8 R | =16 R | =1 S | =128 R | =128 R | =320 R | =8 R |
| R1888 | | | | | | | |
| YMC17/03/ | =8 R | =16 R | =1 S | =128 R | =128 R | =320 R | 1 S |
| R3279 | | | | | | | |
| YMC17/03/ | =8 R | =16 R | 2 S | =128 R | =128 R | =320 R | =8 R |
| R4077 | | | | | | | |
| YMC17/04/ | =8 R | =16 R | =1 S | =128 R | =128 R | 160 R | 2 S |
| R488 | | | | | | | |
| YMC17/04/ | =8 R | =16 R | =1 S | =128 R | =128 R | =320 R | =8 R |
| R640 | | | | | | | |
| YMC/17/05/ | =8 R | =16 R | 2 S | =128 R | =128 R | =320 R | 4 I |
| R1095 | | | | | | | |
| YMC16/01/ | =8 R | =16 R | =1 S | =128 R | =128 R | =320 R | 2 S |
| P11 | | | | | | | |
| YMC16/01/ | =8 R | =16 R | =1 S | =128 R | =128 R | 160 R | 2 S |
| R123 | | | | | | | |
| YMC16/01/ | =8 R | =16 R | =1 S | =128 R | =128 R | 160 R | 2 S |
| R198 | | | | | | | |
| YMC16/01/ | =8 R | =16 R | =1 S | =128 R | =128 R | 160 R | 2 S |
| R353 | | | | | | | |
| YMC16/01/ | =8 R | =16 R | =1 S | =128 R | =128 R | =320 R | =8 R |
| R405 | | | | | | | |
| YMC16/01/ | =8 R | =16 R | =1 S | =128 R | =128 R | 160 R | 1 S |
| R397 | | | | | | | |
| YMC16/01/ | =8 R | =16 R | 4 S | =128 R | =128 R | =20 S | 2 S |
| R380 | | | | | | | |
| YMC16/12/ | =8 R | =16 R | =16 R | =128 R | =128 R | =320 R | 4 I |
| R4637 | | | | | | | |
| YMC17/01/ | =8 R | =16 R | =16 R | =128 R | =128 R | =320 R | 4 I |
| R2812 | | | | | | | |
| YMC17/02/ | =8 R | =16 R | =16 R | =128 R | =128 R | =320 R | 4 I |
| R541 | | | | | | | |
| YMC17/02/ | =8 R | =16 R | =1 S | =128 R | =128 R | =320 R | 2 S |
| R2392 | | | | | | | |
| YMC17/03/ | =8 R | =16 R | =1 S | =128 R | =128 R | =320 R | =0.5 S |
| R348 | | | | | | | |
| YMC17/03/ | | | | | | | |
| R5305 | | | | | | | |
| YMC17/03/ | | | | | | | |
| R3095 | | | | | | | |
| YMC17/03/ | | | | | | | |
| R3428 | | | | | | | |
| YMC17/03/ | =8 R | =16 R | =16 R | =128 R | =128 R | =320 R | 2 S |
| R4607 | | | | | | | |
| YMC17/03/ | =8 R | =16 R | =1 S | =128 R | =128 R | =320 R | =8 R |
| P971 | | | | | | | |
| YMC16/03/ | =8 R | =16 R | =1 S | =128 R | =128 R | =320 R | 4 I |
| R4461 | | | | | | | |
| YMC16/05/ | =8 R | =16 R | =1 S | =128 R | =128 R | =320 R | 2 S |
| R2210 | | | | | | | |
| YMC16/07/ | =8 R | =16 R | =1 S | =128 R | =128 R | =320 R | 2 S |
| R2512 | | | | | | | |
| YMC16/09/ | =8 R | =16 R | =1 S | =128 R | =128 R | 160 R | 1 S |
| R2471 | | | | | | | |
| YMC16/10/ | =8 R | =16 R | =1 S | =128 R | =128 R | 160 R | 1 S |
| R2537 | | | | | | | |
| YMC16/12/ | =8 R | =16 R | =16 R | =128 R | =128 R | =320 R | 2 S |
| P503 | | | | | | | |
| YMC15/02/ | =8 R | =16 R | =1 S | =128 R | =128 R | =320 R | 2 S |
| T28 | | | | | | | |
| YMC15/02/ | =8 R | =16 R | =16 R | =128 R | =128 R | =320 R | 4 I |
| R436 | | | | | | | |
| YMC15/03/ | =8 R | =16 R | =1 S | =128 R | =128 R | =320 R | 2 S |
| R1604 | | | | | | | |
| YMC15/09/ | =8 R | =16 R | =1 S | =128 R | =128 R | =320 R | 4 I |
| R1869 | | | | | | | |
| YMC14/06/ | =8 R | =16 R | =1 S | =128 R | =128 R | =320 R | 2 S |
| R2359 | | | | | | | |
| YMC14/08/ | =8 R | =16 R | 2 S | =128 R | =128 R | =320 R | 2 S |
| T90 | | | | | | | |
| YMC14/08/ | =8 R | =16 R | =1 S | =128 R | =128 R | =320 R | =8 R |
| R1169 | | | | | | | |

As shown in Table 10, the collected 57 Acinetobacter baumannii strains were found to be multi-drug-resistant strains having resistance to various antibiotics.

2. Collection of Bacteriophage Specimens

2-1. Collection of Specimens to Construct Phage Bank

Raw water was obtained by causing sewage to pass through a first sedimentation tank at the sewage treatment facility of the Severance Hospital (Korea), and then removing suspended substances and sediments therefrom. The sewage was limited to sewage that was present at a preliminary stage of a chemical treatment facility. To the collected sample was added 58 g of sodium chloride per L. Then, centrifugation was performed at 10,000 g for 10 minutes and filtration was performed through a 220 nm Millipore filter. To the obtained filtrate was added polyethylene glycol (PEG, molecular weight of 8000) at 1000 w/v, and the resultant was stored refrigerated at 4° C. for 12 hours. The filtrate stored refrigerated for 12 hours was centrifuged at 12,000 g for 20 minutes, and the precipitate was resuspended in phage dilution buffer (SM buffer). To the resuspension was then added the same amount of chloroform, and the resultant was stored frozen. This was repeated three times to collect 300 mL of bacteriophage suspension.

2-2. Selection of Lytic Phage and Measurement of Lysis Titer

Separation and purification of lytic phage were performed by a spot test method (Mazzocco A et al. In Bacteriophages, Clokie and Kropinski AM, eds. Humana Press. 2009). The obtained strains were inoculated on MacConkey Agar medium and then cultured overnight at 35° C. in outside air. After the culture, strains susceptible to phage were selected by observing formation of clear plaque. The susceptible strains were inoculated on MacConkey Agar medium and cultured at 35° C. for 12 hours. A suspension of each strain was prepared in a 1 ml saline tube with a turbidity of 0.5 McFarland, and mixed with H top agar (3 ml), 100 μl of sensitive bacteria, and a phage solution (each of 1 μl, 10 μl, and 50 μl). The mixture was applied to LB agar, and then cultured at 35° C. for 12 hours. Plaque was observed, and then the plaque was collected with a Pasteur pipette. The collected plaque was diluted in SM buffer solution, and repeatedly purified three times using the susceptible strain suspension again. The thus obtained pure bacteriophage, YMC16/01/R2016_ABA_BP, was diluted in SM buffer solution, and repeatedly purified three times using the susceptible strain suspension again. The thus obtained pure bacteriophage, YMC16/01/R2016_ABA_BP, was diluted in SM buffer solution and stored.

Each of the 57 antibiotic-resistant Acinetobacter baumannii strains identified in item no. 1. above was inoculated on MacConkey Agar medium and cultured. Then, the bacteriophage YMC16/01/R2016_ABA_BP, which had been purified by the above process, was inoculated in an amount of 5 μl into each smeared resistant strain. Then, plaque formation was checked and a titer range thereof was checked. The lysis of each strain is shown in Table 11 below. In Table 11 below, an evaluation result of plaque activity against the collected strains is indicated by + and −, in which ‘+’ means clear plaque and ‘-’ means that lysis has not occurred.

TABLE 11

| Host strain | Lysis | Host strain | Lysis |
|---|---|---|---|
| YMC16/12/B11422 | ++ | YMC16/01/R198 | + |
| YMC16/12/B13325 | ++ | YMC16/01/R353 | + |
| YMC17/01/P518 | ++ | YMC16/01/R397 | + |
| YMC17/01/B8053 | ++ | YMC17/03/R3095 | ++ |
| YMC17/01/B12075 | ++ | YMC17/03/R3428 | ++ |
| YMC17/02/B4520 | ++ | YMC17/03/P971 | ++ |
| YMC17/02/B4039 | ++ | YMC16/03/R4461 | + |
| YMC17/02/P523 | ++ | YMC16/07/R2512 | ++ |
| YMC17/03/R585 | + | YMC16/10/R2537 | ++ |
| YMC17/03/B4730 | + | YMC15/02/T28 | ++ |
| YMC17/03/R1888 | + | YMC15/02/R436 | + |
| YMC17/03/R3279 | + | YMC15/03/R1604 | + |
| YMC17/03/R4077 | + | YMC15/09/R1869 | + |
| YMC17/04/R488 | + | YMC14/06/R2359 | + |
| YMC17/04/R640 | + | YMC14/08/T90 | ++ |
| YMC16/01/R123 | ++ | YMC14/08/R1169 | + |

As shown in Table 11, it was found that the bacteriophage YMC16/01/R2016_ABA_BP according to the present invention lyses antibiotic-resistant Acinetobacter baumannii strains.

3. Electron Microscopic Analysis of Lytic Bacteriophage Against Antibiotic-Resistant Acinetobacter baumannii Strains

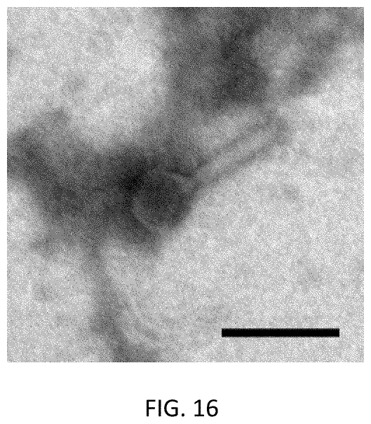

The bacteriophage YMC16/01/R2016_ABA_BP purified by the method of item no. 2. above was inoculated and cultured in culture medium (20 ml of LB medium) for susceptible strains, and then filtered through a 220 nm Millipore filter. To the supernatant was added polyethylene glycol (MW 8,000) in an amount of 10% (w/v), and then the resultant was stored refrigerated overnight. Subsequently, centrifugation was performed for 20 minutes at 12,000 g, and then a shape of the bacteriophage YMC16/01/R2016_ABA_BP was analyzed using an energy-filtering transmission electron microscope. The result is illustrated in FIG. 16.

As illustrated in FIG. 16, in a case where classification is made on a shape basis, the bacteriophage YMC16/01/R2016_ABA_BP according to the present invention was classified as belonging to the family Myoviridae that has a long tail with a hexagonal head.

4. Analysis of Adsorption Capacity and One-Step Growth Curve of Bacteriophage

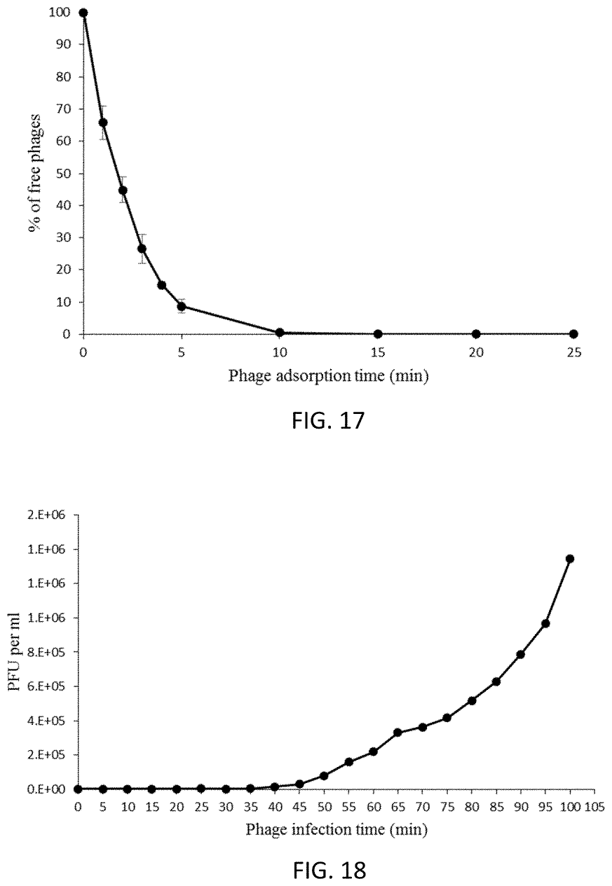

The antibiotic-resistant Acinetobacter baumannii strain was cultured to an OD value of 0.5. To the Acinetobacter baumannii strain was then added the bacteriophage YMC16/01/R2016_ABA_BP purified in item no. 2. above at an MOI of 0.001 and culture was performed at room temperature. Then, sample was collected 1 ml each at 1, 2, 3, 4, and 5 minutes, diluted in LB medium, and then adsorption capacity of the bacteriophage was evaluated through plaque analysis. The results are illustrated in FIG. 17.

In addition, the antibiotic-resistant Acinetobacter baumannii strain was cultured to an OD value of 0.3, and then centrifuged at 7,000 g for 5 minutes at 4° C., to precipitate the cells. Then, the cells were diluted in 0.5 ml of LB medium. To the dilute was added the bacteriophage YMC16/01/R2016_ABA_BP purified in item no. 2. above at an MOI of 0.001 (titer of 10⁸ pfu/cell), and culture was performed at 37° C. for 5 minutes. The cultured mixed sample was centrifuged at 13,000 g for 1 minute to obtain a pellet. The obtained pellet was diluted in 10 ml of LB medium and cultured at 37° C. Samples were collected every 10 minutes during the culture, and a one-step growth curve of the bacteriophage was evaluated through plaque analysis. The results are illustrated in FIG. 18.

As illustrated in FIG. 17, about 100% of the bacteriophage YMC16/01/R2016_ABA_BP was adsorbed to the Acinetobacter baumannii strain within 10 minutes after inoculation of the bacteriophage.

In addition, as illustrated in FIG. 18, the one-step growth curve showed a high burst size of 448 PFU/infected cells.

From the above results, it can be seen that the bacteriophage YMC16/01/R2016_ABA_BP according to the present invention can be adsorbed in a relatively short time to an antibiotic-resistant Acinetobacter baumannii strain and can show a high burst size of 448 PFU/infected cells, indicating that this bacteriophage exerts a lytic effect on an antibiotic-resistant strain.

5. Verification of In Vivo Lysis Ability of Bacteriophage Against Antibiotic-Resistant Acinetobacter Genus Bacteria

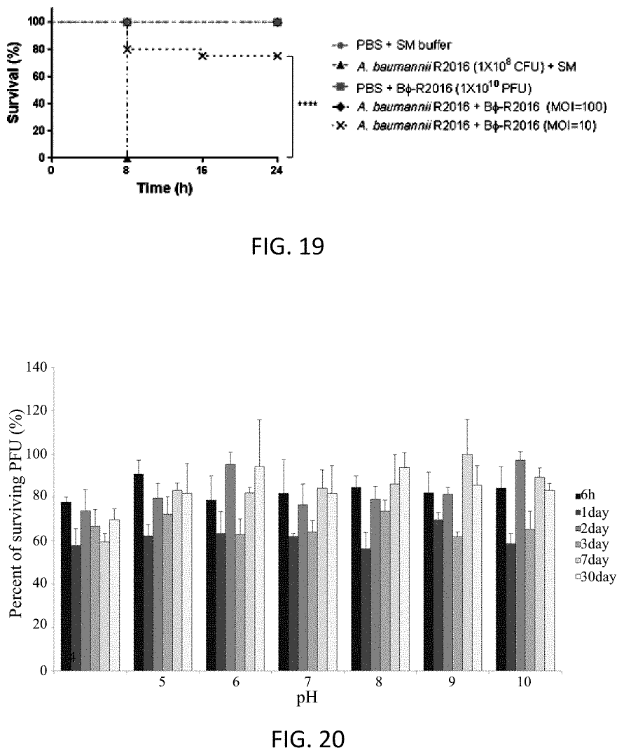

200 third- to fourth-instar Galleria mellonella larvae were prepared, and then divided into groups, each containing 10 larvae. Each larva was injected through its proleg with a carbapenem-resistant Acinetobacter baumannii strain at a minimum lethal dose (MLD), and then subjected to mixed inoculation with the bacteriophage YMC16/01/R2016_ABA_BP purified in item no. 2. above at an MOI of 10 or an MOI of 100. Then, survival of the larvae was checked every 12 or 24 hours until 72 hours, and the results are illustrated in FIG. 19.

As illustrated in FIG. 19, it was found that in a case where the larvae injected with the carbapenem-resistant Acinetobacter baumannii strain are treated with the bacteriophage YMC16/01/R2016_ABA_BP according to the present invention, survival of the larvae increases, in which the survival of the larvae further increases as the MOI value increases. In addition, it was found that even in a case where the larvae are injected with only the bacteriophage YMC16/01/R2016_ABA_BP without injection of the carbapenem-resistant Acinetobacter baumannii strain, no toxicity is seen when survival thereof is compared with that of a healthy control group.

From the above results, it can be seen that the bacteriophage YMC16/01/R2016_ABA_BP according to the present invention also has lytic properties in vivo against an antibiotic-resistant Acinetobacter baumannii strain, and thus can effectively prevent, ameliorate, or treat an infectious disease caused by the Acinetobacter baumannii strain.

6. Evaluation of Stability of Bacteriophage Against Antibiotic-Resistant Acinetobacter baumannii Strain

It was identified whether the bacteriophage YMC16/01/R2016_ABA_BP according to the present invention maintains stability without being destroyed under alkaline and temperature conditions.

1 μl of the bacteriophage YMC16/01/R2016_ABA_BP purified by the method of item no. 2 above was added to 40 μl of SM buffer, which had been adjusted to a pH of 4, 5, 6, 7, 8, 9, or 10, and then incubated at 37° C. for 1 hour. Then, plaque analysis was performed with the antibiotic-resistant Acinetobacter baumannii bacteria using the method of item no. 4 above. The results are illustrated in FIG. 20.

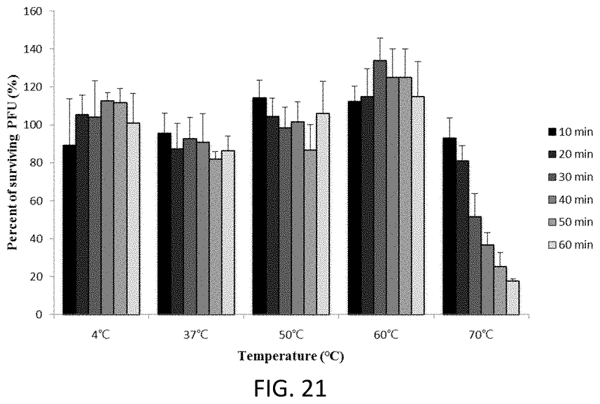

In addition, during 1-hour incubation of the bacteriophage YMC16/01/R2016_ABA_BP solution at 4° C., 37° C., 50° C., 60° C., and 70° C., respectively, each sample was collected every 10 minutes and plaque analysis was performed with the Acinetobacter baumannii strain using the method of item no. 4 above. The results are illustrated in FIG. 21.

As illustrated in FIG. 20, the bacteriophage YMCT6/01/R2016_ABA_BP according to the present invention exhibited high stability in all conditions which are acidic, neutral, and alkaline.

In addition, as illustrated in FIG. 21, the bacteriophage YMCT6/01/R2016_ABA_BP exhibited very high stability up to a temperature as high as 60° C.

7. Whole-Genome Sequencing of Bacteriophage Against Antibiotic-Resistant Acinetobacter Genus Bacteria

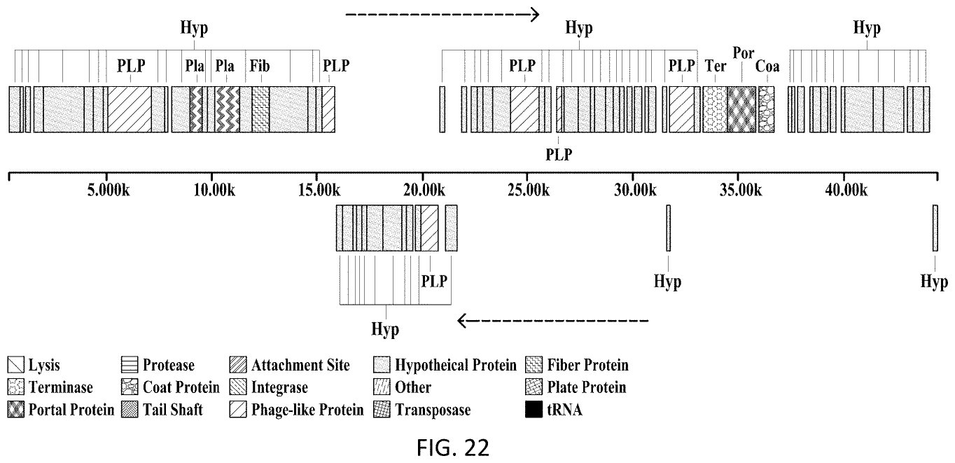

To characterize the bacteriophage YMC16/01/R2016_ABA_BP according to the present invention, whole-genome sequencing thereof was performed through the Illumina sequencer (Roche) based on a whole-genome sequencing method which is obvious to those skilled in the art. The results are shown in FIG. 22 and Table 12.

TABLE 12

| | | | | | | | | | NCBI | |
|---|---|---|---|---|---|---|---|---|---|---|
| | | | Initi- | | | | | | blastP | NCBI-Bank |
| Genome | Range | | ation | | Length | Putative | Annotation | E- | identity | accession |
| no. | Start | End | codon | Strand | (bp) | function | source | value | (%) | number |
| ORF1 | 376 | 882 | ATG | + | 507 | Putative RNA | Acinetobacter phage | 5E−98 | 94 | ARB06827.1 |
| | | | | | | polymerase | WCHABP12 | | | |
| ORF2 | 879 | 1064 | ATG | + | 186 | Hypothetical protein | Acinetobacter phage | 8E−31 | 90 | ADO14379.1 |
| | | | | | | | AB1 | | | |
| ORF3 | 1112 | 1393 | ATG | + | 282 | Putative capsid | Acinetobacter phage | 3E−55 | 100 | YP_009055482.1 |
| | | | | | | protein | YMC-13-01-C62 | | | |
| ORF4 | 1471 | 1941 | ATG | + | 471 | Hypothetical protein | Acinetobacter phage | 3E−108 | 96 | ADO14377.1 |
| | | | | | | | AB1 | | | |
| ORF5 | 1938 | 3881 | ATG | + | 1944 | Hypothetical protein | Acinetobacter phage | 0 | 98 | ADO14374.1 |
| | | | | | | | AB1 | | | |
| ORF6 | 3894 | 4343 | ATG | + | 450 | Hypothetical protein | Acinetobacter phage | 2E−59 | 58 | ADO14373.1 |
| | | | | | | | AB1 | | | |
| ORF7 | 4389 | 4814 | ATG | + | 426 | Hypothetical protein | Acinetobacter phage | 1E−37 | 46 | ADO14372.1 |
| | | | | | | | AB1 | | | |
| ORF8 | 4814 | 5056 | GTG | + | 243 | Putative tail-fiber/ | Acinetobacter phage | 3E−52 | 99 | YP_009055476.1 |
| | | | | | | lysozyme protein | YMC-13-01-C62 | | | |
| ORF9 | 5056 | 7107 | ATG | + | 2052 | Lysozyme like | Acinetobacter phage | 0 | 100 | YP_009055475.1 |
| | | | | | | domain protein | YMC-13-01-C62 | | | |
| ORF10 | 7115 | 7711 | ATG | + | 597 | Hypothetical protein | Acinetobacter phage | 2E−139 | 98 | ADO14454.1 |
| | | | | | | | AB1 | | | |
| ORF11 | 7650 | 7991 | ATG | + | 342 | Hypothetical protein | Acinetobacter phage | 5E−60 | 99 | ARB06749.1 |
| | | | | | | | WCHABP12 | | | |
| ORF12 | 8100 | 8990 | GTG | + | 891 | Hypothetical protein | Acinetobacter phage | 0 | 94 | ADO14453.1 |
| | | | | | | | AB1 | | | |
| ORF13 | 8947 | 9618 | ATG | + | 672 | Putative baseplate | Acinetobacter phage | 2E−157 | 100 | YP 009055472.1 |
| | | | | | | assembly protein | YMC-13-01-C62 | | | |
| ORF14 | 9764 | 10117 | ATG | + | 354 | Hypothetical protein | Acinetobacter phage | 3E−81 | 99 | ADO14451.1 |
| | | | | | | | AB1 | | | |
| ORF15 | 10114 | 11298 | ATG | + | 1185 | Putative baseplate | Acinetobacter phage | 0 | 99 | AFV51558.1 |
| | | | | | | J-like protein | IME-AB2 | | | |
| ORF16 | 11298 | 11924 | ATG | + | 627 | Hypothetical protein | Acinetobacter phage | 8E−149 | 99 | ADO14449.1 |
| | | | | | | | AB1 | | | |
| ORF17 | 11902 | 12747 | GTG | + | 846 | Putative tail fiber | Acinetobacter phage | 0 | 99 | YP_009203603.1 |
| | | | | | | protein | YMC11/12/R2315 | | | |
| ORF18 | 12749 | 14572 | ATG | + | 1824 | Putative tail fiber | Acinetobacter phage | 2E−77 | 88 | ARQ94726.1 |
| | | | | | | protein | WCHABP1 | | | |
| ORF19 | 14648 | 14968 | ATG | + | 321 | Hypothetical protein | Acinetobacter phage | 7E−54 | 96 | ADO14446.1 |
| | | | | | | | AB1 | | | |
| ORF20 | 14952 | 15227 | ATG | + | 276 | Hypothetical protein | Acinetobacter phage | 1E−56 | 95 | ADO14445.1 |
| | | | | | | | AB1 | | | |
| ORF21 | 15214 | 15822 | ATG | + | 609 | Putative endolysin | Acinetobacter phage | 5E−143 | 98 | ARB06760.1 |
| | | | | | | | WCHABP12 | | | |
| ORF22 | 15915 | 16145 | ATG | − | 231 | rIIB lysis inhibitor | Caulobacter phage | 2 | 33 | AXQ68725.1 |
| | | | | | | | CcrPW | | | |
| ORF23 | 16138 | 16710 | ATG | − | 573 | Putative nucleoside | Acinetobacter phage | 5E−71 | 64 | AFV51550.1 |
| | | | | | | triphosphate | IME-AB2 | | | |
| | | | | | | pyrophospho- | | | | |
| | | | | | | hydrolase | | | | |
| ORF24 | 16698 | 16859 | ATG | − | 162 | Hypothetical protein | Acinetobacter phage | 3E−29 | 96 | ADO14441.1 |
| | | | | | | | AB1 | | | |
| ORF25 | 16856 | 17077 | ATG | − | 222 | Hypothetical protein | Acinetobacter phage | 2E−07 | 43 | YP_009055458.1 |
| | | | | | | | YMC-13-01-C62 | | | |
| ORF26 | 17074 | 17367 | ATG | − | 294 | Hypothetical protein | Acinetobacter phage | 7E−61 | 96 | ADO14439.1 |
| | | | | | | | AB1 | | | |
| ORF27 | 17368 | 18123 | ATG | − | 756 | Hypothetical protein | Acinetobacter phage | 2E−169 | 97 | ADO14438.1 |
| | | | | | | | AB1 | | | |
| ORF28 | 18120 | 19019 | ATG | − | 900 | Hypothetical protein | Psychrobacter phage | 1E−70 | 43 | YP_007673324.1 |
| | | | | | | | pOW20-A | | | |
| ORF29 | 19016 | 19198 | ATG | − | 183 | Hypothetical protein | Acinetobacter phage | 2E−36 | 100 | YP 009055454.1 |
| | | | | | | | YMC-13-01-C62 | | | |
| ORF30 | 19198 | 19530 | ATG | − | 333 | Hypothetical protein | Acinetobacter phage | 1E−68 | 94 | ADO14435.1 |
| | | | | | | | AB1 | | | |
| ORF31 | 19623 | 19892 | ATG | − | 270 | Hypothetical protein | Acinetobacter phage | 6E−47 | 88 | ADO14434.1 |
| | | | | | | | AB1 | | | |
| ORF32 | 19947 | 20759 | ATG | − | 813 | Putative | Acinetobacter phage | 0 | 100 | YP_009055451.1 |
| | | | | | | transcriptional | YMC-13-01-C62 | | | |
| | | | | | | regulator | | | | |
| ORF33 | 20859 | 21053 | ATG | + | 195 | Hypothetical protein | Acinetobacter phage | 3E−14 | 52 | ADO14431.1 |
| | | | | | | | AB1 | | | |
| ORF34 | 21106 | 21693 | ATG | − | 588 | Putative HNH | Acinetobacter phage | 3E−61 | 50 | ASJ78942.1 |
| | | | | | | homing | AbP2 | | | |
| | | | | | | endonuclease | | | | |
| ORF35 | 21887 | 22075 | ATG | + | 189 | Hypothetical protein | Acinetobacter phage | 1E−21 | 86 | ADO14428.1 |
| | | | | | | | AB1 | | | |
| ORF36 | 22259 | 22594 | ATG | + | 336 | Hypothetical protein | Acinetobacter phage | 8E−76 | 100 | YP_009055447.1 |
| | | | | | | | YMC-13-01-C62 | | | |
| ORF37 | 22609 | 22821 | ATG | + | 213 | Hypothetical protein | Acinetobacter phage | 2E−38 | 87 | ADO14425.1 |
| | | | | | | | AB1 | | | |
| ORF38 | 22834 | 23313 | ATG | + | 480 | Hypothetical protein | Acinetobacter phage | 2E−113 | 100 | YP_009055445.1 |
| | | | | | | | YMC-13-01-C62 | | | |
| ORF39 | 23306 | 24172 | ATG | + | 867 | Putative primosomal | Acinetobacter phage | 0 | 99 | AFV51535.1 |
| | | | | | | protein | IME-AB2 | | | |
| ORF40 | 24178 | 25521 | ATG | + | 1344 | Putative replicative | Acinetobacter phage | 0 | 99 | YP_009055443.1 |
| | | | | | | DNA helicase | YMC-13-01-C62 | | | |
| ORF41 | 25532 | 25801 | ATG | + | 270 | Hypothetical protein | Acinetobacter phage | 4E−35 | 63 | ADO14422.1 |
| | | | | | | | AB1 | | | |
| ORF42 | 25864 | 26091 | ATG | + | 228 | Hypothetical protein | Acinetobacter phage | 3E−58 | 91 | ADO14421.1 |
| | | | | | | | AB1 | | | |
| ORF43 | 26088 | 26237 | ATG | + | 150 | Hypothetical protein | Acinetobacter phage | 0.32 | 41 | YP_009055440.1 |
| | | | | | | | YMC-13-01-C62 | | | |
| ORF44 | 26309 | 26521 | ATG | + | 213 | Putativebacteriophage- | Acinetobacter phage | 2E−41 | 99 | AFV51531.1 |
| | | | | | | associated | IME-AB2 | | | |
| | | | | | | immunity protein | | | | |
| ORF45 | 26518 | 26631 | ATG | + | 114 | Hypothetical protein | Acinetobacter phage | 3E−14 | 95 | ADO14419.1 |
| | | | | | | | AB1 | | | |
| ORF46 | 26619 | 27386 | ATG | + | 768 | Hypothetical protein | Acinetobacter phage | 2E−134 | 79 | ADO14418.1 |
| | | | | | | | AB1 | | | |
| ORF47 | 27383 | 27958 | ATG | + | 576 | Hypothetical protein | Acinetobacter phage | 1E−137 | 98 | ADO14417.1 |
| | | | | | | | AB1 | | | |
| ORF48 | 27955 | 28119 | GTG | + | 165 | Hypothetical protein | Acinetobacter phage | 2E−24 | 89 | ADO14416.1 |
| | | | | | | | AB1 | | | |
| ORF49 | 28116 | 28697 | ATG | + | 582 | Hypothetical protein | Escherichia phage | 1E−10 | 56 | YP_009018683.1 |
| | | | | | | | EB49 | | | |
| ORF50 | 28684 | 29070 | ATG | + | 387 | Hypothetical protein | Acinetobacter phage | 1E−21 | 42 | ADO14414.1 |
| | | | | | | | AB1 | | | |
| ORF51 | 29067 | 29327 | ATG | + | 261 | Hypothetical protein | Acinetobacter phage | 3E−56 | 100 | YP_009055433.1 |
| | | | | | | | YMC-13-01-C62 | | | |
| ORF52 | 29308 | 29595 | GTG | + | 288 | tRNA endonuclease- | Vibrio phage | 2E−17 | 48 | AUR89331.1 |
| | | | | | | like domain protein | 1.122.A._10N.286.46.F8 | | | |
| ORF53 | 29736 | 29975 | ATG | + | 240 | Hypothetical protein | Acinetobacter phage | 5E−44 | 96 | ADO14411.1 |
| | | | | | | | AB1 | | | |
| ORF54 | 30048 | 30386 | ATG | + | 339 | Hypothetical protein | Acinetobacter phage | 2E−79 | 100 | YP 009055430.1 |
| | | | | | | | YMC-13-01-C62 | | | |
| ORF55 | 30713 | 31039 | ATG | + | 327 | Hypothetical protein | Acinetobacter phage | 4E−74 | 100 | AJT61457.1 |
| | | | | | | | YMC11/12/R1215 | | | |
| ORF56 | 31042 | 31221 | ATG | + | 180 | Fis family | Acinetobacter phage | 2E−06 | 45 | ARB06798.1 |
| | | | | | | transcriptional | WCHABP12 | | | |
| | | | | | | regulator | | | | |
| ORF57 | 31326 | 31589 | ATG | + | 264 | Hypothetical protein | Acinetobacter phage | 2E−32 | 96 | ASJ78929.1 |
| | | | | | | | AbP2 | | | |
| ORF58 | 31641 | 32834 | GTG | + | 1194 | ParB/sulfiredoxin | Vibrio phage | 4E−138 | 58 | AUR95847.1 |
| | | | | | | | 1.213.O._10N.222.54.F10 | | | |
| ORF59 | 32827 | 33192 | ATG | + | 366 | DNA binding domain | uncultured | 2E−12 | 41 | BAQ88996.1 |
| | | | | | | | Mediterranean | | | |
| | | | | | | | phage uvMED | | | |
| ORF60 | 33161 | 34462 | ATG | + | 1302 | Putative phage | Acinetobacter phage | 0 | 94 | YP_006383766.1 |
| | | | | | | terminase | AP22 | | | |
| | | | | | | large subunit | | | | |
| ORF61 | 34466 | 35896 | ATG | + | 1431 | Putative portal | Acinetobacter phage | 0 | 96 | ARB06806.1 |
| | | | | | | protein | WCHABP12 | | | |
| ORF62 | 35899 | 36669 | ATG | + | 771 | Putative head protein | Acinetobacter phage | 0 | 99 | ASJ78923.1 |
| | | | | | | | AbP2 | | | |
| ORF63 | 37359 | 37523 | ATG | + | 165 | Hypothetical protein | Acinetobacter phage | 7E−32 | 100 | YP_009055500.1 |
| | | | | | | | YMC-13-01-C62 | | | |
| ORF64 | 37560 | 37670 | ATG | + | 111 | Hypothetical protein | Acinetobacter phage | 3E−27 | 100 | YP_009055499.1 |
| | | | | | | | YMC-13-01-C62 | | | |
| ORF65 | 37752 | 38105 | ATG | + | 354 | Hypothetical protein | Acinetobacter phage | 4E−83 | 100 | YP_009055498.1 |
| | | | | | | | YMC-13-01-C62 | | | |
| ORF66 | 38095 | 38517 | ATG | + | 423 | | | | | |
| ORF67 | 38510 | 38902 | ATG | + | 393 | Hypothetical protein | Acinetobacter phage | 1E−89 | 100 | AJT61472.1 |
| | | | | | | | YMC11/12/R1215 | | | |
| ORF68 | 38899 | 39261 | ATG | + | 363 | Hypothetical protein | Acinetobacter phage | 2E−84 | 100 | YP_009055495.1 |
| | | | | | | | YMC-13-01-C62 | | | |
| ORF69 | 39360 | 39635 | ATG | + | 276 | Hypothetical protein | Acinetobacter phage | 6E−61 | 100 | YP 009055494.1 |
| | | | | | | | YMC-13-01-C62 | | | |
| ORF70 | 40045 | 41379 | ATG | + | 1335 | Hypothetical protein | Acinetobacter phage | 0 | 81 | ADO14388.1 |
| | | | | | | | AB1 | | | |
| ORF71 | 41387 | 41866 | ATG | + | 480 | Hypothetical protein | Acinetobacter phage | 2E−110 | 100 | YP_009055490.1 |
| | | | | | | | YMC-13-01-C62 | | | |
| ORF72 | 41876 | 42895 | ATG | + | 1020 | Hypothetical protein | Acinetobacter phage | 0 | 100 | YP_009055489.1 |
| | | | | | | | YMC-13-01-C62 | | | |
| ORF73 | 42975 | 43313 | ATG | + | 339 | Hypothetical protein | Acinetobacter phage | 3E−21 | 43 | ADO14384.1 |
| | | | | | | | AB1 | | | |
| ORF74 | 43313 | 43762 | ATG | + | 450 | Hypothetical protein | Acinetobacter phage | 1E−84 | 80 | ADO14383.1 |
| | | | | | | | AB1 | | | |
| ORF75 | 43778 | 44053 | ATG | + | 276 | Hypothetical protein | Acinetobacter phage | 2E−58 | 98 | YP 006383783.1 |
| | | | | | | | AP22 | | | |
| ORF76 | 44221 | 44445 | ATG | − | 225 | Hypothetical protein | Acinetobacter phage | 4E−47 | 100 | AFV51493.1 |
| | | | | | | | IME-AB2 | | | |

As shown in FIG. 22 and Table 12, the bacteriophage YMC16/01/R2016_ABA_BP contained linear dsDNA and was composed of 76 ORFs.

As a result of comparing the sequence of the bacteriophage YMC16/01/R2016_ABA_BP according to the present invention with sequences of the existing bacteriophages, no bacteriophage having similarity to the bacteriophage according to the present invention was detected. From the above results, it can be seen that the bacteriophage YMC16/01/R2016_ABA_BP according to the present invention corresponds to a novel bacteriophage that has not been previously discovered.

Although the present invention has been described in detail above, the scope of the present invention is not limited thereto. It will be obvious to those skilled in the art that various modifications and changes can be made without departing from the technical spirit of the present invention described in the claims.

[Accession Number (1)]

Bacteriophage YMC14/01/P117_ABA_BP

Depositary institution name: Korean Culture Center of Microorganisms (Korea)

Accession number: KFCC11800P

Accession date: Nov. 15, 2018

[Accession Number (2)]

Bacteriophage YMC16/12/R4637_ABA_BP

Depositary institution name: Korean Culture Center of Microorganisms (Korea)

Accession number: KFCC11801P

Accession date: Nov. 15, 2018

[Accession Number (3)]

Bacteriophage YMC16/01/R2016_ABA_BP

Depositary institution name: Korean Culture Center of Microorganisms (Korea)

Accession number: KFCC11803P

Accession date: Nov. 15, 2018

1. A bacteriophage that has a specific killing ability against Acinetobacter genus bacteria and belongs to the family Myoviridae.

2. The bacteriophage according to claim 1,
wherein the Acinetobacter genus bacteria are at least one selected from the group consisting of Acinetobacter baumannii, Acinetobacter calcoaceticus, Acinetobacter haemolyticus, Acinetobacter junii, Acinetobacter johnsonii, Acinetobacter lwoffii, Acinetobacter radioresistens, Acinetobacter ursingii, Acinetobacter schindleri, Acinetobacter parvus, Acinetobacter baylyi, Acinetobacter bouvetii, Acinetobacter towneri, Acinetobacter tandoii, Acinetobacter grimontii, Acinetobacter tjernbergiae, and Acinetobacter gerneri.

3. The bacteriophage according to claim 1,
wherein the Acinetobacter genus bacteria are Acinetobacter baumannii.

4. The bacteriophage according to claim 1,
wherein the Acinetobacter genus bacteria are antibiotic-resistant bacteria.

5. The bacteriophage according to claim 4,
wherein the antibiotics are carbapenem-based antibiotics.

6. The bacteriophage according to claim 4,
wherein the antibiotics are at least one selected from the group consisting of amikacin, ampicillin, ampicillin-sulbactam, aztreonam, ciprofloxacin, ceftazidime, cefazolin, ertapenem, cefepime, cefoxitin, cefotaxime, gentamicin, levofloxacin, minocycline, imipenem, meropenem, piperacillin, piperacillin-tazobactam, cortrimoxa, and tigecycline.

7. The bacteriophage according to claim 1,
wherein the bacteriophage is any one of a bacteriophage which is designated YMC14/01/P117_ABA_BP and has an accession number of KFCC11800P; a bacteriophage which is designated YMC16/12/R4637_ABA_BP and has an accession number of KFCC11801P; or a bacteriophage which is designated YMC16/01/R2016_ABA_BP and has an accession number of KFCC11803P.

8. The bacteriophage according to claim 1,
wherein the bacteriophage includes a genome represented by SEQ ID NO: 1, 8, or 13.

9. The bacteriophage according to claim 1,
wherein the bacteriophage includes any one protein of SEQ ID NOs: 2 to 4, 9, 10, and 14 to 16.

10. The bacteriophage according to claim 1,
wherein the bacteriophage includes a genome represented by any one of SEQ ID NOs: 5 to 7, 11, 12, and 17 to 19.

11. A composition of antibiotics, disinfection or cleaning, comprising as an active ingredient:
the bacteriophage according to claim 1.

12. A method for preventing or treating a disease caused by Acinetobacter genus bacteria, comprising:
a step of administering, to an individual, the bacteriophage according to claim 1.

13. The method according to claim 12,
wherein the disease caused by Acinetobacter genus bacteria is a disease selected from the group consisting of hepatitis C, hand-foot-and-mouth disease, gonorrhea, chlamydia, chancroid, genital herpes, condylomata acuminata, vancomycin-resistant Staphylococcus aureus infection, vancomycin-resistant Enterococci infection, methicillin-resistant Staphylococcus aureus infection, multi-drug-resistant Pseudomonas aeruginosa infection, multi-drug-resistant Acinetobacter baumannii infection, carbapenem-resistant Enterobacteriaceae infection, intestinal infection, acute respiratory infection, and Enterovirus infection.


SEQUENCE LISTING (19 sequences)

SEQ ID NO: 1 - DNA, 44653 bp, Unknown
  Bacteriophage YMC14/01/P117_ABA_BP
ctgacatatggcccacaatatcgtaataagccttgcgaccatcaaaaaccattagcaaaaacttattaatattccatgcagttgcatagagaatatttgaggccgccttgatgatgattaactttcttaggtctgcatcagttaaacgataagaagcaaatgcaccaccatcagaaaaagagtaggtattaaatggctggtaatctggttggaagccaaatgtttttgcattagggtcggcagtttttgcaaaacgagaaacgccaactttatctgcccaaatatctaagccaaaaccctcagcgcttgataagcgataaactttttcgtaaaaatcatcaataaaatattgaggatcgattacttcattaatgccatcaataagcgccaatattgtggggctatgtgcgtactgcgacattatcgtatctttaatattctccattagctctccttaatagaaatcctgaaagaatttaaagaagggtattggtcaataccaatagggatttcattaacccatgtggcattgtctgtacttgccacaattccttttaatccgattttcggcataccacagatataatcggctgggataaaatccttgccaatacgcaccttattttcaccagaagcagcgctagtcagaatatgattaatgacttcttgttcaatggtgtaagaaatcgcatctttatctttaacgataacttttatccaaatatcaagaaaatcaggacgtaaaatcttaattggatattgtggcgggttggatgggtagtcttcagcaattactgtcacgtctgtattgccattccaagagcaaccagtaccagctttaataaaagcagcttttgccacatcataatcattgccaccaacaacggaaacacaaatagaatttcgaattaatggataatttgttgagcctactgttacagttgcatcagttggatttgaaattacataagcatctacaacgtcacgaaccgccaaaactgcgccaagtgtagcggaatcagtcattttagaattgattgctacagactcttttcggcgcacctcaaaatcaacacgactctcttcgtcatatcccatcaccgcgctatcttcattgtaaacacgatcaagaccattaatggatgttgggataacaacaatagagtttggtagtgcttcaatagctccagcctcatcacaagttactgtaatcgtcactttgccatttaaaccaatattataagttccattggttcgccaagttcgaccagacaaatcctgaacagcaaaaccttctggaattggcgcaccagacaagccctcaaacacaacagggcattgagaacgggtagcaagctttcttgttaaaaaataaagctcaccgatagcatcttgaaaaataccttgtgcgtatcgcggatcatattggttagctagataaacaagcttgtcataacaatctttaattacagcagtccatgaagtgtttaattgatattgaggcgaaccttgaacgcgagaaacattgcccccaaaggagtcatcaatcatttcccatgtgccattgttgatttgttcggtatctggaaccgagtaaccaacatcagttatttctatctttgggatcataagcccactactccgctaagatttttatcattcgtaaactcaatagatccagacgctacacgatccttatccaaagccaattttacattaaccgatactacaccagaaacaagtaatgatctttcttgtaagtggcgctgatataaagaaattggatagccaaatttacccatgattgactctaaataaggtattccatcattcttgttgtaatagtcatctccaagaaaaacacgacatgaacacgcaatatcttgcgcttgttggtactcttcagtagctacagcaatatttccttgcgtgtccaacgctaagtcccaagtttttgggtttagaaacatggtcttcatataatattcacccccataattcttagcacaattaataaagtgaaaaatgccgccactgcataagcaatacttcgcgctgaatgaatcacaataaatgccgcaacaaaaccaataaagaaccagaataactcatgtaattgcataattaagcctttggaccgtctgtatcgctagagcctctttgaaccccgccatgtttatgagtaaacaagctaatagctttagtgattatatcagttaaagattcaattatgcctgatactgttaattttccattaggtattacaacttctggcgcatcaagaacaatctttgaatttgctttgatatggatttcattgcctttaaaccagatatattgttcagcctcttccttgctccaagtcattgttaagaatgcgtccgactggtcgcaaatgcgatttacgtttggtccaactcttgaacgagttctctttaacaaagatgtgtcacgagtcgcaaatacgcaaaatcctatgtcaccaacctgcggatcacatataaccgcattctttccaccttgtattcggtagtaaggcacattatgaatttcgcctagctctagattgttgttatccgcaccaatacgataaagcataggtttaacagaaacaaaaccgactggaccattgtcgtttgaataaacttctgttatctccacgacttcaccagtgtttagttcactggaaagaattgagagaatattagccttaaattctttagcgccgccaagattattgatgttaaagccactagcgttggactgctgcatcttttgaattcctccaagtcgcatttacattcatttgccacttgccttgtgggatatttgcttcaagtgtagcaacaagtccatatacgcgccaatcttgattgacaacatcacctataatgctttctctaatttgcacaatgccaccaaatctaacaagtggatcatacgcacaactaaatgttattcctctttgatctggtgcagggtagccaattaaacccgtcttaggcgtgatgattggtatcttaacttccctatccccacctttcttgcaaataacaattaatctttgctcgatgtacaaatcaaagtcgcacatttgagctaacttttcaattttctcaatatttgagccgttcagtgtggtgtctgttaggatgtgagtaacgccataattttcaaactcatattgcatgtcttgcgctaagaatttaataatatctgctgcatcaacctcttcaccttttgggatagtgaatggtgcagtaggacgcattttttcaactacagccatttggcttgtaattactagcgctacatttggcgcgccatctgtattgactgtcgcaaatgtaatattcccctcataagcaacttttagtggctgcccctgttcgccgacctcaatcttaaccatgtttagaattgcttgcatggtattccattggacgcgcgtaagcttattcactgtagaaaggggtaggccgtaagcggttatttgagcggtcggcgaaatagccccattgccatatgtgatatttgtagatatggctaagccagtagatgataatcggttatcaccttcagccgtaaaagtttgaacgccatcttgcaatgttagcgttattttaatgaccttctttttcacaataaaaagcctccgtattggaggctagtataacctaattaataaaattggtgtgccacgtgaataggctatcttcactgtcaccccgtcaggctaggcaccatgaaatcatacttcgtccgtccatataagtaaataacgagtacctaagcctgtgtattcaggatcactatttccatctatatcaacaaaaataaagccatgccctagataagttcgattcaggcatatccgatttaataatacatcaccatcattgttattagataaactgatatataaattatttaatcgagtttctagggtgattgcccaagtaacaccatttaatgatgtagtgaaaaattggttcgggacttgtgataaaggaatatcgtattgagccatgttatcctccgaaaagtccacgtaaaacactggttcgctgtgttgattgcttagcgccaccatctacagttttagagtcgctaggcgcttttacttcttcaatagagtattccactctagcctccaacacctcttcaagatcaacattaacagtaatcattgttgcaccatcttgagcagagcgagcatgattgatgccgacaatctgataatttagataaacgtactcaggtgtaataatgtgaaagcttaaagttgattttagtagagcttcaatctgacctagaaacaagcttctttcaagcacaccaccactgccttttgacattgaaacagtagccatagaaggagcattcactttgttatatgaagcaaatgagccttgctctactgggtattttgaaatgctagagccagcatcgtaattcatacttacaacagtatcggctaaaacaatagggattccaaattgattgacgatcccccatgtcttgccgaatacaccattaattagagcagcaccgcccaatgaaagaccgacatttgtcaaagcttccactggtataaaatctggtacagaaggcattccagctaacatatatcacctaattaaatgaatttcggaattgatagaattgatctttaaccgcacccattgcatcttggacatttccagtaacggtacttgatgatgtttgaatgttaatatcacctacatttacttggacttcattagccttgtttgccattgcttggttttgtctagctttggttaagtcaataaagtcgctttgtctggttgcttgcagtgcagcagcaacagcttgatttccaattgtagccttattaaatccagccgcatcatgaacgccgccaccagtagttttctgaatagaagcccaacgagccgcaagtacgttattctggaattgctcaggagtagctttaccagcgcgcacagcattcaagccatatttacccctactgataattgccatcgccatagcatcttggttttcagggctaaacatatccttgtcacttaatccagcagcacggatatagtcatcaaagccagaataaataaactgataacgacccattgccgaagatcggcgcttagcaggaataccgcgagatttctgttcatttaaatttgctctttgcaattctttaacttgtccgatcgttagttgagatagttccttcccaaacatttgtttagccgaaatcctagcaccacgataggcaacattgtagccacttggtgcggttgtgcttacttcaccctttgcaatcaagtccaagatagcattgccaccttttgatgttgcgcttgcaattgttggagcagaagaagcaggggtgtttggatttacaagattaccaacagcattagctaacgttccttgtttttgcccaaatacgtcatcaacaaaatcagcggctcttgagtagtagtttttaagcatatcccaagcttgtaaggctgcccctttaaaatcaccagacactaatttatcaagaatctcagcatagcctttaagcgttggtattgcattattcagcatgtctttgcctagattgctgaaagcatttttcaagttatcaacagataaagtcgaatcatcaatatactttttgaatgcgccccaatcgaacaaggacttgccaccttctgcccaagttttatagtcgtcatagagcaacccaaatgccgcgcctaaagcacctacaactagaataaatggggaaaatggagcgataaacgctaaagccgcaattgtagcctttgttagaattggtatgagaatagcgccaatagtaaaagatattgctgtgaaaacaccctttacttgatcttcatgttcttgcaagtattcaaagataccaagcgcaacttcactaagcttcacaaataacgggatgatagcatctgccatcattgttttaagtgattcccaatgctgacctagcaatgctcggttttgtgccaattgacgagatgcttttaattcttcttcggaggacttgtacatcttagattgatattcaagcatcttctccatttctttacgcccttgcacaagcgtattaaatgtgccctcatcaattcccatttttgaggcaatagagaatgcttgctcgcggtccattttagagaatgaatccgcaaggtctaacatcacatcatcagtttttcttagcttgccttgagcatcgaccatgccaacgcccaaagcgttcataaatggcaatagggtagtatcgcccatagtgacaaaatcattcatccccatgtttagggatttgattgattcagtcatgccttgagcagatccacccattgcaccagcagcgccttgccaatttttaatggtgcttgctgacattcctagattgcgctcaagatgataaagctcgtcattaagcttttgaacttgatcaaccattttaagaataccagtagcagcggcagctacgctaaaccaccgcgctaaagtctttgttacgttgccaacgatcttatcggtttcgctgacagacttattcagcttgtcgttattcgacttggctttttcggcttcacgattatattgtgagccgtccaacccaagctttacaattatcgactcaacaacattttcagccatttttacctctgttcttgactagcaaaatatttaatttttgcttcgttgtagtcggcgacttgcttagcttcaatcatgtttagcgcaccctcaagaccgatacaagtgtttaactggtgatagtcacacattccagttaataatgctctatacactgtttcagacatatgcaaaggggcagccaaaacaccatcttttaattcttgattactcatcgcgtccttgcgataagaataatcaagcactaagcttcgccttgttctaaaaaatcaatatgaatcccaattgcgtgagcagctagaatggtaaagttcttaaagtctttaatctcctgatcccacatacaaatacgtggttcaccagacttgggaacaatttgtacacagcgatcaagtaaatcaaattttaattcgcgtgaaatttcatgtggaatacgacctagcacggtaaagactgccgcgccaatttcaatcattccagccattgattttgtatcaagattcaatacatcaacgtcttttaaattaacaccactattcgcggcttgttctaaaagacgatgtgcccattcatcagcttgaatcgctggcatttccgtgattttaaaaactttgcctttatcgcgaccatcttcaatagtaactgtttttgtctttaagccttcagacatgattttagcctctaaaaaataaaaaggggattgctccccttatggttagttaacttcgattaagccttctgaaataccttcaatgcggtatgtgctaccagctaataaagcccctacaccagtaccgccagccttgcccacaaaacaacctttgattgtttgagagcgcttaacagaaggatagtaagcacgaagctcaatcggcaatacgtccatcatcttaacaaagtttttgtaaatgttttcaaaaaccatgacagatttactgttagcagctaaagaaattgtactgttggttttgtgtggaatgaagcccattgacaatttgccatctacacctacttcagtttgtgcaaacgtcacatcctcaaatgacaaaaacgcatctgtttgtgccccttcaagcgtaatccagtcgtcataaattccagcacagcgcattgatatgaccgtatttgcggatgtaattgtgttcggattaagacccattgacataagttaatctccttactgaacgttagtagcagtcatttctagacgttgcatgctagagccgtccgtatagaataacttgataataaatgattcacgagccacgcgagtttgcgagtctggcaaagtaatagatagcgcccaaccttgtgcgaataattgactagcagcatcaaagccagcttcttgattcacttggaatttttgagcattagacaagtttacaccagcgcggataccaccaaagttaataccttgatcaattggatcttgtgagtatgctcgaactgtagcaataccttgatcattgtatgggattgtcttttgagcttggaacatgttcatatatgcaagctgcaattgagtacgcaagaacacttggaagtcaaagttatcaatccatttgtactgacccgttacagagccattgccagcaaactggaatcggtcattagcagtagcccaagcgccatagaatgagtagccatttttaactaatgtttccgcatcagcttcatttgttacgtctggaactaatccatcttgactacggaaagcagtagttgtgcgcccgttagcttcttgatagttaattgaaccagaaacaccacagaagaaagctgctttgtcaaatgttccatagattggaacaacaccgctagtattttcttttgcccactcaccaaaagatgcaccactttgaccaagagcaacaggatcaagaccccacgtatacagcttgaatcggctattttgttgagtaatccaagtagcaaaagcttttagagcatcgtcatcaaatacgccatttgcataagtgaaattcacaaagttactgctaaactgaattgcgctcaatgcggcagtttcaggcgtatcttgcgttgtatgattgtttaggattgcaccagtgtcttgagttagtttcaacttatcagccacagtaccagtcgcaaagctaatagttgagccttcgcccgtagtacctgattgaattacaaaacctttagttgtggactggtaaacacaagtcaaagttaatgctgtaccgattagggccgccgcatcgctatatgagtttgcagtacttaaatcaacagtaacgttttttgatacaccatcaacaacgatattcagactgccgttaatcaatttaagatcagcaatgcttgtaccagtaatatcgccgccaataatagtagctgggtattcatctgaattgtatgttgcaatgaatagtgagtttggtcgagtagttgcgttattaaagccgttaaagtaaacggtcgcaaacttataaacatcactttctagaccgtagtgctgaccgactagagtattagaaaaatattcatagttaggatacacagcttcatttacaaataaagttgtatttaatcctagtgggttgccaccgccaccaattacggcagggtagacagcagcaatattacttgctgggattgagttaaactgcattgatagactccaaatcgatttgagaaacgtcttccagtatattaactgttttttcgtactttgtattgtattgcaagtcagcttcaatcataaagcgcaattcatacattccagcctcattaacaaaagataaatcgcgtatacgcggattacctaatggtacgcaattaactagcgtatcagtagtataagggctattccaaagcgttaatatttcttgtgctcgatccattgagttatcaccataaaaatctaactgcattgtgccgtgcatagagttaaagataaattgcttgtcaccatcataattaacggacttttgatccaagtggcgaccatgcataaaagtcatgataatggcgtttttagggattgggtttaggttgttataacctctaattactgtcgtatcatttgcaggcagattaaaggttttaagcagatactgacgcatatctgtataaaggtcggttaatatgctcatggtgcaaatactccttgattgaaaggcttggcatcagatccagcaaaaccaaaataacgagcatcatgtggcgattccttaccagtattctgaactagcactttcacccatccgctatagttattttcaccttcaacggcgatttcatcacaatatgactcaagaacagctttaacttgccattctgtaggaaatgcctcaccgtatggattcatcacaataatagacgtacctttttgcatggcgcgtctaatcgctggaatcatgccatcagcatagatagataagaattgtccttgttgattggcaaagcccagatgctttaaatcctcaacgctcattgactgagcttgaacaactttaggttgtacaatgtactgcggcacttgtgtgccatctggtgtgacagtaaaaccttcagcaaccttcaatagtgcttctgtattactatttactgccgttgtcatgctattagctatacttcttaatcttagactcattaaaaaaagccccatcgtttgatgaggctagtttaacattattactaaaaagtttttacaagattgtatgtaatagaaatatcatcaacatcctcaaatattagtttactatttggaaagaatttactaataaggtcgactgcatttaagccacatgaatgaatactcatatagtaaaaatcatggatcattttcttcggagcggttattttaataacatctaaatttgtaacctcaccattttcactttcaaccaaatcatcatacttattatttaccatcctaaaaatttctatatgattcataccaaacccctttatttgcatattcttattatatctatttactatcgagaatgcaatatgtgatttaattaatctataatttttgttgtgaaaagcgtttgatcgaatataagaagtgccttaatgtaatcatctgaacatttcgcaagatcaacatcattcaattgtcttgattcggcatctttgaaaaattcaatggctttttcaatcttctggtaaagtttctctgaatttacttttatagatactttcatagtttaccctcaacaacttcgtatttgattgagtcgtgcattagcatggtatcacgcagcggagaatcgctccctttcttagcaatcgtgtatggtgcgttagggggttgcgaccaaatcataatagaatactgaatctcattctgcatactcaatccaaccaaattgagtgtatgctctaatgtatatccagccttaataccttttttggttaattctacccaagcttctttttgctctcttacagtggttctcataaatggacgacttggaatattcttaaatccatattcgtttttataggccacagtagcaacgctagtgccatctggatattttgatccttctaagacaccagcttttacatattgatcattactagatatcaagcggtttaatgcttggtctagtgagcctgtacgttttattgacatgagtagccctccacttttgctaagtttagcatcattcatcaccaactcataaacatgaataaaattcacctcatttaaatttgatgaataattctaagtatgcaaaatttgcatagatgaacaagttaacaataagtgaaatcaaagatgaattattttcacttaacgatttttaatcaaatgctataatttatctgttacttacaaccagtgtgaaactaagtaaatcggttatgtggtcggaacaactataacggcagcgcatagcttaaacaatcctgtactgtgagggtcaagcaaacaggaatctgttggaatagtcagaaatggcgtgctaggcagagcttggactgagttaaatcagggtaactagcaaccgaaatatgtttaacaatgatttcggactttaatcgaagggtcggctccagtagcatactttgataaagttaatgttcaagatagtttttttaaaaaagactgtcttaggatgaatattaactttttctctcagaatctcaccaatagcatatatctatatagtattagttaacttaaataagatttaggggtaaatcatggctaaaaaacaaaaaagtccgaaattgaatcttgaaactcgacaacttcgtgactttgaaaattggttaataggcaagggcgcagacattgttgccattaagaacaaaggtgaatcaattagatttatctttgatggacgatgtggactgatctacaacacaatgcgcgcaaatccactcggaactcaattagctaaagtctttctgataacccaaaaataacccattcggttttgtttcgctttaactttgcaatacgtttgcataataaaaaccccgtcaatcgacagggtttaaatgtgaaatcaatatttttgatatgggctaaccatcccagccttctgagctaagaccatagccttctttaaagatgagatccttattcataattaccaaccaaaaccatgcttatcaaagaattttaaaaattgatgtaatacaaattcaacaaggtcttcttgattttcagcatattcaaggcctgtgaagcacttatcttttctggttaatacagcaatagcagaaccttccacttgaatttcagcaaaaaaacaaccagctttaacggtgtgaatttctacgctgccagaaatttgtgaagaagtttcaatttcacattcaggaaactcacgcttgaaatatccaataactttatcaatattcataaatacacctattaattaatagttgccccacccaaaaggataaggtcctttagtgcgtttaactggcatcggagcagtagcagcaacccaagttgctgtacgatacgcagcagtcttggcatagaactcagcaccatacggcgtagatttaagccattgctccatagcacctgaacccatagggtagtcggtactaacagatatagagccttctgtagccgatgaaatgcgtcctacaagtccagtgtttccgccattgatcctgttttgcaattcagctttgtgagccaccaatagccaataccataacttacgattcttcaagctaatgcaagaattttcgctattattaagcaagccttcgatagattcaaaaaagtaagttaattgctcgtttgaataactattgaattgcggaaatgcaagcttaaatgcagcaggatcgaaaacgaaaacattagacattagttttcctcaagtttgccctttttgtctagctctgctggatctagggcttcaagtccagttttaactttggctttttcttttgcctcggctttgactttagacgaagattcatccgcatagatcaactggtttactagaagatcatggttagcaaatcgagcgcgccaagcatcccacaattctttatctacgccacttgttaaaccaacttttctaaagccaccatcaatatacatagaactaggggagttccacccgttaataacttgtgattttccttccatttccatcaaaataggaaaaggttgaagtaatgaaatagttacagttgccattgtttaagcctcttatttgataacgttatcttaacctaaaaaaaatgctaccgctagggtagcactttcttgttattgattagggagtttcgtttggaactgttgctgtaacaacaaacattgggcggcgaataatcgcgccgtaagttgtttgacaacgcttttgaatccaaccagatgccttctgaatcattggatgaacacgcattttttcagcataagccaattgaattgtgtcacttccttgatacttgcgaagaaccaactgaacaacctgacctgaagctgtcgaatattcaggaactgagtaaatgcttaaattagggaagctctttttcaagtagtcagcaacattaagaccgtaatcgttagctgctgtaatgagcgcttcttgctctggtgacattaacaaaataaggtcgtcagtacgcttaactaagccgttcgcttgcttagccaattgtttaaacattttttgaatgtcggcaacaatctttaatggaagctcgaacgtccaagcatctggaataggaattgctggcaataggttcgggtcgttcagaacaccatagttttgcaaaccagctacaccaaaagcatagatttcatgttgcatcttgtttagtgattcaacaatacccaactctaattcagcagcccagctaagtttagcttcgccataaatagccgcctctttttcaccaacgcgcaagaactgttggtaattgtaagattggcggttgtggtagtttacgtttgcgctagttgtgccattatcattccagtcgccataggtaacgggcttaccaccattctcaacaacagggaatgtcataacagaagttgtccagtcgcctttctgaacttcttgtagagcctctgttaacgagtttggagagaataaaatgcgcgtaacttctggatcaataaattgcgtaaacatctgcaaaacaccagcgttaggcgttgtaagcattgcagtatcttgagcaataatcattttgtcgcgaggagttgcgatacgaggcgtgccttcagggtagtgaacgccgtaacgcgcaagctcttgaggtttaaagctttgattctttgtagtcatctgatcaccttatttagtaatagtagctaagtcatttgcaacagcgccaatcgcaactttaaatccagtcgcgtagtgatcagctagtgtgtctgcgctagaaaccgcaattgagccatctgcattagatgcaaaaacattttgaccaacggtcgtaggagtagcgatctggatatagaagtcgcctttatcgtgcaacgtaatttcctttccagcaggaattgtcatagacgcctgaccaagccactgagtaataaccgcaatattgccagaacgactaacaaaaccaacaggctttgttccactaattacattagtaaccaatccagtatcggggtcagcttcggcaaatcgagcaataaccacaccagaagcacctgctcggaattgagcgcggccagacaatagggtatggcgagggttttgcgatgcaaaatcaccagcaacaccagtacccatcttaatgttaacttgttgttgtaaagtcatatatacctcaacctaatttaatgtgagcaaaaatgtcgctaggagcatcatataaagatgaatccatagccatagatttaggagcttctgtaaccttctgcgacttaacaagagcagccaagccggcagtattaataccagtagtgtctacacctttctgtttaagcgcataggcataaacttcatgatctgagctaaagccatcaagagcaataacacccactaaaggctcaacttcgcgaccagccttaaagatgttcatgatagagccgcgaatttcagcagcatcttgagcaatttcttttttgtcgtctttcttggcttctcggtcgtctttgtcagttttttctaacgctttacgctccttatctgccttttcgccatcgtcttcatctttggctttgtcatcatcagaatcttcaacgatttctacatcatcttcatcttctgcctttttgtcgtcctcgtcctcggctttcttgtcatcttcatcaagagccaaagaaccgtgaacggccacaatagtttttttaacatcttccaaagaatccatgcctaactgtttttgcagttttgcaaggcttccgtttttaagtttgattgtcatcaattgaccctctattgaatctgcgataatcgcgtcacttccaatcctaccacgttcaacgatagcaacgtgattaccatgaatattcgtcataataccatcataaggtacaccattaaaagtacccgacttcatgacagcatcataagcgtaacctgcgcttaattctcctaatgccttgctttcgatatagtcaatcccttcttgatcaaatacgcgcaatgatgaccatactcgaccttcgtcatccattgttatgtctgtaccaattgaaccaatagttgactctttttctggttgagatgcgtcaacaggaatatggcgtttaagaagctgcaagcccttgaatgtgtctaatgatttacgcaattcgtcaggatcacgcaacagcatgtattctttgtttggatctaagcctaattccttccatcgtggaattgacgaaccaagataaggatttactgcggcttttgtgataatggttttatcaacaattagatggccattgcggtcgtagctacgagctgatttatccattgcatgttctggctctttcttttcctcttcaagccattgatcaaacaattcttccaacttatcaaagtcagtatcattagccattccagattcacgatatgcaatagcaatcgcttgatctttcggttttcctgaatcaattaactcacgaatgttcttatgaataacatcctttgagctacctttaattagaggcatcaaacatcctccactttaatttttagtcttttgattaaatattctattgctctgtcatctgaccaaaaaccaaaaacaaccatgaatttgcagaaattcacaacaaaattggtttgccatgttttttcatagctaagttttacctttatatctgcgatattcatatcactcacctatacaatttttcttattatgcaataaaaaagccactggttaaagtgactttattctaaagaagtcgcgcggcttgcaaacacaaccctgccatcgtttaccatcccaaaaatcataagcataaatatatccgcaatagtaagcactatcctttggtgtattatctcgtaagttttttatgtaaattcttcctgctttatgtgtcgccccatcaggcgcattcctcttaatctcgtcaatgttcatttacaaactccaaagcattttgaaatacacaccaatcaattttaaatcctttattgagcatatctgatttcgcttcatttagttcgtattgattttcgcaaactatataatcaacaccgaatatctttttattattcataaagtggattaatacataatttccaaccttctcatgactatcccttgtcaaccatattcttgacattgcaaaatcaaatttatggtgaaatcctttctttctagctaaatctatcgacttaatcattcttcaatcctcatcttgtcgcaatgtatgcataagtaagctttatagatccagcaatattggtagatatggttgcattctttgagtgtggtcattggtcactccatcgctttcttgatttcatgaatgcagaacttcaaagcaaatactctttgatcatttccgttctttaggttctgctcttttgtcaattcaagttggctgacaagctgactagctgcatttcttaacttgtcgttttcaatctttgagttatgtaattcttgagctaggcgatctacttctaagattgcttgctctcttgtaagctcttggtcagcaaagcaagtgccgcctgcatggcaataaccgtctgcgccacagtaagggcttccacccttacagcgcatcacagcattaacccatatgtcatcgtccttacttagcaagtcgtgctcacatgggatcttgattgctttcatccttcccccttgagcgcttgctctatatcacaaatcgtttcttcaatagattcactataagtttctaatgattcgttatttaatgatctaagctcttttgttatttctaaagctaaatccaccctcttttgcatcttcaacatatttatgccttgctgagtgtataatgtttgcagctcgtcacgctcttgcttgatctttttaaagtaaacttcatgaccaatcacttcaccgtgatgagatgctttaagctcctccactttcgcttgctggtgctttaatcccaatgaataaatatcaaatgcttcagtcctgtccgctgaaacattcatatagttttcttcccaccattcatcaaactttagttcaaactctttaaactcactcatggctggctcctttttctgcatcacacatttcacatttatctatatgcccccacccatcatctcgaatgaagccaaaccccttacaagccttacatttgactttctttttctcacccaccaagaaatatcgatctttctggttgtaggtaatatcaatagaacctgagtaatagcgccttaacgccccatcaatatgaaattcgtgtggacctacacaaaacatccaccccgaatccccgccgcactttgtaaaccatgtgaaatatgcttctctccatttcacataacggccagacagatgaggagtcaacaattcaattaaacgtgctctaagcatctccatgcttgctgacatatctccatagtgatattcaagattgtagctatactcgcctgtgttatatctagttggcatgagattcaccgcctccgtatattgattcgtggtctttgattgcctgctccaattcatcccagccgcttacatttggatgctttgctttattccgcgctaggtagtctttggcaaacttagtaccaccatgccacttgatcagatcaatcgactccaccaagcgtttgagatccgccatgttcacaagttcaattcttggattaaaacgatctgaatacttttttgctttggtgcagtagcataatgttgagtagtaacactccatatatttgcttgggatgccttcaacaacctctcgcgccttatccaatccttgctcacgaataaactgctctggtttcatttaaaaatactccaatacatcaaagggcagtaaattgcatgatttttaatatctcttccacaaaattgacatttaatcatattaaatcatccattctggtttttttacctttaggtttttatgtggttcatcaggaaagataaaagtcacataaactttgctttcttcataatgataacatccctctggcatgttcgctttaagccattctaattttgccttgctcatttaattctccatgcaatattctattgcttgttctaatgaatgaaacatatcaacatgattttctaactcatacacatgatacagccaatctaagtgatctatttttattgttcttccattaacaaaaatagattcaccatcaaaaaccacttctgctttaatcatttcaaaccccttctgctgaataactaaatcttaccactattaaatataattctcaatagtaaatctaaaatagtttagacaaaataaaaccacccgttaggatggttctaaataaacttagatgaagtgaacatgggagagtcgaactcgccaataaaacagtattttacaatggttgatcaatcccaccgccacaaaaaccaattgagtaattaattcaatcttataatgtgaactgcaatattctagcacctaagctattcacttctctaagttgcctactttcaggcggtcatcggtttctcgtgccaataccgaaaacaacagaacattttagccatctgtcttgcctattgatcggcaacgtgcgaatagttgctttgctatgtccctttgaaggtactcctagtctaatgatattcactgtgtcgcccacagattcgatcaattacagaagcgacttcacacatccgcaaaaattacaaaccaaccaaggcgcgcagtaatttttaccaatgctacatgatattgtagtcatactccatgatttaaataataccaaaacaatctaaaaatacaaatcagttgttttgatatgggttattggttttacaaatttacagttagtgaaacggattgccgtgtcgatgtgcgctttcacttatgcttatcacatccaattaactgtattatgccatactttctggaatttcgataattagctttttcctacagcgacaattgatttcgatgccagtgtttgtccacttgccatcaagaaataagccttccttgatgttaaatctagttccatttgcctttacgtggctttgacgaaaagtctttcctgcatgactgtgaagccagtaagcttccgttatgcccaattcctcagccctagcattctcaaaagcctgattcaacttagctgtctgatcttttgcaatattctttgccctgcgatctgttacgccgtcaatctctttgagttgcttgattagtgactcaacatcatagccattcttaacactacgccaaaccgcagagcgaactttatctaaatactcattaccaatagacttgattagagcaacgttttcacctaaagcaatttgcgcttgatcttccatatatttcgttggtcggaagtttacagtaaagccacgcttgcgaagaatacccaaaagtcgcttgtcgtagttggttttagccttacctactaactcttgcgccacttgagtcgatagtttatctaaacgatcttgccaccgactaaccaacccatcaataacatgtcccatccaatccaaaatgccatccatagcaatttcagaccgcataggcttaactacgtcctgaattaaatcagaacgcatttcatccatcatgccttgcatttgctccctgtaccatttggtcagggatgcattaggcgctattgactcaagggttatttccatatctatttttcaacaacatctatgttttcgttttcagcataaggatcacgctctggcgcactaccttctaattgataaccactgtcttcatcatcattcaaagcttgacgcgcttcttcgttgtcgatcacaccttcttgaatgtaaatctgagcagtttgtgctttcttcaagtttacgtctgcttgttcattatcgtccaattgataaagcggattaaagacaaacttaatactttcatcaatatttccaaacaagctcaattgcatacagtcaaggataactttgatctgcggcaaaatatacgcttcttgctgtgctgatacgcaatcgtaaaataccctaatctcaccatcagaagtattaccaagacccgccgttggggttccaaatatcttcaatactggaatttttgaagggtaggcaagcatctgtgtaaacttgtctagcaatgtatcaagcccagataatggcgtattgatctggaagaactcttcagattttccatccaaagccaccacgccttgattatctcgacctaaagccaatgcttttaagcgaccaatcaactgatttgcgccgccttcatcacccgctaagattgcagacatatcggtcttgataccagtcaagctaaacatggtaatcaatttagctacagcgtccacaatagattgatgacgctgaacgtatggcaacatcaattgcgacattgacatgccataaaaattataagctggcttaagcatttgcccaacgggacgcataattaatgtaagcaatcggctatgatgtacatttttacccataacccaccactgcttaggcacaaagaaatcactttctagtggattatttgcattgtacatactaggcgtggaccaaagcggctcaataaccgtgaagccttttaacgagcctttcttaatgcctttctcgttaatcaatagaggcagatcggtcttatcctcttgcccttcgatctgaatgtataactgagagccaccatagtagtaatcattctcgatgtgcttacggacaaggttgcgaatgtccagcctgttaaattcttccatgagaatgtctacgcgcttctggtcatcacccttgacctcaccccattcgcgtgtcatctcttgagcaaatgtttcagcaatctacgcgcttctggtcatcacccttgacctcaccccattcgcgtgtcatctcttgagcaaatgtttcagcaactaggcgatattcagtagactgagccaattgagctaatacttgatagcctaaaaagttgctataaaactgtggctctaatccagcatattgcgaagctgtacaaaacccatccattgccatagcgctatgaccatctggtgcaacaccatcaggcaaagaaggcgctttgtactgaatcgccacaccttcattatgcgcttgcatagcatttaacaaagcgttccatttcggctttcgctttggcgcttcttctttctttctgaaccaatcaaacatgttttaccttccgaaagccgattcaatatcggcgatgttaatcttcatacccatgccacgtttcatgactttttcaagtgcatagcgcaatgcatcgacatagtggttatctttgtcaatgataataggtaatacctcatctgtcagcctgtcttttttgtacgagtaatcacggaactcttttagcgtttctttgcatcgtgaatgaatgtagactttcttaaacgatttgatgaattcaataccatcctcaacagaacctttgcccttctcaacaggcgcaattctacgcaaacctttcttttttaaatgactaatagattcaggacgagcgctatccgcataaacagcatacttcttaatgtctggtattttcttctctaaaaattcaacagtatcgtctagctcaagaccaactttcccgccttcgtgttcaatccacaaagaatcatcatgaatccagactttgacacatgccgttggatcttgagcaaaaccaaagtccaatccaatataaggaccatgccacttttgagggtctggcgtaaaatcctttttctcgtatttacccttgaagatttgtgcctcagacatttcaagataagcgccttcccatatccagcgataagttacatcgtcaagatttgcttgatctcgtttacgttccaagtcgagaacatgagggaaccacggattatcagaatagttcatctcagcaccaataccgattagaacgccatcatcatcccgtatttcttcatgtctaaaccgtgttgaggttgggctatctcgtctttcagggttccacgttacccacacttcagaataaacaatcttgccattaggtaatacaatttcttcacgtacagttggtaatagcttacgccaagccatctcgctaaccgtttcagcctcatccacccaactaagcaaaatacgcgctttagatttgatgctatctaagttatgacgcaagccacagaaagaatagtaaatatttttattcttggttataatatagttttcacccatgtcgtaataatcgtctaaaaaggggacggatttaatcgcctgcctaacctcctccattgaagaatcagcaagagtattcatatattcacgaccgcagaggatagtcccaccaacacccatctcggaaaacatataacccttaattgcggtcattagggcgaacgtccttgtttttgctgatccgcgaccaccccacgaggaacggtagcgaacaccttgcgctgaaaatacagggattaacttaggcggtatctgtatctgtgttttcgacattaggcgctactagctcaatgatggttggtttatagttcacggattcgccattcgtggtgacatccacagcctgcttttcttgccatcctgcttggacttttaaccaaaaaatagcagcaggtgtgctaccgttctgcgcctgctgaaataagcattgcgccattttcatgttggcctgaattttcgcgcggtctaaaatttctcggtaatacttatatagcgtttttttatcaatgccgatatatgcagaaatctgatcatgcgggatgcccattgatgcaagcgcgcgtacttcggctttttgtgcttcggttggttcatgtggtttagtcattaaacttctctcccgtgctttcaagtacagcatctttgcctgtcaatgtctgccaacggttaataataacatcacagtatttcgggtcgagttccataagataggctttgcggttggttttttcgcatccgattaatgttgatccactgccgccgaataggtctaaaacgcgaccattttgtttacaaaaatcattaatccctctctcagatagctcagttggtttttgggttgggtgaacgtatgagcttgatccatcctttttaacatcccatactgccataccacgcttaccattaaacctaggtctgcctttcacacaaaacagagcaatctcataatctaaagcataacttccttcaaggtcaccaattccgccaccatgtttataccatatcaatgtttgtttatagaacgactcaaattgctctctccattgtggataaacgtggtgtgaagtccaaataaaagctgcagtgtcatccttcataaatgaataaataactggagcaatatcaagtattacatcatcatttttaataacatcaaatttatcgttcattccgtttgaatatgaaacaccataaggaggatcagtaaaaacaagatcagctttctcgccttgcattaacctctccaccgcatcaatgctcgtactatccccgcacatcaaacggtgcttgcccaaaatccaaacatcaccttcttttgtttttggttctgctggcggctcgatcacttcgccatctgcatcttcttcatattcaggcgcgatttcatcaatctcaagctcttcgatttcatcaagtgaaaagcctgttagctcaacatcaaaatccatttctcgcaatgcttcaaattcaatacgcagtgcatcaaagtcccactcggaattgagagccagtttgttgtctgcaataatatacgctttcttttgcgcttcggttaagtgtgaagcatcaacgcaaggtaattcctcaatacctagcttctttgcagccataacgcgaccatgccctgcaatgataccgttatcgccgtcaatgatgatcgggtttaaaaaaccaaattctttaatgctggacgctatctgcaacacctgctcgtcgctgtgcaatcgcgaattgttagcgtatgggattaggtcggatgtcttgaccttttttatagaagtggaagatgccattttatttcccaaaaactaaaatttaataataccacaaaaagaaaaaccgcgttaaagcggtttaatatccttgaaccaattacagcaataaacccatctatgcgcccaataatcccagccatagtttttattcttctttaagtagattgtctttccttcatctacacaatagtgcgtagctttcttagttggcttgctcttcctaatttcttcaatatccataatgcaatcctcatagcggtttaagttcggtaattctaaatcttcggtcaagtatccactctccaaacatttttgaataaatatgcagcccatattcagtgcttttaaaataagttacccctgcatactcaatataatgtgtcgcacctttcggagcattggctctgatttcttcaattttcatctacccaccctctttaaaatcccacccaaccccgagcgactaatcccaagcattttagccgcctcagtttgattacctctcgtcttaaccaaagccctttccaaaatagctttttcaaacgttttctaatcttataaaagctttcacctttggctatagcttcgtcaatttcttgatctacggttgttttcattgctagtaaaactccttaacattagccgcacgattttcttcatccttacaaatctggtatgactcattagcaaagtttgatgcaataaccgctttatcctcacctttgtaaacagggaatgagtaagcccaatcaacattggcatccattaaactagccagcatattgtctacaccccgcatctcctcaaccacttctttagcttgatgctttgcataaccttcttgacgcatcagcatagtagcctctgcaaagtcatatacagcatcacaaaacccagcattagcaaaacttggaactgtcaataacgcaattaataattttttcatcacaaaatcccttccacaccatttaaaaacatcttcacatcacccactgttaaattaccttctttatgtaaagcctttaacgtcttgatgtatcccgcaatatcatcaatcttctgttgctggtgattgtaaattttctcgcattgcttatatgcgcttatatcaaatctatcacccagacacacctttcgatagattgcattaaattcactcacaatcaatctccttccagcatttacggcaaaccttgtaagtctcgccagttaaattactttcctcgtattcccattcatgtcggcagaataggcgctttagaaatttatagatcattttttattctccataagtctgcgaacatagaacccaaaatttggatgggataaaattttatgcacaacttctttaagatcccaaatatcattagcgatataatttccacttatcgtgtttagttttattggaaatgcatgttgcttatcaatttctattgacataactgcttgtgtacaagataaacttctattgcttttaactaaaataaatccacccagtacaccagttttctcgcatatagctttgtttgcttcaataatcacttcaataatctgatcaagatttctatcgtaattttttgaagcttcaagaccttttttaatagcttccataaaatccatcacttaccccttaaccaattaaatacaaccataccagcgtgatatacaatcaacactgaacctctaagcaaatctgttgccgcgtactttaatacccttagatttacccttaactcttttttgtgatccatttttcaataccttgtatttactcaccacaatcaatggaaataaaaccacactcaagattagagataaaacaccagctaaagcccaaaacaactcaaccaacaatttaggtgcatgtttgaataacttagctacatcaacaattgccaaccataaagctttaactataaccattttatctctcctctttatattctcaatagtaaattatatttataattaatgcaaggattttaagcaaaaaaaatccgtatcttggggttagatacggatgtaaaagtttcttcagttcgataaatatagcaaatttttagttaattgcaagtaacaattttaattgcttgctctacacaattaaccacattaacttgaccattccataagtcatgccattcgatttgatctggcgttaatttttgcgctgaaactggtttagctccatcttttatctctaataaaaagtttttaccgcgatagcccaccaaaatgtcgggacacccttttccaacacttgaaagaatttgaactgtacacccaatctttctaagtgctgccactatttctggttgatttgcatctatcttagctgctctcacgcacatcagttcctatataaaatggtcctttagttttgtacttttcctcaaattcttcaatagtcatatatccaacataataactaaatccaaattcaaactcattagaccaatcttcattaaattttaaaacatcatcaaaagttaatctgtcctcacttgcctcaattataaaaaattgtttgattagtcgattaggctcaaaggcttcaaccccacgaactgcatgtaaatgtaattttttcatggacgttttctcaatgagtctttccaatcgccattatatttatgcctgacaaccatcatatcaccactacatgcatcattaattccccataagccaatgccggatgtattaaaaaatatatctggtttttctttaaagacaagcccaaaccccaaactgtctacacaaaaccatttataatcaataggaaccatcctccaatcaatctcaaattcttctaacacttcaggtcgacttttatctttccgcttttcatcttcagcaagcatcaattctgcaagactaaaagaaacatcaacaatctgatcaactgttattgtctcaaattttgttaaaagaacttttgaatattcatgcgccaattctaatttattcattctcatcttcctttctcatttcttcatatctcaataaagagtcaattgcctcgcgaacatctttatccacatctttaaccccgcgcccaccagcgcaaagcaatttcttaactgcatgttgaatacatggatccgtaacatcaaacaatttcaacacacgataaacatcaagctcaagcaaattagatacatctttaaagtaatgagagtgttttttattgtttaatgatttcatttcctcattaacaattccagattcatcaataaaaacttcttgtctttgccataaaactttatgagaacgatcgatgtttccacgcttaaatgactcatcaaaaagattgtctttactccattcgctatatttccagtaaggatctttttgactccatgctaatctacctgcgtttttataaaaacaaatcccatgattaaacatatctatattaaattttatggcaatttcagcaccctctggaatttcaattttattaatatattttgggcaacaatacatattatcacgatgataatgatacttgccatcttcagtttctaataaccactctttcatttctcatcctcacataaaacatagtttctttcgtcatcttctttcttagcttctagctcaatgtgaattttcttccattgctctattagcatttctttcgtttcatcatcagctaaaggctcagagccattaatcttgtcgcgtaactttttaatattcacccttcaactcctaatccaaattcaggattcattagcttaattgatttaaattccatttcatgtctaaagcttttgaatgttgaaaagcatgttaagcaacaatgattagtcttaaccacaaattcaccgttacccacttctttcaaataagcttcaagctcatgattaaaaccaagccatttctttttaaaaccaccatcattgacataaatgtcagattcaacatcacccaagaactgccacaaatgcataacagtatcagtagccaattgaaactcacaatcaaccgtttcgcctgttttctcattaaccgcatgaattcgactaacgatatgaaaactttgctctgccggtatcgtctgcgtctttaatgctaaagccaaatcacccttatgcgccttcatccagactttaagcggtgtatgagtagactcttgtggatctacaccattaagcaaatgatgaagctcaataatacttttagcctcattaacctcccaagtcatttgaaacttgtgaatcttctgctttacaacctgcttcccgttgtattttttattccgctttttcatacagcacctttgatttttcttagctgctctacgacttgctttgcttctgctttggtgcgccaacaccccatttgagttcgacattcatctgtatttgtcccctcgtaaatgtcagagtcataaccagaatttgtatgagaaatatagaaatattcctcacccaccttcggctcaaaaggcttcggcagctcaagttcaagcttgacggtttgtggtttgatgcggaatttatataaaccattcacaacctcttgtacagtaaaataaacaataggtacccactcttcattttcttctgaaaacccttcagcctctttgccatcagccaaagctcgcaacgcctccgcgccgctaatcaaatctaagcctttagtgataggctccaacccttcatctgtattagttgttgatctcacccacttaccctcaaacataaagtaaaaaactccatcacagcttttaaagtagaatccatctaagacgcttttacttatgtaatttgcatccttcacatcattacgcttcaacacaacaaggtctcgaagctgaggaaatgtaatttcctttaatgtgtctttatccaaagcttcacaagcacaacccattgatccatcggcgaaagctaaaacccatttagtgtttgcttcgtatttgccaaaaagcaaatctagttcatagccaagctgtataaacaactcctgagcctctttgctctcagcttcattgtttacgcgaattttataattttccatcacacaccccataccatattatgaattaacttacttaaccccattactgcaaaaatcacgatcaaataaaacaaaaccatgacttttgcaaatcctataaactctttgctcattggttgttaatctggttatttacgatcactgtttggctaccgcctttatttgacaatgaccagaacaaactccaaaaccagaacaacacagaccaacctaaaaacaaattcattgcaaagattgcccatttacttgcatgcccacgcataaagccaataatgctaggcgcgaaatacataattactgaaacaactaaaaccaattccataataaaaccatctgttaatttatattctcaatagtaaacactatccacaataattgcaactattttttaaattatttgcatgtgactatgggttttacactccgatttgtcacactccattttttaaactggtttttaattcttactccccatgaatgagcctcatagacagtaaatttttcaccagcaaaataaacccgatcaccgactttgtatttcatttctcaaccctcacaattggcttaattctaaatctcgcgtcaaatatatttaagacaaccttgtttggatagtaacgctgacgcaagccaaccttaccaacagcaacaaattcataattcgaagtcataaaatcaccatcccaacagctcgaacttactagaacaacccttgtcttatattccgaatcataaaaacatgtttgattaataattgattcgcacatcattttatggaatttgtaaaatctatcatctgactttctaaaccaacaatttagtatattattcatactctacaccccttctctatctcttcatccgttgcgtgacgcaaacacttagtgcctgcacgtaaactatcaattttatgaacaacactgtccttgctatattcctccttaaatacaacaaggtcgcctaattcatatatttcattttgtcggcggtattcgagtaaagctgcttttaattcatcatggtgcttttttaaccctatttgcgacatttttaagctttttgctacattatacccgcccaaattatcaattaaattcatttaaaaatcctcacatgtccaaatattctaaattaagattactaaatcttgagtattgtagttcagttgctaatctaactgttccaacctcaccatcacgcgctttggctacaataatttcggcaataccctgatcttttgagttcttatcataatactcatcacgatatataaacatgatcacatctgcgtcctcttcaatagatcccgactcacgaatgtcagacattagaggtcgtctatcttttctagattctaagcttcggcttaattgcgctaaacaaaaaacaggacaatcaaattcttttgctatagccttaagaccttttgatattgcaccaactgccaagtgattgttgtcagtagacaatggtgatttaagtaatgttaaatagtcaacgaatactgcaccaacataaccatattttgctttcatgattcggcaattttttctaatatctgacaagctaggtcttgccctatcatcaattaaaagttttgatgccttcaacttattagttgctttatagaaaaactcccaatcctcatttttaatatcagagtcacgaatatctcttaaatttattgaggctagactagcaaccaatctttgcccaattttttctttcgacatttcagcagattggaataaaactggatgctgttgcataaaagaaacactcaacatcaagctttgagcaaatgcggttttccccattgatggtctggcagcaataataactaaatcactttttgatatttgaccaagctgtctatccaagtccataaatccagtttttacacctacgtcaaccttctctccacgttttcttgcctccatcgttttatcaatagacgcaaatagagaaacagaaacatcataagcatccgataaagtcggcacttcatcctgattgcttaaattctccagaatgctctcagatttcgatatagcctcattaatgtcgtattgggtagtatctatcgaaatagccttaattctttctcctgcctcaaacagcgctcttctggaggcatattcatttaactggtcagcatgttgctcaagaaaatgagccagacctattgttgagtttaaatttatcaagaaatcttcatcaacaattttcgagtcatttgcatttaacttgattgagtcataaaccataacaatgtcatgacctgacccagcatcaaaaagctttttaaagtgcttaaagataattttatgttgtgttgcataaaaactatccgtagttaatttgcttacaacatcatcaataccaccattgagagacataagggcggataataccgattgttcaatttgtattgagtacaagttttcattcattttatttaatccaaataattgattggtacagctacagctcttcttactacattttcttgggcttgaactttatcagcactattattgctaacttgctcaatccaatagtcggttttccagtattgcttgtttaaccacgaactaggtgctgctataaactcaccattgttctttgtccacatttcatctttcttaaactcctctaaaaccttcatgatcatttcaaaatctttttggtatttagtaaaagttttattagctgcttctttagttcctttccgcttacattttggatattcattccaaaacttctcaaatttttctgaaaaactactttgttttttatctgtagtattctctgtagtagtctctgtatatgtattacgtttcaacgtatggggtggtacgttagaacgtaggagggtggtactttctaacgtaggagggtggtacattgaaacggatgatacaatgtctgcatcaagaccaataaaaagaacattaaatagtgttgagttgtcagtttttacggttctaaactctcgaattacaacaccaaactcacaaagtcgatcaaatgcctctttaacctgtcttttagttaatccaaattgttttgcaaagctctcataagatcgctgcaataaatctgccttaaacttttttctaactccaatcgttgcacctgtatcttcatcacgtacaatttttggtctataccagtaaacaatctcggataaaattacgatagcattcaggtcgggctttccatttttaaacttaaaaactgagaaccaattaaaaggaattacattcccttcaatattcatcaaccctattttgtctactgtttcatttccactgctatacattgttatcaccactcaaaacaatttcgaagtaaaatttgtttaacttaatcggcatttcaacaccaatactcactagtttttttacaatgtgagcatcaacaacaccattgagtattgaattcttaacagccatgcagaacctaaaatcctctaaatctctactgtgttttgttaagttgcatgttgaacaagaacaaacaagattctcaatactttcgttaattatctttgactttggtatgaaatgatcaatatgcatttccatatatgtttcaatttcaaccccacaataacaacattttttgtcatgagagcgccaaagttcctttcttaactctttagtgaatttgtagccactagggaagtatttaaaagcaacatcaaaaggcatatcattcaaagttttagacttacgaatcatatttgggtgattttctatcaataaatcattgttaagcaatgtattttccataattttacaaacctatctaaactgaccactaacaccgaaatacattggcataggctcattgcctttctttcgatcaatgtaagcgtcttgaatttcttcaatcatgaattcatctttttcatggtcgtaaaagttctttacttcaccaaattgattgacaaattcggggtattctttttcaaaccaagtcataaagaataaagcgtcttttgatatttcattcatcagttgtctcctgattaaaaagatgatttcttaattttattaaactcatcttcaccaatgcagcaaataataatttgctctatcatatggaatagctcaggttgtaactccttgttaaccatatcaagtgctgatattgcacagtcataagctgtggcaatatcgccaaaaagatcatttttttcctttccagattttagcaattctgccattttatcaggtgaaacagtgtagaatgctggcttgcatcggtttaggacacaaacaacatcatgctctgctgccttgactggattcttttttaattcgctaatcgaagttgtgattgctgtataaatttctgtaaccatttcgctctcctaaactatatggtcatcttataggtcttttaaagttaagtaaaataaataattcaactatacaaaataagaaaaactgatataaaggtaaaataaaaagccccgaagggcttagtggttatttttctttttttaatttccagcaatcaaaacattcattgttataaactcgtcttagcaatgatccgcatttgcaaggcttggtatgctcatagatcgtttacccatacttcttgcatttgaccttgcaattacatttgggcttatatcctttctaggctctttatcgctaattttgaaaggtattgctgcatatccaaagcggtcagggttcataaatccgtcattaattacatggttcattttttacctacattaaatattaacaatatctagtggtgtttatattatcaaatatattctcaatagtaaataggttggagtagttagctctttttgtgttagaatgaatttgcggatagagtgacggctcgaaaaccaattaacctgattggcctccgcatcattacatcaggttttcaatgaggattgaattatggctaaactagtttatggcgttgggattaacgacaaatcaaaaccctccatggttgatggaaagttaataaaagaatatgtcgtttggaaaaatatgatttttcgatgctattcagaagaatataaaaagaaatacccaacttatattggatgcactgtttcagaaaatttcaagaattattcgtatttttataattggtataacaaccaaaccaacctagatggcaatcttaattttgaacttgacaaagatttgctaataaaaggcaataaaatttactcagaagacacatgcactcttttgccaaaagaaattaattaccttttaaacaaaaataaagccaagcgaggggagtacccaatcggagtttctttgcataatgcaacaggaagatttgttgcaaaaattaaaaaagaagcaaagttgttttgtattggttattttaataatccagtagatgcctttattgcatacaaaaaagaaaaggaatctcatattatattcatgacaaataagtatagagatctattaagcgattctgcttatacggctcttttaaattatatggttaaattggatgattaactcaatcatccaatagtaaatagtacaggttaaataaaacccgactagcgggttaaattcctaaaagtttttttgtatcggggtgaagcttgactatagccaaacccttttcaaagcttggatgtagtcgtttaccagttgctagaccgtgaatgtgaacagttgaaatccccgtctttcttgctaattcagcttgattgatttcacggtattctaaaacttcttcaataatttgtttccagtccataagaccctccattttttgttagtttaaattattaaattagctattgcaaatattaaataatagtttactatagggaatatcttaacaggagaaacaaagatgaaaatcgaaaatatcgctattgtgtgtaaagatttaaaccaaagagatgaagcggaaaaattattaaaatttcatggactcggaaatgatatgtgtccaactggagatcagaatattaccgttgattttggtcaaacattttggttttcagatttttctgttggacgcaaaattagttttgatgaatttatgcaaagatattcagaaaacgatatgatcaaaaaaatccaccaagccaaaaaagaacttggtttaaataatagtgaactatctttaaagatgggtaaatctcgcccctatatcgctaagatgcttaaccagccgcaaagcgagaaggttcagaataaagttattaaagaaattgacgaacttttggaatttgagcagcgctgcaaggaaaaagagtatgcacgatgcgcttctgaattaaatattccagtatctataaatgatctttcagttcaaaaacaccaagatggcgagaaagacaaagaaatttcggatctcaaaaagatcatcgaaagtaaagacttggcaatcagtggactaatcaaacaaaacgatgaagctaagaaaattcatgataaggatattaatacactggtcgaggtagaaggaaatcttattggtgcaaaaattgatttggatcgaacaaaagttgaattggattactttgttaatcaatacaagcaatctgaagaggatgtaaatagccttaaatcgaagatcaagcgcgagcgaatcattattttaattgttattgcaattttaacggcgctattcttcttgaagggttctattggtggttgagttttgtaagatttgttgtaagtacaagttgtttttaaataattactgtagtgagtgtggaatgaaatgaatatattagagttggcaaaaatcattgataattctttttcacaagatgagtttttactaactactcaaaaggttggcggtgtttcaagcatctcaatatccccaagaactggacaagaaattgaattttgtaaagttggtgctgcttatggagttaaaataaatgatgaagagtttgaattaagatctggcgagtttgagtcgctattatcgccttatcttggaaggcgaatgttgttaaacttaatgaattaaaattagctccttcgggagcttttttaatatttgcgcttgacattaatactcaattcatttactattgagaatataaacaatgggtaaacattatgaaaaatgcattgattttagctgcttctgttgcgttagctgcatgttcaggtaaaccagttattactggtccttatgaagttgaatcattagatatggaacataacgtagctgccattaaatctggtgatttagttttagaagttgagtttgaaggtagattctttaaagatggcaacggattccaatcatggaatgatgttgaggttgaatctgtaaatgacgttaaagtttacaatgaagatggtgaaacagaaaactatgttttgcctagcgaagaagtatcaaacattgttcaagttattgaaaatgaaattgcggagaaaatgtaatgagttggcttgacgatgtgaaagaacatggtgcagacgcttacttctggtcaatcgaaacagcttatcaaatgaatgtgattgactctaaagaatacagaaagcgtatgcatgaatatagagatattcagcatcaagaaagacaggaaacattacttaaattattggagatttctaaatgagcaatctaactcagcagcaaaaacaaaatgcattagctattaaacaaacgcttagcaacccatcggtgatgaagcgtattgaggaaatgatagggcgtaaatctgatagctatattacgtctgtaatgcaggttattaattcaaacgggttattggcgcaatcaactcccgaatcggttataggtgctgtttatacggcttgtgcgcttaatttgccattaaataacaatcttggttttgcttatattgttccgtttaaaaatcgacaaactggcaatcaagaagcacaatttcagatggggtggaaaggttacttgcagttagcacagcgctctggtcagattaaaaagattgcctcaattgcagtttatgacaccgatacagaagaaagcgtaaaggctcgtttaacttcattcattccacaaaaagttagtggcgaagttattgggtatctggcttatcttgagactgtgacagggtttgaagctcatttaacaatgaccaatgaagagcttgagcagcatgcaagcaagtatagccagacgtataagactgcaaagtcgaaagggcaaagctattctgtatggcatcagaattgggacgcaatgtgtcaaaagactgttattaagttgcttatctcaaaatatgcgcctatgtcggttgagcttcagcaggcgattgaatttgatcaagctgtgattaatgaagatggcgaagcttcatatgttgataatgatcaagaaagcgaaaagccattagctcgattgatttctgaggatcaattcccgcaattcgtggcggctattgaagctggtagcttaactaaagagcatgctttaaatccagatatttacgcattaagcgaagaacaaaagaaaatagttgaggcgttatgaatctaatttttcgatgttctgagcttcatcgactaatgggaaatgcaaagtctattgactctgcatttctcacagaagaagttcaagcaataaaggcgaagaaaaaacgtaccgatgaagaacaaaagattttagatgatcttttggatagaactttatcggctactgctaatacactggtcaaggaaaaagttaggcagttaagacataaagctccaagtaagtttactggaagtaaagaaacaagaaaaggtaatttagttgaagatgacgcaatcttgtttctaatgcaacagaagtttattagtgctgaaaagaatatgattcgttttactaatgactggatcactggcgaaccagacattattactaacacagcaatacgtgacacaaaatgcccgtggtcatattggactatggagtattttaaagaggatattgagagcaaagctttagatgctggttatgattggcagcaattagggtatatgtggttgttaagagagaatcacgattacgagcagaaaataattaatgaggcttatcttgattttatcctaatgccaacacctaaagagtgcctaactcgacatgatgatgaatatttgcatatcgactatgttttggaaatgaatcctaatgatcgaatttcatcgtatagagttgaatatgatcaaaagaaaattgacttaattaaactaaaagtagaaatggctagagaatatgccaagactttggtttttggaggataaatgaaaatcaaggaatcattagtctttgtgtttgttttttggatgcaaaaaggtgggcataacttaaatgagcgtaaaggcaatattgttatgcgtaaaaacggaaaggtggctgaaatttatttttcagatgccgattattatgaaattaacgactattgcaaggaacgatatgaattgtttttaacgcaatggcttaaatatggagtggaatttatcaagaatttaaaacgtgaaggcgcgttagaggtgatgcgaaatagagggcaacgatatttggagttaaagaaatgagtaaatatgattggtcgaaagtttctgaagaagccaattatgtagctacagataaagatatggttcaagtggaatttacagaaaaaccaatcatctgtcttaattctggaatgtggattgatggcaccataaatggttatcacgattacactcataatgacaaattgtcagattttaaaggtgactggaaagactcactagaggaaaggccaaaatgaacccaatagcaatgattttattatcctttggattaagttttgtttttgtggttgtcttatatttatatgaggcatataagcggaaacgatacgtgcaaagaaagcaagatgatcaacgtaaatttaatcctattcgtgaaaagcaatggtggaagtaaacatggaacaattaattaaacaaatcgaacaatgggcgtcagatcgtaatattattaaaggttcaaagccaattgatcaagctatgaagctgtttagcgaatttggagagcttgctgataatgtgggtaaagggcgcgacattaaagacgattgcggcgatatctttattgtgcttactattatttctagtcaatttaaagatggactaatggagaaagtatgcaatgaaagctacttgccatttattgattgggctgaggatcaaaagccagaatgtgtttgtaatttaaaggatttagttgttatgcttcttcgtgatttaagcatattggctcaaaatttagatacttatgacatttcggatgaagtttattcttgtgttgtgtgtctcaaacgaatcgctgagctatctgaaacaacattagaagaatgcgtgcaactcgcatatgacgatataaaacacagaaaggggattatgataaatggtgtatttatcaaggaatccgaccctgtttatgcacaagtaatttcagaaattgaaggaaattataatgcttgagaaatttctaaaatcactaggatttctaggtattgacgcttcaaatatgaattatcaaacttgtgaaaagaatcattataagtttgaggatgaaaatatagataagcattggcgttgtttctatgctttttataaaaatggttttgaggaaggttacaagcgcggatggaatgcggcttgtgatgaatttggcgctacagcaattagactaatcaataaataaagacaagccccgaaatggggctttcttaattcacgcattgaacgcacaccacaacgtgaatgttatttttgattgaatgggcagtgcagccactacaaactacgcaaagcacgctcaaaaataagagcgatttcagcaatcttttgcgctttatccgatccattaatgattcgtcttgcaccaacataatcctttttagattgatagatgtaatcagatagctttttaccagtaaaccaaccttccttcatgccagttaaaagaatttggattgcatattcttttttcataacatcgtctggatttgacaaaaagtcatgtcctagttttttagaagctttctcgtaattggattgccacgtgagctgaacatagccacgaccatagtatagatgcgagaactcatcaaacagataaactgaattttttgatccatctttgaaggtgtacaattgacctttactatttttataccatgttccataaggtcgacctttcccttttctgtattcagaaataggcaacatggttttcgctgtttcccaccatgttgtcgccaatacatatgcaccttgtgaataagatatagacttgtctttatccatcgcatccacgatgaagttaatttcagcgacttggctttctgttagccttcctagagagtctctaagaatggcgaatccacccttagtcattttcatgtttcttcacctttaaatattcatgaataaaacgtactgttaagcagccaagccctagacggactaatatttcagcgtcttgcatgtgaccataaaaatccctgccttgcagtgtattgataaacattgctgttacaccaatagcaagcataaacataattacatcgacatgcactggaaaactaatctttggatggaacaccacaaacattaaactgccagtaatcaaaaaaaagcttagataattaagaaacgtcatcatcgagcttcactcccgttttagattcaatttctaccttactacgcttttcgatagctttaattagcatttttgctaacgtaatgccacccatgccataaataaagccaaaaacctcgacatatccaccgccagtcaaaagacctgctgttggtgttgaaagtgctgcacacgagataacaccaacaatccagctaataactcgttcagctaacggcttgccagaaggcactagggcggtgattgttgcaccagccacccccataaatatgacgcctacatgggcttttagccactccagaatcatgttaacaaaatccactatagattcatcctatataagttttacttataatagcaaaactacgaagggtttgtgttttcacattaatattaagtttacggcgggtgctgcgctatagctaatctcaatactctcttggggcattaatatgtaaataccaacttttactgtgccgccgccagctgcaactggatcgccctcagtccacgagtctgtacctctacgcactaatgcttgggttacagtaccaccctgtacgcgaattttaactgggagttgatttgtattagtgtaagaaaatggcgaagcgccaactgtcaagcttgtgcgtggctttcttgctgatataaattgcccagtttgactatcggtaacacgttggatctgagcatcaacaccattattaattaaaccgccgattccagtattccagtggttcactgtaacgcgatcaataacggttccacgcgctccagcctgaatctctaagcgctcgaaatatccaccatcaatttttaagcatctggcatcactatgcccgctaacaagaaacttgttcgacgaatagcatccaagatagctactgtaaattcctgcatccgaaacatcgccatttacagcgtctttattctcaaaaccaatgccgacaaatgtgttatatctacagtttgtcccaaccaacaaaccatactgtgtgcatgcttcaggtgatccattgataaaagtattttgatctgcaccatttgctgctaattgccaaccaataggattcccctctgcatagcaagatgtaaataaattattagagcaagcattaatactaacgcccgcacgtgttccagcagttaattgaaatccgcgatatggtagacttccatagacataactagtaattaaattattaaaagtagatagcatgcaaccagcaaggcggaatgccgctgttccatcaacaccagttgagccatttactttaatatcattgataattgagcgagcaaacccctgaccaaaaaatccgtttgtgaatgtagcgccagtaattgaaaatccagaaacaaataacccgttaacaaattgatcggtgggcgacccactagaaaaagcattagcactaaaagcatccgctgtgccagtgtaatttagtgttgttttgttggatcctgcgccgataatcctaaattttggtgttttaatgtttgggaatgcgctataagttactttgccttgtggtaatacgagtgtcgcatctcttgcaatagcctcattcactgcactgacaatacttgccgatgcatcataaacagatgttccgttaaaaatagcagaacgctgtgatttggggatgtaatcaattagataaacggtcttctctgtagcggcttgtagtttttgtccgatagtggagtctgaatattctttcaaataagaatatttaatatagtcagaaccatctaagccgagtagattgttattaatttcttgctgatttaaaccactggaatccgtcactaagctagatggaagcccaatagaccacccagccatatcaacattcggatttactgtattattcgcaacggtagagcgaacaatatcaccattatcaagcatcacacgggcatttaatggataaccaccaatagcatcagcaaaagcttgatcaaattcataaggcaagcccttattcaagtgaacaatgtgttgaccgtataaatttagaattccattaaaatcatttcgctcaggcggaataccaccctcagaaatcttttgttgagtgattggcgggaatccagcttgcatagttgcattttgtggattagcgccaactgattctggaataatgtttttatcgccattttcagcgaatggggttgtaatgagtgttggattggtcatgttaatatcctattgcaagccatgaaatgtttaatgtgttagctgtgccgtttcgcaaagttaagccagttcttgtccaagttacaccacaaccagcatcagccaatggatcgaacgacacgctattcaatgtagcttgagcatttaagcataagcttggaaatgcggttgtaaaatttacagtagcgtttgagtcaccgttaacagaagtagagccatatttaacaatgattgtagttccattacctagtggaatggaaaagctaccattattagcaattagacttgatgaaaacttatttaaaaagttattcatattgtcatttaatactttgccctgagcggcactcaaagcagatccaacatcattactatttaagacatttaaaacctttgtgatacctgcggttgtacttgtggcagttggaatacttccttgcccgctatagacttcccaagctccaccaagcccattatttgggtttaccgtgttgttagctacaaggcttctaaattcttttgtggtatcatcggattgaacgattgcgcctaacgcatagccaccaaattcatctatgacatcttgtgaccacgtataacggttgccattttgaccatagatgacatgctcagacagggcattaagcaccccgttaaaatcttgtccttttggcgccttgccaccatctttgatttttgtcatggtaattaagggcgaaccttcattccatgtaaaatcctctggatcttgaccgacttgccgaaccttttgaatagggttcttaaccccattagcagcgaatgctatcgggataaatataggattagccactatagaaaactccattgttaaagttgcttaaatctgaaccattaaagccaaatgttctatccacatcaatctctttatatgatattccaacacctgagggcattggcaacattttaagagtatagacaataagccgatcaaatggtgttaatgcaaactcaaaaacatattcgg

SEQ ID NO: 2 - protein, 682 aa, Unknown
  Bacteriophage YMC14/01/P117_ABA_BP ORF7
MAENVVESIIVKLGLDGSQYNREAEKAKSNNDKLNKSVSETDKIVGNVTKTLARWFSVAAAATGILKMVDQVQKLNDELYHLERNLGMSASTIKNWQGAAGAMGGSAQGMTESIKSLNMGMNDFVTMGDTTLLPFMNALGVGMVDAQGKLRKTDDVMLDLADSFSKMDREQAFSIASKMGIDEGTFNTLVQGRKEMEKMLEYQSKMYKSSEEELKASRQLAQNRALLGQHWESLKTMMADAIIPLFVKLSEVALGIFEYLQEHEDQVKGVFTAISFTIGAILIPILTKATIAALAFIAPFSPFILVVGALGAAFGLLYDDYKTWAEGGKSLFDWGAFKKYIDDSTLSVDNLKNAFSNLGKDMLNNAIPTLKGYAEILDKLVSGDFKGAALQAWDMLKNYYSRAADFVDDVFGQKQGTLANAVGNLVNPNTPASSAPTIASATSKGGNAILDLIAKGEVSTTAPSGYNVAYRGARISAKQMFGKELSQLTIGQVKELQRANLNEQKSRGIPAKRRSSAMGRYQFIYSGFDDYIRAAGLSDKDMFSPENQDAMAMAIISRGKYGLNAVRAGKATPEQFQNNVLAARWASIQKTTGGGVHDAAGFNKATIGNQAVAAALQATRQSDFIDLTKARQNQAMANKANEVQVNVGDINIQTSSSTVTGNVQDAMGAVKDQFYQFRNSFN

SEQ ID NO: 3 - protein, 80 aa, Unknown
  Bacteriophage YMC14/01/P117_ABA_BP ORF8
VLDYSYRKDAMSNQELKDGVLAAPLHMSETVYRALLTGMCDYHQLNTCIGLEGALNMIEAKQVADYNEAKIKYFASQEQR

SEQ ID NO: 4 - protein, 202 aa, Unknown
  Bacteriophage YMC14/01/P117_ABA_BP ORF74
MKMTKGGFAILRDSLGRLTESQVAEINFIVDAMDKDKSISYSQGAYVLATTWWETAKTMLPISEYRKGKGRPYGTWYKNSKGQLYTFKDGSKNSVYLFDEFSHLYYGRGYVQLTWQSNYEKASKKLGHDFLSNPDDVMKKEYAIQILLTGMKEGWFTGKKLSDYIYQSKKDYVGARRIINGSDKAQKIAEIALIFERALRSL

SEQ ID NO: 5 - DNA, 2049 bp, Unknown
  Bacteriophage YMC14/01/P117_ABA_BP ORF7
atggctgaaaatgttgttgagtcgataattgtaaagcttgggttggacggctcacaatataatcgtgaagccgaaaaagccaagtcgaataacgacaagctgaataagtctgtcagcgaaaccgataagatcgttggcaacgtaacaaagactttagcgcggtggtttagcgtagctgccgctgctactggtattcttaaaatggttgatcaagttcaaaagcttaatgacgagctttatcatcttgagcgcaatctaggaatgtcagcaagcaccattaaaaattggcaaggcgctgctggtgcaatgggtggatctgctcaaggcatgactgaatcaatcaaatccctaaacatggggatgaatgattttgtcactatgggcgatactaccctattgccatttatgaacgctttgggcgttggcatggtcgatgctcaaggcaagctaagaaaaactgatgatgtgatgttagaccttgcggattcattctctaaaatggaccgcgagcaagcattctctattgcctcaaaaatgggaattgatgagggcacatttaatacgcttgtgcaagggcgtaaagaaatggagaagatgcttgaatatcaatctaagatgtacaagtcctccgaagaagaattaaaagcatctcgtcaattggcacaaaaccgagcattgctaggtcagcattgggaatcacttaaaacaatgatggcagatgctatcatcccgttatttgtgaagcttagtgaagttgcgcttggtatctttgaatacttgcaagaacatgaagatcaagtaaagggtgttttcacagcaatatcttttactattggcgctattctcataccaattctaacaaaggctacaattgcggctttagcgtttatcgctccattttccccatttattctagttgtaggtgctttaggcgcggcatttgggttgctctatgacgactataaaacttgggcagaaggtggcaagtccttgttcgattggggcgcattcaaaaagtatattgatgattcgactttatctgttgataacttgaaaaatgctttcagcaatctaggcaaagacatgctgaataatgcaataccaacgcttaaaggctatgctgagattcttgataaattagtgtctggtgattttaaaggggcagccttacaagcttgggatatgcttaaaaactactactcaagagccgctgattttgttgatgacgtatttgggcaaaaacaaggaacgttagctaatgctgttggtaatcttgtaaatccaaacacccctgcttcttctgctccaacaattgcaagcgcaacatcaaaaggtggcaatgctatcttggacttgattgcaaagggtgaagtaagcacaaccgcaccaagtggctacaatgttgcctatcgtggtgctaggatttcggctaaacaaatgtttgggaaggaactatctcaactaacgatcggacaagttaaagaattgcaaagagcaaatttaaatgaacagaaatctcgcggtattcctgctaagcgccgatcttcggcaatgggtcgttatcagtttatttattctggctttgatgactatatccgtgctgctggattaagtgacaaggatatgtttagccctgaaaaccaagatgctatggcgatggcaattatcagtaggggtaaatatggcttgaatgctgtgcgcgctggtaaagctactcctgagcaattccagaataacgtacttgcggctcgttgggcttctattcagaaaactactggtggcggcgttcatgatgcggctggatttaataaggctacaattggaaatcaagctgttgctgctgcactgcaagcaaccagacaaagcgactttattgacttaaccaaagctagacaaaaccaagcaatggcaaacaaggctaatgaagtccaagtaaatgtaggtgatattaacattcaaacatcatcaagtaccgttactggaaatgtccaagatgcaatgggtgcggttaaagatcaattctatcaattccgaaattcatttaattag

SEQ ID NO: 6 - DNA, 243 bp, Unknown
  Bacteriophage YMC14/01/P117_ABA_BP ORF8
gtgcttgattattcttatcgcaaggacgcgatgagtaatcaagaattaaaagatggtgttttggctgcccctttgcatatgtctgaaacagtgtatagagcattattaactggaatgtgtgactatcaccagttaaacacttgtatcggtcttgagggtgcgctaaacatgattgaagctaagcaagtcgccgactacaacgaagcaaaaattaaatattttgctagtcaagaacagaggtaa

SEQ ID NO: 7 - DNA, 609 bp, Unknown
  Bacteriophage YMC14/01/P117_ABA_BP ORF74
atgaaaatgactaagggtggattcgccattcttagagactctctaggaaggctaacagaaagccaagtcgctgaaattaacttcatcgtggatgcgatggataaagacaagtctatatcttattcacaaggtgcatatgtattggcgacaacatggtgggaaacagcgaaaaccatgttgcctatttctgaatacagaaaagggaaaggtcgaccttatggaacatggtataaaaatagtaaaggtcaattgtacaccttcaaagatggatcaaaaaattcagtttatctgtttgatgagttctcgcatctatactatggtcgtggctatgttcagctcacgtggcaatccaattacgagaaagcttctaaaaaactaggacatgactttttgtcaaatccagacgatgttatgaaaaaagaatatgcaatccaaattcttttaactggcatgaaggaaggttggtttactggtaaaaagctatctgattacatctatcaatctaaaaaggattatgttggtgcaagacgaatcattaatggatcggataaagcgcaaaagattgctgaaatcgctcttatttttgagcgtgctttgcgtagtttgtag

SEQ ID NO: 8 - DNA, 42555 bp, Unknown
  Bacteriophage YMC16/12/R4637_ABA_BP
caacttagtcactatctattttccccacaagattaataacagttggcttattaacgctttcaccattactagtgatgtcagtttcaaccttgtcactatatccatgattggcaagcattagcttagtaatcgtcccgctaaattgctgactaagaccgccattaattaaaagcatttcttgcttcatcatgattgcgtctaacgtgtccgcaaattcaggacttaaagccttgtattcatagatggttgaacgcccaattcccaaataacaaccaagcccagctatactaggaacaatattttcaatttccttgtacccggtaattaaatattcttttgctttttcaataagttcaggtgtaagaccgctaggtctacccacttcattactcatagcctaacctctctttagcctcccaaaataataccacaaaaagaaaaaccgcgttaaagcggttttatttgaagtaaatatgcttcgtattcatctactgacaagccactagggtgataacgaaaaccccacgagtttgaagtaagataaataattgcttcaccacattcatcatgatttaacccataatgtgttgcgtcatctggagcattagctctaatttcttcaatggtcattcttcaaactcccccaaataagtcacagataaaagataagtttcttcatccatagtatgttggattgctacacgttttatatctgttaaaacctcttcagtatatggaaattcatgaatcggcttggcaatgatcattgatgatatttttttgtctgcataccgttccaactaaatatttatgcttaatggtcatttatcaaatcctcaatccaagaagccgacataaacaaacacaagatatgaatcacacctgaaatcaatccgtcaacataattgtgatttactaaaatgttgtataaagcaatgctaaaccacaacaaacaccaaacccaataaacaccagcaaatagtgtaaatctaaccttgctcttgtatattgattctggatataaaatataatccgttttatctaaattaaatttcatcgactcaccctcttcaaaatcccacccaaccccgagcgactaatcccaagcatcttagccgcttcagtttgatttcctctagtcttaaccaaagccctttccaaaatagctttttcaaaacgttttctaatcttataaaagctttcacctttggctatagcttcgtcaatttcttgatctacggttgttttcattgctagtaaaactccttaacattagccgcacgattttcttcatccttacaaatctggtatgactcattagcaaagtttgatgcaataaccgctttatcctcacctttgtaaacagggaatgagtaagcccaatcaacattggcatccattaaactagccagcatattgtctacaccccgcatctcctcaaccacttctttagcttgatgctttgcataaccttcttgacgcatcagcatagtagcctctgcaaagtcatatacagcatcacaaaacccagcattagcaaaacttggaactgtcaataacgcaattaataattttttcatcacaaaatcccttccacaccatttaaaaacatcttcacatcacccactgttaaattaccttctttatgtaaagcctttaacgtcttgatgtgtcccgcaatatcatcaatcttctgttgctggtgattgtaaattttctcgcattgcttatatgcgcttatatcaaatctatcacccagacacacctttcgatagattgcattaaattcactcacaatcaatctccttccagcatttacggcaaaccttgtaagtctcgccagttaaattactttcctcgtattcccattcatgtcggcagaataggcgctttagaaatttatagatcattttttattctccataagtctgcgaacatagaacccaaaatttggatgggataaaattttatgcacaacttctttaagatcccaaatatcattagcgatataatttccacttatcgtgtttagttttattggaaatgcatgttgcttatcaatttctattgacataactgcttgtgtacaatataaacttctattgcttttaactaaaataaatccacccagtacaccagttttctcgcatatagctttgtttgcttcaataatcacttcaataatctgatcaagatttctatcgtaattttttgaagcttcaagaccttttttaatagcttccataaaatccatcacttaccccttaaccaattaaatacaaccataccagcgtgatatacaatcaacactgaacctctaagcaaatctgttgccgcgtactttaatacccttagatttacccttaactcttttttgtgatccatttttcaataccttgtatttacgcaccacaatcaatggaaataaaaccacactcaagattagagataaaacaccagctaaagcccaaaacaactcaaccaacaatttaggtgcatgtttgaataacttagctacatcaacaattgccaaccataaagctttaactataaccattttatctctcctctttatattctcaatagtaaattatatttataattaatgcaaggattttaagcaaaaaaaatccgtatcttggggttagatacggatgtaaaagtttcttcagttcgataaatatagcaaatttttagttaattgcaagtaacaattttaattgcttgctctacacaattaaccacattaacttgaccattccataagtcatgccattcgatttgatctggcgttaatttttgcgctgaaactggtttagctccatcttttatctctaataaaaagtttttaccgcgatagcccaccaaaatgtcgggacacccttttccaacacttgaaagaatttgaactgtacacccaatctttctaagtgctgccactatttctggttgatttgcatctatcttagctgctctcacgcacatcagttcctatataaaatggtcctttagttttgtacttttcctcaaattcttcaatagtcatatatccaacataataactaaatccaaattcaaactcattagaccaatcttcattaaattttaaaacatcatcaaaagttaatctgtcctcacttgcctcaattataaaaaattgtttgattagtcgattaggctcaaaggcttcaaccccacgaactgcatgtaaatgtaattttttcatggacgttttctcaatgagtctttccaatcgccattatatttatgcctgacaaccatcatatcaccactacatgcatcattaattccccataagccaatgccggatgtattaaaaaatatatctggtttttctttaaagacaagcccaaaccccaaactgtctacacaaaaccatttataatcaataggaaccatcctccaatcaatctcaaattcttctaacacttcaggtcgacttttatctttccgctttttcatacagcacctttgatttttcttagctgctctacgacttgctttgcttctgctttggtgcgccaacaccccatttgagttcgacattcatctgtatttgtcccctcgtaaatgtcagagtcataaccagaatttgtatgagaaatatagaaatattcctcacccaccttcggctcaaaaggcttcggcagctcaagttcaagcttgacggtttgtggtttgatgcggaatttatataaaccattcacaacctcttgtacagtaaaataaacaataggtacccactcttcattttcttctgaaaacccttcagcctctttgccatcagccaaagctcgcaacgcctccgcgccgctaatcaaatctaagcctttagtgataggctccaacccttcatctgtattagttgttgatctcacccacttaccctcaaacataaagtaaaaaactccatcacagcttttaaagtagaatccatctaagacgcttttacttatgtaatttgcatccttcacatcattacgcttcaacacaacaaggtctcgaagctgaggaaatgtaatttcctttaatgtgtctttatccaaagcttcacaagcacaacccattgatccatcggcgaaagctaaaacccatttagtgtttgcttcgtatttgccaaaaagcaaatctagttcatagccaagctgtataaacaactcctgagcctctttgctctcagcttcattgtttacgcgaattttataattttccatcacacaccccataccatattatgaattaacttacttaaccccattactgcaaaaatcacgatcaaataaaacaaaaccatgacttttgcaaatcctataaactctttgctcattggttgttaatctggttatttacgatcactgtttggctaccgcctttatttgacaatgaccagaacaaactccaaaaccagaacaacacagaccaacctaaaaacaaattcattgcaaagattgcccatttacttgcatgcccacgcataaagccaataatgctaggcgcgaaatacataattactgaaacaactaaaaccaattccataataaaaccatctgttaatttatattctcaatagtaaacactatccacaataattgcaactattttttaaattatttgcatgtgactatgggttttacactccgatttgtcacactccattttttaaactggtttttaattcttactccccatgaatgagcctcatagacagtaaatttttcaccagcaaaataaacccgatcaccgactttgtatttcatttctcaaccctcacaattggcttaattctaaatctcgcgtcaaatatatttaagacaaccttgtttggatagtaacgctgacgcaagccaaccttaccaacagcaacaaattcataattcgaagtcataaaatcaccatcccaacagctcgaacttactagaacaacccttgtcttatattccgaatcataaaaacatgtttgattaataattgattcgcacatcattttatggaatttgtaaaatctatcatctgactttctaaaccaacaatttagtatattattcatactctacaccccttctctatctcttcatccgttgcgtgacgcaaacacttagtgcctgcacgtaaactatcaattttatgaacaacactgtccttgctatattcctccttaaatacaacaaggtcgcctaattcatatatttcattttgtcggcggtattcgagtaaagctgcttttaattcatcatggtgcttttttaaccctatttgcgacatttttaagctttttgctacattatacccgcccaaattatcaattaaattcatttaaaaatcctcacatgtccaaatattctaaattaagattactaaatcttgagtattgtagttcagttgctaatctaactgttccaacctcaccatcacgcgctttggctacaataatttcggcaataccctgatcttttgagttcttatcataatactcatcacgatatataaacatgatcacatctgcgtcctcttcaatagatcccgactcacgaatgtcagacattagaggtcgtctatcttttctagattctaagcttcggcttaattgcgctaaacaaaaaacaggacaatcaaattcttttgctatagccttaagaccttttgatattgcaccaactgccaagtgattgttgtcagtagacaatggtgatttaagtaatgttaaatagtcaacgaatactgcaccaacataaccatattttgctttcatgattcggcaattttttctaatatctgacaagctaggtcttgccctatcatcaattaaaagttttgatgccttcaacttattagttgctttatagaaaaactcccaatcctcatttttaatatcagagtcacgaatatctcttaaatttattgaggctagactagcaaccaatctttgcccaattttttctttcgacatttcagcagattggaataaaactggatgctgttgcataaaagaaacactcaacatcaagctttgagcaaatgcggttttccccattgatggtctggcagcaataataactaaatcactttttgatatttgaccaagctgtctatccaagtccataaatccagtttttacacctacgtcaaccttctctccacgttttcttgcctccatcgttttatcaatagacgcaaatagagaaacagaaacatcataagcatccgataaagtcggcacttcatcctgattgcttaaattctccagaatgctctcagatttcgatatagcctcattaatgtcgtattgggtagtatctatcgaaatagccttaattctttctcctgcctcaaacagcgctcttctggaggcatattcatttaactggtcagcatgttgctcaagaaaatgagccagacctattgttgagtttaaatttatcaagaaatcttcatcaacaattttcgagtcatttgcatttaacttgattgagtcataaaccataacaatgtcatgacctgacccagcatcaaaaagctttttaaagtgcttaaagataattttatgttgtgttgcataaaaactatccgtagttaatttgcttacaacatcatcaataccaccattgagagacataagggcggataataccgattgttcaatttgtattgagtacaagttttcattcattttatttaatccaaataattgattggtacagctacagctcttcttactacattttcttgggcttgaactttatcagcactattattgctaacttgctcaatccaatagtcggttttccagtattgcttgtttaaccacgaactaggtgctgctataaactcaccattgttctttgtccacatttcatctttcttaaactcctctaaaaccttcatgatcatttcaaaatctttttggtatttagtaaaagttttattagctgcttctttagttcctttccgcttacattttggatattcattccaaaacttctcaaatttttctgaaaaactactttgttttttatctgtagtattctctgtagtagtctctgtatatgtattacgtttcaacgtatggggtggtacgttagaacgtaggagggtggtactttctaacgtaggagggtggtacattgaaacggatgatacaatgtctgcatcaagaccaataaaaagaacattaaatagtgttgagttgtcagtttttacggttctaaactctcgaattacaacaccaaactcacaaagtcgatcaaatgcctctttaacctgtcttttagttaatccaaattgttttgcaaagctctcataagatcgctgcaataaatctgccttaaacttttttctaactccaatcgttgcacctgtatcttcatcacgtacaatttttggtctataccagtaaacaatctcggataaaattacgatagcattcaggtcgggctttccatttttaaacttaaaaactgagaaccaattaaaaggaattgcattcccttcaatattcatcaaccctattttgtctactgtttcatttccactgctatacattgttatcaccactcaaaacaatttcgaagtaaaatttgtttaacttaatcggcatttcaacaccaatactcactagtttttttacaatgtgagcatcaacaacaccattgagtattgaattcttaacagccatgcagaacctaaaatcctctaaatctctactgtgttttgttaagttgcatgttgaacaagaacaaacaagattctcaatactttcgttaattatctttgactttggtatgaaatgatcaatatgcatttccatatatgtttcaatttcaaccccacaataacaacattttttgtcatgagagcgccaaagttcctttcttaactctttagtgaatttgtagccactagggaagtatttaaaagcaacatcaaaaggcatatcattcaaagttttagacttacgaatcatatttgggtgattttctatcaataaatcattgttaagcaatgtattttccataattttacaaacctatctaaactgaccactaacaccgaaatacattggcataggctcattgcctttctttcgatcaatgtaagcgtcttgaatttcttcaatcatgaattcatctttttcatggtcgtaaaagttctttacttcaccaaattgattgacaaattcggggtattctttttcaaaccaagtcataaagaataaagcgtcttttgatatttcattcatcagttgtctcctgattaaaaagatgatttcttaattttattaaactcatcttcaccaatgcagcaaataataatttgctctatcatatggaatagctcaggttgtaactccttgttaaccatatcaagtgctgatattgcacagtcataagctgtggcaatatcgccaaaaagatcatttttttcctttccagattttagcaattctgccattttatcaggtgaaacagtgtagaatgctggcttgcatcggtttaggacacaaacaacatcatgctctgctgccttgactggattcttttttaattcgctaatcgaagttgtgattgctgtataaatttctgtaaccatttcgctctcctaaactatatggtcatcttataggtcttttaaagttaagtaaaataaataattcaactatacaaaataagaaaaactgatataaaggtaaaataaaaagccccgaagggtttagtggttatttttctttttttaatttccagtaatcaaaacattcattgttataaactcgtcttagcaatgatccgcatttgcaaggcttggtatgctcatagatcgtttacccatacttcttgcatttgaccttgcaattacatttgggcttatatcctttctaggctctttatcgctaattttgaaaggtattgctgcatatccaaagcggtcagggttcataaatccgtcattaattacatggttcattttttacctacattaaatattaacaatatctagtggtgtttatattatcaaatatattctcaatagtaaataggttggagtagttagctctttttgtgttagaatgaatttgcggatagagtgacggctcgaaaaccaattaacctgattggcctccgcatcattacatcaggttttcaatgaggattgaattatggctaaactagtttatggcgttgggattaacgacaaatcaaaaccctccatggttgatggaaagttaataaaagaatatgtcgtttggaaaaatatgatttttcgatgctattcagaagaatataaaaagaaatacccaacttatattggatgcactgtttcagaaaatttcaagaattattcgtatttttataattggtataacaaccaaaccaacctagatggcaatcttaattttgaacttgacaaagatttgctaataaaaggcaataaaatttactcagaagacacatgcactcttttgccaaaagaaattaattaccttttaaacaaaaataaagccaagcgaggggagtacccaatcggagtttctttgcataatgcaacaggaagatttgttgcaaaaattaaaaaagaagcaaagttgttttgtattggttattttaataatccagtagatgcctttattgcatacaaaaaagaaaaggaatctcatattatattcatgacaaataagtatagagatctattaagcgattctgcttatacggctcttttaaattatatggttaaattggatgattaactcaatcatccaatagtaaatagtacaggttaaataaaacccgactagcgggttaaattcctaaaagtttttttgtatcggggtgaagcttgactatagccaaacccttttcaaagcttggatgtagtcgtttaccagttgctagaccgtgaatgtgaacagttgaaatccccgtctttcttgctaattcagcttgattgatttcacggtattctaaaacttcttcaataatttgtttccagtccataagaccctccattttttgttagtttaaattattaaattagctattgcaaatattaaataatagtttactatagggaatatcttaacaggagaaacaaagatgaaaatcgaaaatatcgctattgtgtgtaaagatttaaaccaaagagatgaagcggaaaaattattaaaatttcatggactcggaaatgatatgtgtccaactggagatcagaatattaccgttgattttggtcaaacattttggttttcagatttttctgttggacgcaaaattagttttgatgaatttatgcaaagatattcagaaaacgatatgatcaaaaaaatccaccaagccaaaaaagaacttggtttaaataatagtgaactatctttaaagatgggtaaatctcgcccctatatcgctaagatgcttaaccagccgcaaagcgagaaggttcagaataaagttattaaagaaattgacgaacttttggaatttgagcagcgctgcaaggaaaaagagtatgcacgatgcgcttctgaattaaatattccagtatctataaatgatctttcagttcaaaaacaccaagatggcgagaaagacaaagaaatttcggatctcaaaaagatcatcgaaagtaaagacttggcaatcagtggactaatcaaacaaaacgatgaagctaagaaaattcatgataaggatattaatacactggtcgaggtagaaggaaatcttattggtgcaaaaattgatttggatcgaacaaaagttgaattggattactttgttaatcaatacaagcaatctgaagaggatgtaaatagccttaaatcgaagatcaagcgcgagcgaatcattattttaattgttattgcaattttaacggcgctattcttcttgaagggttctattggtggttgagttttgtaagatttgttgtaagtacaagttgtttttaaataattactgtagtgagtgtggaatgaaatgaatatattagagttggcaaaaatcattgataattctttttcacaagatgagtttttactaactactcaaaaggttggcggtgtttcaagcatctcaatatccccaagaactggacaagaaattgaattttgtaaagttggtgctgcttatggagttaaaataaatgatgaagagtttgaattaagatctggcgagtttgagtcgctattatcgccttatcttggaaggcgaatgttgttaaacttaatgaattaaaattagctccttcgggagcttttttaatatttgcgcttgacattaatactcaattcatttactattgagaatataaacaatgggtaaacattatgaaaaatgcattgattttagctgcttctgttgcgttagctgcatgttcaggtaaaccagttattactggtccttatgaagttgaatcattagatatggaacataacgtagctgccattaaatctggtgatttagttttagaagttgagtttgaaggtagattctttaaagatggcaacggattccaatcatggaatgatgttgaggttgaatctgtaaatgacgttaaagtttacaatgaagatggtgaaacagaaaactatgttttgcctagcgaagaagtatcaaacattgttcaagttattgaaaatgaaattgcggagaaaatgtaatgagttggcttgacgatgtgaaagaacatggtgcagacgcttacttctggtcaatcgaaacagcttatcaaatgaatgtgattgactctaaagaatacagaaagcgtatgcatgaatatagagatattcagcatcaagaaagacaggaaacattacttaaattattggagatttctaaatgagcaatctaactcagcagcaaaaacaaaatgcattagcaatcaagcaaacgcttagcaatccatcggtgatgaagcgcattgaggaaatgatagggcgtaaatctgatagctatattacgtctgtaatgcaagttatcaactcaaacgcattattggcgcaatcaacacctgaatcggttataggttcggtttatacggcttgtgcgcttaatttgccactaaataacaatcttggttttgcttatattgttccgtttaaaaatcgacaaactggcaatcaagaagcacaatttcaaatggggtggaaaggctatttacaattagctcagcgctctggtcagattaaaaagatcgcttcaattgcggtttatgacactgacacagaggaaagcgtaaaggctcgtttaacttcattcattccgcagaaggttagtggcgaggttattgggtatctggcttatcttgagactgtgacagggtttgaagctcatttaacaatgactaatgaagagcttgagcagcatgcaagcaagtatagccagacgtataagactgcaaagtcgaaagggcaaagctattctgtatggcatcagaattgggatgcaatgtgtcaaaagactgtcattaagttgcttatctcaaaatatgcgcctatgtcggttgagcttcagcaggcgattgaatttgatcaagctgtgattaatgaagatggcgaagcttcatatgttgataatgatcaagaaagcgaaaagccattagctcgattgatttctgaggatcaattcccgcaattcgtggcggctattgaagctggtagcttaactaaagagcatgctttaaatccagatatttacgcattaagcgaagaacaaaagaaaatagttgaggcgttatgaatctaatttttcgatgttctgagcttcatcgactaatgggaaatgcaaagtctattgactctgcatttctcacagaagaagttcaagcaataaaggcgaagaaaaaacgtaccgatgaagaacaaaagattttagatgatcttttggatagaactttatcggctactgctaagacactggtcaaggaaaaagttaggcagttaagacataaagctccaagtaagtttactggcagtaaagaaacaagaaaaggtaatttagttgaagatgacgcaatcttgtttctaatgcaacagaagtttattagtgctgaaaagaatatgattcgttttactaatgactggatcactggcgaaccagacattattaccagcacagcaatacgtgacacaaaatgcccgtggtcatattggactatggagtattttaaagaggatattgagagcaaggctttagatgctggttatgattggcaacagttggggtatatgtggttgttaagagaaaataatgattatgggccgaaaataattaatgaggcctatcttgattttattcttatgccaaccccaaaagaatgcttaactagatatgacgatgaatatttgcatatcgattatgttttggaactagagccaagtgagcgtatttcatcatataaagttgagtatggtcaaaagaaaattgacttaattaaactaaaagtcgaaatggctagagaatatgccaagactttagtttttggaggataaatgaaaatcaaggaatcattagtctttatgtttgttgtgtggatgcagaaaggtgggcataacttaaacgagcgtaaaggcaatattgttatgcgtaaaaatggaaaggtggctgaaatttatttttcagatgccgattatcatgaaattaacgactattgcaaagaacgatatgaattgtttttaacgcaatggcttaaatatggagtggaatttatcaagaatttaaaacgcgaaggtgcgttagaagtgatgcgtaatagagggcagcgatatttggagttgaaaagatgaatttaaatgagtttgatattaaattaagatctgaaacttggtgtacaggatatgaaatttcaagttatggaagtgaaataaaaatatccactttagatcttaagcaggctagctcatggtgtattgagaatataccgttaggtgtaaagtgggagttattcttggatgaatcaaaatgaacccaatagcaatgattttaatatcctttgttctgagttttgtttttgtagttgtcttatatttatatgaggcgtataagcgaaaacgatacgtgcaaagaaagcaagatgatcaacgtaaatttaatcctattcgtgaaaaggtgtggtggaaaaagtaatggaacaattaattaaacaaatcgagcaatgggcatcagatcgtaatattatcaaaggttcaaagccaattgatcaagctatgaagctgtttagcgaatttggagagcttgcagataacgtaggtaaaggtcgagattgtcgagacgatgttggcgattgcggagtagttctcgcgattatttccgctcaacttggtgaaactattgcagatttttgcattgaagaaattcattacccgtatggcgataaatcaactgtgtccgaactttgtggccaactatatgagtttgtatgtcatgcaaaaaacggagtatttagtaagaatcatcttgcttgggcgcttgaatttttgcatagaattgcactaaatcaaggatcttcactagaagaatgtttacaaatcgcttatgaagacataaaacacagaaaggggattatgataaatggtgtatttatcaaggaatcagaccctgtttatgcacaagtaatttcagaaattgaaggaaattataatgcttgataaatttgtgaagtcattgggttttcttggtgtagatgcatctaatatgaattatcaagattgcgacaagcatcgttataaatttgaagatgagaatatagataagcattggcgttgcttctatgctttttataaaaatggttttgaaaaaggtcacaagtttggatggaatgccgcttgtgatgaatttggatctacggcaataaggttgattaagaaataaagacaagccccgcaatggggctttcttaattcacacattgtacgcaaacaacaacgtgaatgttgtttttgattgagtgggcagtgcagcccactatattaaccgccagtgccaagcttaagattattcgcaacacgattcatccaccctttaccaaaattagagaatgagcttaatgacgtgtaaaaagaaatacgaaaagaatagaaccttaataacaattcattttcatcaaatgactttatagccgccaatgtgttaggtccaataattccatcatctttaacaccagctgcttgttgaagtgtcttgatgccacgactaacgccactatttaccgatacatcaaaaacatggaatttcaagccttctggtaataagtcgcactttgccttatcccagtaatcgcgcttataaattgcctttgcttgatcaagagtaagatttttaatatccaagtttggatagctatttgcagcaattccatacttagtaccctttagctgaccaactccaactttgccacctgtccagttgccagcatcgtttttattgagtgtaaatccgccctcatggccgatagtcgtatcaaatgctttgtcaaagttcatgtttcttcacctttaagtattcatgaataaatcggactgttaagcagccaagacctagcctaaccaatatttcagcgtcttgcatgtgtccgtaaaaatctctaccttgcagtgtattgatgaacattgccgtaacgccaatggcaagcataaacataattacatcgacatgcactggaaagctaattttggggtggaataccacaaacattaaactgccagtaatcaagaaaaagcttaggtaattaagaaacgtcatcatcgagcttcactcccgttttagattcaatttctaccttactacgcttttcgatagctttaattagcatttttgctaacgtaatgccacccatgccgtaaataaagccgaaaacttcgacatatccaccaccagttaaaagtccagccgttggagttgatagcgcagcacacaaaataacaccaacaatccagctaataactcgttcagccagcggcttgccagaaggtactagggcggtgactgttgctccagccacccccataaatatgacacctacatgagcttttagccactccagaatcatgttaacaaaatccactacagattcatcctatataagttttacttatattatcaataaaacattaatgattctattatgttgaatatttcataaaagcaaacctgtcagatgtatctgtggttaaaaaaggtgtgtcagttattcctaacgatacaagtttgtatgttcctgaataatcagtagtatatgagggagaataaataacagtagaaagcattgtttcggtatactctaaaaccataccaagttttgcagcagaagggattgatccactttgcactcccatttttgaaacagtaccgccactttttctgctttcttgaatataagcatatgaaacagactcgatataatttccttttccactaatattggcgtctgagtatgtattaaagtcaccaccaagccatgaattcaacactctataagagccgtttggattaattctgattgtattgttgacaacttttgaaggatcattaaggacaaagttcaataagttgtaagtattatccagagtaggaaatgttgagtttatattaatatcaaaaacacaatttttaatgtactcaagatacatcaacaaaccatgtctttgatttttaaataatgaaaaatcatatatatttcctgaaaaaacccagccgtcaaaataatcgttaggtgtacttgttgcaacatctcgatttaagatcgtatttactttaatcgaattgtttaagaattttaaattttctctaacatacataccaacaaatctaatcatgctaactatttggtttaacaagataccatttgttgctgtaaatgagtttgatcttattaaaacatgatccaaaacttcagcagaataagtaatgttaggaacagtatctccttcaggccatacggtaaatatggatgcgctaagatcggatgggtaacttaatcctctaactcctgaatttgtatactcttgtaaagttggcgcttcaataaaagttacattattatcataaaactcaagatcatttaattttgcagtttgatttggtatcgaccagaatccaagagctgatcttgatattgttgcagtattattgtacgctttttgatcataaactatttcgttaggatctaaagattgatctgtccgtaaaattgcactaaatagcatatttggatacccatttatgcggttattgtaaaaccactgatttgatccgtgcaattcacaagcgcaagagtttaatttgcctttaactgttgtaaatataaaatcgttatcatgaacttttatattcttaccaatacaatagatagtagaatgatcatggaataatgatgtatcagacattaaattgataaatgtatttccaaatatttcaatatttgttgatgtttttgaagattgaaaggcatttgcaaaatcaccgccctcaaaagtcaagccgtaaattttcatattatcaattgatgcttttatagcccatctatagccgtcctgtggagcatctggacttgatgtttcagaaaagtctaattttcctccgccataaatgttaatatttttgtgcttatttcttccgtaccaatcatttgaatctgctcgatacatggcactaagaatgttgcacttcggttgttcggcagtacctctgtaaaatgagccaaattttaatactgaatttggttgaataaccagatcaacatttgatactaaacgaatagcatttttttcattaaactgaacaggatcaatcatttttccagttcgaggacattgactgttaaaataaaaaacaccatcacaaacaacagttccacttaatgaattaacttccaatgcatacatgagcttgtcaaaattaagtgtgtcgtcagtaacgccatctccataacaccctgacatgtacggcgtgtatatattaaatggaacttctctaacccatccgttgaagcacaaaacaccgtcattttcactagatctagaagagtcataataaaattcaccgccaccatttagacgattgctagacttatcaaagtgtcgtacaacaacaatttctccctgttttggattattaatacttgatagatcggagatagaatctacaaaaggaatggagtttttaataacactccagccattcatatttacattcggattattaatattattagcaactgttgattgaacaatatcgccattagcgagcatcaaacgggcatttaaaggatagccacctatcttatttgcatgctcttgatcgaactcataagacatgccgttgttaagatgtactaaatgttgcgtaactaggttgaacattccattaaaatcatcacgctcaggaggaataccaccctcagaaatcttttgttgggtgattggcgggaatccagtttgcatagttgcattttgtgggttagcgccaactgattcagggatgatatttttatcgccattttcagcgaatggggttgtaatgagtgttggattagtcatgttaatatcctattgcaagccatgtaatgtttagctgattggctgtgccatttcttagtgtcaacccaaatcgagtccaagttacaccacaaccagcatcagcataactatcaaatgtcacactattcaaagttgcttgagcatttaaacatgaatttgggaacgcttcagcaaaattaactgttgcgtttgaatctccatttacgggtgcatacccatatttgatgattagtgtaaatccagttgctccaaatggtatggaaaggcttccattattggcggttaagttgtaggcgaatttattaaaaaatgcatttatattggtacttagtgtgccaagcgcatcacctaaaaccttaccttgtgcagcacttagagctgacccagtatcgttacttgttaataaattcaatacttttaatatgcccgcagtggtgcttgatgcaatgggaatacttccttgacctgtgtaaacagaccaagcattagcaagtccgttatttgggtttacggcattgtttggaataaggctaataaattcttttgtcaagtcatcagattgaataatggcgccagcagcatagccgccatagttatttatcacatcttgcgaccacgtatagcgcccgccattttgaccgtaaataacatgctcagacaaagcattaagtacaccgtttacatcttggccctttggagctttcccgccatctttgattttcgtcatggtaattaaaggcgcaccttcattccatgtaaaatcctctggatcttgtccagtttgacgaactttttgaataatgtttttaatcccgttagcagcaaatgctattgggataaatataggattagccactatagaaaactccattattgaaattgcttaattctgagccattaaagccaaatgttctatccacatcaacttctttgtatgatataccaacacctgaaggcattgggagcatatttagagtataaacaataagccgatcaaaaggggttaatgcaaactcaaaaacatattgagcggccatatgtcctgtaatgtcgtaataagctttgcgaccaccaaaaaccattaataaaaacttattaatattccaagcacttgcataaagaatatttgaagcagctttaataatgattagttttcttaggtctgcatcggttaaacgataggaagcaaatgcgccaccatcagaaaaaggataggtattgaaaggttggtaatctggctgaaaaccaaatgtttttgcatttggatcggcagttttagcaaaacgagatacaccaactttatctgcccaaatatctaagccaaaaccttcagcgcttgataaacgataaactttttcgtaaaagtcatcaataaaatattgaggatcaattacttcattaatgccatcaataagtgccaatattgtgggactgtgtgcgtactgcgacattatcgtatctttaatattctccattagctttcctcaatagaaattctaaaagaatttaaagaagggtattggtcaataccaatagggatttcattaacccatgtggcattatctgtgcttgctacaattccctttaatccgatttttggcatgccgcaaatatagtcggctgggataaagtctttaccaatacgaaccttattttcaccagaagcagcactggttaaaatatgattaataacttcttgttcaatagtgtaagaaatcgcatctttatctttaacaataacttttacccaaatatcaagaaaatcggggcgtaaaattttaataggatatttaggaggattggacgggtagtcttcggcaattactgtaacatttgtgtttccattccaagaacagccagttccagctttgataaaagcggcttttgccacatcataatcattaccaccaacaaccgaaacgcaaatagaatttctaattaatggataatttgttgagcctactgttactgttgcatcagttgggtttgagattacataagcatctacaacatcacgaaccgccaaaactgcgccaagtgtagcagagtcagtcattttggaattgattgctacagactcttttcggcgtacctcaaaatcgacacggttttcttcgtcataccccatcaccgcgctatcttcgttgtagacacgatcaagaccattaatggatgttgggataacaacaatagagttaggcagtgcttcaatggctccagcctcatcacaagttactgtaatcgtcactttgccatttgagccgatattataagttccattggttcgccaagttcgaccagataaatcttgaacggcaaatccttctggaattggcgctccagataagccctcaaacacaacagggcattgcgaacgagtagcaagctttcttgtcaaaaaataaagttcaccaatagcatcctgaaaaataccttgtgcataacgcggatcatattggttagctagataaacaagcttgtcgtagcaatctttaatgactgccgtccatgaagtgtttaattggtattgtggagaaccttgaacgcgagaaacattacccccaaaggagtcatcaatcatttcccatgtgccattgttgatttgttcggtatctggaaccgagtaaccaacatcagttatttctatctttgggatcataagcccactactccactaagatttttatcattcgtaaactcaatagatccagacgctacacgatccttatctaaagccaattttacattaaccgatactacaccagaaacaagcaatgatctttcttgtaaatggcgctggtacagggaaattggatagccaaatttacccatgattgactctaaataagggatgccatcattcttattgtaatagtcatcttcaagaaaaacacggcatgaacacgcaatgtcttgcgcctgttgatattcttcagtagctacggcaatatttccttgtgtgtctaaggctaaatcccaagtttttgggtttagaaacatggttttcatataatattcacccccatgatatttagcacgaccaataaagtaaaaaatgccgcaaccgcataagcaatacttcgcgctgaatgcaaaacaataaatgctgcaacaaaaccaataaagaaccagaacaactcatgtaattgcataattaagccttcggaccgtctgtatcgctagagcctctttgaaccccgccatgtttgtgagtaaacaagctaatacctttagtgattatatcagttaaagattcgattatgcctgatactgttaattttcctgtgatactgacttcaggcgcatcaagaacaatctttgaatttgctttgatatggatttcatcgcccttaaaccagatatattgttcggcttcttccttactccaagtcattgttaagaatgcgtccgactggtcgcaaatgcggtttacgtttggtccgactcttgatcttgttcgttttagcaaagatgtgtcacgagtggcaaatacgcaaaatcctatgtcgccaatctgcggatcacatataactgcattctttccaccttgtattcggtagtaaggcacattgtgaatttcacctaactccaagttattattgtctgcaccaatacggtaaagcataggtttaacagaaacaaagccgactggaccattgtcatttgaataaacttctgttatctctacaacttcaccagtgtttaactcactggaaagaattgagagaatattagctttaaattctttagcgccgccaagattattaatattaaagccactagcgttggactgctgcatcttttgagttcctccaagttgcattcacattcatttgccacttgccttgtgggatatttgcctcaagtgtagcaacaagaccatatacgcgccaatcttgattgacaacatcacctataatactttccctaatttgaacaataccaccaaatctcacaagcggatcatacgcacaactaaatgttattcctctttgatctggagcggggtagccaatcaagcctgtcttaggtgtaatgactggtatcttaacttccctatctccacctttcttgcaaataacaattaatctttgctcgatgtacaaatcaaagtcgcacatttgagctaacttctcaattttttctatgtttgagccgttcagtgtggtgtctgttaggatgtgagtaacgccgtagttttcaaactcatattgcatgtcttgcgctaagaatttaataatatctgctgcatcgacctcttcaccttttggaattgtgaatggtgcagtagggcgcattttttcgactacagccatctggcttgtaattactagcgctacatttggcgcgccatctgtattgactgtcgcaaatgtaatattcccctcataagcaacttttagtggctgtccttgttcgccgacctcaatcttaaccatgtttagaattgcttgcatggtattccattggacgcgcgtaagcttattcactgtagaaaggggtaggccataagcggttatttgagcagtcggtgaaatagccccattgccatatgtgatatttgtggatatagctaaaccagtagatgacaatcggttgtcaccttcggctgtaaaagtttgaacgccatcttgcaatgttagcgttattttaatgaccttctttttcacaataaaaagcctccgtattggaggctagtataacctaattaataaaactggtgtgccacgatgaataggctttcttcatcttcaccccgtcaggctaggcactataaaatcatacttcgtctgtccatataagtaaataacgagtgcctaagcctgtatattcaggatcactatttccatctatgtcaacaaagataaagccatgccctaagtaagttcgatttagacatatccgatttaacaatacatcaccatcattattattagataaactgatatacaagttatttaatcgagtttctagcgtgattgcccaagtaacgccatttaatgatgtggtgaaaaactggttcgggacttgtgctaacggtatttgataaatcataatccaagaatacctcttactacatttccaactatattatcaccaccaagaatactagtagattgtttagcaccaccatctacagttttggagtcgctaggcgctttaacttcttcaatagagtattccacttttgcctccagcacctcttcaagatcaacattaacggtaatcattgttgcgccatcttgagcagagcgggcgtggttaattccaacaatttgataatttagataaacgtactcaggtgtaataatgtgaaagcttacagttgattttagtagagcttcaatttgccctagaaacaagcttctttcaagcacaccaccactgccttttgacattgagacggtagccatagacggcgcatttactttgttatatgaggcaaatgaaccttgctctactgggtattttgaaatgctagagccagcatcataattcatgctcacaacagtatcagctaaaacaatagggatgccaaattgattgacaatcccccatgtcttgccaaatacaccattaattagagcagcgccacccagcgaaagtccgacatttgtcaaagcttccactggtataaaatctggcacagaaggcattccagctaacataaatcacctaattaaatgaatttcggaattgatagaattggtctttaatcgcacccattgcatcttgcacattgccagtaacagtgcttgatgatgtttggatgttaatatcacctacatttacttggacttcattagctttgtttgcagtagattggttttgtctagctttggttaagtcaataaagtcgccttgacgagtggattggagtgcattctgagcagcttcagcattcgcagtactgtagtggctttgaaatatcgactgctgtctttttgcaaattgagcagcgctttctccacttcttttgccattttgtcgcattgctaattcagtcgcactagaaacagctccaccttttaaggctggtataacccctggtcctatgttatgcatcatgtataggttttcgccagtcacaggtaaacctgctgcccgcaacatgtccgcattttttttagccaacaaggcggtagccatagtgttaatgcgcttgttgtatcgggggtcattggcttttctgaaattacttttattaatttcagtcattcctatttccttaccctcacttgttttggctaatgagttccatgtgctttgaataaattgtcctgttcctatagctccagttggtgacattgcgcctttccaaccagcttccatcttaacaaaacctctcagtactttttcatctattccatatttctttgaagcgtctgaaatatcttggtcaactccgccaccaaacttgaatttgtaatttccagaattactaccaccactcacgcttggagtagttgaagccgaatcagaaggattaatcaaattaccaacagtattagccaatgttccttgttcttgaccagttaagtcatcaacaaaatcagcagctcttgagtagtagtttttaagcatatcccaagcttgtgatgctgctcccttaaaatcaccagacactaatttatcaagaatttcagcataacctttaagcgttggtattgcattattcagcatgtctttgccgagattactaaaagcatttttcaagttatcaacagataaagtcgagtcatcaatatactttctaaatgcaccccaatcgaataaagacttaccaccttctgcccaagttttgtagtcatcatagagcaagccaaatgcggcacctagtgcgcctacaactagaataaatggagcaaatggagcgataaacgctaaagccgcaatcgtagcctttgccaaaattgggatgagaatagcgccaactacaaaagatatgcctttgaataccgcctgtactgcttgttggttttcttgaagatactcaaatattcctaacgcaacttcactaagtttcacaaataacgggatgatagcatttgccatcattgttttaagtgattcccaatgctgacctagcaatgcacggttttgcgctaattggcgagatgcttttaattcttcttcggaggacttgtacatcttagactgatattcaagcatcttctccatttctttacgcccctgtacaagcgtattgaatgtgccttcatcaattcccatttttgaggcaatagaaaatgcttgctcacggtccattttagagaatgaatccgcaaggtctaacatcacatcatcagtttttcttagtttaccttgagcatcgaccatgccgacacccaaagcattcatgaatggcaatagggtggtatcgcccattgtgacaaaatcattcatgcccatgtttaaggacttgattgattcagtcatgccttgagcagacccgcccattgcgccagcagcgccttgccagttcttaattgtctgcgatgacattcctaaatttttctcaagaaaataaagctcatcattaagcttttgaacttgatcaaccatttttagaatgccagtagcagtggcagctacgctaaaccaccgcgctaaagtctttgttacgttaccaacaatcttatcggtttcgctgacagacttattcagcttgtcattatttgacttagctttttcggcttcacgattatattgtgagccgtccaacccaagctttacaattatcgactcaacaatattttcagccatttttacctctgttcctgactagcaaaatatttaatttttgcttcgttgtagtcggcgacttgcttaacttcaatcatgtttagcgcaccctcaagaccgatacaagtgtttaattggtggtaattacacattccagtcaataacgccctatacacagtttcagacacatttaaagggttagctaaaacaccttccttcaaatcctgattactcatcgcgtccttgcgataagaataatcaagtactaagcttcgccttgttctaaaaaatcaatatgaatcccaattgcgtgagcagctagaattgtaaagtttttaaagtctttaatctcctgatcccacatacaaatacgtggttcaccagacttaggaacaatttgcacacagcgatcaagtaaatcaaattttaattcgcgtgaaatttcatgcggaatgcgacccaatacggtaaagactgccgcgccaatttcaatcattccagccattgattttgtatcaagatttaatacatcgacatcttttaaattaacgccactatttgcggcttgctctaaaagacgatgtgcccattcatcggcctgaatcgctggcatttccgtgattttgaaaactttgcctttatcacgaccatcttcaatagtaactgtttttgtctttaagccttcagacatgattttagcctctaaaaaatagaaaggggatttctccccttatggttagttggtttcttcgataccgttagacatcatattaaagttatatgtatgtccatttaaaagctgtgcaataccagtaccaccagatttagttaccattgttcctgtaagcgtttgtttgcgcttaacagaagggtagctaatctggaactcacaacggcgtacttccatattttgaataaagtcattatagacagtttccaaaactggtacggatcggctattcgcttcaagtgatacagtaataggggtttcatgcggaatgaagcccatagactgcttaccatcaacaccaacacgcgtttgagccaatgtaacatcagagaatgttacaaatgcgtcagactgtgcgccttcaatattgatccagtcatcatacaagccagcacagcggaattttacaatactgtttgccgctgtaattgtatttggattatatcccattggcataagttaatctccttactgaacgttagtgacagtcatttctaaacgttgcatactagagccgtccgtatagaaaaacttaatgataaatgattcacgcgcaacacgcgtttgagagtcaggcaaggtaatagatagtgcccaaccacgagtaaatagcaagctagcagcatcaaaaccagcttcttgattgacttcaaatttttgagcgttagataagttcacaccagcacggataccgccaaagttaacaccttgatcaattggatattgcagatacgctcttaaagtttctcgaccatcatcgttgtaaggaacagtcggccttgccataaacatattaaccgaagccaattgaaattgagtacgcaagaatacttggaagtcaaagttatcaatccacttatactgaccagttacagagccattgccagcaaattgaaaacgatcattggcagttgcccaagcgccatagaatgaataaccatttttaactaatgtttctgcatcagcttcattggttacgtctggaaccaatccatcttgactgcggaaagcagtagttgtgcgcccgttagtttcttgatagttaattgaaccagaaacaccacagaagaaagcagctttatcaaaagttccgtagattggaacaacaccgctagtattttcttttgcccactcaccaaatgaagcaccgctttgaccaagagcaacaggatcaagaccccatgtgtaaagcttgaatcggctattttgttgagtgatccaagtggcaaaggcttttagagcatcgtcatcaaatacgccattcgcataagtgaaattcacaaagttactgctaaactgaattgcgctcaatgccgcagtttcaggcgtatcttgcgttgtatggttgtttagtattgcgccagtgtcttgagttaatttcagcttatcagccacagtaccagtcgcaaaactaatagttgagccttcgcctgtagtgccagattgaattacaaaacctttggttgtggattggtaaacacaagtcaaagttaatgctgtaccaattagagcagccgcatcgctatatgagtttgcggtgcttaaatcaacagtaacgttctttgatacaccatcaacaacgatatttaaactaccgttaattaacttaagattagcaatgcttgtaccagtaatatcaccgccgataatagtggcagggtattcatctgaattgtatgttgcaatgaatagcgaatttggtcgagtagttgcgctattaaagccgttaaagtaaacggtagcaaacttataaacatcactttctaaaccgtagtgctgaccaactagagtattagaaaaatattcatagtttggatatacagcttcatttacaaataaagttgtatttaatcctagtgggttgccaccgccaccaatcacggcagggtagacagcagcaatattacttgctgggattgagttaaactgcattgatagactccaaatcgatttgagaaacgtcttccagtatattaactgttttttcgtactttgtattgtattgcaagtcagcttcaatcataaagcgcaattcatacattccagcctcattaacaaaagataaatcgcgtatacgcggattacctaatggtacgcaattaactagcgtatcagtagtataagggctattccaaagcgttaatatttcttgtgctcgatccattgagttatcaccataaaaatctaactgcattgtgccgtgcatagagttaaagataaattgcttgtcaccatcataattaacggacttttgatccaagtggcgaccatgcataaaagtcatgataatggcgtttttagggattgggtttaggttgttataacctctaattactgtcgtatcatttgcaggcagattaaaggttttaagcagatactgacgcatatctgtataaaggtcggttaatatgctcatggtgcaaatactccttgattgaaaggcttggcatcagatccagcaaaaccaaaataacgagcatcatgtggcgattccttaccagtattctgaactagcactttcacccatccgctatagttattttcaccttcaacggcgatttcatcacaatatgactcaagaacagctttaacttgccattctgtaggaaatgcctcaccgtatggattcatcacaataatagacgtacctttttgcatggcgcgtctaatcgctggaatcatgccatcagcatagatagataagaattgtccttgttgattggcaaagcccagatgctttaaatcctcaacgctcattgactgagcttgaacaactttaggttgtacaatgtactgcggcacttgtgtgccatctggtgtgacagtaaaaccttcagcaaccttcaatagtgcttctgtattactatttactgccgttgtcatgctattagctatacttcttaatcttagactcattaaaaaaagccccatcgtttgatgaggctagtttaacattattactaaaaagtttttacaagattgtatgtaatagaaatatcatcaacatcctcaaatattagtttactatttggaaagaatttactaataaggtcgactgcatttaagccacatgaatgaatactcatatagtaaaaatcatggatcattttcttcggagcggttattttaataacatctaaatttgtaacctcaccattttcactttcaaccaaatcatcatacttattatttaccatcctaaaaatttctatatgattcataccaaacccctttatttgcatattcttattatatctgtttactatcgagaatgcaatatgtgatttaattaatctataatttttgttgtgaaaagcgtttgatcgaatataagaagtgccttaatgtaatcatctgaacatttcgcaagatcaacatcattcaattgtcttgattcggcatctttgaaaaattcaatggctttttcaatcttctggtaaagcttttctgaatttacttttattgatactttcataatttaccctcaacaacttcgtatttaatactgtcatgcatgagtaaactttgccttaatggggcgtcaaatcctttggctgcaatggtcgctggggcgttaggcgggtttgtccagctcataatagaatactgaatctcattctgcatactcaacccaaccaaattaagcgtatgctctaatgtgtatccagccttaatacctttcttggttaactctacccaagccgccttttgctctcttacggtggttctcataaatggacgacttggaatatttttaaacccatattcattcttataagccacagtagcaacgctagtgccgtctggatattttgagccaggcattaccccagcttttacatactgatcattactagaaatcaagcggtttaatgcttggtctagtgagcctgtgcgttttattgacataataaaaagccccatattgaatggggctagtatagcattcagtctaataattttccaaatcagacaagacccattctagcttgtgcatttgcattgcatacaccatagctaaattatgagcaacattgtgctccatttcatttttcgcactatctctttcatttatagcattaactctaagagtatccaatcttgctatttctgctttaattactgcttctgatttcattttgctttccttcttaaaaaacccatcatggcatgacgggctttaaatgtgaaatcaatatttttgatatgggttaaaggtcccagccttctgagccaatgccatagccgtctttaaagatgagatccttgttcgaccaatcaaacaaagatttcccaccagccatatagacatcgtaatcatctaaaagtagtttaagggattcaactattttttccataaatacacctattaattaataattaccccacccaaaaggataaggtcatttattgcgcttaactggcatcggagcagttgcagcaacccacaatgccgtgcgatatggcgcagtaaacgcatagaatttagcaccataaggcgtttgtttaagccattgctctaaagcaccgctacccatagagtagtcagtgctaatagatacagagccttcagtagcagagctaatacgaccaactaagccactgtttccgtcattgatcctattttgcaattctgcgttatgagccaccaaaagataaaacctcttcttgcgatcttttaaactaaagcaagatgtttcagtgttatcgacaatagtgttttctacttcctcaaagaagtttgttagctgttcattagtgaactttgcaaactgcggataagcaagcttaaacgcagcaggatcaaaaacgaaaacattagacattagttttcctcaagtttgccctttttgtctagttctgctggatctagggcttcaagtccagttttaaccttagctttttcttttgcttcggctttgactttagacgaagattcatccgcatagattaactggtttactagaagatcatgattggcgaatcgagcgcgccaagcatcccacaattctttatctacgccacttgttaaaccaacttttctaaagccaccatcaatatacatagaactaggggaattccacccgttaataacttgcgacttcccttccatttccatcaaaataggaaaaggttgaagtaatgaaatagttacagttgccattgtttaagcctcttatttgataacgttatcttaacctaaaaaaaatgctaccgctagggtagcactttcttgttattgattagggagtttcgtttggaactgttgctgtaacaacaaacattgggcggcgaataatcgcgccgtaagttgtttgacaacgcttttgaatccaaccagatgccttctgaatcattggatgaacacgcattttttcagcataagccaattgaattgtgtcacttccttgatacttgcgaagaaccaactgaacaacctgacctgaagctgtcgaatattcaggaactgagtaaatgcttaaattagggaagctctttttcaagtagtcagcaacattaagaccgtaatcgttagctgctgtaatgagcgcttcttgctctggtgacattaacaaaataaggtcgtcagtacgcttaactaagccgttcgcttgcttagccaattgtttaaacattttttgaatgtcggcaacaatctttaatggaagctcgaacgtccaagcatctggaataggaattgctggcaataggttcgggtcgttcagaacaccatagttttgcaaaccagctacaccaaaagcatagatttcatgttgcatcttgtttagtgattcaacaatacccaactctaattcagcagcccagctaagtttagcttcgccataaatagccgcctctttttcaccaacgcgcaagaactgttggtaattgtaagattggcggttgtggtagtttacgtttgcgctagttgtgccattatcattccagtcgccataggtaacgggcttaccaccattctcaacaacagggaatgtcataacagaagttgtccagtcgcctttctgaacttcttgtagagcctctgttaacgagtttggagagaataaaatgcgcgtaacttctggatcaataaattgcgtaaacatctgcaaaacaccagcgttaggcgttgtaagcattgcagtatcttgagcaataatcattttgtcgcgaggagttgcgatacgaggcgtgccttcagggtagtgaacgccgtaacgcgcaagctcttgaggtttaaagctttgattctttgtagtcatctgatcaccttatttagtaatagtagctaagtcatttgcaacagcgccaatcgcaactttaaatccagtcgcgtagtgatcagctagtgtgtctgcgctagaaaccgcaattgagccatctgcattagatgcaaaaacattttgaccaacggccgtaggagtagcgatctggatatagaagtcgcctttatcgtgcaacgtaatttcctttccagcaggaattgtcatagacgcctgaccaagccactgagtaataaccgcaatattgccagaacgactaacaaaaccaacaggctttgttccactaattacattagtaaccaatccagtatcggggtcagcttcggcaaatcgagcaataaccacaccagaagcacctgctcggaattgagcgcggccagacaatagggtatggcgagggttttgcgatgcaaaatcaccagcaacaccagtacccatcttaatgttaacttgttgttgtaaagtcatatatacctcaacctaatttaatgtgagcaaaaatgtcgctaggagcatcatataaagatgaatccatagccatagatttaggagcttctgtaaccttctgcgacttaacaagagcagccaagccggcagtattaataccagtagtgtctacacctttctgtttaagcgcataggcataaacttcatgatctgagctaaagccatcaagagcaataacacccactaaaggctcaacttcgcgaccagccttaaagatgttcatgatagagccgcgaatttcagcagcatcttgagcaatttcttttttgtcgtctttcttggcttctcggtcgtctttgtcagttttttctaacgctttacgctccttatctgccttttcgccatcgtcttcatctttggctttgtcatcatcagaatcttcaacgatttctacatcatcttcatcttctgcctttttgtcgtcctcgtcctcggctttcttgtcatcttcatcaagagccaaagaaccgtgaacggccacaatagtttttttaacatcttccaaagaatccatgcctaactgtttttgcagttttgcaaggcttccgtttttaagtttgattgtcatcaattgaccctctattgaatctgcgataatcgcgtcacttccaatcctaccacgttcaacgatagcaacgtgattaccatgaatattcgtcataataccatcataaggtacaccattaaaagtacccgacttcatgacagcatcataagcgtaacctgcgcttaattctcctaatgccttgctttcgatatagtcaatcccttcttgatcaaatacgcgcaatgatgaccatactcgaccttcgtcatccattgttatgtctgtaccaattgaaccaatagttgactctttttctggttgagatgcgtcaacaggaatatggcgtttaagaagctgcaagcccttgaatgtgtctaatgatttacgcaattcgtcaggatcacgcaacagcatgtattctttgtttggatctaagcctaattccttccatcgtggaattgacgaaccaagataaggatttactgcggcttttgtgataatggttttatcaacaattagatggccattgcggtcgtagctacgagctgatttatccattgcatgttctggctctttcttttcctcttcaagccattgatcaaacaattcttccaacttatcaaagtcagtatcattagccattccagattcacgatatgcaatagcaatcgcttgatctttcggttttcctgaatcaattaactcacgaatgttcttatgaataacatcctttgagctacctttaattagaggcatcaaacatcctccactttaatttttagtcttttgattaaatattctattgctctgtcatctgaccaaaaaccaaaaacaaccatgaatttgcagaaattcacaacaaaattggtttgccatgttttttcatagctaagttttacctttatatctgcgatattcatatcactcacctatacaatttttcttattatgcaataaaaaagccactggttaaagtgactttattctaaagaagtcgcgcggcttgcaaacacaaccctgccatcgtttaccatcccaaaaatcataagcataaatatatccgcaatagtaagcactatcctttggtgtattatctcgtaagttttttatgtaaattcttcctgctttatgtgtcgccccatcaggcgcattcctcttaatctcgtcaatgttcatttacaaactccaaagcattttgaaatacacaccaatcaattttaaatcctttattgagcatatctgatttcgcttcatttagttcgtattgattttcgcaaactatataatcaacaccgaatatctttttattattcataaagtggattaatacataatttccaaccttctcatgactatcccttgtcaaccatattcttgacattgcaaaatcaaatttatggtgaaatcctttctttctagctaaatctatcgacttaatcattcttcaatcctcatcttgtcgcaatgtatgcataagtaagctttatagatccagcaatattggtagatatggttgcattctttgagtgtggtcattggtcactccatcgctttcttgatttcatgaatgcagaacttcaaagcaaatactctttgatcatttccgttctttaggttctgctcttttgtcaattcaagttggctgacaagctgactagctgcatttcttaacttgtcgttttcaatctttgagttatgtaattcttgagctaggcgatctacttctaagattgcttgctctcttgtaagctcttggtcagcaaagcaagtgccgcctgcatggcaataaccgtctgcgccacagtaagggcttccacccttacagcgcatcacagcattaacccatatgtcatcgtccttacttagcaagtcgtgctcacatgggatcttgattgctttcatccttcccccttgagcgcttgctctatatcacaaatcgtttcttcaatagattcactataagtttctaatgattcgttatttaatgatctaagctcttttgttatttctaaagctaaatccaccctcttttgcatcttcaacatatttatgccttgctgagtgtataatgtttgcagctcgtcacgctcttgcttgatctttttaaagtaaacttcatgaccaatcacttcaccgtgatgagatgctttaagctcctccactttcgcttgctggtgctttaatcccaatgaataaatatcaaatgcttcagtcctgtccgctgaaacattcatatagttttcttcccaccattcatcaaactttagttcaaactctttaaactcactcatggctggctcctttttctgcatcacacatttcacatttatctatatgcccccacccatcatctcgaatgaagccaaaccccttacaagccttacatttgactttctttttctcacccaccaagaaatatcgatctttctggttgtaggtaatatcaatagaacctgagtaatagcgccttaacgccccatcaatatgaaattcgtgtggacctacacaaaacatccaccccgaatccccgccgcactttgtaaaccatgtgaaatatgcttctctccatttcacataacggccagacagatgaggagtcaacaattcaattaaacgtgctctaagcatctccatgcttgctgacatatctccatagtgatattcaagattgtagctatactcgcctgtgttatatctagttggcatgagattcaccgcctccgtatattgattcgtggtctttgattgcctgctccaattcatcccagccgcttacatttggatgctttgctttattccgcgctaggtagtctttggcaaacttagtaccaccatgccacttgatcagatcaatcgactccaccaagcgtttgagatccgccatgttcacaagttcaattcttggattaaaacgatctgaatacttttttgctttggtgcagtagcataatgttgagtagtaacactccatatatttgcttgggatgccttcaacaacctctcgcgccttatccaatccttgctcacgaataaactgctctggtttcatttaaaaatactccaatacatcaaagggcagtaaattgcatgatttttaatatctcttccacaaaattgacatttaatcatattaaatcatccattctggtttttttacctttaggtttttatgtggttcatcaggaaagataaaagtcacataaactttgctttcttcataatgataacatccctctggcatgttcgctttaagccattctaattttgccttgctcatttaattctccatgcaatattctattgcttgttctaatgaatgaaacatatcaacatgattttctaactcatacacatgatacagccaatctaagtgatctatttttattgttcttccattaacaaaaatagattcaccatcaaaaaccacttctgctttaatcatttcaaaccccttctgctgaataactaaatcttaccactattaaatataattctcaatagtaaatctaaaatagtttagacaaaataaaaccacccgttaggatggttctaaataaacttagatgaagtgaacatgggagagtcgaactcgccaataaaacagtattttacaatggttgatcaatcccaccgccacaaaaaccaattgagtaattaattcaatcttataatgtgaactgcaatattctagcacctaagctattcacttctctaagttgcctactttcaggcggtcatcggtttctcgtgccaataccgaaaacaacagaacattttagccatctgtcttgcctattgatcggcaacgtgcgaatagttgctttgctatgtccctttgaaggtactcctagtctaatgatattcactgtgtcgcccacagattcgatcaattacagaagcgacttcacacatccgcaaaaattacaaaccaaccaaggcgcgcagtaatttttaccaatgctacatgatattgtagtcatactccatgatttaaataataccaaaacaatctaaaaatacaaatcagttgttttgatatgggttattggttttacaaatttacagttagtgaaacggattgccgtgtcgatgtgcgctttcacttatgcttatcacatccaattaactgtattatgccatactttctggaatttcgataattagctttttcctacagcgacaattgatttcgatgccagtgtttgtccacttgccatcaagaaataagccttccttgatgttaaatctagttccatttgcctttacgtggctttgacgaaaagtctttcctgcatgactgtgaagccagtaagcttccgttatgcccaattcctcagccctagcattctcaaaagcctgattcaacttagctgtctgatcttttgcaatattctttgccctgcgatctgttacgccgtcaatctctttgagttgcttgattagtgactcaacatcatagccattcttaacactacgccaaaccgcagagcgaactttatctaaatactcattaccaatagacttgattatagcaacgttttcacctaaagcaatttgcgcttgatcttccatatatttcgttggtcggaagtttacagtaaagccacgcttgcgaagaatacccaaaagtcgcttgtcgtagttggttttagccttacctactaactcttgcgccacttgagtcgatagtttatctaaacgatcttgccaccgactaaccaacccatcaataacatgtcccatccaatccaaaatgccatccatagcaatttcagaccgcataggcttaactacgtcctgaattaaatcagaacgcatttcatccatcatgccttgcatttgctccctgtaccatttggtcagggatgcattaggcgctattgactcaagggttatttccatatctatttttcaacaacatctatgttttcgttttcagcataaggatcacgctctggcgcactaccttctaattgataaccactgtcttcatcatcattcaaagcttgacgcgcttcttcgttgtcgatcacaccttcttgaatgtaaatctgagcagtttgtgctttcttcaagtttacgtctgcttgttcattatcgtccaattgataaagcggattaaagacaaacttaatactttcatcaatatttccaaacaagctcaattgcatacagtcaaggataactttgatctgcggcaaaatatacgcttcttgctgtgctgatacgcaatcgtaaaataccctaatctcaccatcagaagtattaccaagacccgccgttggggttccaaatatcttcaatactggaatttttgaagggtaggcaagcatctgtgtaaacttgtctagcaatgtatcaagcccagataatggcgtattgatctggaagaactcttcagattttccatccaaagccaccacgccttgattatctcgacctaaagccaatgcttttaagcgaccaatcaactgatttgcgccgccttcatcacccgctaagattgcagacatatcggtcttgataccagtcaagctaaacatggtaatcaatttagctacagcgtccacaatagattgatgacgctgaacgtatggcaacatcaattgcgacattgacatgccataaaaattataagctggcttaagcatttgcccaacgggacgcataattaatgtaagcaatcggctatgatgtacatttttacccataacccaccactgcttaggcacaaagaaatcactttctagtggattatttgcattgtacatactaggcgtggaccaaagcggctcaataaccgtgaagccttttaacgagcctttcttaatgcctttctcgttaatcaatagaggcagatcggtcttatcctcttgcccttcgatctgaatgtataactgagagccaccatagtagtaatcattctcgatgtgcttacggacaaggttgcgaatgtccagcctgttaaattcttccatgagaatgtctacgcgcttctggtcaccacccttgacctcaccccattcgcgtgtcatctcttgagcaaatgtttcagcaactaggcgatattcagtagactgagccaattgagctaatacttgatagcctaaaaagttgctataaaactgtggctctaatccagcatattgcgaagctgtacaaaacccatccattgccatagcgctatgaccatctggtgcaacaccatcaggcaaagaaggcgctttgtactgaatcgccacaccttcattatgcgcttgcatagcatttaacaaagcgttccatttcggctttcgctttggcgcttcttctttctttctgaaccaatcaaacatgttttaccttccgaaagccgattcgatatcggcgatgttaatcttcatacccatgccacgtttcatgactttttcaagcgcatagcgtaatgcatcgacatagtggttatctttgtcaatgataataggtaatacttcatctgtcagcctgtcttttttgtacgagtaatcacggaactcttttagcgtttctttgcatcgtgaatgaatatagactttcttaaacgacttaataaactcaataccatcctcaacagagcctttacccttctcaacgggcgcaattctacgcaagccttttttctttaaatgactaatagactcaggacgggcactatccgcataaacagcatacttcttaatgtctggtattttcttttctaaaaattcaacagtatcgtctagctcaagaccaactttcccaccttcgtgttcaatccacaaacaatcatcatgaatccagactttgacacatgccgttggatcctgagcaaaaccaaagtccaatccgatgtaaggaccatgccatttttgaggatctggcgtaaaatcctttttctcgtatttgcccttaaagatttgcgcttcagacatttcaagataagcgccttcccatatccagcgataagttacatcatcaagattggcttgatctcgtctgcgctctaagtcaagaacacgaggaaaccacggattatcagaatagttcatttccacacacataccaataagttcgcctgtttcatcatcgtatatttcgggatgattaaaccgaacatccgtagggctgccgcgcttctcagggttataggtaatatatacttgtgaattttcctctcgcactgttggcaatagttttctatacgccatctcagatacgttctcagcttcatcaacccagcacagtaaaatacgtgctttggatttgatactgtccagattgtgtctaagacctgcaaacgtgtattcaaccagtctgtttttagtcctgatgtagttctcgcccatatcgtagtaatcattcagaaaaggaacagacctgatagcctgcttgatttcctctaatgatgagtctgctagggagtttaagtgttcacgcgcacaaagaatcacaccactctggccactctcagcaaacatatagcccttgattgcagtcatcagcgcaaatgatcgagtcttaccactacctcgaccgcctttactcacaaggtaacgcagatttggagtagtaaataatgggattagttttggaggtatctgaatgttagcttctgtcatccaatatacctcgcacctttggatctattttgttgcatccacattggctgtaagttagacaaagcattgataatttctggacggaactcacctttgcttaccattaccgcaatcggtattttgtgatcaatctcccattcgccaaaattatcccaactcatgccgtctttaaactggaattccattctttgaattaaatcatctgttgtatatccaatcttctcaaaggtgcaaaaatcctttggcttattggttgcttttagaacccgcctaagcattgatctaactttatgatttacagaatatgtaatatcattgctatatctttcagcatggtaacttcgtcttttacttaaatgatggtccctgttcttttctctccatgctctactcctttccctaacctgatctctattttctctttcgtagtttgcttgatattccttaactttatccttgtttttttcataccaagcctttctgtttgctctagcgcagtccttgcaaatagtgcaaaaaccatcattcttagatttatcttttgagaacaaatcaaatggtttctcaataaggcacttattaca

SEQ ID NO: 9 - protein, 170 aa, Unknown
  Bacteriophage YMC16/12/R4637_ABA_BP ORF37
MNFDKAFDTTIGHEGGFTLNKNDAGNWTGGKVGVGQLKGTKYGIAANSYPNLDIKNLTLDQAKAIYKRDYWDKAKCDLLPEGLKFHVFDVSVNSGVSRGIKTLQQAAGVKDDGIIGPNTLAAIKSFDENELLLRFYSFRISFYTSLSSFSNFGKGWMNRVANNLKLGTGG

SEQ ID NO: 10 - protein, 696 aa, Unknown
  Bacteriophage YMC16/12/R4637_ABA_BP ORF49
MAENIVESIIVKLGLDGSQYNREAEKAKSNNDKLNKSVSETDKIVGNVTKTLARWFSVAATATGILKMVDQVQKLNDELYFLEKNLGMSSQTIKNWQGAAGAMGGSAQGMTESIKSLNMGMNDFVTMGDTTLLPFMNALGVGMVDAQGKLRKTDDVMLDLADSFSKMDREQAFSIASKMGIDEGTFNTLVQGRKEMEKMLEYQSKMYKSSEEELKASRQLAQNRALLGQHWESLKTMMANAIIPLFVKLSEVALGIFEYLQENQQAVQAVFKGISFVVGAILIPILAKATIAALAFIAPFAPFILVVGALGAAFGLLYDDYKTWAEGGKSLFDWGAFRKYIDDSTLSVDNLKNAFSNLGKDMLNNAIPTLKGYAEILDKLVSGDFKGAASQAWDMLKNYYSRAADFVDDLTGQEQGTLANTVGNLINPSDSASTTPSVSGGSNSGNYKFKFGGGVDQDISDASKKYGIDEKVLRGFVKMEAGWKGAMSPTGAIGTGQFIQSTWNSLAKTSEGKEIGMTEINKSNFRKANDPRYNKRINTMATALLAKKNADMLRAAGLPVTGENLYMMHNIGPGVIPALKGGAVSSATELAMRQNGKRSGESAAQFAKRQQSIFQSHYSTANAEAAQNALQSTRQGDFIDLTKARQNQSTANKANEVQVNVGDINIQTSSSTVTGNVQDAMGAIKDQFYQFRNSFN

SEQ ID NO: 11 - DNA, 513 bp, Unknown
  Bacteriophage YMC16/12/R4637_ABA_BP ORF37
atgaactttgacaaagcatttgatacgactatcggccatgagggcggatttacactcaataaaaacgatgctggcaactggacaggtggcaaagttggagttggtcagctaaagggtactaagtatggaattgctgcaaatagctatccaaacttggatattaaaaatcttactcttgatcaagcaaaggcaatttataagcgcgattactgggataaggcaaagtgcgacttattaccagaaggcttgaaattccatgtttttgatgtatcggtaaatagtggcgttagtcgtggcatcaagacacttcaacaagcagctggtgttaaagatgatggaattattggacctaacacattggcggctataaagtcatttgatgaaaatgaattgttattaaggttctattcttttcgtatttctttttacacgtcattaagctcattctctaattttggtaaagggtggatgaatcgtgttgcgaataatcttaagcttggcactggcggttaa

SEQ ID NO: 12 - DNA, 2091 bp, Unknown
  Bacteriophage YMC16/12/R4637_ABA_BP ORF49
atggctgaaaatattgttgagtcgataattgtaaagcttgggttggacggctcacaatataatcgtgaagccgaaaaagctaagtcaaataatgacaagctgaataagtctgtcagcgaaaccgataagattgttggtaacgtaacaaagactttagcgcggtggtttagcgtagctgccactgctactggcattctaaaaatggttgatcaagttcaaaagcttaatgatgagctttattttcttgagaaaaatttaggaatgtcatcgcagacaattaagaactggcaaggcgctgctggcgcaatgggcgggtctgctcaaggcatgactgaatcaatcaagtccttaaacatgggcatgaatgattttgtcacaatgggcgataccaccctattgccattcatgaatgctttgggtgtcggcatggtcgatgctcaaggtaaactaagaaaaactgatgatgtgatgttagaccttgcggattcattctctaaaatggaccgtgagcaagcattttctattgcctcaaaaatgggaattgatgaaggcacattcaatacgcttgtacaggggcgtaaagaaatggagaagatgcttgaatatcagtctaagatgtacaagtcctccgaagaagaattaaaagcatctcgccaattagcgcaaaaccgtgcattgctaggtcagcattgggaatcacttaaaacaatgatggcaaatgctatcatcccgttatttgtgaaacttagtgaagttgcgttaggaatatttgagtatcttcaagaaaaccaacaagcagtacaggcggtattcaaaggcatatcttttgtagttggcgctattctcatcccaattttggcaaaggctacgattgcggctttagcgtttatcgctccatttgctccatttattctagttgtaggcgcactaggtgccgcatttggcttgctctatgatgactacaaaacttgggcagaaggtggtaagtctttattcgattggggtgcatttagaaagtatattgatgactcgactttatctgttgataacttgaaaaatgcttttagtaatctcggcaaagacatgctgaataatgcaataccaacgcttaaaggttatgctgaaattcttgataaattagtgtctggtgattttaagggagcagcatcacaagcttgggatatgcttaaaaactactactcaagagctgctgattttgttgatgacttaactggtcaagaacaaggaacattggctaatactgttggtaatttgattaatccttctgattcggcttcaactactccaagcgtgagtggtggtagtaattctggaaattacaaattcaagtttggtggcggagttgaccaagatatttcagacgcttcaaagaaatatggaatagatgaaaaagtactgagaggttttgttaagatggaagctggttggaaaggcgcaatgtcaccaactggagctataggaacaggacaatttattcaaagcacatggaactcattagccaaaacaagtgagggtaaggaaataggaatgactgaaattaataaaagtaatttcagaaaagccaatgacccccgatacaacaagcgcattaacactatggctaccgccttgttggctaaaaaaaatgcggacatgttgcgggcagcaggtttacctgtgactggcgaaaacctatacatgatgcataacataggaccaggggttataccagccttaaaaggtggagctgtttctagtgcgactgaattagcaatgcgacaaaatggcaaaagaagtggagaaagcgctgctcaatttgcaaaaagacagcagtcgatatttcaaagccactacagtactgcgaatgctgaagctgctcagaatgcactccaatccactcgtcaaggcgactttattgacttaaccaaagctagacaaaaccaatctactgcaaacaaagctaatgaagtccaagtaaatgtaggtgatattaacatccaaacatcatcaagcactgttactggcaatgtgcaagatgcaatgggtgcgattaaagaccaattctatcaattccgaaattcatttaattag

SEQ ID NO: 13 - DNA, 44576 bp, Unknown
  Bacteriophage YMC16/01/R2016_ABA_BP
ttatcaaagtatgctactggagccgacccttcgattaaagtccgaaatcattgttaaacatatttcggttgctagttaccctgatttaactcagtccaagctctgcctagcacgccatttctgactattccaacagattcctgtttgcttgaccctcacagtacaggattgtttaagctatgcgctgccgttatagttgttccgaccacataaccgatttacttagtttcacactggttgtaagtaacagataaattatagcatttgattaaaaatcgttaagtgaaaataattcatctttgatttcacttattgttaacttgttcatctatgcaaattttgcatacttagaattattcatcaaatttaaatgaggtgaattttattcatgtttatgagttggtgatgaatgatgctaaacttagcaaaagtggagggctactcatgtcaataaaacgtacaggctcactagaccaagcattaaaccgcttgatatctagtaatgatcaatatgtaaaagctggtgtcttagaaggatcaaaatatccagatggcactagcgttgctactgtggcctataaaaacgaatatggatttaagaatattccaagtcgtccatttatgagaaccactgtaagagagcaaaaagaagcttgggtagaattaaccaaaaaaggtattaaggctggatatacattagagcatacactcaatttggttggattgagtatgcagaatgagattcagtattctattatgatttggtcgcaaccccctaacgcaccatacacgattgctaagaaagggagcgattctccgctgcgtgataccatgctaatgcacgactcaatcaaatacgaagttgttgagggtaaactatgaaagtatctataaaagtaaattcagagaaactttaccagaagattgaaaaagccattgaatttttcaaagatgccgaatcaagacaattgaatgatgttgatcttgcgaaatgttcagatgattacattaaggcacttcttatattcgatcaaacgcttttcacaacaaaaattatagattaattaaatcacatattgcattctcgatagtaaatagatataataagaatatgcaaataaaggggtttggtatgaatcatatagaaatttttaggatggtaaataataagtatgatgatttggttgaaagtgaaaatggtgaggttacaaatttagatgttattaaaataaccgctccgaagaaaatgatccatgatttttactatatgagtattcattcatgtggcttaaatgcagtcgaccttattagtaaattctttccaaatagtaaactaatatttgaggatgttgatgatatttctattacatacaatcttgtaaaaactttttagtaataatgttaaactagcctcatcaaacgatggggctttttttaatgagtctaagattaagaagtatagctaatagcatgacaacggcagtaaatagtaatacagaagcactattgaaggttgctgaaggttttactgtcacaccagatggcacacaagtgccgcagtacattgtacaacctaaagttgttcaagctcagtcaatgagcgttgaggatttaaagcatctgggctttgccaatcaacaaggacaattcttatctatctatgctgatggcatgattccagcgattagacgcgccatgcaaaaaggtacgtctattattgtgatgaatccatacggtgaggcatttcctacagaatggcaagttaaagctgttcttgagtcatattgtgatgaaatcgccgttgaaggtgaaaataactatagcggatgggtgaaagtgctagttcagaatactggtaaggaatcgccacatgatgctcgttattttggttttgctggatctgatgccaagcctttcaatcaaggagtatttgcaccatgagcatattaaccgacctttatacagatatgcgtcagtatctgcttaaaacctttaatctgcctgcaaatgatacgacagtaattagaggttataacaacctaaacccaatccctaaaaacgccattatcatgacttttatgcatggtcgccacttggatcaaaagtccgttaattatgatggtgacaagcaatttatctttaactctatgcacggcacaatgcagttagatttttatggtgataactcaatggatcgagcacaagaaatattaacgctttggaatagcccttatactactgatacgctagttaattgcgtaccattaggtaatccgcgtatacgcgatttatcttttgttaatgaggctggaatgtatgaattgcgctttatgattgaagctgacttgcaatacaatacaaagaaaaaacagttaatatactggaagacgtttctcaaatcgatttggagtctatcaatgcagtttaactcaatcccagcaagtaatattgctgctgtctaccctgccgtaattggtggcggtggcaacccactaggattaaatacaactttatttgtaaatgaagctgtgtatcctaactatgaatatttttctaatactctagtcggtcagcactacggtctagaaagtgatgtttataagtttgcgaccgtttactttaacggctttaataacgcaactactcgaccaaactcactattcattgcaacatacaattcagatgaatacccagctactattattggcggcgatattactggtacaagcattgctgatcttaaattgattaacggcagtctgaatatcgttgttgatggtgtatcaaaaaacgttactgttgatttaagtactgcaaactcatatagcgatgcggcggccctaatcggtacagcattaactttgacttgtgtttaccagtccacaactaaaggttttgtaattcaatcaggtactacgggcgaaggctcaactattagctttgcgactggtactgtggctgataagttgaaactaactcaagacactggtgcaatcctaaacaatcatacaacgcaagatacgcctgaaactgccgcattgagcgcaattcagtttagcagtaactttgtgaatttcacttatgcaaatggcgtatttgatgacgatgctctaaaagcttttgctacttggattactcaacaaaatagccgattcaagctgtatacgtggggtcttgatcctgttgctcttggtcaaagtggtgcatcttttggtgagtgggcaaaagaaaatactagcggtgttgttccaatctatggaacatttgacaaagcagctttcttctgtggtgtttctggttcaattaactatcaagaagctaacgggcgcacaactactgctttccgtagtcaagatggattagttccagacgtaacaaatgaagctgatgcggaaacattagttaaaaatggctactcattctatggcgcttgggctactgctaatgaccgattccagtttgctggcaatggctctgtaacgggtcagtacaaatggattgataactttgacttccaagtgttcttgcgtactcaattgcagcttgcatatatgaacatgttccaagctcaaaagacaatcccatacaatgatcaaggtattgctacagttcgagcatactcacaagatccaattgatcaaggtattaactttggtggtatccgcgctggtgtaaacttgtctaatgctcaaaaattccaagtgaatcaagaagctggctttgatgctgctagtcaattattcgcacaaggttgggcgctatctattactttgccagactcgcaaactcgcgtggctcgtgaatcatttattatcaagttattctatacggacggctctagcatgcaacgtctagaaatgactgctactaacgttcagtaaggagattaacttatgtcaatgggtcttaatccgaacacaattacatccgcaaatacggtcatatcaatgcgctgtgctggaatttatgacgactggattacgcttgaaggggcacaaacagatgcgtttttgtcatttgaggatgtgacgtttgcacaaactgaagtaggtgtagatggcaaattgtcaatgggcttcattccacacaaaaccaacagtacaatttctttagctgctaacagtaaatctgtcatggtttttgaaaacatttacaaaaactttgttaagatgatggacgtattgccgattgagcttcgtgcttactatccttctgttaagcgctctcaaacaatcaaaggttgttttgtgggcaaggctggcggtactggtgtaggggctttattagctggtagcacataccgcattgaaggtatttcagaaggcttaatcgaagttaactaaccataaggggagcaatcccctttttattttttagaggctaaaatcatgtctgaaggcttaaagacaaaaacagttactattgaagatggtcgcgataaaggcaaagtttttaaaatcacggaaatgccagcgattcaagctgatgaatgggcacatcgtcttttagaacaagccgcgaatagtggtgttaatttaaaagacgttgatgtattgaatcttgatacaaaatcaatggctggaatgattgaaattggcgcggcagtctttaccgtgctaggtcgtattccacatgaaatttcacgcgaattaaaatttgatttacttgatcgctgtgtacaaattgttcccaagtctggtgaaccacgtatttgtatgtgggatcaggagattaaagactttaagaactttaccattctagctgctcacgcaattgggattcatattgattttttagaacaaggcgaagcttagtgcttgattattcttatcgcaaggacgcgatgagtaatcaagaattaaaagatggtgttttggctgcccctttgcatatgtctgaaacagtgtatagagcattattaactggaatgtgtgactatcaccagttaaacacttgtatcggtcttgagggtgcgctaaacatgattgaagctaagcaagtcgccgactacaacgaagcaaaaattaaatattttgctagtcaagaacagaggtaaaaatggctgaaaatgttgttgagtcgataattgtaaagcttgggttggacggctcacaatataatcgtgaagccgaaaaagccaagtcgaataacgacaagctgaataagtctgtcagcgaaaccgataagatcgttggcaacgtaacaaagactttagcgcggtggtttagcgtagctgccgctgctactggtattcttaaaatggttgatcaagttcaaaagcttaatgacgagctttatcatcttgagcgcaatctaggaatgtcagcaagcaccattaaaaattggcaaggcgctgctggtgcaatgggtggatctgctcaaggcatgactgaatcaatcaaatccctaaacatggggatgaatgattttgtcactatgggcgatactaccctattgccatttatgaacgctttgggcgttggcatggtcgatgctcaaggcaagctaagaaaaactgatgatgtgatgttagaccttgcggattcattctctaaaatggaccgcgagcaagcattctctattgcctcaaaaatgggaattgatgagggcacatttaatacgcttgtgcaagggcgtaaagaaatggagaagatgcttgaatatcaatctaagatgtacaagtcctccgaagaagaattaaaagcatctcgtcaattggcacaaaaccgagcattgctaggtcagcattgggaatcacttaaaacaatgatggcagatgctatcatcccgttatttgtgaagcttagtgaagttgcgcttggtatctttgaatacttgcaagaacatgaagatcaagtaaagggtgttttcacagcaatatcttttactattggcgctattctcataccaattctaacaaaggctacaattgcggctttagcgtttatcgctccattttccccatttattctagttgtaggtgctttaggcgcggcatttgggttgctctatgacgactataaaacttgggcagaaggtggcaagtccttgttcgattggggcgcattcaaaaagtatattgatgattcgactttatctgttgataacttgaaaaatgctttcagcaatctaggcaaagacatgctgaataatgcaataccaacgcttaaaggctatgctgagattcttgataaattagtgtctggtgattttaaaggggcagccttacaagcttgggatatgcttaaaaactactactcaagagccgctgattttgttgatgacgtatttgggcaaaaacaaggaacgttagctaatgctgttggtaatcttgtaaatccaaacacccctgcttcttctgctccaacaattgcaagcgcaacatcaaaaggtggcaatgctatcttggacttgattgcaaagggtgaagtaagcacaaccgcaccaagtggctacaatgttgcctatcgtggtgctaggatttcggctaaacaaatgtttgggaaggaactatctcaactaacgatcggacaagttaaagaattgcaaagagcaaatttaaatgaacagaaatctcgcggtattcctgctaagcgccgatcttcggcaatgggtcgttatcagtttatttattctggctttgatgactatatccgtgctgctggattaagtgacaaggatatgtttagccctgaaaaccaagatgctatggcgatggcaattatcagtaggggtaaatatggcttgaatgctgtgcgcgctggtaaagctactcctgagcaattccagaataacgtacttgcggctcgttgggcttctattcagaaaactactggtggcggcgttcatgatgcggctggatttaataaggctacaattggaaatcaagctgttgctgctgcactgcaagcaaccagacaaagcgactttattgacttaaccaaagctagacaaaaccaagcaatggcaaacaaggctaatgaagtccaagtaaatgtaggtgatattaacattcaaacatcatcaagtaccgttactggaaatgtccaagatgcaatgggtgcggttaaagatcaattctatcaattccgaaattcatttaattaggtgatatatgttagctggaatgccttctgtaccagattttataccagtggaagctttgacaaatgtcggtctttcattgggcggtgctgctctaattaatggtgtattcggcaagacatgggggatcgtcaatcaatttggaatccctattgttttagccgatactgttgtaagtatgaattacgatgctggctctagcatttcaaaatacccagtagagcaaggctcatttgcttcatataacaaagtgaatgctccttctatggctactgtttcaatgtcaaaaggcagtggtggtgtgcttgaaagaagcttgtttctaggtcagattgaagctctactaaaatcaactttaagctttcacattattacacctgagtacgtttatctaaattatcagattgtcggcatcaatcatgctcgctctgctcaagatggtgcaacaatgattactgttaatgttgatcttgaagaggtgttggaggctagagtggaatactctattgaagaagtaaaagcgcctagcgactctaaaactgtagatggtggcgctaagcaatcaacacagcgaaccagtgttttacgtggacttttcggaggataacatggctcaatacgatattcctttatcacaagtcccgaaccaatttttcactacatcattaaatggtgttacttgggcaatcaccctagaaactcgattaaataatttatatatcagtttatctaataacaatgatggtgatgtattattaaatcggatatgcctgaatcgaacttatctagggcatggctttatttttgttgatatagatggaaatagtgatcctgaatacacaggcttaggtactcgttatttacttatatggacggacgaagtatgatttcatggtgcctagcctgacggggtgacagtgaagatagcctattcacgtggcacaccaattttattaattaggttatactagcctccaatacggaggctttttattgtgaaaaagaaggtcattaaaataacgctaacattgcaagatggcgttcaaacttttacggctgaaggtgataaccgattatcatctactggcttagccatatctacaaatatcacatatggcaatggggctatttcgccgaccgctcaaataaccgcttacggcctacccctttctacagtgaataagcttacgcgcgtccaatggaataccatgcaagcaattctaaacatggttaagattgaggtcggcgaacaggggcagccactaaaagttgcttatgaggggaatattacatttgcgacagtcaatacagatggcgcgccaaatgtagcgctagtaattacaagccaaatggctgtagttgaaaaaatgcgtcctactgcaccattcactatcccaaaaggtgaagaggttgatgcagcagatattattaaattcttagcgcaagacatgcaatatgagtttgaaaattatggcgttactcacatcctaacagacaccacactgaacggctcaaatattgagaaaattgaaaagttagctcaaatgtgcgactttgatttgtacatcgagcaaagattaattgttatttgcaagaaaggtggggatagggaagttaagataccaatcatcacgcctaagacgggtttaattggctaccctgcaccagatcaaagaggaataacatttagttgtgcgtatgatccacttgttagatttggtggcattgtgcaaattagagaaagcattataggtgatgttgtcaatcaagattggcgcgtatatggacttgttgctacacttgaagcaaatatcccacaaggcaagtggcaaatgaatgtaaatgcgacttggaggaattcaaaagatgcagcagtccaacgctagtggctttaacatcaataatcttggcggcgctaaagaatttaaggctaatattctctcaattctttccagtgaactaaacactggtgaagtcgtggagataacagaagtttattcaaacgacaatggtccagtcggttttgtttctgttaaacctatgctttatcgtattggtgcggataacaacaatctagagctaggcgaaattcataatgtgccttactaccgaatacaaggtggaaagaatgcggttatatgtgatccgcaggttggtgacataggattttgcgtatttgcgactcgtgacacatctttgttaaagagaactcgttcaagagttggaccaaacgtaaatcgcatttgcgaccagtcggacgcattcttaacaatgacttggagcaaggaagaggctgaacaatatatctggtttaaaggcaatgaaatccatatcaaagcaaattcaaagattgttcttgatgcgccagaagttgtaatacctaatggaaaattaacagtatcaggcataattgaatctttaactgatataatcactaaagctattagcttgtttactcataaacatggcggggttcaaagaggctctagcgatacagacggtccaaaggcttaattatgcaattacatgagttattctggttctttattggttttgttgcggcatttattgtgattcattcagcgcgaagtattgcttatgcagtggcggcatttttcactttattaattgtgctaagaattatgggggtgaatattatatgaagaccatgtttctaaacccaaaaacttgggacttagcgttggacacgcaaggaaatattgctgtagctactgaagagtaccaacaagcgcaagatattgcgtgttcatgtcgtgtttttcttggagatgactattacaacaagaatgatggaataccttatttagagtcaatcatgggtaaatttggctatccaatttctttatatcagcgccacttacaagaaagatcattacttgtttctggtgtagtatcggttaatgtaaaattggctttggataaggatcgtgtagcgtctggatctattgagtttacgaatgataaaaatcttagcggagtagtgggcttatgatcccaaagatagaaataactgatgttggttactcggttccagataccgaacaaatcaacaatggcacatgggaaatgattgatgactcctttgggggcaatgtttctcgcgttcaaggttcgcctcaatatcaattaaacacttcatggactgctgtaattaaagattgttatgacaagcttgtttatctagctaaccaatatgatccgcgatacgcacaaggtatttttcaagatgctatcggtgagctttattttttaacaagaaagcttgctacccgttctcaatgccctgttgtgtttgagggcttgtctggtgcgccaattccagaaggttttgctgttcaggatttgtctggtcgaacttggcgaaccaatggaacttataatattggtttaaatggcaaagtgacgattacagtaacttgtgatgaggctggagctattgaagcactaccaaactctattgttgttatcccaacatccattaatggtcttgatcgtgtttacaatgaagatagcgcggtgatgggatatgacgaagagagtcgtgttgattttgaggtgcgccgaaaagagtctgtagcaatcaattctaaaatgactgattccgctacacttggcgcagttttggcggttcgtgacgttgtagatgcttatgtaatttcaaatccaactgatgcaactgtaacagtaggctcaacaaattatccattaattcgaaattctatttgtgtttccgttgttggtggcaatgattatgatgtggcaaaagctgcttttattaaagctggtactggttgctcttggaatggcaatacagacgtgacagtaattgctgaagaccacccatccaacccgccacaatatccaattaagattttacgtcctgattttcttgatatttggataaaagttatcgttaaagataaagatgcgatttcttacaccattgaacaagaagtcattaatcatattctgactagcgctgcttctggtgaaaataaggtgcgtattggcaaggattttatcccagccgattatatctgtggtatgccgaaaatcggattaaaaggaattgtggcaagtacagacaatgccacatgggttaatgaaatccctattggtattgaccaatacccttctttaaattctttcaggatttctattaaggagagctaatggagaatattaaagatacgataatgtcgcagtacgcacatagccccacaatattggcgcttattgatggcattaatgaagtaatcgatcctcaatattttattgatgatttttacgaaaaagtttatcgcttatcaagcgctgagggttttggcttagatatttgggcagataaagttggcgtttctcgttttgcaaaaactgccgaccctaatgcaaaaacatttggcttccaaccagattaccagccatttaatacctactctttttctgatggtggtgcatttgcttcttatcgtttaactgatgcagacctaagaaagttaatcatcatcaaggcggcctcaaatattctctatgcaactgcatggaatattaataagtttttgctaatggtttttgatggtcgcaaggcttattacgatattgtgggccatatgtcagccgaatatgtttttgagtttgcattaacaccatttgatcggcttattgtctatactcttaaaatgttgccaatgccctcaggtgttggaatatcatataaagagattgatgtggatagaacatttggctttaatggttcagatttaagcaactttaacaatggagttttctatagtggctaatcctatatttatcccgatagcattcgctgctaatggggttaagaaccctattcaaaaggttcggcaagtcggtcaagatccagaggattttacatgggatgaaggttcgcccttaattaccatgacaaaaatcaaagatggtggcaaggcgccaaaaggacaagattttaacggggtgcttaatgccctgtctgagcatgtcatctatggtcaaaatggcaaccgttatacgtggtcacaagatgtcatagatgaatttggtggctatgcgttaggcgcaatcgttcaatccgatgataccacaaaagaatttagaagccttgtagctaacaacacggtaaacccaaataatgggcttggtggagcttgggaagtctatagcgggcaaggaagtattccaactgccacaagtacaaccgcaggtatcacaaaggttttaaatgtcttaaatagtaatgatgttggatctgctttgagtgccgctcagggcaaagtattaaatgacaatatgaataactttttaaataagttttcatcaagtctaattgctaataatggtagcttttccattccactaggtaatggaactacaatcattgttaaatatggctctacttctgttaacggtgactcaaacgctactgtaaattttacaaccgcatttccaagcttatgcttaaatgctcaagctacattgaatagcgtgtcgttcgatccattggctgatgctggttgtggtgtaacttggacaagaactggcttaactttgcgaaacggcacagctaacacattaaacatttcatggcttgcaataggatattaacatgaccaatccaacactcattacaaccccattcgctgaaaatggcgataaaaacattattccagaatcagttggcgctaatccacaaaatgcaactatgcaagctggattcccgccaatcactcaacaaaagatttctgagggtggtattccgcctgagcgaaatgattttaatggaattctaaatttatacggtcaacacattgttcacttgaataagggcttgccttatgaatttgatcaagcttttgctgatgctattggtggttatccattaaatgcccgtgtgatgcttgataatggtgatattgttcgctctaccgttgcgaataatacagtaaatccgaatgttgatatggctgggtggtctattgggcttccatctagcttagtgacggattccagtggtttaaatcagcaagaaattaataacaatctactcggcttagatggttctgactatattaaatattcttatttgaaagaatattcagactccactatcggacaaaaactacaagccgctacagagaagaccgtttatctaattgattacatccccaaatcacagcgttctgctatttttaacggaacatctgtttatgatgcatcggcaagtattgtcagtgcagtgaatgaggctattgcaagagatgcgacactcgtattaccacaaggcaaagtaacttatagcgcattcccaaacattaaaacaccaaaatttaggattatcggcgcaggatccaacaaaacaacactaaattacactggcacagcggatgcttttagtgctaatgctttttctagtgggtcgcccaccgatcaatttgttaacgggttatttgtttctggattttcaattactggcgctacattcacaaacggattttttggtcaggggtttgctcgctcaattatcaatgatattaaagtaaatggctcaactggtgttgatggaacagcggcattccgccttgctggttgcatgctatctacttttaataatttaattactagttatgtctatggaagtctaccatatcgcggatttcaattaactgctggaacacgtgcgggcgttagtattaatgcttgctctaataatttatttacatcttgctatgcagaggggaatcctattggttggcaattagcagcaaatggtgcagatcaaaatacttttatcaatggatcacctgaagcatgcacacagtatggtttgttggttgggacaaactgtagatataacacatttgtcggcattggttttgagaataaagacgctgtaaatggcgatgtttcggatgcaggaatttacagtagctatcttggatgctattcgtcgaacaagtttcttgttagcgggcatagtgatgccagatgcttaaaaattgatggtggatatttcgagcgcttagagattcaggctggagcgcgtggaaccgttattgatcgcgttacagtgaaccactggaatactggaatcggcggtttaattaataatggtgttgatgctcagatccaacgtgttaccgatagtcaaactgggcaatttatatcagcaagaaagccacgcacaagcttgacagttggcgcttcgccattttcttacactaatacaaatcaactcccagttaaaattcgcgtacagggtggtactgtaacccaagcattagtgcgtagaggtacagactcgtggactgagggcgatccagttgcagctggcggcggcacagtaaaagttggtatttacatattaatgccccaagagagtattgagattagctatagcgcagcacccgccgtaaacttaatattaatgtgaaaacacaaacccttcgtagttttgctattataagtaaaacttatataggatgaatctatagtggattttgttaacatgattctggagtggctaaaagcccatgtaggcgtcatatttatgggggtggctggtgcaacagtcaccgccctagtgccttctggcaagccgttagctgaacgagttattagctggattgttggtgttatctcgtgtgcagcactttcaacaccaacagcaggtcttttgactggcggtggatatgtcgaggtttttggctttatttatggcatgggtggcattacgttagcaaaaatgctaattaaagctatcgaaaagcgtagtaaggtagaaattgaatctaaaacgggagtgaagctcgatgatgacgtttcttaattatctaagcttttttttgattactggcagtttaatgtttgtggtgttccatccaaagattagttttccagtgcatgtcgatgtaattatgtttatgcttgctattggtgtaacagcaatgtttatcaatacactgcaaggcagggatttttatggtcacatgcaagacgctgaaatattagtccgtctagggcttggctgcttaacagtacgttttattcatgaatatttaaaggtgaagaaacatgaaaatgactaagggtggattcgccattcttagagactctctaggaaggctaacagaaagccaagtcgctgaaattaacttcatcgtggatgcgatggataaagacaagtctatatcttattcacaaggtgcatatgtattggcgacaacatggtgggaaacagcgaaaaccatgttgcctatttctgaatacagaaaagggaaaggtcgaccttatggaacatggtataaaaatagtaaaggtcaattgtacaccttcaaagatggatcaaaaaattcagtttatctgtttgatgagttctcgcatctatactatggtcgtggctatgttcagctcacgtggcaatccaattacgagaaagcttctaaaaaactaggacatgactttttgtcaaatccagacgatgttatgaaaaaagaatatgcaatccaaattcttttaactggcatgaaggaaggttggtttactggtaaaaagctatctgattacatctatcaatctaaaaaggattatgttggtgcaagacgaatcattaatggatcggataaagcgcaaaagattgctgaaatcgctcttatttttgagcgtgctttgcgtagtttgtagtggctgcactgcccattcaatcaaaaataacattcacgttgtggtgtgcgttcaatgcgtgaattaagaaagccccatttcggggcttgtctttatttattgattagtctaattgctgtagcgccaaattcatcacaagccgcattccatccgcgcttgtaaccttcctcaaaaccatttttataaaaagcatagaaacaacgccaatgcttatctatattttcatcctcaaacttataatgattcttttcacaagtttgataattcatatttgaagcgtcaatacctagaaatcctagtgattttagaaatttctcaagcattataatttccttcaatttctgaaattacttgtgcataaacagggtcggattccttgataaatacaccatttatcataatcccctttctgtgttttatatcgtcatatgcgagttgcacgcattcttctaatgttgtttcagatagctcagcgattcgtttgagacacacaacacaagaataaacttcatccgaaatgtcataagtatctaaattttgagccaatatgcttaaatcacgaagaagcataacaactaaatcctttaaattacaaacacattctggcttttgatcctcagcccaatcaataaatggcaagtagctttcattgcatactttctccattagtccatctttaaattgactagaaataatagtaagcacaataaagatatcgccgcaatcgtctttaatgtcgcgccctttacccacattatcagcaagctctccaaattcgctaaacagcttcatagcttgatcaattggctttgaacctttaataatattacgatctgacgcccattgttcgatttgtttaattaattgttccatgtttacttccaccattgcttttcacgaataggattaaatttacgttgatcatcttgctttctttgcacgtatcgtttccgcttatatgcctcatataaatataagacaaccacaaaaacaaaacttaatccaaaggataataaaatcattgctattgggttcattttggcctttcctctagtgagtctttccagtcacctttaaaatctgacaatttgtcattatgagtgtaatcgtgataaccatttatggtgccatcaatccacattccagaattaagacagatgattggtttttctgtaaattccacttgaaccatatctttatctgtagctacataattggcttcttcagaaactttcgaccaatcatatttactcatttctttaactccaaatatcgttgccctctatttcgcatcacctctaacgcgccttcacgttttaaattcttgataaattccactccatatttaagccattgcgttaaaaacaattcatatcgttccttgcaatagtcgttaatttcataataatcggcatctgaaaaataaatttcagccacctttccgtttttacgcataacaatattgcctttacgctcatttaagttatgcccacctttttgcatccaaaaaacaaacacaaagactaatgattccttgattttcatttatcctccaaaaaccaaagtcttggcatattctctagccatttctacttttagtttaattaagtcaattttcttttgatcatattcaactctatacgatgaaattcgatcattaggattcatttccaaaacatagtcgatatgcaaatattcatcatcatgtcgagttaggcactctttaggtgttggcattaggataaaatcaagataagcctcattaattattttctgctcgtaatcgtgattctctcttaacaaccacatataccctaattgctgccaatcataaccagcatctaaagctttgctctcaatatcctctttaaaatactccatagtccaatatgaccacgggcattttgtgtcacgtattgctgtgttagtaataatgtctggttcgccagtgatccagtcattagtaaaacgaatcatattcttttcagcactaataaacttctgttgcattagaaacaagattgcgtcatcttcaactaaattaccttttcttgtttctttacttccagtaaacttacttggagctttatgtcttaactgcctaactttttccttgaccagtgtattagcagtagccgataaagttctatccaaaagatcatctaaaatcttttgttcttcatcggtacgttttttcttcgcctttattgcttgaacttcttctgtgagaaatgcagagtcaatagactttgcatttcccattagtcgatgaagctcagaacatcgaaaaattagattcataacgcctcaactattttcttttgttcttcgcttaatgcgtaaatatctggatttaaagcatgctctttagttaagctaccagcttcaatagccgccacgaattgcgggaattgatcctcagaaatcaatcgagctaatggcttttcgctttcttgatcattatcaacatatgaagcttcgccatcttcattaatcacagcttgatcaaattcaatcgcctgctgaagctcaaccgacataggcgcatattttgagataagcaacttaataacagtcttttgacacattgcgtcccaattctgatgccatacagaatagctttgccctttcgactttgcagtcttatacgtctggctatacttgcttgcatgctgctcaagctcttcattggtcattgttaaatgagcttcaaaccctgtcacagtctcaagataagccagatacccaataacttcgccactaactttttgtggaatgaatgaagttaaacgagcctttacgctttcttctgtatcggtgtcataaactgcaattgaggcaatctttttaatctgaccagagcgctgtgctaactgcaagtaacctttccaccccatctgaaattgtgcttcttgattgccagtttgtcgatttttaaacggaacaatataagcaaaaccaagattgttatttaatggcaaattaagcgcacaagccgtataaacagcacctataaccgattcgggagttgattgcgccaataacccgtttgaattaataacctgcattacagacgtaatatagctatcagatttacgccctatcatttcctcaatacgcttcatcaccgatgggttgctaagcgtttgtttaatagctaatgcattttgtttttgctgctgagttagattgctcatttagaaatctccaataatttaagtaatgtttcctgtctttcttgatgctgaatatctctatattcatgcatacgctttctgtattctttagagtcaatcacattcatttgataagctgtttcgattgaccagaagtaagcgtctgcaccatgttctttcacatcgtcaagccaactcattacattttctccgcaatttcattttcaataacttgaacaatgtttgatacttcttcgctaggcaaaacatagttttctgtttcaccatcttcattgtaaactttaacgtcatttacagattcaacctcaacatcattccatgattggaatccgttgccatctttaaagaatctaccttcaaactcaacttctaaaactaaatcaccagatttaatggcagctacgttatgttccatatctaatgattcaacttcataaggaccagtaataactggtttacctgaacatgcagctaacgcaacagaagcagctaaaatcaatgcatttttcataatgtttacccattgtttatattctcaatagtaaatgaattgagtattaatgtcaagcgcaaatattaaaaaagctcccgaaggagctaattttaattcattaagtttaacaacattcgccttccaagataaggcgataatagcgactcaaactcgccagatcttaattcaaactcttcatcatttattttaactccataagcagcaccaactttacaaaattcaatttcttgtccagttcttggggatattgagatgcttgaaacaccgccaaccttttgagtagttagtaaaaactcatcttgtgaaaaagaattatcaatgatttttgccaactctaatatattcatttcattccacactcactacagtaattatttaaaaacaacttgtacttacaacaaatcttacaaaactcaaccaccaatagaacccttcaagaagaatagcgccgttaaaattgcaataacaattaaaataatgattcgctcgcgcttgatcttcgatttaaggctatttacatcctcttcagattgcttgtattgattaacaaagtaatccaattcaacttttgttcgatccaaatcaatttttgcaccaataagatttccttctacctcgaccagtgtattaatatccttatcatgaattttcttagcttcatcgttttgtttgattagtccactgattgccaagtctttactttcgatgatctttttgagatccgaaatttctttgtctttctcgccatcttggtgtttttgaactgaaagatcatttatagatactggaatatttaattcagaagcgcatcgtgcatactctttttccttgcagcgctgctcaaattccaaaagttcgtcaatttctttaataactttattctgaaccttctcgctttgcggctggttaagcatcttagcgatataggggcgagatttacccatctttaaagatagttcactattatttaaaccaagttcttttttggcttggtggatttttttgatcatatcgttttctgaatatctttgcataaattcatcaaaactaattttgcgtccaacagaaaaatctgaaaaccaaaatgtttgaccaaaatcaacggtaatattctgatctccagttggacacatatcatttccgagtccatgaaattttaataatttttccgcttcatctctttggtttaaatctttacacacaatagcgatattttcgattttcatctttgtttctcctgttaagatattccctatagtaaactattatttaatatttgcaatagctaatttaataatttaaactaacaaaaaatggagggtcttatggactggaaacaaattattgaagaagttttagaataccgtgaaatcaatcaagctgaattagcaagaaagacggggatttcaactgttcacattcacggtctagcaactggtaaacgactacatccaagctttgaaaagggtttggctatagtcaagcttcaccccgatacaaaaaaacttttaggaatttaacccgctagtcgggttttatttaacctgtactatttactattggatgattgagttaatcatccaatttaaccatataatttaaaagagccgtataagcagaatcgcttaatagatctctatacttatttgtcatgaatataatatgagattccttttcttttttgtatgcaataaaggcatctactggattattaaaataaccaatacaaaacaactttgcttcttttttaatttttgcaacaaatcttcctgttgcattatgcaaagaaactccgattgggtactcccctcgcttggctttatttttgtttaaaaggtaattaatttcttttggcaaaagagtgcatgtgtcttctgagtaaattttattgccttttattagcaaatctttgtcaagttcaaaattaagattgccatctaggttggtttggttgttataccaattataaaaatacgaataattcttgaaattttctgaaacagtgcatccaatataagttgggtatttctttttatattcttctgaatagcatcgaaaaatcatatttttccaaacgacatattcttttattaactttccatcaaccatggagggttttgatttgtcgttaatcccaacgccataaactagtttagccataattcaatcctcattgaaaacctgatgtaatgatgcggaggccaatcaggttaattggttttcgagccgtcactctatccgcaaattcattctaacacaaaaagagctaactactccaacctatttactattgagaatatatttgataatataaacaccactagatattgttaatatttaatgtaggtaaaaaatgaaccatgtaattaatgacggatttatgaaccctgaccgctttggatatgcagcaatacctttcaaaattagcgataaagagcctagaaaggatataagcccaaatgtaattgcaaggtcaaatgcaagaagtatgggtaaacgatctatgagcataccaagccttgcaaatgcggatcattgctaagacgagtttataacaatgaatgttttgattgctggaaattaaaaaaagaaaaataaccactaagcccttcggggctttttattttacctttatatcagtttttcttattttgtatagttgaattatttattttacttaactttaaaagacctataagatgaccatatagtttaggagagcgaaatggttacagaaatttatacagcaatcacaacttcgattagcgaattaaaaaagaatccagtcaaggcagcagagcatgatgttgtttgtgtcctaaaccgatgcaagccagcattctacactgtttcacctgataaaatggcagaattgctaaaatctggaaaggaaaaaaatgatctttttggcgatattgccacagcttatgactgtgcaatatcagcacttgatatggttaacaaggagttacaacctgagctattccatatgatagagcaaattattatttgctgcattggtgaagatgagtttaataaaattaagaaatcatctttttaatcaggagacaactgatgaatgaaatatcaaaagacgctttattctttatgacttggtttgaaaaagaataccccgaatttgtcaatcaatttggtgaagtaaagaacttttacgaccatgaaaaagatgaattcatgattgaagaaattcaagacgcttacattgatcgaaagaaaggcaatgagcctatgccaatgtatttcggtgttagtggtcagtttagataggtttgtaaaattatggaaaatacattgcttaacaatgatttattgatagaaaatcacccaaatatgattcgtaagtctaaaactttgaatgatatgccttttgatgttgcttttaaatacttccctagtggctacaaattcactaaagagttaagaaaggaactttggcgctctcatgacaaaaaatgttgttattgtggggttgaaattgaaacatatatggaaatgcatattgatcatttcataccaaagtcaaagataattaacgaaagtattgagaatcttgtttgttcttgttcaacatgcaacttaacaaaacacagtagagatttagaggattttaggttctgcatggctgttaagaattcaatactcaatggtgttgttgatgctcacattgtaaaaaaactagtgagtattggtgttgaaatgccgattaagttaaacaaattttacttcgaaattgttttgagtggtgataacaatgtatagcagtggaaatgaaacagtagacaaaatagggttgatgaatattgaagggaatgtaattccttttaattggttctcagtttttaagtttaaaaatggaaagcccgacctgaatgctatcgtaattttatccgagattgtttactggtatagaccaaaaattgtacgtgatgaagatacaggtgcaacgattggagttagaaaaaagtttaaggcagatttattgcagcgatcttatgagagctttgcaaaacaatttggattaactaaaagacaggttaaagaggcatttgatcgactttgtgagtttggtgttgtaattcgagagtttagaaccgtaaaaactgacaactcaacactatttaatgttctttttattggtcttgatgcagacattgtatcatccgtttcaatgtaccaccctcctacgttagaaagtaccaccctcctacgttctaacgtaccaccccatacgttgaaacgtaatacatatacagagactactacagagaatactacagataaaaaacaaagtagtttttcagaaaaatttgagaagttttggaatgaatatccaaaatgtaagcggaaaggaactaaagaagcagctaataaaacttttactaaataccaaaaagattttgaaatgatcatgaaggttttagaggagtttaagaaagatgaaatgtggacaaagaacaatggtgagtttatagcagcacctagttcgtggttaaacaagcaatactggaaaaccgactattggattgagcaagttagcaataatagtgctgataaagttcaagcccaagaaaatgtagtaagaagagctgtagctgtaccaatcaattatttggattaaataaaatgaatgaaaacttgtactcaatacaaattgaacaatcggtattatccgcccttatgtctctcaatggtggtattgatgatgttgtaagcaaattaactacggatagtttttatgcaacacaacataaaattatctttaagcactttaaaaagctttttgatgctgggtcaggtcatgacattgttatggtttatgactcaatcaagttaaatgcaaatgactcgaaaattgttgatgaagatttcttgataaatttaaactcaacaataggtctggctcattttcttgagcaacatgctgaccagttaaatgaatatgcctccagaagagcgctgtttgaggcaggagaaagaattaaggctatttcgatagatactacccaatacgacattaatgaggctatatcgaaatctgagagcattctggagaatttaagcaatcaggatgaagtgccgactttatcggatgcttatgatgtttctgtttctctatttgcgtctattgataaaacgatggaggcaagaaaacgtggagagaaggttgacgtaggtgtaaaaactggatttatggacttggatagacagcttggtcaaatatcaaaaagtgatttagttattattgctgccagaccatcaatggggaaaaccgcatttgctcaaagcttgatgttgagtgtttcttttatgcaacagcatccagttttattccaatctgctgaaatgtcgaaagaaaaaattgggcaaagattggttgctagtctagcctcaataaatttaagagatattcgtgactctgatattaaaaatgaggattgggagtttttctataaagcaactaataagttgaaggcatcaaaacttttaattgatgatagggcaagacctagcttgtcagatattagaaaaaattgccgaatcatgaaagcaaaatatggttatgttggtgcagtattcgttgactatttaacattacttaaatcaccattgtctactgacaacaatcacttggcagttggtgcaatatcaaaaggtcttaaggctatagcaaaagaatttgattgtcctgttttttgtttagcgcaattaagccgaagcttagaatctagaaaagatagacgacctctaatgtctgacattcgtgagtcgggatctattgaagaggacgcagatgtgatcatgtttatatatcgtgatgagtattatgataagaactcaaaagatcagggtattgccgaaattattgtagccaaagcgcgtgatggtgaggttggaacagttagattagcaactgaactacaatactcaagatttagtaatcttaatttagaatatttggacatgtgaggatttttaaatgaatttaattgataatttgggcgggtataatgtagcaaaaagcttaaaaatgtcgcaaatagggttaaaaaagcaccatgatgaattaaaagcagctttactcgaataccgccgacaaaatgaaatatatgaattaggcgaccttgttgtatttaaggaggaatatagcaaggacagtgttgttcataaaattgatagtttacgtgcaggcactaagtgtttgcgtcacgcaacggatgaagagatagagaaggggtgtagagtatgaataatatactaaattgttggtttagaaagtcagatgatagattttacaaattccataaaatgatgtgcgaatcaattattaatcaaacatgtttttatgattcggaatataagacaagggttgttctagtaagttcgagctgttgggatggtgattttatgacttcgaattatgaatttgttgctgttggtaaggttggcttgcgtcagcgttactatccaaacaaggttgtcttaaatatatttgacgcgagatttagaattaagccaattgtgagggttgagaaatgaaatacaaagtcggtgatcgggtttattttgctggtgaaaaatttactgtctatgaggctcattcatggggagtaagaattaaaaaccagtttaaaaaatggagtgtgacaaatcggagtgtaaaacccatagtcacatgcaaataatttaaaaaatagttgcaattattgtggatagtgtttactattgagaatataaattaacagatggttttattatggaattggttttagttgtttcagtaattatgtatttcgcgcctagcattattggctttatgcgtgggcatgcaagtaaatgggcaatctttgcaatgaatttgtttttaggttggtctgtgttgttctggttttggagtttgttctggtcattgtcaaataaaggcggtagccaaacagtgatcgtaaataaccagattaacaaccaatgagcaaagagtttataggatttgcaaaagtcatggttttgttttatttgatcgtgatttttgcagtaatggggttaagtaagttaattcataatatggtatggggtgtgtgatggaaaattataaaattcgcgtaaacaatgaagctgagagcaaagaggctcaggagttgtttatacagcttggctatgaactagatttgctttttggcaaatacgaagcaaacactaaatgggttttagctttcgccgatggatcaatgggttgtgcttgtgaagctttggataaagacacattaaaggaaattacatttcctcagcttcgagaccttgttgtgttgaagcgtaatgatgtgaaggatgcaaattacataagtaaaagcgtcttagatggattctactttaaaagctgtgatggagttttttactttatgtttgagggtaagtgggtgagatcaacaactaatacagatgaagggttggagcctatcactaaaggcttagatttgattagcggcgcggaggcgttgcgagctttggctgatggcaaagaggctgaagggttttcagaagaaaatgaagagtgggtacctattgtttattttactgtacaagaggttgtgaatggtttatataaattccgcatcaaaccacaaaccgtcaagcttgaacttgagctgccgaagccttttgagccgaaggtgggtgaggaatatttctatatttctcatacaaattctggttatgactctgacatttacgaggggacaaatacagatgaatgtcgaactcaaatggggtgttggcgcaccaaagcagaagcaaagcaagtcgtagagcagctaagaaaaatcaaaggtgctgtatgaaaaagcggaataaaaaatacaacgggaagcaggttgtaaagcagaagattcacaagtttcaaatgacttgggaggttaatgaggctaaaagtattattgagcttcatcatttgcttaatggtgtagatccacaagagtctactcatacaccgcttaaagtctggatgaaggcgcataagggtgatttggctttagcattaaagacgcagacgataccggcagagcaaagttttcatatcgttagtcgaattcatgcggttaatgagaaaacaggcgaaacggttgattgtgagtttcaattggctactgatactgttatgcatttgtggcagttcttgggtgatgttgaatctgacatttatgtcaatgatggtggttttaaaaagaaatggcttggttttaatcatgagcttgaagcttatttgaaagaagtgggtaacggtgaatttgtggttaagactaatcattgttgcttaacatgcttttcaacattcaaaagctttagacatgaaatggaatttaaatcaattaagctaatgaatcctgaatttggattaggagttgaagggtgaatattaaaaagttacgcgacaagattaatggctctgagcctttagctgatgatgaaacgaaagaaatgctaatagagcaatggaagaaaattcacattgagctagaagctaagaaagaagatgacgaaagaaactatgttttatgtgaggatgagaaatgaaagagtggttattagaaactgaagatggcaagtatcattatcatcgtgataatatgtattgttgcccaaaatatattaataaaattgaaattccagagggtgctgaaattgccataaaatttaatatagatatgtttaatcatgggatttgtttttataaaaacgcaggtagattagcatggagtcaaaaagatccttactggaaatatagcgaatggagtaaagacaatctttttgatgagtcatttaagcgtggaaacatcgatcgttctcataaagttttatggcaaagacaagaagtttttattgatgaatctggaattgttaatgaggaaatgaaatcattaaacaataaaaaacactctcattactttaaagatgtatctaatttgcttgagcttgatgtttatcgtgtgttgaaattgtttgatgttacggatccatgtattcaacatgcagttaagaaattgctttgcgctggtgggcgcggggttaaagatgtggataaagatgttcgcgaggcaattgactctttattgagatatgaagaaatgagaaaggaagatgagaatgaataaattagaattggcgcatgaatattcaaaagttcttttaacaaaatttgagacaataacagttgatcagattgttgatgtttcttttagtcttgcagaattgatgcttgctgaagatgaaaagcggaaagataaaagtcgacctgaagtgttagaagaatttgagattgattggaggatggttcctattgattataaatggttttgtgtagacagtttggggtttgggcttgtctttaaagaaaaaccagatatattttttaatacatccggcattggcttatggggaattaatgatgcatgtagtggtgatatgatggttgtcaggcataaatataatggcgattggaaagactcattgagaaaacgtccatgaaaaaattacatttacatgcagttcgtggggttgaagcctttgagcctaatcgactaatcaaacaattttttataattgaggcaagtgaggacagattaacttttgatgatgttttaaaatttaatgaagattggtctaatgagtttgaatttggatttagttattatgttggatatatgactattgaagaatttgaggaaaagtacaaaactaaaggaccattttatataggaactgatgtgcgtgagagcagctaagatagatgcaaatcaaccagaaatagtggcagcacttagaaagattgggtgtacagttcaaattctttcaagtgttggaaaagggtgtcccgacattttggtgggctatcgcggtaaaaactttttattagagataaaagatggagctaaaccagtttcagcgcaaaaattaacgccagatcaaatcgaatggcatgacttatggaatggtcaagttaatgtggttaattgtgtagagcaagcaattaaaattgttacttgcaattaactaaaaatttgctatatttatcgaactgaagaaacttttacatccgtatctaaccccaagatacggattttttttgcttaaaatccttgcattaattataaatataatttactattgagaatataaagaggagagataaaatggttatagttaaagctttatggttggcaattgttgatgtagctaagttattcaaacatgcacctaaattgttggttgagttgttttgggctttagctggtgttttatctctaatcttgagtgtggttttatttccattgattgtggtgagtaaatacaaggtattgaaaaatggatcacaaaaaagagttaagggtaaatctaagggtattaaagtacgcggcaacagatttgcttagaggttcagtgttgattgtatatcacgctggtatggttgtatttaattggttaaggggtaagtgatggattttatggaagctattaaaaaaggtcttgaagcttcaaaaaattacgatagaaatcttgatcagattattgaagtgattattgaagcaaacaaagctatatgcgagaaaactggtgtactgggtggatttattttagttaaaagcaatagaagtttatcttgtacacaagcagttatgtcaatagaaattgataagcaacatgcatttccaataaaactaaacacgataagtggaaattatatcgctaatgatatttgggatcttaaagaagttgtgcataaaattttatcccatccaaattttgggttctatgttcgcagacttatggagaataaaaaatgatctataaatttctaaagcgcctattctgccgacatgaatgggaatacgaggaaagtaatttaactggcgagacttacaaggtttgccgtaaatgctggaaggagattgattgtgagtgaatttaatgcaatctatcgaaaggtgtgtctgggtgatagatttgatataagcgcatataagcaatgcgagaaaatttacaatcaccagcaacagaagattgatgatattgcgggatacatcaagacgttaaaggctttacataaagaaggtaatttaacagtgggtgatgtgaagatgtttttaaatggtgtggaagggattttgtgatgaaaaaattattaattgcgttattgacagttccaagttttgctaatgctgggttttgtgatgctgtatatgactttgcagaggctactatgctgatgcgtcaagaaggttatgcaaagcatcaagctaaagaagtggttgaggagatgcggggtgtagacaatatgctggctagtttaatggatgccaatgttgattgggcttactcattccctgtttacaaaggtgaggataaagcggttattgcatcaaactttgctaatgagtcataccagatttgtaaggatgaagaaaatcgtgcggctaatgttaaggagttttactagcaatgaaaacaaccgtagatcaagaaattgacgaagctatagccaaaggtgaaagcttttataagattagaaaacgtttgaaaaagctattttggaaagggctttggttaagacgagaggtaatcaaactgaggcggctaaaatgcttgggattagtcgctcggggttgggtgggattttaaagagggtgggtagatgaaaattgaagaaatcagagccaatgctccgaaaggtgcgacacattatattgagtatgcaggggtaacttattttaaaagcactgaatatgggctgcatatttattcaaaaatgtttggagagtggatacttgaccgaagatttagaattaccgaacttaaaccgctatgaggattgcattatggatattgaagaaattaggaagagcaagccaactaagaaagctacgcactattgtgtagatgaaggaaagacaatctacttaaagaagaataaaaactatggctgggattattgggcgcatagatgggtttattgctgtaattggttcaaggatattaaaccgctttaacgcggtttttctttttgtggtattattaaattttagtttttgggaaataaaatggcatcttccacttctataaaaaaggtcaagacatccgacctaatcccatacgctaacaattcgcgattgcacagcgacgagcaggtgttgcagatagcgtccagcattaaagaatttggttttttaaacccgatcatcattgacggcgataacggtatcattgcagggcatggtcgcgttatggctgcaaagaagctaggtattgaggaattaccttgcgttgatgcttcacacttaaccgaagcgcaaaagaaagcgtatattattgcagacaacaaactggctctcaattccgagtgggactttgatgcactgcgtattgaatttgaagcattgcgagaaatggattttgatgttgagctaacaggcttttcacttgatgaaatcgaagagcttgagattgatgaaatcgcgcctgaatatgaagaagatgcagatggcgaagtgatcgagccgccagcagaaccaaaaacaaaagaaggtgatgtttggattttgggcaagcaccgtttgatgtgcggggatagtacgagcattgatgcggtggagaggttaatgcaaggcgagaaagctgatcttgtttttactgatcctccttatggtgtttcatattcaaacggaatgaacgataaatttgatgttattaaaaatgatgatgtaatacttgatattgctccagttatttattcatttatgaaggatgacactgcagcttttatttggacttcacaccacgtttatccacaatggagagagcaatttgagtcgttctataaacaaacattgatatggtataaacatggtggcggaattggtgaccttgaaggaagttatgctttagattatgagattgctctgttttgtgtgaaaggcagacctaggtttaatggtaagcgtggtatggcagtatgggatgttaaaaaggatggatcaagctcatacgttcacccaacccaaaaaccaactgagctatctgagagagggattaatgatttttgtaaacaaaatggtcgcgttttagacctattcggcggcagtggatcaacattaatcggatgcgaaaaaaccaaccgcaaagcctatcttatggaactcgacccgaaatactgtgatgttattattaaccgttggcagacattgacaggcaaagatgctgtacttgaaagcacgggagagaagtttaatgactaaaccacatgaaccaaccgaagcacaaaaagccgaagtacgcgcgcttgcatcaatgggcatcccgcatgatcagatttctgcatatatcggcattgataaaaaaacgctatataagtattaccgagaaattttagaccgcgcgaaaattcaggccaacatgaaaatggcgcaatgcttatttcagcaggcgcagaacggtagcacacctgctgctattttttggttaaaagtccaagcaggatggcaagaaaagcaggctgtggatgtcaccacgaatggcgaatccgtgaactataaaccaaccatcattgagctagtagcgcctaatgtcgaaaacacagatacagataccgcctaagttaatccctgtattttcagcgcaaggtgttcgctaccgttcctcgtggggtggtcgcggatcagcaaaaacaaggacgttcgccctaatgaccgcaattaagggttatatgttttccgagatgggtgttggtgggactatcctctgcggtcgtgaatatatgaatactcttgctgattcttcaatggaggaggttaggcaggcgattaaatccgtcccctttttagacgattattacgacatgggtgaaaactatattataaccaagaataaaaatatttactattctttctgtggcttgcgtcataacttagatagcatcaaatctaaagcgcgtattttgcttagttgggtggatgaggctgaaacggttagcgagatggcttggcgtaagctattaccaactgtacgtgaagaaattgtattacctaatggcaagattgtttattctgaagtgtgggtaacgtggaaccctgaaagacgagatagcccaacctcaacacggtttagacatgaagaaatacgggatgatgatggcgttctaatcggtattggtgctgagatgaactattctgataatccgtggttccctcatgttctcgacttggaacgtaaacgagatcaagcaaatcttgacgatgtaacttatcgctggatatgggaaggcgcttatcttgaaatgtctgaggcacaaatcttcaagggtaaatacgagaaaaaggattttacgccagaccctcaaaagtggcatggtccttatattggattggactttggttttgctcaagatccaacggcatgtgtcaaagtctggattcatgatgattctttgtggattgaacacgaaggcgggaaagttggtcttgagctagacgatactgttgaatttttagagaagaaaataccagacattaagaagtatgctgtttatgcggatagcgctcgtcctgaatctattagtcatttaaaaaagaaaggtttgcgtagaattgcgcctgttgagaagggcaaaggttctgttgaggatggtattgaattcatcaaatcgtttaagaaagtctacattcattcacgatgcaaagaaacgctaaaagagttccgtgattactcgtacaaaaaagacaggctgacagatgaggtattacctattatcattgacaaagataaccactatgtcgatgcattgcgctatgcacttgaaaaagtcatgaaacgtggcatgggtatgaagattaacatcgccgatattgaatcggctttcggaaggtaaaacatgtttgattggttcagaaagaaagaagaagcgccaaagcgaaagccgaaatggaacgctttgttaaatgctatgcaagcgcataatgaaggtgtggcgattcagtacaaagcgccttctttgcctgatggtgttgcaccagatggtcatagcgctatggcaatggatgggttttgtacagcttcgcaatatgctggattagagccacagttttatagcaactttttaggctatcaagtattagctcaattggctcagtctactgaatatcgcctagttgctgaaacatttgctcaagagatgacacgcgaatggggtgaggtcaagggtgatgaccagaagcgcgtagacattctcatggaagaatttaacaggctggacattcgcaaccttgtccgtaagcacatcgagaatgattactactatggtggctctcagttatacattcagatcgaagggcaagaggataagaccgatctgcctctattgattaacgagaaaggcattaagaaaggctcgttaaaaggcttcacggttattgagccgctttggtccacgcctagtatgtacaatgcaaataatccactagaaagtgatttctttgtgcctaagcagtggtgggttatgggtaaaaatgtacatcatagccgattgcttacattaattatgcgtcccgttgggcaaatgcttaagccagcttataatttttatggcatgtcaatgtcgcaattgatgttgccatacgttcagcgtcatcaatctattgtggacgctgtagctaaattgattaccatgtttagcttgactggtatcaagaccgatatgtctgcaatcttagcgggtgatgaaggcggcgcaaatcagttgattggtcgcttaaaagcattggctttaggtcgagataatcaaggcgtggtggctttggatggaaaatctgaagagttcttccagatcaatacgccattatctgggcttgatacattgctagacaagtttacacagatgcttgcctacccttcaaaaattccagtattgaagatatttggaaccccaacggcgggtcttggtaatacttctgatggtgagattagggtattttacgattgcgtatcagcacagcaagaagcgtatattttgccgcagatcaaagttatccttgactgtatgcaattgagcttgtttggaaatattgatgaaagtattaagtttgtctttaatccgctttatcaattggacgataatgaacaagcagacgtaaacttgaagaaagcacaaactgctcagatttacattcaagaaggtgtgatcgacaacgaagaagcgcgtcaagctttgaatgatgatgaagacagtggttatcaattagaaggtagtgcgccagagcgtgatccttatgctgaaaacgaaaacatagatgttgttgaaaaatagatatggaaataacccttgagtcaatagcgcctaatgcatccctgaccaaatggtacagggagcaaatgcaaggcatgatggatgaaatgcgttctgatttaattcaggacgtagttaagcctatgcggtctgaaattgctatggatggcattttggattggatgggacatgttattgatgggttggttagtcggtggcaagatcgtttagataaactatcgactcaagtggcgcaagagttagtaggtaaggctaaaaccaactacgacaagcgacttttgggtattcttcgcaagcgtggctttactgtaaacttccgaccaacgaaatatatggaagatcaagcgcaaattgctttaggtgaaaacgttgctctaatcaagtctattggtaatgagtatttagataaagttcgctctgcggtttggcgtagtgttaagaatggctatgatgttgagtcactaatcaagcaactcaaagagattgacggcgtaacagatcgcagggcaaagaatattgcaaaagatcagacagctaagttgaatcaggcttttgagaatgctagggctgaggaattgggcataacggaagcttactggcttcacagtcatgcaggaaagacttttcgtcaaagccacgtaaaggcaaatggaactagatttaacatcaaggaaggcttatttcttgatggcaagtggacaaacactggcatcgaaatcaattgtcgctgtaggaaaaagctaattatcgaaattccagaaagtatggcataatacagttaattggatgtgataagcataagtgaaagcgcacatcgacacggcaatccgtttcactaactgtaaatttgtaaaaccaataacccatatcaaaacaactgatttgtatttttagattgttttggtattatttaaatcatggagtatgactacaatatcatgtagcattggtaaaaattactgcgcgccttggttggtttgtaatttttgcggatgtgtgaagtcgcttctgtaattgatcgaatctgtgggcgacacagtgaatatcattagactaggagtaccttcaaagggacatagcaaagcaactattcgcacgttgccgatcaataggcaagacagatggctaaaatgttctgttgttttcggtattggcacgagaaaccgatgaccgcctgaaagtaggcaacttagagaagtgaatagcttaggtgctagaatattgcagttcacattataagattgaattaattactcaattggtttttgtggcggtgggattgatcaaccattgtaaaatactgttttattggcgagttcgactctcccatgttcacttcatctaagtttatttagaaccatcctaacgggtggttttattttgtctaaactattttagatttactattgagaattatatttaatagtggtaagatttagttattcagcagaaggggtttgaaatgattaaagcagaagtggtttttgatggtgaatctatttttgttaatggaagaacaataaaaatagatcacttagattggctgtatcatgtgtatgagttagaaaatcatgttgatatgtttcattcattagaacaagcaatagaatattgcatggagaattaaatgagcaaggcaaaattagaatggcttaaagcgaacatgccagagggatgttatcattatgaagaaagcaaagtttatgtgacttttatctttcctgatgaaccacataaaaacctaaaggtaaaaaaaccagaatggatgatttaatatgattaaatgtcaattttgtggaagagatattaaaaatcatgcaatttactgccctttgatgtattggagtatttttaaatgaaaccagagcagtttattcgtgagcaaggattggataaggcgcgagaggttgttgaaggcatcccaagcaaatatatggagtgttactactcaacattatgctactgcaccaaagcaaaaaagtattcagatcgttttaatccaagaattgaacttgtgaacatggcggatctcaaacgcttggtggagtcgattgatctgatcaagtggcatggtggtactaagtttgccaaagactacctagcgcggaataaagcaaagcatccaaatgtaagcggctgggatgaattggagcaggcaatcaaagaccacgaatcaatatacggaggcggtgaatctcatgccaactagatataacacaggcgagtatagctacaatcttgaatatcactatggagatatgtcagcaagcatggagatgcttagagcacgtttaattgaattgttgactcctcatctgtctggccgttatgtgaaatggagagaagcatatttcacatggtttacaaagtgcggcggggattcggggtggatgttttgtgtaggtccacacgaatttcatattgatggggcgttaaggcgctattactcaggttctattgatattacctacaaccagaaagatcgatatttcttggtgggtgagaaaaagaaagtcaaatgtaaggcttgtaaggggtttggcttcattcgagatgatgggtgggggcatatagataaatgtgaaatgtgtgatgcagaaaaaggagccagccatgagtgagtttaaagagtttgaactaaagtttgatgaatggtgggaagaaaactatatgaatgtttcagcggacaggactgaagcatttgatatttattcattgggattaaagcaccagcaagcgaaagtggaggagcttaaagcatctcatcacggtgaagtgattggtcatgaagtttactttaaaaagatcaagcaagagcgtgacgagctgcaaacattatacactcagcaaggcataaatatgttgaagatgcaaaagagggtggatttagctttagaaataacaaaagagcttagatcattaaataacgaatcattagaaacttatagtgaatctattgaagaaacgatttgtgatatagagcaagcgctcaagggggaaggatgaaagcaatcaagatcccatgtgagcacgacttgctaagtaaggacgatgacatatgggttaatgctgtgatgcgctgtaagggtggaagcccttactgtggcgcagacggttattgccatgcaggcggcacttgctttgctgaccaagagcttacaagagagcaagcaatcttagaagtagatcgcctagctcaagaattacataactcaaagattgaaaacgacaagttaagaaatgcagctagtcagcttgtcagccaacttgaattgacaaaagagcagaacctaaagaacggaaatgatcaaagagtatttgctttgaagttctgcattcatgaaatcaagaaagcgatggagtgaccaatgaccacactcaaagaatgcaaccatatctaccaatattgctggatctataaagcttacttatgcatacattgcgacaagatgaggattgaagaatgattaagtcgatagatttagctagaaagaaaggatttcaccataaatttgattttgcaatgtcaagaatatggttgacaagggatagtcatgagaaggttggaaattatgtattaatccactttatgaataataaaaagatattcggtgttgattatatagtttgcgaaaatcaatacgaactaaatgaagcgaaatcagatatgctcaataaaggatttaaaattgattggtgtgtatttcaaaatgctttggagtttgtaaatgaacattgacgagattaagaggaatgcgcctgatggggcgacacataaagcaggaagaatttacataaaaaacttacgagataatacaccaaaggatagtgcttactattgcggatatatttatgcttatgatttttgggatggtaaacgatggcagggttgtgtttgcaagccgcgcgacttctttagaataaagtcactttaaccagtggcttttttattgcataataagaaaaattgtataggtgagtgatatgaatatcgcagatataaaggtaaaacttagctatgaaaaaacatggcaaaccaattttgttgtgaatttctgcaaattcatggttgtttttggtttttggtcagatgacagagcaatagaatatttaatcaaaagactaaaaattaaagtggaggatgtttgatgcctctaattaaaggtagctcaaaggatgttattcataagaacattcgtgagttaattgattcaggaaaaccgaaagatcaagcgattgctattgcatatcgtgaatctggaatggctaatgatactgactttgataagttggaagaattgtttgatcaatggcttgaagaggaaaagaaagagccagaacatgcaatggataaatcagctcgtagctacgaccgcaatggccatctaattgttgataaaaccattatcacaaaagccgcagtaaatccttatcttggttcgtcaattccacgatggaaggaattaggcttagatccaaacaaagaatacatgctgttgcgtgatcctgacgaattgcgtaaatcattagacacattcaagggcttgcagcttcttaaacgccatattcctgttgacgcatctcaaccagaaaaagagtcaactattggttcaattggtacagacataacaatggatgacgaaggtcgagtatggtcatcattgcgcgtatttgatcaagaagggattgactatatcgaaagcaaggcattaggagaattaagcgcaggttacgcttatgatgctgtcatgaagtcgggtacttttaatggtgtaccttatgatggtattatgacgaatattcatggtaatcacgttgctatcgttgaacgtggtaggattggaagtgacgcgattatcgcagattcaatagagggtcaattgatgacaatcaaacttaaaaacggaagccttgcaaaactgcaaaaacagttaggcatggattctttggaagatgttaaaaaaactattgtggccgttcacggttctttggctcttgatgaagatgacaagaaagccgaggacgaggacgacaaaaaggcagaagatgaagatgatgtagaaatcgttgaagattctgatgatgacaaagccaaagatgaagacgatggcgaaaaggcagataaggagcgtaaagcgttagaaaaaactgacaaagacgaccgagaagccaagaaagacgacaaaaaagaaattgctcaagatgctgctgaaattcgcggctctatcatgaacatctttaaggctggtcgcgaagttgagcctttagtgggtgttattgctcttgatggctttagctcagatcatgaagtttatgcctatgcgcttaaacagaaaggtgtagacactactggtattaatactgccggcttggctgctcttgttaagtcgcagaaggttacagaagctcctaaatctatggctatggattcatctttatatgatgctcctagcgacatttttgctcacattaaattaggttgaggtatatatgactttacaacaacaagttaacattaagatgggtactggtgttgctggtgattttgcatcgcaaaaccctcgccataccctattgtctggccgcgctcaattccgagcaggtgcttctggtgtggttattgctcgatttgccgaagctgaccccgatactggattggttactaatgtaattagtggaacaaagcctgttggttttgttagtcgttctggcaatattgcggttattactcagtggcttggtcaggcgtctatgacaattcctgctggaaaggaaattacgttgcacgataaaggcgacttctatatccagatcgctactcctacgaccgttggtcaaaatgtttttgcatctaatgcagatggctcaattgcggtttctagcgcagacacactagctgatcactacgcgactggatttaaagttgcgattggcgctgttgcaaatgacttagctactattactaaataaggtgatcagatgactacaaagaatcaaagctttaaacctcaagagcttgcgcgttacggcgttcactaccctgaaggcacgcctcgtatcgcaactcctcgcgacaaaatgattattgctcaagatactgcaatgcttacaacgcctaacgctggtgttttgcagatgtttacgcaatttattgatccagaagttacgcgcattttattctctccaaactcgttaacagaggctctacaagaagttcagaaaggcgactggacaacttctgttatgacattccctgttgttgagaatggtggtaagcccgttacctatggcgactggaatgataatggcacaactagcgcaaacgtaaactaccacaaccgccaatcttacaattaccaacagttcttgcgcgttggtgaaaaagaggcggctatttatggcgaagctaaacttagctgggctgctgaattagagttgggtattgttgaatcactaaacaagatgcaacatgaaatctatgcttttggtgtagctggtttgcaaaactatggtgttctgaacgacccgaacctattgccagcaattcctattccagatgcttggacgttcgagcttccattaaagattgttgccgacattcaaaaaatgtttaaacaattggctaagcaagcgaacggcttagttaagcgtactgacgaccttattttgttaatgtcaccagagcaagaagcgctcattacagcagctaacgattacggtcttaatgttgctgactacttgaaaaagagcttccctaatttaagcatttactcagttcctgaatattcgacagcttcaggtcaggttgttcagttggttcttcgcaagtatcaaggaagtgacacaattcaattggcttatgctgaaaaaatgcgtgttcatccaatgattcagaaggcatctggttggattcaaaagcgttgtcaaacaacttacggcgcgattattcgccgcccaatgtttgttgttacagcaacagttccaaacgaaactccctaatcaataacaagaaagtgctaccctagcggtagcattttttttaggttaagataacgttatcaaataagaggcttaaacaatggcaactgtaactatttcattacttcaaccttttcctattttgatggaaatggaaggaaaatcacaagttattaacgggtggaactcccctagttctatgtatattgatggtggctttagaaaagttggtttaacaagtggcgtagataaagaattgtgggatgcttggcgcgctcgatttgctaaccatgatcttctagtaaaccagttgatctatgcggatgaatcttcgtctaaagtcaaagccgaggcaaaagaaaaagccaaagttaaaactggacttgaagccctagatccagcagagctagacaaaaagggcaaacttgaggaaaactaatgtctaatgttttcgttttcgatcctgctgcatttaagcttgcatttccgcaattcaatagttattcaaacgagcaattaacttacttttttgaatctatcgaaggcttgcttaataatagcgaaaattcttgcattagcttgaagaatcgtaagttatggtattggctattggtggctcacaaagctgaattgcaaaacaggatcaatggcggaaacactggacttgtaggacgcatttcatcggctacagaaggctctatatctgttagtaccgactaccctatgggttcaggtgctatggagcaatggcttaaatctacgccgtatggtgctgagttctatgccaagactgctgcgtatcgtacagcaacttgggttgctgctactgctccgatgccagttaaacgcactaaaggaccttatccttttgggtggggcaactattaattaataggtgtatttatgaatattgataaagttattggatatttcaagcgtgagtttcctgaatgtgaaattgaaacttcttcacaaatttctggcagcgtagaaattcacaccgttaaagctggttgtttttttgctgaaattcaagtggaaggttctgctattgctgtattaaccagaaaagataagtgcttcacaggccttgaatatgctgaaaatcaagaagaccttgttgaatttgtattacatcaatttttaaaattctttgataagcatggttttggttggtaattatgaataaggatctcatctttaaagaaggctatggtcttagctcagaaggctgggatggttagcccatatcaaaaatattgatttcacatttaaaccctgtcgattgacggggtttttattatgcaaacgtattgcaaagttaaagcgaaacaaaaccgaatgggttatttttgggttatcagaaagactttagctaattgagttccgagtggatttgcgcgcattgtgttgtagatcagtccacatcgtccatcaaagataaatctaattgattcacctttgttcttaatggcaacaatgtctgcgcccttgcctattaaccaattttcaaagtcacgaagttgtcgagtttcaagattcaatttcggacttttttgttttttagccatgatttacccctaaatcttatttaagttaactaatactatatagatatatgctattggtgagattctgagagaaaaagttaatattcatcctaagacagtcttttttaaaaaaactatcttgaacattaact

SEQ ID NO: 14 - protein, 80 aa, Unknown; identical to SEQ ID NO: 3
  Bacteriophage YMC16/01/R2016_ABA_BP ORF8

SEQ ID NO: 15 - protein, 682 aa, Unknown; identical to SEQ ID NO: 2
  Bacteriophage YMC16/01/R2016_ABA_BP ORF9

SEQ ID NO: 16 - protein, 202 aa, Unknown; identical to SEQ ID NO: 4
  Bacteriophage YMC16/01/R2016_ABA_BP ORF21

SEQ ID NO: 17 - DNA, 243 bp, Unknown; identical to SEQ ID NO: 6
  Bacteriophage YMC16/01/R2016_ABA_BP ORF8

SEQ ID NO: 18 - DNA, 2049 bp, Unknown; identical to SEQ ID NO: 5
  Bacteriophage YMC16/01/R2016_ABA_BP ORF9

SEQ ID NO: 19 - DNA, 609 bp, Unknown; identical to SEQ ID NO: 7
  Bacteriophage YMC16/01/R2016_ABA_BP ORF21